(19) United States
(12) Patent Application Publication
(10) Pub. No.: US 2013/0078216 A1
(43) Pub. Date: Mar. 28, 2013

(54) LIVER TARGETING MOLECULES

(71) Applicants: Grainne Dunlevy, Cambridge (GB); Steven Holmes, Cambridge (GB); Zhi Hong, Durham, NC (US); Armin Sepp, Cambridge (GB); Adam Walker, Cambridge, PA (US)

(72) Inventors: Grainne Dunlevy, Cambridge (GB); Steven Holmes, Cambridge (GB); Zhi Hong, Durham, NC (US); Armin Sepp, Cambridge (GB); Adam Walker, Cambridge, PA (US)

(21) Appl. No.: 13/522,407

(22) PCT Filed: Jan. 13, 2011
(86) PCT No.: PCT/EP2011/050420

§ 371 Date: Jul. 16, 2012

Related U.S. Application Data
(60) Provisional application No. 61/294,942, filed on Jan. 14, 2010.

(51) Int. Cl. A61K 39/395; C07K 16/28

(52) U.S. Cl. CPC A61K 39/39541; C07K 16/28
USPC 424/85.4; 424/135.1; 530/387.3; 530/391.7; 536/23.53; 435/320.1; 435/69.6; 435/328; 435/252.33; 435/254.23

(57) ABSTRACT

The present invention relates to molecules that can be targeted to the liver. These liver targeting molecules (e.g.fusions and conjugates) comprise proteins, antibodies or antibody fragments such as immunoglobulin (antibody) single variable domains (dAbs) and also one or more additional molecules which it is desired to deliver to the liver such as interferons. The invention further relates to uses, formulations, compositions and devices comprising such liver targeting molecules. The invention also relates to immunoglobulin (antibody) single variable domains which bind to hepatocytes.

The present invention relates to molecules that can be targeted to the liver. These liver targeting molecules (e.g.fusions and conjugates) comprise proteins, antibodies or antibody fragments such as immunoglobulin (antibody) single variable domains (dAbs) and also one or more additional molecules which it is desired to deliver to the liver such as interferons. The invention further relates to uses, formulations, compositions and devices comprising such liver targeting molecules. The invention also relates to immunoglobulin (antibody) single variable domains which bind to hepatocytes.

### BACKGROUND OF THE INVENTION

Liver disease is a term describing a number of disease states including (but not limited to) the following:

1.) Hepatitis, an inflammation of the liver caused in many cases by viral infection;

2.) Cirrhosis, which involves fibroid deposition following tissue remodelling in the liver typically after viral infection or exposure to liver-toxic agents such as alcohol; and

3.) Liver cancer, including primary hepatocellular carcinoma (HCC) and secondary tumour formation following metastasis of tumours at extra-hepatic sites.

Chronic infection with hepatitis C virus (HCV) is one of the major causes of cirrhosis and HCC. Global burden of HCV related disease is high with endemic infection in many countries. According to WHO figures an estimated 170 million people (3% of the global population) are infected with an estimated 3-4 million new cases annually (reviewed, for example, by Soriano, Peters and Zeuzem. Clinical Infectious Diseases. 2009; 48:313-20). Approximately 70% of infected individuals develop chronic infection with 20% of this group progressing to cirrhosis within a 20 year period. Liver cirrhosis following HCV infection is also associated with increased risk of developing liver cancer and it is estimated that annually 3-4% of patients with HCV induced cirrhosis go on to develop HCC (reviewed, for example, by Webster et al. Lancet Infect Dis 2009; 9:108-17).

Current standard in HCV therapy consists of combination regimens of pegylated interferon-α (PEG-IFN-α) and the nucleoside analogue Ribavirin (RBV). The main aim of anti HCV therapy is to produce sustained virologic response (SVR) currently defined as failure to detect HCV RNA in peripheral blood, using highly sensitive PCR detection methods, 24 weeks after treatment ends. SVR is currently achievable in a large proportion of patients infected with HCV genotypes 2 and 3 using current standard therapy, however the proportion of patients infected with genotypes 1 and 4 achieving SVR is typically much lower (reviewed, for example, in Deutsch and Hadziyannis. Journal of Viral Hepatitis 2008; 15:2-11) due in part to compliance issues as a result of side effects associated with PEG-IFN-α treatment. Alternatives to IFN therapy are currently being developed and typically involve inhibition of viral targets (protease, polymerase and helicase proteins) with small molecule compounds.

However issues with viral resistance and side effects have hampered development and widespread use of these compounds in many cases. IFN therapy, on the other hand, is not associated with viral resistance therefore novel IFN-based therapeutics with better efficacy and tolerability profiles could represent an opportunity to significantly improve upon the current standard of HCV therapy.

IFN associated side effects are thought to be due in part to induction of IFN-responsive genes following systemic exposure to IFN-a (reviewed, for example, in Myint et al. Metab Brain Dis 2009; 24:55-(68). Since the primary site of HCV infection is in the liver (specifically hepatocytes) it could therefore be of potential benefit to avoid exposure of peripheral blood cells to IFN, thereby potentially reducing side effects associated with IFN therapy. IFN-α specifically targeted to the liver may also exhibit improved antiviral efficacy as a biproduct of directing the therapeutic molecule to the site of HCV infection, thus increasing concentrations at the hepatocyte,which could in turn allow treatment with lower total doses of IFN enabling dose intensification. In animal models of human hepatitis B virus (HBV) infection IFN-β directed to the liver specific antigen Asialoglycoprotein receptor (ASGPR) displayed significantly improved antiviral efficacy in vivo (Eto & Takahashi Nature Medicine 1999; 5:577-581).

The asialglycoprotein receptor binds asialoglycoproteins i.e. glycoproteins from which a sialic acid residue has been removed to expose one or more (typically) galactose residues. The ASGPR is expressed on liver cells which remove target glycoproteins from the circulation. The ASGPR molecule is hetero-oligomeric comprising two different subunits: H1 and H2.

There is thus a need to provide new therapeutic compositions which target molecules, including IFN, to the liver to treat and/or prevent liver diseases.

An antibody based approach to target molecules, including IFN for HCV treatment, may therefore provide a method of developing novel therapeutics with improved efficacy and tolerability profiles for use in treatment of a range of liver diseases.

### SUMMARY OF THE INVENTION

The present invention provides compositions and methods for targeting molecules to hepatocytes in the liver.

In one embodiment the invention a provides liver targeting composition which comprise a single molecule (e.g., as a single fusion or conjugate) which comprises (a) a ligand such as an antibody or an antibody fragment (e.g., a domain antibody (dAb)) which binds to liver cells, for example liver hepatocytes (e.g. to the ASGPR receptor on hepatocytes) and also (b) one or more therapeutic molecules for delivery to the liver. In particular the invention provides a liver targeting composition comprising a single molecule (such as a fusion or conjugate) comprising a ligand, such as an antibody or an antibody fragment (e.g. a domain antibody) which binds to the H1 subunit of ASGPR.

These liver targeting compositions can also comprise further proteins or polypeptides e.g. half life extending proteins or polypeptides e.g., a further dAb e.g., a dAb which binds to serum albumin or e.g., polyethyleneglygol PEG. These may be fused or conjugated to the single molecule, and may be fused or conjugated to the ligand, or to the therapeutic molecule, or to both the ligand and the therapeutic molecule. Methods of extending and/or measuring the in vivo half-life of molecules are known to those skilled in the art and are described in detail in, for example, WO2006/059110 and WO2008/096158.

In one embodiment the liver targeting composition comprises an antibody fragment (a) which is a single immunoglobulin variable domain (domain antibody (dAb)) which binds specifically to a hepatocyte e.g., to the ASGPR receptor on the hepatocytes, especially to the H1 subunit thereof. The dAb can be a human Vh or a human V Kappa. The dAb can also bind to a human and/or mouse ASGPR receptor.

Compositions of the invention also include ligands, for example a single immunoglobulin variable domain (dAb) which binds specifically to a hepatocyte e.g. to the ASGPR receptor on hepatocytes. For example the dAb provided by the invention can be a human Vh or a human V Kappa. The dAb can also bind to a human and/or mouse ASGPR receptor and/or ASGPR receptors from other animals.

In one embodiment, the dAb which binds to the ASGPR receptor on hepatocytes binds to human and/or mouse ASGPR, with high affinity as measured by Biacore [using the HBS-P buffer system (0.01M Hepes, pH7.4, 0.15M NaCl, 0.05% surfactant P20)] in the region of 1 pM to about 100 nM, for example about 1 pM to about 10 nM. or example about 1 pM to about 1 nM. In one embodiment the dAb will bind to both the human and to the mouse ASGPR with high affinity, as aforementioned.

For example, the dAb provided by the invention which specifically binds to the ASGPR receptor on hepatocytes can be one which comprises an amino acid sequence that is at least 80% identical (e.g., 85%, 90%, 95% or 100% identical) to the amino acid sequence encoded by the nucleotide sequences identified as: (anti human ASGPR VH dAbs) DOM26h-1 (Seq ID No: 155); DOM26h-10 (Seq ID No: 157); DOM26h-100 (Seq ID No: 159); DOM26h-101 (Seq ID No: 161); DOM26h-102 (Seq ID No: 163); DOM26h-103 (Seq ID No: 165); DOM26h-104 (Seq ID No: 167); DOM26h-105 (Seq ID No: 169); DOM26h-106 (Seq ID No: 171); DOM26h-107 (Seq ID No: 173); DOM26h-108 (Seq ID No: 175); DOM26h-109 (Seq ID No: 177); DOM26h-11 (Seq ID No: 179); DOM26h-110 (Seq ID No: 181); DOM26h-111 (Seq ID No: 183); DOM26h-112 (Seq ID No: 185); DOM26h-113 (Seq ID No: 187); DOM26h-114 (Seq ID No: 189); DOM26h-115 (Seq ID No: 191); DOM26h-116 (Seq ID No: 193); DOM26h-117 (Seq ID No: 195); DOM26h-118 (Seq ID No: 197); DOM26h-119 (Seq ID No: 199); DOM26h-12; (Seq ID No: 201) DOM26h-120 (Seq ID No: 203); DOM26h-121 (Seq ID No: 205); DOM26h-122 (Seq ID No: 207); DOM26h-123 (Seq ID No: 209); DOM26h-124; (Seq ID No: 211); DOM26h-125 (Seq ID No: 213); DOM26h-126 (Seq ID No: 215); DOM26h-127 (Seq ID No: 217); DOM26h-128 (Seq ID No: 219); DOM26h-129 (Seq ID No: 221); DOM26h-130 (Seq ID No: 223); DOM26h-131 (Seq ID No: 225); DOM26h-132 (Seq ID No: 227); DOM26h-133 (Seq ID No: 229); DOM26h-134 (Seq ID No: 231); DOM26h-135 (Seq ID No: 233); DOM26h-136 (Seq ID No: 235); DOM26h-137 (Seq ID No: 237); DOM26h-138 (Seq ID No: 239); DOM26h-139 (Seq ID No: 241); DOM26h-140 (Seq ID No: 243); DOM26h-141 (Seq ID No: 245); DOM26h-142 (Seq ID No: 247); DOM26h-143 (Seq ID No: 249); DOM26h-144 (Seq ID No: 251); DOM26h-145 (Seq ID No: 253); DOM26h-146 (Seq ID No: 255); DOM26h-147 (Seq ID No: 257); DOM26h-148 (Seq ID No: 259); DOM26h-149 (Seq ID No: 261); DOM26h-15 (Seq ID No: 263); DOM26h-150 (Seq ID No: 265); DOM26h-151 (Seq ID No: 267); DOM26h-152 (Seq ID No: 269); DOM26h-153 (Seq ID No: 271); DOM26h-154 (Seq ID No: 273); DOM26h-155 (Seq ID No: 275); DOM26h-156 (Seq ID No: 277); DOM26h-157 (Seq ID No: 279); DOM26h-158 (Seq ID No: 281); DOM26h-159 (Seq ID No: 283); DOM26h-159-1 (Seq ID No: 285); DOM26h-159-2 (Seq ID No: 287); DOM26h-159-3 (Seq ID No: 289); DOM26h-159-4 (Seq ID No: 291); DOM26h-159-5 (Seq ID No: 293); DOM26h-160 (Seq ID No: 295); DOM26h-168 (Seq ID No: 297); DOM26h-169 (Seq ID No: 299); DOM26h-17 (Seq ID No: 301); DOM26h-170 (Seq ID No: 303); DOM26h-171 (Seq ID No: 305); DOM26h-172 (Seq ID No: 307); DOM26h-173 (Seq ID No: 309); DOM26h-174 (Seq ID No: 311); DOM26h-175 (Seq ID No: 313); DOM26h-176 (Seq ID No: 315); DOM26h-177 (Seq ID No: 317); DOM26h-178 (Seq ID No: 319); DOM26h-179 (Seq ID No: 321); DOM26h-180 (Seq ID No: 323); DOM26h-181 (Seq ID No: 325); DOM26h-182 (Seq ID No: 327); DOM26h-183 (Seq ID No: 329); DOM26h-184 (Seq ID No: 331); DOM26h-185 (Seq ID No: 333); DOM26h-186 (Seq ID No: 335); DOM26h-187 (Seq ID No: 337); DOM26h-188 (Seq ID No: 339); DOM26h-189 (Seq ID No: 341); DOM26h-19 (Seq ID No: 343); DOM26h-190 (Seq ID No: 345); DOM26h-191 (Seq ID No: 347); DOM26h-192 (Seq ID No: 349); DOM26h-193 (Seq ID No: 351); DOM26h-194 (Seq ID No: 353); DOM26h-195 (Seq ID No: 355); DOM26h-196 (Seq ID No: 357); DOM26h-197 (Seq ID No: 359); DOM26h-198 (Seq ID No: 361); DOM26h-199 (Seq ID No: 363); DOM26h-2 (Seq ID No: 365); DOM26h-20 (Seq ID No: 367); DOM26h-200 (Seq ID No: 369); DOM26h-201 (Seq ID No: 371); DOM26h-202 (Seq ID No: 373); DOM26h-203 (Seq ID No: 375); DOM26h-204 (Seq ID No: 377); DOM26h-205 (Seq ID No: 379); DOM26h-206 (Seq ID No: 381); DOM26h-207 (Seq ID No: 383); DOM26h-208 (Seq ID No: 385); DOM26h-209 (Seq ID No: 387); DOM26h-21 (Seq ID No: 389); DOM26h-210 (Seq ID No: 391); DOM26h-211 (Seq ID No: 393); DOM26h-212 (Seq ID No: 395); DOM26h-213 (Seq ID No: 397); DOM26h-214 (Seq ID No: 399); DOM26h-215 (Seq ID No: 401); DOM26h-216 (Seq ID No: 403); DOM26h-217 (Seq ID No: 405); DOM26h-218 (Seq ID No: 407); DOM26h-219 (Seq ID No: 409); DOM26h-22 (Seq ID No: 411); DOM26h-220 (Seq ID No: 413); DOM26h-221 (Seq ID No: 415); DOM26h-222 (Seq ID No: 417); DOM26h-223 (Seq ID No: 419); DOM26h-23 (Seq ID No: 421); DOM26h-24 (Seq ID No: 423); DOM26h-29-1 (Seq ID No: 425); DOM26h-4 (Seq ID No: 427); DOM26h-41 (Seq ID No: 429); DOM26h-42 (Seq ID No: 431); DOM26h-43 (Seq ID No: 433); DOM26h-44 (Seq ID No: 435); DOM26h-45 (Seq ID No: 437); DOM26h-46 (Seq ID No: 439); DOM26h-47 (Seq ID No: 441); DOM26h-48 (Seq ID No: 443); DOM26h-49 (Seq ID No: 445); DOM26h-50 (Seq ID No: 447); DOM26h-51 (Seq ID No: 449); DOM26h-52 (Seq ID No: 451); DOM26h-53 (Seq ID No: 453); DOM26h-54 (Seq ID No: 455); DOM26h-55 (Seq ID No: 457); DOM26h-56 (Seq ID No: 459); DOM26h-57 (Seq ID No: 461); DOM26h-58 (Seq ID No: 463); DOM26h-59 (Seq ID No: 465); DOM26h-60 (Seq ID No: 467); DOM26h-61 (Seq ID No: 469); DOM26h-62 (Seq ID No: 471); DOM26h-63 (Seq ID No: 473); DOM26h-64 (Seq ID No: 475); DOM26h-65 (Seq ID No: 477); DOM26h-66 (Seq ID No: 479); DOM26h-67 (Seq ID No: 481); DOM26h-68 (Seq ID No: 483); DOM26h-69 (Seq ID No: 485); DOM26h-70 (Seq ID No: 487); DOM26h-71 (Seq ID No: 489); DOM26h-72 (Seq ID No: 491); DOM26h-73 (Seq ID No: 493); DOM26h-74 (Seq ID No: 495); DOM26h-75 (Seq ID No: 497); DOM26h-76 (Seq ID No: 499); DOM26h-77 (Seq ID No: 501); DOM26h-78 (Seq ID No: 503); DOM26h-79 (Seq ID No: 505); DOM26h-80 (Seq ID No: 507); DOM26h-81 (Seq ID No: 509); DOM26h-82 (Seq ID No: 511); DOM26h-83 (Seq ID No: 513); DOM26h-84 (Seq ID No: 515); DOM26h-85 (Seq ID No: 517); DOM26h-86 (Seq ID No: 519); DOM26h-87 (Seq ID No: 521); DOM26h-88 (Seq ID No: 523); DOM26h-89 (Seq ID No: 525); DOM26h-90 (Seq ID No: 527); DOM26h-91 (Seq ID No: 529); DOM26h-92 (Seq ID No: 531); DOM26h-93 (Seq ID No: 533); DOM26h-94 (Seq ID No: 535); DOM26h-95 (Seq ID No: 537); DOM26h-96 (Seq ID No: 539); DOM26h-97 (Seq ID No: 541); DOM26h-98 (Seq ID No: 543); DOM26h-99 (Seq ID No: 545); DOM26h-99-1 (Seq ID No: 547); DOM26h-99-2 (Seq ID No: 549); (anti human ASGPR Vκ Clones) DOM26h-161 (Seq ID No: 551); DOM26h-162 (Seq ID No: 553); DOM26h-163 (Seq ID No: 555); DOM26h-164 (Seq ID No: 557); DOM26h-165 (Seq ID No: 559); DOM26h-166 (Seq ID No: 561); DOM26h-167 (Seq ID No: 563); DOM26h-224 (Seq ID No: 565); DOM26h-25 (Seq ID No: 567); DOM26h-26 (Seq ID No: 569); DOM26h-27 (Seq ID No: 571); DOM26h-28 (Seq ID No: 573); DOM26h-29 (Seq ID No: 575); DOM26h-30 (Seq ID No: 577); DOM26h-31 (Seq ID No: 579); DOM26h-32 (Seq ID No: 581); DOM26h-33 (Seq ID No: 583); DOM26h-34 (Seq ID No: 585); DOM26h-35 (Seq ID No: 587); DOM26h-36 (Seq ID No: 589); DOM26h-37 (Seq ID No: 591); DOM26h-38 (Seq ID No: 593); DOM26h-39 (Seq ID No: 595); DOM26h-40 (Seq ID No: 597); DOM26h-6 (Seq ID No: 599); DOM26h-8 (Seq ID No: 601); DOM26h-9 (Seq ID No: 603).

In another embodiment, the dAb provided by the invention which specifically binds to the ASGPR receptor on hepatocyes may be one which comprises an amino acid sequence that is at least 80% identical (e.g. 85%, 90%, 95% or 100% identical) to the affinity-matured dAb clone sequences encoded by the nucleotide sequences identified in FIG. 32 as DOM26h-161-84 (Seq ID No: 867); DOM26h-161-86 (Seq ID No: 869); DOM26h-161-87 (Seq ID No: 871); DOM26h-196-61 (Seq ID No: 873); DOM26h-210-2 (Seq ID No: 875); DOM26h-220-1 (Seq ID No: 877); or DOM26h-220-43 (Seq ID No: 879).

In another example, the dAb which binds to the ASGPR receptor on hepatocytes is one which comprises an amino acid sequence that is at least 80% identical (e.g. 85%, 90%, 95% or 100% identical) to the amino acid sequence encoded by the nucleotide sequences identified as: (anti mouse ASGPR VH dAbs) DOM26m-10 (Seq ID No: 605); DOM26m-13 (Seq ID No: 607); DOM26m-16 (Seq ID No: 609); DOM26m-165 (Seq ID No: 611); DOM26m-17 (Seq ID No: 613); DOM26m-27 (Seq ID No: 615); DOM26m-28 (Seq ID No: 617); DOM26m-29 (Seq ID No: 619); DOM26m-30 (Seq ID No: 621); DOM26m-31 (Seq ID No: 623); DOM26m-32 (Seq ID No: 625); DOM26m-33 (Seq ID No: 627); DOM26m-33-1 (Seq ID No: 629); DOM26m-33-10 (Seq ID No: 631); DOM26m-33-11 (Seq ID No: 633); DOM26m-33-12 (Seq ID No: 635); DOM26m-33-2 (Seq ID No: 637); DOM26m-33-3 (Seq ID No: 639); DOM26m-33-4 (Seq ID No: 641); DOM26m-33-5 (Seq ID No: 643); DOM26m-33-6 (Seq ID No: 645); DOM26m-33-7 (Seq ID No: 647); DOM26m-33-8 (Seq ID No: 649); DOM26m-33-9 (Seq ID No: 651); DOM26m-34 (Seq ID No: 653); DOM26m-35 (Seq ID No: 655); DOM26m-36 (Seq ID No: 657); DOM26m-37 (Seq ID No: 659); DOM26m-38 (Seq ID No: 661); DOM26m-39 (Seq ID No: 663); DOM26m-4 (Seq ID No: 665); DOM26m-40 (Seq ID No: 667); DOM26m-41 (Seq ID No: 669); DOM26m-42 (Seq ID No: 671); DOM26m-43 (Seq ID No: 673); DOM26m-44 (Seq ID No: 675); DOM26m-45 (Seq ID No: 677); DOM26m-46 (Seq ID No: 679); DOM26m-47 (Seq ID No: 681); DOM26m-48 (Seq ID No: 683); DOM26m-52 (Seq ID No: 685); DOM26m-52-1 (Seq ID No: 687); DOM26m-52-2 (Seq ID No: 689); DOM26m-52-3 (Seq ID No: 691); DOM26m-52-4 (Seq ID No: 693); DOM26m-52-5 (Seq ID No: 695); DOM26m-52-6 (Seq ID No: 697); DOM26m-52-7 (Seq ID No: 699); DOM26m-6 (Seq ID No: 701); DOM26m-60 (Seq ID No: 703); DOM26m-61-1 (Seq ID No: 705); DOM26m-61-2 (Seq ID No: 707); DOM26m-61-3 (Seq ID No: 709); DOM26m-61-4 (Seq ID No: 711); DOM26m-61-5 (Seq ID No: 713); DOM26m-61-6 (Seq ID No: 715); DOM26m-7 (Seq ID No: 717); DOM26m-73 (Seq ID No: 719); DOM26m-74 (Seq ID No: 721); DOM26m-75 (Seq ID No: 723); DOM26m-76 (Seq ID No: 725); DOM26m-77 (Seq ID No: 727); DOM26m-78 (Seq ID No: 729); DOM26m-79 (Seq ID No: 731); DOM26m-8 (Seq ID No: 733); DOM26m-80 (Seq ID No: 735); DOM26m-81 (Seq ID No: 737); DOM26m-82 (Seq ID No: 739); DOM26m-83 (Seq ID No: 741); DOM26m-9 (Seq ID No: 743); (anti mouse ASGPR Vκ dAbs) DOM26m-1 (Seq ID No: 745); DOM26m-100 (Seq ID No: 747); DOM26m-101 (Seq ID No: 749); DOM26m-102 (Seq ID No: 751); DOM26m-103 (Seq ID No: 753); DOM26m-106 (Seq ID No: 755); DOM26m-108 (Seq ID No: 757); DOM26m-109 (Seq ID No: 759); DOM26m-109-1 (Seq ID No: 761); DOM26m-109-2 (Seq ID No: 763); DOM26m-12 (Seq ID No: 765); DOM26m-18 (Seq ID No: 767); DOM26m-19 (Seq ID No: 769); DOM 26m-2 (Seq ID No: 771); DOM26m-20 (Seq ID No: 773); DOM26m-20-1 (Seq ID No: 775); DOM26m-20-2 (Seq ID No: 777); DOM26m-20-3 (Seq ID No: 779); DOM26m-20-4 (Seq ID No: 781); DOM26m-20-5 (Seq ID No: 783); DOM26m-20-6 (Seq ID No: 785); DOM26m-22 (Seq ID No: 787); DOM26m-23 (Seq ID No: 789); DOM26m-24 (Seq ID No: 791); DOM26m-25 (Seq ID No: 793); DOM26m-26 (Seq ID No: 795); DOM26m-3 (Seq ID No: 797); DOM26m-50 (Seq ID No: 799); DOM26m-50-1 (Seq ID No: 801); DOM26m-50-2 (Seq ID No: 803); DOM26m-50-3 (Seq ID No: 805); DOM26m-50-4 (Seq ID No: 807); DOM26m-50-5 (Seq ID No: 809); DOM26m-50-6 (Seq ID No: 811); DOM26m-51 (Seq ID No: 813); DOM26m-53 (Seq ID No: 815); DOM26m-54 (Seq ID No: 817); DOM26m-55 (Seq ID No: 819); DOM26m-56 (Seq ID No: 821); DOM26m-57 (Seq ID No: 823); DOM26m-58 (Seq ID No: 825); DOM26m-59 (Seq ID No: 827); DOM26m-61 (Seq ID No: 829); DOM26m-63 (Seq ID No: 831); DOM26m-64 (Seq ID No: 833); DOM26m-66 (Seq ID No: 835); DOM26m-69 (Seq ID No: 837); DOM26m-85 (Seq ID No: 839); DOM26m-86 (Seq ID No: 841); DOM26m-87 (Seq ID No: 843); DOM26m-89 (Seq ID No: 845); DOM26m-90 (Seq ID No: 847); DOM26m-91 (Seq ID No: 849); DOM26m-92 (Seq ID No: 851); DOM26m-93 (Seq ID No: 853); DOM26m-94 (Seq ID No: 855); DOM26m-95 (Seq ID No: 857); DOM26m-96 (Seq ID No: 859); DOM26m-97 (Seq ID No: 861); DOM26m-98 (Seq ID No: 863); DOM26m-99 (Seq ID No: 865).

In an embodiment the ligand e.g. the dAb, can be one which competes for binding to the ASGPR receptor with any one of the DOM 26 dAbs described herein (with an amino acid sequence shown in FIGS. 15, 16, 19 and 20).

In yet another aspect there is provided a dAb which binds to ASGPR comprising at least one CDR selected from the group consisting of: CDR1, CDR2, and CDR3, wherein the CDR1, CDR2, or CDR3 is at least 80% identical (e.g. 85%, 90%, 95% or 100% identical) to a CDR1, CDR2, or CDR3 sequence in any one of the amino DOM 26 amino acid sequences as described herein. The CDRs can be identified in the amino acid sequences as follows: V kappa sequences: CDR1 is residues 24-34, CDR2 is residues 50-56, CDR3 is residues 89-97; for V H sequences: CDR1 is residues 31-35, CDR2 is residues 50-65, CDR3 is residues 95-102.

In one embodiment, the dAbs of the present invention show cross-reactivity between human ASGPR and ASGPR from another species such as mouse, dog or cynomolgus macaque. In one embodiment, the dAbs of the present invention show cross-reactivity between human and mouse ASGPR. In this embodiment, the variable domains specifically bind human and mouse ASGPR. In one embodiment the invention provides a variable domain which is cross reactive for human and mouse ASGPR and which is an amino acid sequence selected from: DOM 26m-52, DOM 26h-99, DOM 26h-161, DOM 26h-163, DOM 26h-186, DOM 26h-196, DOM 26h-210, and DOM 26h-220 or an amino acid sequence which is at least 80% identical (e.g. 85%, 90%, 95% or 100%) identical to an amino acid sequence selected from: DOM 26m-52, DOM 26h-99, DOM 26h-161, DOM 26h-163, DOM 26h-186, DOM 26h-196, DOM 26h-210, and DOM 26h-220.

As described above, cross-reactivity is particularly useful, since drug development typically requires testing of lead drug candidates in animal systems, such as mouse models, before the drug is tested in humans. The provision of a drug that can bind to a human protein as well as the species homologue such as the equivalent mouse protein allows one to test results in these systems and make side-by-side comparisons of data using the same drug. This avoids the complication of needing to find a drug that works against, for example, a mouse ASGPR and a separate drug that works against human ASGPR, and also avoids the need to compare results in humans and mice using non-identical or surrogate drugs.

In another embodiment the invention provides a liver targeting composition which comprise a single molecule (e.g. present as a single fusion or conjugate) which comprises (a) a dAb which binds to the ASGPR receptor on hepatocytes, e.g. any one of the ASGPR dAbs as described herein and also (b) one or more therapeutic molecules for delivery to the liver.

In one embodiment of the above the molecule (b) which it is desired to deliver to the liver can be an interferon, for example it can be interferon alpha 2, interferon alpha 5, interferon alpha 6, or Consensus interferon, or it can be a mutant or derivative of any of these which retains some interferon activity.

In another embodiment the present invention provides a composition which comprises any one of the liver targeting compositions as described herein, and also a further drug for delivery to the liver for example Ribavirin and/or a drug for systemic delivery. Such a composition can be a combined preparation for simultaneous, separate or sequential use in therapy, e.g to treat or prevent a liver disease or condition such as an inflammatory liver disease e.g. fibrosis or a viral liver disease e.g. Hepatitis (e.g. Hepatitis C), or Cirrhosis or liver cancer.

In one embodiment, the drug which it is desired to deliver to the liver may comprise one or more of the following: Nexavar® (also known as Sorafenib)—a small molecule used in the treatment of primary hepatocellular carcinoma; Erbitux® (also known as Cetuximab)—a monoclonal antibody used in the treatment of primary liver cancers, or bowel cancer metastases in the liver; Avastin® (also known as bevacizumab) and Herceptin® (also known as trastuzumab), which are used to treat bowel or breast cancer metastases respectively in the liver.

Nexavar could, for example, be conveniently chemically conjugated to an antibody or dAb or the like which binds to the ASGPR receptor. Erbitux®, Avastin® or Herceptin® containing-fusions could conveniently be prepared by fusing a nucleotide sequence encoding the Erbitux®, Avastin® or Herceptin® antibody with a nucleotide sequence encoding an antibody, dAb or the like which binds to the ASGPR receptor.

The therapeutic molecule(s) for delivery to the liver (e.g. interferon) when present as a fusion (or conjugate) with a liver targeting dAb can be linked to either the N-terminal or C-terminal of the dAb or at points within the dAb sequence. In one embodiment one or more interferon molecules e.g. interferon alpha 2 are present as a fusion (or conjugate) at the N terminal of the dAb.

An amino acid or chemical linker may also optionally be present joining the therapeutic molecule(s) for delivery to the liver (e.g. interferon) with the dAb. The linker can be for example a TVAAPR or TVAAPS linker sequence, a helical linker or it can be a gly-ser linker.

Alternatively the linker can be e.g. a PEG linker. The linker can also be a peptide linker, a linker containing a functionality such as a protease cleavage site, or a chelating group e.g. for attachment of a radioisotope or other imaging agent.

In certain embodiments, the dAbs, fusions (or conjugates) of the invention can comprise further molecules e.g. further peptides or polypeptides, such as half-life extending polypeptides (e.g. a dAb or antibody fragment which binds to serum albumin), or one or more PEG molecules.

As used herein, “fusion” refers to a fusion protein that comprises as one moiety a dAb that binds to hepatocytes (e.g. to the ASGPR on hepatocytes) and one or more further molecules which are therapeutic molecules which it is desired to deliver to the liver (e.g. interferon). The dAb that binds to hepatocytes (e.g. to the ASGPR on hepatocytes) and the therapeutic molecules are present as discrete parts (moieties) of a single continuous polypeptide chain. The dAb and the therapeutic molecules can be directly bonded to each other through a peptide bond or linked through a suitable amino acid, or peptide or polypeptide linker. Additional moieties e.g. peptides or polypeptides (e.g. third, fourth) and/or linker sequences, can be present as appropriate. The dAb can be in an N-terminal location, C-terminal location or it can be internal relative to the therapeutic molecules.

As used herein, “conjugate” refers to a composition comprising a dAb that binds to hepatocytes (e.g. to the ASGPR on hepatocytes) to which one or more therapeutic molecules for delivery to the liver are covalently or non-covalently bonded. The therapeutic molecule can be covalently bonded to the dAb directly or indirectly through a suitable linker moiety. The therapeutic molecule can be bonded to the dAb at any suitable position, such as the amino-terminus, the carboxyl-terminus or through suitable amino acid side chains (e.g., the 8 amino group of lysine, or thiol group of cysteine). Alternatively, the therapeutic molecule can be noncovalently bonded to the dAb directly (e.g., electrostatic interaction, hydrophobic interaction) or indirectly (e.g., through noncovalent binding of complementary binding partners (e.g., biotin and avidin), wherein one partner is covalently bonded to insulinotropic/incretin molecule and the complementary binding partner is covalently bonded to the dAb). The dAb can be in an N-terminal location, C-terminal location or it can be internal relative to the therapeutic molecule.

The invention also provides compositions comprising nucleic acids encoding the fusions described herein for example comprising any one of the nucleic acids encoding the DOM 26 dAbs as shown in FIGS. 13-14 and 17-18.

Also provided are host cells e.g. non-embryonic host cells e.g. prokaryotic or eukaryotic hosts cells such as bacterial host cells (e.g. E. coli) or or yeast host cells or mammalian cells that comprise these nucleic acids.

The invention further provides a method for producing a fusion protein of the present invention which method comprises maintaining a host cell such as those described above that comprises a recombinant nucleic acid and/or construct that encodes a fusion of the invention under conditions suitable for expression of said recombinant nucleic acid, whereby a fusion protein is produced.

The invention also provides pharmaceutical compositions comprising the compositions of the invention.

The invention further provides a composition of the invention for use in medicine, e.g. for use in the treatment or prevention of e.g. a liver disease or condition or disorder such as a viral liver disease (e.g. Hepatitis e.g. Hepatitis C), cirrhosis, or liver cancer, and which comprises administering to said individual a therapeutically effective amount of a composition of the invention.

The invention also provides a method for treating (therapeutically or prophylactically) a patient or subject having a disease or disorder, such as those described herein e.g. a liver disease or condition or disorder such as a viral liver disease (e.g. Hepatitis e.g. Hepatitis C), cirrhosis, or liver cancer, and which comprises administering to said individual a therapeutically effective amount of a composition of the invention.

The compositions e.g. pharmaceutical compositions, of the invention may be administered alone or in combination with other molecules or moieties e.g. polypeptides, therapeutic proteins and/or molecules (e.g., other proteins (including antibodies), peptides, or small molecule drugs.

The invention also provides compositions of the invention for use in the treatment of a liver disease or condition or disorder such as a viral liver disease (e.g. Hepatitis e.g. Hepatitis C), cirrhosis, or liver cancer.

The invention also provides for use of a composition of the invention in the manufacture of a medicament for treatment of a liver disease or condition or disorder such as a viral liver disease (e.g. Hepatitis e.g. Hepatitis C), cirrhosis, or liver cancer.

The invention also relates to use of any of the compositions described herein for use in therapy, diagnosis or prophylaxis of a liver disease or condition such as a viral liver disease (e.g. Hepatitis e.g. Hepatitis C), cirrhosis, or liver cancer disease or disorder. The invention also relates to prophylactic use of any of the compositions described herein after infection with a liver infecting blood borne pathogen.

The compositions of the invention, e.g. the dAb component of the composition, can be further formatted to have a larger hydrodynamic size to further extend the half life, for example, by attachment of a PEG group, serum albumin, transferrin, transferrin receptor or at least the transferrin-binding portion thereof, an antibody Fc region, or by conjugation to an antibody domain. For example, the dAb that binds serum albumin can be formatted as a larger antigen-binding fragment of an antibody (e.g., formatted as a Fab, Fab′, F(ab)₂, F(ab′)₂, IgG, scFv).

In other embodiments of the invention described throughout this disclosure, instead of the use of a “dAb” in a fusion of the invention, it is contemplated that the skilled addressee can use a domain that comprises the CDRs of a dAb that binds specifically to hepatocytes e.g. the ASGPR receptor on hepatocytes (e.g., the CDRs can be grafted onto a suitable protein scaffold or skeleton, eg an affibody, an SpA scaffold, an LDL receptor class A domain or an EGF domain). The disclosure as a whole is to be construed accordingly to provide disclosure of such domains in place of a dAb.

In certain embodiments, the invention provides a composition according to the invention that comprises a dual-specific ligand or multi-specific ligand that comprises a first dAb according to the invention that binds hepatocytes (e.g. the ASGPR receptor on hepatocytes) and a second dAb that has the same or a different binding specificity from the first dAb and optionally in the case of multi-specific ligands further dAbs. The second dAb (or further dAbs) may optionally bind a different target.

Thus, in one aspect, the invention provides the compositions of the invention for delivery by parenteral administration e.g. by subcutaneous, intramuscular or intravenous injection, inhalation, nasal delivery, transmucossal delivery, oral delivery, delivery to the GI tract of a patient, rectal delivery or ocular delivery. In one aspect, the invention provides the use of the compositions of the invention in the manufacture of a medicament for delivery by subcutaneous injection, inhalation, intravenous delivery, nasal delivery, transmucossal delivery, oral delivery, delivery to the GI tract of a patient, rectal delivery, transdermal or ocular delivery.

In one aspect, the invention provides a method for delivery to a patient by subcutaneous injection, pulmonary delivery, intravenous delivery, nasal delivery, transmucossal delivery, oral delivery, delivery to the GI tract of a patient, rectal or ocular delivery, wherein the method comprises administering to the patient a pharmaceutically effective amount of a fusion or conjugate of the invention.

In one aspect, the invention provides an oral, injectable, inhalable, or nebulisable formulation comprising a fusion or conjugate of the invention.

The formulation can be in the form of a tablet, pill, capsule, liquid or syrup.

The term “subject” or “individual” is defined herein to include animals such as mammals, including, but not limited to, primates (e.g., humans), cows, sheep, goats, horses, dogs, cats, rabbits, guinea pigs, rats, mice or other bovine, ovine, equine, canine, feline, rodent or murine species.

The invention also provides a kit for use in administering compositions according to the invention to a subject (e.g., human patient), comprising a composition of the invention, a drug delivery device and, optionally, instructions for use. The composition can be provided as a formulation, such as a freeze dried formulation or a slow release formulation. In certain embodiments, the drug delivery device is selected from the group consisting of a syringe, an inhaler, an intranasal or ocular administration device (e.g., a mister, eye or nose dropper), and a needleless injection device.

The compositions (e.g dAbs and liver targeting compositions) of this invention can be lyophilized for storage and reconstituted in a suitable carrier prior to use. Any suitable lyophilization method (e.g., spray drying, cake drying) and/or reconstitution techniques can be employed. It will be appreciated by those skilled in the art that lyophilisation and reconstitution can lead to varying degrees of antibody activity loss and that use levels may have to be adjusted to compensate. In a particular embodiment, the invention provides a composition comprising a lyophilized (freeze dried) composition as described herein. Preferably, the lyophilized (freeze dried) composition loses no more than about 20%, or no more than about 25%, or no more than about 30%, or no more than about 35%, or no more than about 40%, or no more than about 45%, or no more than about 50% of its activity (e.g., binding activity for serum albumin) when rehydrated. Activity is the amount of composition required to produce the effect of the composition before it was lyophilized. The activity of the composition can be determined using any suitable method before lyophilization, and the activity can be determined using the same method after rehydration to determine amount of lost activity.

The invention also provides sustained or slow release formulations comprising the compositions of the invention, such sustained release formulations can comprise the composition of the invention in combination with, e.g. hyaluronic acid, microspheres or liposomes and other pharmaceutically or pharmacalogically acceptable carriers, excipients and/or diluents.

In one aspect, the invention provides a pharmaceutical composition comprising a composition of the invention, and a pharmaceutically or physiologically acceptable carrier, excipient or diluent.

### BRIEF DESCRIPTION OF THE DRAWINGS:

FIG. 1: shows binding of P-GalNAc-PAA-biotin to human (His)₆-ASGPR H1 ( [symbol] ), mouse (His)₆-ASGPR H1 ( [symbol] ) and human (His)₆-GP6 irrelevant control antigen ( [symbol] ) Antigens were immobilised on a biacore CM5 chip surface and 100 nM ligand passed over at a flow rate of 10 μl min⁻¹. Sensorgram illustrates that ligand binds to human and mouse (His)₆-ASGPR H1 antigens but not (His)₆-GP6 irrlevant control antigen.

FIG. 2: shows 4-12% Bis-Tris gel loaded with 2 μg of Ni-NTA purified human (His)₆-ASGPR H1 (lane 2) or mouse (His)₆-ASGPR H1 (lane 3) expressed in HEK293E. 10 μl Mark 12 molecular weight standards (Invitrogen) were loaded in lane 1 and molecular masses (in kilodaltons) of individual marker bands are given to the left of lane 1. Gel was stained with 1× SureBlue. Gel illustrates that human and mouse (His)₆-ASGPR H1 migrate close to the expected molecular mass based on amino acid sequence.

FIG. 3: Vκ and V_{H} dAbs selected against recombinant human and mouse ASGPR proteins binding specifically to the target antigen. Antigens were coated on the surface of a CM5 BlAcore chip and protein L purified Vκ dAb DOM26m-20 (top panel) or protein A purified V_{H} dAb DOM26h-61 (bottom panel) was passed over the chip surface at a concentration of 2.5 μM using a flow-rate of 10 μl per second. In the top panel binding of dAb to (His)₆-mouse ASGPR H1 ( [symbol] )or human c-kit (His)₆ negative control antigen ( [symbol] ) is shown. In the bottom panel dAb binding to (His)₆-human ASGPR H1 ( [symbol] )or human c-kit (His)₆ negative control antigen ( [symbol] ) is shown.

FIG. 4: shows dAb clones selected against recombinant (His)₆-mouse ASGPR H1 antigen binding specifically to murine liver cell lines in a flow cytometry cell binding assay. Binding of dAbs with c-terminal FLAG epitope tags cross-linked with anti-FLAG M2 monoclonal antibody to murine hepatoma cell line Hepalc1c7 (top panel) or murine fibroblast negative control cell line L929 (bottom panel) was tested in this assay. Goat polyclonal antibody specific for mouse IgG (GaM-FITC) was used as secondary detection reagent. VκD (human germ-line Vκ sequence with a c-terminal FLAG epitope tag) was used as a non-specific dAb binding control. Results obtained with anti-FLAG M2 in the absence of dAb (FLAG only) and secondary detection reagent in the absence of dAb or anti-FLAG M2 (GaM-FITC) are also shown together with unstained cells. For each dAb a half-log dilution series was tested starting at 10 μM final concentration in the assay (right hand bar in each series).

FIG. 5: shows binding and localisation of anti-mouse ASGPR dAb DOM26m-33 following incubation with Hepal cic7 murine liver cell line. After incubation for 30 minutes in the presence of 5 μM DOM26m-33 with a c-terminal FLAG epitope tag cells were fixed with 4% paraformaldehyde/0.2% saponin and stained with monoclonal anti-FLAG M2 Cy3 conjugate to determine dAb localisation or rabbit polyclonal antibody specific for either EEA1 or LAMP1 to determine localisation of early endosome and lysosome respectively. The top panel shows similarity between the pattern of localisation for DOM26m-33 and EEA1, with some overlap in the observed staining pattern. The bottom panel shows that the pattern of localisation for DOM26m-33 and LAMP 1 are distinct, with no overlap in the observed staining pattern.

FIG. 6: shows BlAcore sensorgram from epitope mapping experiment to determine whether mouse ASGPR specific dAbs DOM26m-33 and DOM26m-52 bind to distinct epitopes within the antigen. dAbs were passed over BlAcore CM5 chip surface coated with (His)₆ mouse ASGPR H1 at a concentration of 1 μM dAb using a flow rate of 10 μl per second. Injection events are as follows:

1=injection of dAb 1

2=injection of dAb 2

3=co injection of dAb 1 followed by dAb 2

4=co injection of dAb 2 followed by dAb 1

*=regeneration of chip surface with 15 second pulse of 0.1 M glycine, pH 2.0

In this experiment co injection of DOM26m-33 and DOM26m-52 inhibits binding (in comparison to dAb injected alone) by >20%, therefore DOM26m-33 and DOM26m-52 bind to partially overlapping epitopes within mouse ASGPR H1 subunit. Antibody binding of mouse ASGPR H1 was unaffected by regeneration with 0.1 M glycine, pH 2.0 in these experiments.

FIG. 7: shows localisation of ¹¹¹In labelled dAbs in balb/c mice at 3 hours post injection. Following intravenous dosing of 12 MBq of radiolabelled dAb via tail vein injection mice were imaged using a nanospect camera. Images show that at 3 hours signal is observed in kidney and bladder with all three dAb molecules, whereas liver localisation in only observed with anti murine ASGPR dAb DOM26m-33.

FIG. 8: shows biodistribution of ¹¹¹In labelled dAbs 3 hours after dosing intravenously in balb/c mice via the tail vein. Approximately 0.5 MBq radiolabelled dAb was injected in each case. Results show accumulation of radiolabelled dAb in mouse liver is 12.4 times higher in mice injected with DOM26m-33 than in mice injected with Vκ dummy and 4.9 times higher than in mice injected with V_{H} dummy

FIG. 9: shows 4-12% Bis-Tris gel loaded with 2 μg per lane of protein L purified mIFNa2-dAb fusions reduced with 10 mM DTT. Lane designations as follows:

mIFNa2-Vκ dummy (lane 2),

mIFNa2-Vκ dummy with C-terminal cysteine point mutation (lane 3)

mIFNa2-V_{H} dummy (lane 4)

mIFNa2-V_{H} dummy with C-terminal cysteine point mutation (lane 5)

mIFNa2-DOM26m-33 (lane 6)

mIFNa2-DOM26m-33 with C-terminal cysteine point mutation (lane 7)

10 μl Mark 12 molecular weight standards (Invitrogen) were loaded in lane 1 and molecular masses (in kilodaltons) of individual marker bands are given to the left of lane 1. Gel was stained with 1× SureBlue. Gel illustrates that mouse IFNa2-dAb fusions migrate close to the expected molecular mass of approximately 33 KDa.

FIG. 10: shows activity of mouse IFN-dAb fusions in CHO ISRE-Luc transient transfection assay. CHO-Kl cells were incubated with the indicated concentrations of mouse IFN-alpha standard or mouse IFN-dAb fusion protein. Top panel shows results obtained with mouse IFNa2-DOM26m-33 fusion proteins, middle panel shows results obtained with mouse IFNa2-V_{H} dummy 2 fusion proteins and lower panel shows results obtained with mouse IFNa2-Vκ dummy fusion proteins. Symbols denote the following:

▴=mouse IFNa2-dAb fusions

▪=mouse IFNa2-dAb fusions with C-terminal cysteine mutation

▾=mouse IFN-alpha standard.

FIG. 11 a shows binding of mouse mouse IFNa2-DOM26m-33 fusions to (His)6 mouse ASGPR H1 coated on the surface of BlAcore CM5 chip. Traces represent binding of DOM26m-33 only ( [symbol] )shown in all panels for comparison, mouse IFNa2-dAb fusions ( [symbol] ) and mouse IFNa2-dAb fusions with C-terminal cysteine mutation ( [symbol] ).

FIG. 11b shows binding of mouse mouse IFNa2-DOM26m-33 fusions to (His)6 mouse ASGPR H1 coated on the surface of BlAcore CM5 chip.

Traces represent binding of DOM26m-33 only ( [symbol] )shown in all panels for comparison, mouse IFNa2-dAb fusions ( [symbol] ) and mouse IFNa2-dAb fusions with C-terminal cysteine mutation ( [symbol] )

FIG. 11c shows binding of mouse mouse IFNa2-DOM26m-33 fusions to (His)6 mouse ASGPR H1 coated on the surface of BlAcore CM5 chip. Traces represent binding of DOM26m-33 only ( [symbol] )shown in all panels for comparison, mouse IFNa2-dAb fusions ( [symbol] ) and mouse IFNa2-dAb fusions with C-terminal cysteine mutation ( [symbol] ).

FIG. 12: shows murine ASGPR specific dAb clones grouped according to epitopes bound within the antigen.

FIG. 13: shows nucleotide sequences of anti-human Vh ASGPR dAbs. (Seq ID No.s 155-549; odd numbers only)

FIG. 14: shows nucleotide sequences of anti-human V kappa ASGPR dAbs. (Seq ID No.s 551-603; odd numbers only)

FIG. 15: shows amino acid sequences of anti-human Vh ASGPR dAbs. (Seq ID No.s 156-550; even numbers only)

FIG. 16: shows amino acid sequences of anti-human V kappa ASGPR dAbs. (Seq ID No.s 552-604; even numbers only)

FIG. 17: shows nucleotide sequences of anti-mouse Vh ASGPR dAbs. (Seq ID No.s 605-743; odd numbers only)

FIG. 18: shows nucleotide sequences of anti-mouse V kappa ASGPR dAbs. (Seq ID No.s 745-865; odd numbers only)

FIG. 19: shows amino acid sequences of anti-mouse Vh ASGPR dAbs. (Seq ID No.s 606-744; even numbers only)

FIG. 20: shows amino acid sequences of anti-mouse V kappa ASGPR dAbs. (Seq ID No.s 746-866; even numbers only)

FIG. 21 shows binding of ASGPR specific dAbs DOM26h-196 ( [symbol] ) and DOM26h-196-61

( [symbol] )to human (His)₆-ASGPR H1. Biotinylated (His)₆-ASGPR H1 was immobilised on a Biacore streptavidin chip surface and 62 nM dAb passed over at a flow rate of 40 μl.min⁻¹. Sensorgram illustrates that DOM26h-196-61 binds to human (His)₆-ASGPR H1 antigen with higher affinity than that of the DOM26h-196 parental clone.

FIG. 22 shows 4-12% Bis-Tris gel loaded with 2 μg of Ni-NTA purified human (His)₆-ASGPR H1 stalk domain (lane 2), human (His)₆-ASGPR H1 stalk domain treated with PNGase F (lane 3), human (His)₆-ASGPR H1 lectin domain (lane4), human (His)₆-ASGPR H1 lectin domain treated with PNGase F (lane 5). 10 μl Novex Sharp prestained molecular weight standards (Invitrogen) were loaded in lane 1 and molecular masses (in kilodaltons) of individual marker bands are given to the left of lane 1. Gel was stained with lx SureBlue. Gel shows that stalk domain is extensively glycosylated as the protein only runs at the expected molecular mass following treatment with PNGase F, whereas lectin domain runs at the expected molecular mass in the presence or absence of PNGase F digestion.

FIG. 23 shows binding of ASGPR specific dAb DOM26h-196-61 to biotinylated (His)₆-human ASGPR H1 lectin domain residues cysteine 154-leucine 291 [symbol] ), (His)₆-mouse ASGPR H1 full extracellular domain residues serine 60-asparagine 284 ( [symbol] ) and (His)₆-human ASGPR H1 stalk domain residues glutamine 62-cysteine 153 ( [symbol] ) Biotinylated antigens were immobilised on a Biacore streptavidin chip surface and dAb passed over at a concentration of 60 nM and flow rate of 40 μl.min⁻¹. Sensorgram illustrates that DOM26h-196-61 binds to human ASGPR H1 lectin domain and mouse ASGPR H1 extracellular domain but not human ASGPR H1 stalk domain.

FIG. 24 shows localisation of ¹¹¹In labelled dAbs in balb/c mice at 3 hours post injection. Following intravenous dosing of 12 MBq of radiolabelled dAb via tail vein injection mice were imaged using a nanospect camera. Images show that at 3 hours signal is observed in kidney and bladder with all dAb molecules, whereas liver localisation is only observed with anti ASGPR V_{H} dAb DOM26h-196-61 and anti ASGPR V₇₈ , dAb DOM26h-161-84.

FIG. 25 a & b shows biodistribution of ¹¹¹In labelled dAbs 3 hours after dosing intravenously in balb/c mice via the tail vein. Approximately 0.5 MBq radiolabelled dAb was injected in each case. Results show accumulation of radiolabelled ASGPR dAb in mouse liver is considerably higher than that observed with either V₇₈/V_{H} dummy 2 dAbs.

As used herein, “interferon activity” refers to a molecule which, as determined suing the B16-Blue assay (Invirogen) performed as described herein (Example 12), has at least 10, 15, 20, 25, 30, 35, 40, 45 or even 50% of the amount of interferon activity of an equivalent amount of recombinant mouse interferon alpha (e.g. from PBL Biomedical Laboratories).

FIG. 26 shows 4-12% Bis-Tris gel loaded with 2 μg per lane of protein L purified mIFNa2-dAb fusions reduced with 10 mM DTT. Lane designations as follows:

mIFNa2-Vκ dummy (lane 2)

mIFNa2-V_{H} dummy 2 (lane 3)

mIFNa2-DOM26h-161-84 (lane 4)

mIFNa2-DOM26h-196-61 (lane 5)

10 μl Novex Sharp prestained molecular weight standards (Invitrogen) were loaded in lane 1 and molecular masses (in kilodaltons) of individual marker bands are given to the left of lane 1. Gel was stained with 1× SureBlue. Gel illustrates that mouse IFNa2-dAb fusions migrate close to the expected molecular mass of approximately 33 KDa.

FIG. 27 shows activity of mouse IFN-dAb fusions (+/−DOTA conjugation) in B16 mouse IFNα/β reporter cell line. B16 cells were incubated with the indicated concentrations of mouse IFN-alpha standard or mouse IFN-dAb fusion protein and interferon activity assayed by measuring the level of reporter gene expression which is directly proportional to absorbance measured at 640 nm. Top panel shows results obtained with mouse IFNa2-V_{H} dummy 2 fusion protein, bottom panel shows results obtained with mouse IFNa2-DOM26h-196-61 fusion protein. Symbols denote the following:

▴=mouse IFNa2-dAb fusion

▪=mouse IFNa2-dAb fusion conjugated to NHS:DOTA

▾=mouse IFN-alpha standard

FIG. 28 shows binding of mouse IFNa2-dAb fusions to biotinylated (His)₆-human ASGPR H1 lectin domain and (His)₆-mouse ASGPR H1 coated on the surface of a BlAcore streptavidin chip. Fusion proteins were passed over the chip surface at a concentration of 1 μM and a flow rate of 40 μl.min⁻¹.

Top panel shows binding of mouse IFNa2-DOM36h-196-61 fusion protein ( [symbol] ) and mouse IFNa2-V_{H} dummy 2 fusion protein ( [symbol] ) to (His)₆-human ASGPR H1 lectin domain. Bottom panel shows binding of mouse IFNa2-DOM36h-196-61 fusion protein ( [symbol] )and mouse IFNa2-V_{H} dummy 2 fusion protein ( [symbol] ) to (His)₆-mouse ASGPR H1.

FIG. 29 shows localisation of ¹¹¹In labelled mouse IFNa2-dAb fusions in balb/c mice at 3 hours post injection. Following intravenous dosing of 12 MBq of radiolabelled dAb via tail vein injection mice were imaged using a nanospect camera. Images show that at 3 hours signal is observed in liver, kidney and bladder with mouse IFNa2-V_{H} dummy 2 and mouse IFNa2-DOM26h-196-61 fusion proteins, however the liver appears brighter in the image in the right hand panel, indicating a greater level of liver uptake of mouse IFNa2-DOM26h-196-61 compared to mouse IFNa2-V_{H} dummy 2.

FIG. 30 shows biodistribution of ¹¹¹In labelled mouse IFNa2-dAb fusion protein 3 hours after dosing intravenously in balb/c mice via the tail vein. Approximately 0.5 MBq radiolabelled dAb was injected in each case. Results show both mouse IFNa2-DOM26h-196-61 (black bars) and mouse IFNa2-V_{H} dummy 2 (grey bars) accumulate in the liver and kidney, however the liver/kidney ratio of mouse IFNa2-DOM26h-196-61 is approximately 2.2 fold higher than that of mouse IFNa2-V_{H} dummy 2, indicative of successful liver targeting of mouse IFNa2 by genetic fusion to ASGPR dAb DOM26h-196-61.

FIGS. 31 and 32 show the amino acid (Seq ID No.s 868-880; even numbers only) and nucleotide (Seq ID No.s 867-879; odd numbers only) sequences respectively of the various affinity-matured DOM26h clones.

### DETAILED DESCRIPTION OF THE INVENTION

Within this specification the invention has been described, with reference to embodiments, in a way which enables a clear and concise specification to be written. It is intended and should be appreciated that embodiments may be variously combined or separated without departing from the invention. For the avoidance of doubt, it is expressly stated that features of the invention disclosed herein in relation to one embodiment of the invention may be combined with any one or more other features of the invention disclosed in relation to other embodiments of the invention, unless the context dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods.

The term “analogue” as used herein referring to a polypeptide means a modified peptide wherein one or more amino acid residues of the peptide have been substituted by other amino acid residues and/or wherein one or more amino acid residues have been deleted from the peptide and/or wherein one or more amino acid residues have been added to the peptide. Such addition or deletion of amino acid residues can take place at the N-terminal of the peptide and/or at the C-terminal of the peptide or they can be within the peptide.

The term ASGPR receptor as used herein refers to the Asialoglycoprotein receptor present on the surface of hepatocytes (see Meier et al., J. Mol. Biol., 2000, 300, pp 857-865), and more specifically to the H1 subunit thereof.

As used herein “fragment,” when used in reference to a polypeptide, is a polypeptide having an amino acid sequence that is the same as part but not all of the amino acid sequence of the entire naturally occurring polypeptide. Fragments may be “free-standing” or comprised within a larger polypeptide of which they form a part or region as a single continuous region in a single larger polypeptide.

As used herein, “peptide” refers to about two to about 50 amino acids that are joined together via peptide bonds.

As used herein, “polypeptide” refers to at least about 50 amino acids that are joined together by peptide bonds. Polypeptides generally comprise tertiary structure and fold into functional domains.

As used herein, “display system” refers to a system in which a collection of polypeptides or peptides are accessible for selection based upon a desired characteristic, such as a physical, chemical or functional characteristic. The display system can be a suitable repertoire of polypeptides or peptides (e.g., in a solution, immobilized on a suitable support). The display system can also be a system that employs a cellular expression system (e.g., expression of a library of nucleic acids in, e.g., transformed, infected, transfected or transduced cells and display of the encoded polypeptides on the surface of the cells) or an acellular expression system (e.g., emulsion compartmentalization and display). Exemplary display systems link the coding function of a nucleic acid and physical, chemical and/or functional characteristics of a polypeptide or peptide encoded by the nucleic acid. When such a display system is employed, polypeptides or peptides that have a desired physical, chemical and/or functional characteristic can be selected and a nucleic acid encoding the selected polypeptide or peptide can be readily isolated or recovered. A number of display systems that link the coding function of a nucleic acid and physical, chemical and/or functional characteristics of a polypeptide or peptide are known in the art, for example, bacteriophage display (phage display, for example phagemid display), ribosome display, emulsion compartmentalization and display, yeast display, puromycin display, bacterial display, display on plasmid, covalent display and the like. (See, e.g., EP 0436597 (Dyax), U.S. Pat. No. 6,172,197 (McCafferty et al.), U.S. Pat. No. 6,489,103 (Griffiths et al.).)

As used herein, “functional” describes a polypeptide or peptide that has biological activity, such as specific binding activity. For example, the term “functional polypeptide” includes an antibody or antigen-binding fragment thereof that binds a target antigen through its antigen-binding site.

As used herein, “target ligand” refers to a ligand which is specifically or selectively bound by a polypeptide or peptide. For example, when a polypeptide is an antibody or antigen-binding fragment thereof, the target ligand can be any desired antigen or epitope. Binding to the target antigen is dependent upon the polypeptide or peptide being functional.

As used herein an antibody refers to IgG, IgM, IgA, IgD or IgE or a fragment (such as a Fab , F(ab′)₂, Fv, disulphide linked Fv, scFv, closed conformation multispecific antibody, disulphide-linked scFv, diabody) whether derived from any species naturally producing an antibody, or created by recombinant DNA technology; whether isolated from serum, B-cells, hybridomas, transfectomas, yeast or bacteria.

As used herein, “antibody format” refers to any suitable polypeptide structure in which one or more antibody variable domains can be incorporated so as to confer binding specificity for antigen on the structure. A variety of suitable antibody formats are known in the art, such as, chimeric antibodies, humanized antibodies, human antibodies, single chain antibodies, bispecific antibodies, antibody heavy chains, antibody light chains, homodimers and heterodimers of antibody heavy chains and/or light chains, antigen-binding fragments of any of the foregoing (e.g., a Fv fragment (e.g., single chain Fv (scFv), a disulfide bonded Fv), a Fab fragment, a Fab′ fragment, a F(ab′)₂ fragment), a single antibody variable domain (e.g., a dAb, V_{H}, V_{HH}, V_{L}), and modified versions of any of the foregoing (e.g., modified by the covalent attachment of polyethylene glycol or other suitable polymer or a humanized V_{HH}).

The phrase “immunoglobulin single variable domain” refers to an antibody variable domain (V_{H}, V_{HH}, V_{L}) that specifically binds an antigen or epitope independently of other V regions or domains. An immunoglobulin single variable domain can be present in a format (e.g., homo- or hetero-multimer) with other variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” as the term is used herein. A “single immunoglobulin variable domain” is the same as an “immunoglobulin single variable domain” as the term is used herein. A “single antibody variable domain” is the same as an “immunoglobulin single variable domain” as the term is used herein. An immunoglobulin single variable domain is in one embodiment a human antibody variable domain, but also includes single antibody variable domains from other species such as rodent (for example, as disclosed in WO 00/29004, the contents of which are incorporated herein by reference in their entirety), nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. The V_{HH} may be humanized.

A “domain” is a folded protein structure which has tertiary structure independent of the rest of the protein. Generally, domains are responsible for discrete functional properties of proteins, and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

The term “library” refers to a mixture of heterogeneous polypeptides or nucleic acids. The library is composed of members, each of which has a single polypeptide or nucleic acid sequence. To this extent, “library” is synonymous with “repertoire.” Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. In one embodiment, each individual organism or cell contains only one or a limited number of library members. In one embodiment, the nucleic acids are incorporated into expression vectors, in order to allow expression of the polypeptides encoded by the nucleic acids. In an aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of diverse polypeptides.

As used herein, the term “dose” refers to the quantity of fusion or conjugate administered to a subject all at one time (unit dose), or in two or more administrations over a defined time interval. For example, dose can refer to the quantity of fusion or conjugate administered to a subject over the course of one day (24 hours) (daily dose), two days, one week, two weeks, three weeks or one or more months (e.g., by a single administration, or by two or more administrations). The interval between doses can be any desired amount of time.

As used herein, “interferon activity” refers to a molecule which, as determined using the B16-Blue assay (Invivogen) performed as described herein (Example 12), has at least 10, 15, 20, 25, 30, 35, 40, 45 or even 50% of the amount of activity of an equal amount of recombinant mouse interferon alpha (e.g. from PBL Biomedical Laboratories).

The phrase, “half-life,” refers to the time taken for the serum or plasma concentration of the fusion or conjugate to reduce by 50%, in vivo, for example due to degradation and/or clearance or sequestration by natural mechanisms. The compositions of the invention are stabilized in vivo and their half-life increased by binding to serum albumin molecules e.g. human serum albumin (HSA) which resist degradation and/or clearance or sequestration. These serum albumin molecules are naturally occurring proteins which themselves have a long half-life in vivo. The half-life of a molecule is increased if its functional activity persists, in vivo, for a longer period than a similar molecule which is not specific for the half-life increasing molecule.

As used herein, “hydrodynamic size” refers to the apparent size of a molecule (e.g., a protein molecule, ligand) based on the diffusion of the molecule through an aqueous solution. The diffusion, or motion of a protein through solution can be processed to derive an apparent size of the protein, where the size is given by the “Stokes radius” or “hydrodynamic radius” of the protein particle. The “hydrodynamic size” of a protein depends on both mass and shape (conformation), such that two proteins having the same molecular mass may have differing hydrodynamic sizes based on the overall conformation of the protein.

Calculations of “homology” or “identity” or “similarity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In an embodiment, the length of a reference sequence aligned for comparison purposes is at least 30%, or at least 40%, or at least 50%, or at least 60%, or at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “homology” is equivalent to amino acid or nucleic acid “identity”). The percent identity between the two sequences is a function of the number of identical positions shared by the sequences, taking into account the number of gaps, and the length of each gap, which need to be introduced for optimal alignment of the two sequences. Amino acid and nucleotide sequence alignments and homology, similarity or identity, as defined herein may be prepared and determined using the algorithm BLAST 2 Sequences, using default parameters (Tatusova, T. A. et al., FEMS Microbiol Lett, 174:187 -188 (1999).

### Nucleic Acids, Host Cells:

The invention relates to isolated and/or recombinant nucleic acids encoding the compositions of the invention that are described herein.

Nucleic acids referred to herein as “isolated” are nucleic acids which have been separated away from other material (e.g., other nucleic acids such as genomic DNA, cDNA and/or RNA) in its original environment (e.g., in cells or in a mixture of nucleic acids such as a library). An isolated nucleic acid can be isolated as part of a vector (e.g., a plasmid).

Nucleic acids referred to herein as “recombinant” are nucleic acids which have been produced by recombinant DNA methodology, including methods which rely upon artificial recombination, such as cloning into a vector or chromosome using, for example, restriction enzymes, homologous recombination, viruses and the like, and nucleic acids prepared using the polymerase chain reaction (PCR).

The invention also relates to a recombinant host cell e.g. mammalian or microbial, which comprises a (one or more) recombinant nucleic acid or expression construct comprising nucleic acid(s) encoding a composition e.g. fusion, of the invention as described herein. There is also provided a method of preparing a composition, e.g. fusion, of the invention as described herein, comprising maintaining a recombinant host cell e.g.mammalian or microbial, of the invention under conditions appropriate for expression of the fusion polypeptide. The method can further comprise the step of isolating or recovering the fusion, if desired.

For example, a nucleic acid molecule (i.e., one or more nucleic acid molecules) encoding a composition of the invention e.g. a liver targeting composition of the invention, or an expression construct (i.e., one or more constructs) comprising such nucleic acid molecule(s), can be introduced into a suitable host cell to create a recombinant host cell using any method appropriate to the host cell selected (e.g., transformation, transfection, electroporation, infection), such that the nucleic acid molecule(s) are operably linked to one or more expression control elements (e.g., in a vector, in a construct created by processes in the cell, integrated into the host cell genome). The resulting recombinant host cell can be maintained under conditions suitable for expression (e.g., in the presence of an inducer, in a suitable non-human animal, in suitable culture media supplemented with appropriate salts, growth factors, antibiotics, nutritional supplements, etc.), whereby the encoded peptide or polypeptide is produced. If desired, the encoded peptide or polypeptide can be isolated or recovered (e.g., from the animal, the host cell, medium, milk). This process encompasses expression in a host cell of a transgenic animal (see, e.g., WO 92/03918, GenPharm International), especially a transgenic non-human animal.

The compositions, e.g. fusion polypeptides, of the invention described herein can also be produced in a suitable in vitro expression system, e.g. by chemical synthesis or by any other suitable method.

As described and exemplified herein, compositions e.g. fusions and conjugates of the invention, generally bind ASGPR with high affinity.

For example, the fusions or conjugates can bind human ASGPR with an affinity (KD; KD=K_{off}(kd)/Kₒₙ(ka) [as determined by surface plasmon resonance] of about 5 micromolar to about 1 pM , e.g. about 10 nM to about 1 pM e.g. about 1 nM to about 1 pM.

The compositions e.g. dAbs and/or liver targeting compositions, of the invention can be expressed in E. coli or in Pichia species (e.g., P. pastoris). In one embodiment, the a liver targeting fusion is secreted in E. coli or in Pichia species (e.g., P. pastoris); or in mammalian cell culture (e.g. CHO, or HEK 293 cells). Although, the fusions or conjugates described herein can be secretable when expressed in E. coli or in Pichia species or mammalian cells they can be produced using any suitable method, such as synthetic chemical methods or biological production methods that do not employ E. coli or Pichia species.

Generally, the compositions of the invention will be utilised in purified form together with pharmacologically or physiologically appropriate carriers. Typically, these carriers can include aqueous or alcoholic/aqueous solutions, emulsions or suspensions, any including saline and/or buffered media. Parenteral vehicles can include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition). A variety of suitable formulations can be used, including extended release formulations.

The route of administration of pharmaceutical compositions according to the invention may be any of those commonly known to those of ordinary skill in the art. For therapy, compositions of the invention can be administered to any patient in accordance with standard techniques.

The administration can be by any appropriate mode, including by subcutaneous injection, parenterally, intravenously, intramuscularly, intraperitoneally, orally, transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician. Administration can be local or systemic as indicated.

The compositions of this invention can be lyophilised for storage and reconstituted in a suitable carrier prior to use. This technique has been shown to be effective with conventional immunoglobulins and art-known lyophilisation and reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate.

Treatment or therapy performed using the compositions described herein is considered “effective” if one or more symptoms or signs are reduced or alleviated (e.g., by at least 10% or at least one point on a clinical assessment scale), relative to such symptoms present before treatment, or relative to such symptoms in an individual (human or model animal) not treated with such composition or other suitable control. Symptoms will obviously vary depending upon the precise nature of the disease or disorder targeted, but can be measured by an ordinarily skilled clinician or technician.

Similarly, prophylaxis performed using a composition as described herein is “effective” if the onset or severity of one or more symptoms or signs is delayed, reduced or abolished relative to such symptoms in a similar individual (human or animal model) not treated with the composition.

The compositions of the present invention may be administered in conjunction with other therapeutic or active agents e.g. other polypeptides or peptides or small molecules. These further agents can include various drugs, such as for example ribavirin.

The compositions of the invention can be administered and/or formulated together with one or more additional therapeutic or active agents. When a composition of the invention is administered with an additional therapeutic agent, e.g. the liver targeting composition (e.g. a fusion or conjugate) can be administered before, simultaneously, with, or subsequent to administration of the additional agent e.g. ribavirin. Generally, the composition of the invention and the additional agent are administered in a manner that provides an overlap of therapeutic effect.

Compositions of the invention comprising dAbs, provide several further advantages. The Domain antibody component is very stable, is small relative to antibodies and other antigen-binding fragments of antibodies, can be produced in high yields by expression in E. coli or yeast (e.g., Pichia pastoris). Accordingly, compositions of the invention that comprise the dAb that binds hepatocytes (e.g. the ASGPR receptor on hepatocytes) can be produced more easily than therapeutics that are generally produced in mammalian cells (e.g., human, humanized or chimeric antibodies) and dAbs that are not immunogenic can be used (e.g., a human dAb can be used for treating or diagnosing disease in humans).

Additionally, the compositions described herein can have an enhanced safety profile and fewer side effects than the therapeutic molecule(s) e.g. interferon alone alone as a result of the specific targeting to the liver. Similarly, administration of the compositions of the invention can have reduced toxicity toward particular organs and/or bodily tissues outside of the liver than administration of the therapeutic molecule(s) alone and can also have improved efficacy e.g. as a result of specifically directing the therapeutic molecule to the liver at effective doses for systemic delivery, when administration of such molecules might otherwise be toxic to other organs and tissues

### EXAMPLES:

### Example 1

Cloning and Expression of Human and Mouse Asialoglycoprotein H1 Receptor Subunits

Full length human and mouse asialoglycoprotein receptor H1 subunit (ASGPR H1) cDNA was custom synthesised by DNA2.0 (Mealo Park Calif., USA). DNA encoding the extracellular domain (Q62-L291 for human and S60-N284 for mouse) with an N-terminal (His)₆ tag was amplified by PCR using primers DLT007 and DLT008 (human) or DLT009 and DLT010 (mouse). Sequences are shown in Table 1 below.

TABLE 1

| DLT007 | GGATCCACCGGCCATCATCATCATCATCACCAGAACTCCC | Human (His)₆ ASGPR |
|---|---|---|
| | AACTCCAGGAA (Seq ID No.1) | H1 5′ primer |
| DLT008 | AAGCTTTTATTACAGGAGTGGAGGCTCTTGTGA | Human (His)₆ |
| | (Seq ID No. 2) | ASGPR H1 3′ primer |
| DLT0090 | GGATCCACCGGCCATCATCATCATCATCACAGTCAAAATT | Mouse (His)₆ |
| | CCCAATTGCGC (Seq ID No. 3) | ASGPR H1 5′ primer |
| DLT010 | AAGCTTTTATTAATTGGCTTTGTCCAGCTTTGT | Mouse (His)₆ |
| | (Seq ID No. 4) | ASGPR H1 3′ primer |

PCR fragments were inserted into holding vector pCR-Zero Blunt (Invitrogen) by Topoisomerase cloning and sequenced to obtain error-free clones using M13 forward and M13 reverse primers. (His)₆-ASGPR H1 encoding DNA was obtained by gel purification following BamHI/HindIII digestion of pCR-Zero Blunt containing the insert and inserts ligated into the corresponding sites in pDOM50, a mammalian expression vector which is a pTT5 derivative with an N-terminal V-J2-C mouse IgG secretory leader sequence to facilitate expression into the cell media.

Leader sequence (amino acid):
(Seq ID No. 5)
METDTLLLWVLLLWVPGSTG
Leader sequence (nucleotide):
(Seq ID No. 6)
ATGGAGACCGACACCCTGCTGCTGTGGGTGCTGCTGCTGTGGGTGCCCGG
ATCCACCGGGC

Plasmid DNA was prepared using QIAfilter megaprep (Qiagen). 1 μg DNA/ml was transfected with 293-Fectin into HEK293E cells and grown in serum free media. The protein is expressed in culture for 5 days and purified from culture supernatant using Ni-NTA resin and eluted with PBS+0.5 M Imidazole. The proteins were buffer exchanged into PBS.

N-termini of the receptor subunits were determined by Edman sequencing. The N-terminus of the Human (His)₆-ASGPR H1 subunit was identified as:

HHHHHHQNSQLQEEL (Seq ID No. 7) with an additional sequence identified as: LRGLREFTS (Seq ID No. 8) corresponding to a cleavage product. However the sequence corresponding to the intact receptor was present in an approximately 5 fold molar excess compared to that of the cleavage product. The N-terminus of Mouse (His)₆-ASGPR H1 subunit was identified as:

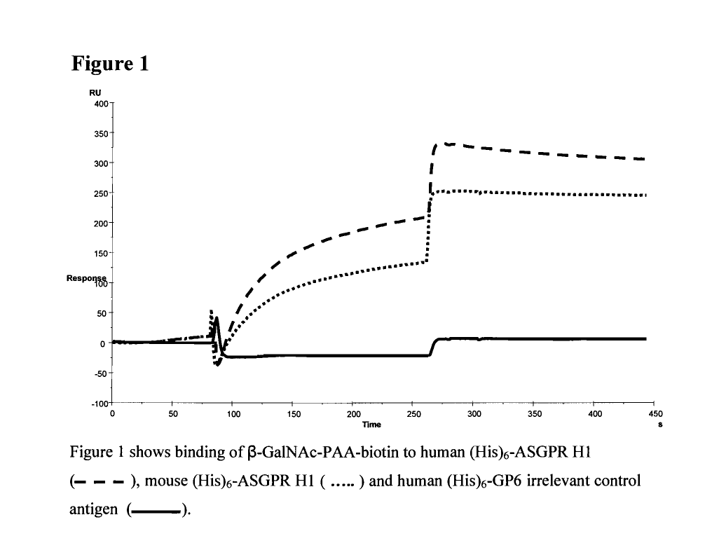

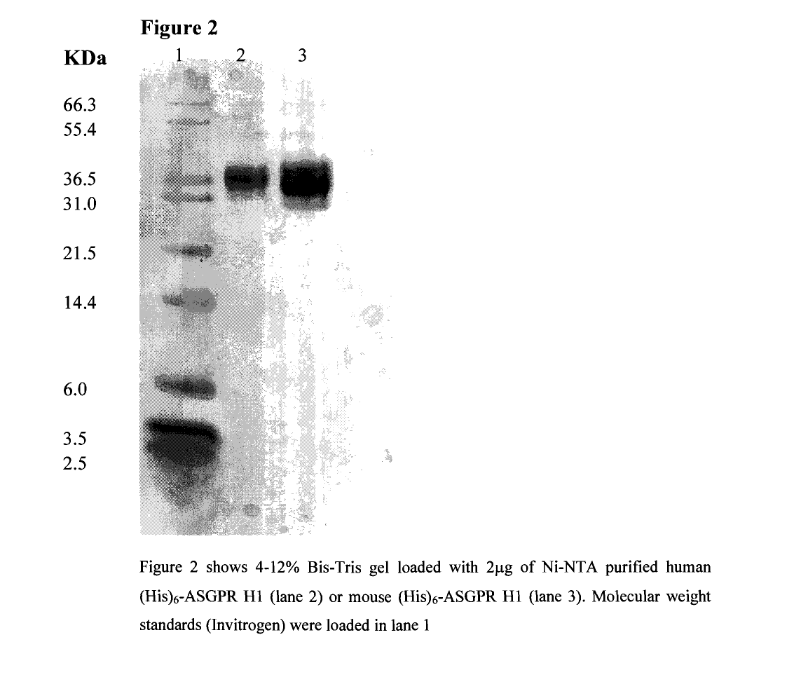

HHHHHHSQNXQLRED (Seq ID No. 9) with no additional sequences identified. To assay for potential ligand binding activity receptor subunits were immobilised on a biacore CM5 chip surface and binding to the synthetic ligand P-GalNAc-PAA-biotin (Glycotech) was analysed (FIG. 1). Purity of HEK293E receptor eluted from Ni-NTA was also analysed by non-reducing SDS-PAGE (FIG. 2). SDS-PAGE analysis shows that human and mouse (His)₆-ASGPR HI subunits migrate close to the expected molecular mass based on amino acid sequence (27.2 KDa for human and 26.5 KDa for mouse. More than one species migrating close to the expected molecular mass was observed in both human and mouse (His)₆-ASGPR HI samples, typical of glycosylated protein samples.

Sequences:

(His)₆-Human ASGPR H1
(Seq ID No. 10)
HHHHHHQNSQLQEELRGLRETFSNFTASTEAQVKGLSTQGGNVGRKMKSL
SEQLEKQQKDLSEDHSSLLLHVKQFVSDLRSLSCQMAALQGNGSERTCCP
VNWVEHERSCYWFSRSGKAWADADNYCRLEDAHLVVVTSWEEQKFVQHHI
GPVNTWMGLHDQNGPWKWVDGTDYETGFKNWRPEQPDDWYGHGLGGGEDC
AHFTDDGRWNDDVCQRPYRWVCETELDKASQEPPLL
(Seq ID No. 11)
CATCATCATCATCATCACCAGAACTCCCAACTCCAGGAAGAACTTCGAGG
ACTGAGGGAGACTTTCTCCAATTTCACCGCAAGCACGGAGGCTCAAGTGA
AGGGCCTCAGCACCCAGGGCGGGAATGTGGGCAGGAAAATGAAATCCCTG
GAGAGCCAGCTCGAAAAGCAGCAGAAAGATCTGTCCGAGGACCACTCAGT
CCTGTTGTTGCACGTGAAACAGTTTGTTTCCGACCTTAGGAGTCTTTCTT
GCCAAATGGCCGCCCTCCAGGGAAACGGGTCCGAGAGAACTTGCTGCCCC
GTCAATTGGGTGGAGCACGAGCGGTCTTGTTATTGGTTTAGCCGAAGCGG
AAAAGCCTGGGCCGATGCAGATAACTACTGCCGGCTTGAGGACGCCCATC
TGGTCGTGGTGACCAGTTGGGAGGAACAGAAATTCGTACAGCATCATATC
GGGCCTGTTAACACATGGATGGGCCTTCATGACCAGAATGGTCCTTGGAA
GTGGGTTGACGGAACCGATTACGAAACCGGATTCAAGAACTGGCGGCCTG
AACAGCCAGACGACTGGTATGGACACGGCCTCGGAGGCGGGGAGGACTGC
GCGCATTTCACAGACGATGGCCGGTGGAATGATGATGTGTGCCAAAGGCC
TTACAGATGGGTCTGCGAGACAGAGCTGGATAAGGCTTCACAAGAGCCTC
CACTCCTG
(His)₆-Mouse ASGPR H1
(Seq ID No. 12)
HHHHHHSQNSQLREDLLALRQNFSNLTVSTEDQVKALSTQGSSVGRKMKL
VESKLEKQQKDLTEDHSSLLLHVKQLVSDVRSLSCQMAAFRGNGSERTCC
WPINVEYEGSCYWFSSSVRPWTEADKYCQLENAHLVVVTSRDEQNFLQRH
LMGPNTWIGLTDQNGPWKWVDGTDYETGFQNWRPEQPDNWYGHGLGGGED
CAHFTTDGRWNDDVCRRPYRWVCETKLDKAN
(Seq ID No. 13)
CATCATCATCATCATCACAGTCAAAATTCCCAATTGCGCGAGGATCTGCT
CGCACTGCGACAGAACTTTAGCAACCTTACCGTGTCTACGGAAGACCAGG
TGAAGGCATTGTCAACTCAGGGGTCATCTGTGGGAAGAAAAATGAAGCTC
GTGGAGTCAAAGCTGGAGAAGCAGCAAAAGGACCTCACCGAAGACCATTC
CTCTCTCCTGCTGCACGTGAAGCAGCTGGTTTCTGACGTAAGGAGCCTGA
GCTGCCAGATGGCTGCTTTTCGAGGTAACGGCTCTGAGCGCACATGCTGT
CCTATTAATTGGGTGGAGTATGAGGGAAGTTGTTACTGGTTCTCAAGCTC
CGTGAGGCCATGGACCGAAGCTGACAAATATTGCCAGCTCGAAAATGCTC
ACCTCGTGGTAGTGACCTCTAGGGATGAGCAAAATTTCCTGCAGCGACAC
ATGGGGCCGCTTAATACCTGGATCGGGCTGACGGACCAGAACGGACCCTG
GAAGTGGGTTGACGGTACCGATTATGAAACTGGATTCCAAAACTGGCGGC
CAGAGCAGCCGGACAACTGGTATGGCCACGGCCTCGGAGGGGGCGAGGAC
TGTGCTCATTTTACAACGGATGGCCGGTGGAACGACGATGTGTGCAGAAG
GCCATATCGGTGGGTCTGCGAGACAAAGCTGGACAAAGCCAAT

### Example 2-Methods for Selecting dAbs

Domantis' 4G and 6G naïve phage libraries, phage libraries displaying antibody single variable domains expressed from the GAS 1 leader sequence (see WO2005/093074) for 4G and additionally with heat/cool preselection for 6G (see WO04/101790) were divided into four pools; pool 1 contained libraries 4VH11-13 and 6VH2, pool 2 contained libraries 4VH14-16 and 6VH3, pool 3 contained libraries 4VH17-19 and 6VH4 and pool 4 contained libraries 4K and 6K. Library aliquots were of sufficient size to allow 10-fold over representation of each library. Selections were carried out using passively coated and biotinylated human and mouse (His)₆-ASGPR H1 antigens. Selections using passively coated antigen were carried out as follows. After coating antigen on immunotubes (Nunc) in TBS supplemented with 5 mM Ca2⁺

(TBS/Ca²⁺) tubes were blocked with 2% Marvel in TBS/Ca²′ (MTBS/Ca²⁺ ). Library aliquots were incubated with antigen-coated immunotubes in MTBS/Ca²⁺ before washing tubes with TBS/Ca²⁺ . Bound phage was then eluted with 1 mg/ml Trypsin. The concentration of antigen during coating was decreased from 1 mg/ml to 40 μg/ml as the rounds progressed and the titres increased as the rounds progressed. Selections using biotinylated antigen were carried out as follows. Library aliquots were incubated with antigen in MTBS/Ca²⁺ for one hour before capture on streptavidin Dynabeads (Invitrogen) or Tosyl activated beads (Invitrogen) coated with neutravidin (Perbio), washed with 0.1% Tween-TBS/Ca²⁺ and TBS/Ca²⁺ then eluted with 1 mg/ml Trypsin. The concentrations of antigen were decreased from 100 nM to 1 nM as the rounds progressed and the titres increased as the rounds progressed. Following both types of selection eluted phage was used to infect log phase TG1 cells (Gibson, 1984) then infected cells were plated on tetracycline plates (15 μg/ml tetracycline). Cells infected with the phage were then grown up in 2xTY with tetracycline overnight at 37° C. before the phage were precipitated from the culture supernatant using PEG-NaCl and used for subsequent rounds of selection.

### Example 3

Screening Selection Outputs for Liver Cell Specific dAbs

After 3 rounds of selection, the dAb genes from each library pool were subcloned from the pDOM4 phage vector into the pDOM10 soluble expression vector. pDOM4 is a derivative of the fd phage vector in which the gene III signal peptide sequence is replaced with the yeast glycolipid anchored surface protein (GAS) signal peptide. It also contains a c-Myc tag between the leader sequence and gene III. In each case after selection a pool of phage DNA from appropriate round of selection is prepared using a QIAfilter midiprep kit (Qiagen), the DNA is digested using the restriction enzymes Sall and Notl and the enriched dAb genes are ligated into the corresponding sites in pDOM10.

The pDOM10 vector is a pUC119-based vector. Expression of proteins is driven by the LacZ promoter. A GAS1 leader sequence (see WO 2005/093074) ensures secretion of isolated, soluble dAbs into the periplasm and culture supernatant of E. coli. dAbs are cloned SalI/NotI in this vector, which appends a FLAG epitope tag at the C-terminus of the dAb.

The ligated DNA is used to transform E. coli TOP 10 cells which are then grown overnight on agar plates containing the antibiotic carbenicillin. The resulting colonies are individually assessed for antigen binding.

The antigen binding of individual dAb clones was assessed either by ELISA or on BlAcore. The ELISA assay took the following format. Human or mouse (His)₆-ASGPR H1 was coated at 1 g/ml onto a Maxisorp (NUNC) plate overnight at 4° C. The plate was then blocked with 2% Tween-TBS/Ca²⁺, followed by incubation with dAb supernatant diluted 1:1 with 0.1% Tween-TBS/Ca²⁺, followed by detection with 1:5000 anti-flag (M2)-HRP (SIGMA). All steps after blocking were carried out at room temperature. The binding of the dAb supernatant to a control antigen (human c-kit-(His)₆) was also analysed at the same time. In some cases dAb supernatants from selections using human antigen were also screened for binding to HepG2 and HeLa cells using the meso scale discovery (MSD) assay. Cells were plated using MULTI-ARRAY 96-well, SECTOR Imager High Bind Plates (Meso-scale) at a density of 1×10⁵ cells per well and left to incubate overnight at 37° C., 5% CO₂. The following day dAb anti-FLAG M2 complexes were prepared at 2× final concentration by dilution of dAb and biotinylated anti FLAG M2 monoclonal antibody (Sigma) in MSD assay buffer (1xPBS with 1 mM MgCl₂, 1 mM CaCl₂, 10% Foetal Bovine Serum and 1% BSA). dAb-anti FLAG complexes were incubated in a 1:1 molar ratio at room temperature for one hour. Cells were then washed 3× with 200 μl PBS before addition of 25 μl per well dAb-anti FLAG complex and incubation for one hour at room temperature for one hour with gentle agitation. Cells were then washed as above and 25 μl per well streptavidin-Sulfotag (Meso-scale) diluted to 1 μg/ml in assay buffer was then added. Cells were then incubated for one hour at room temperature, in the dark with gentle agitiation. Cells were then washed as above before resuspension in 150 μl per well of 1× MSD read buffer without surfactant (Meso-scale) and read on a SECTOR Imager 6000 (Meso-scale) at 620nm emission. Clones DOM26h-25, DOM26h-34, DOM26h-161, DOM26h-162, DOM26h-163, DOM26h-164, DOM26h-165, DOM26h-166 and DOM26h-167 and DOM26h-168 through to DOM26h-224 were screened in this assay.

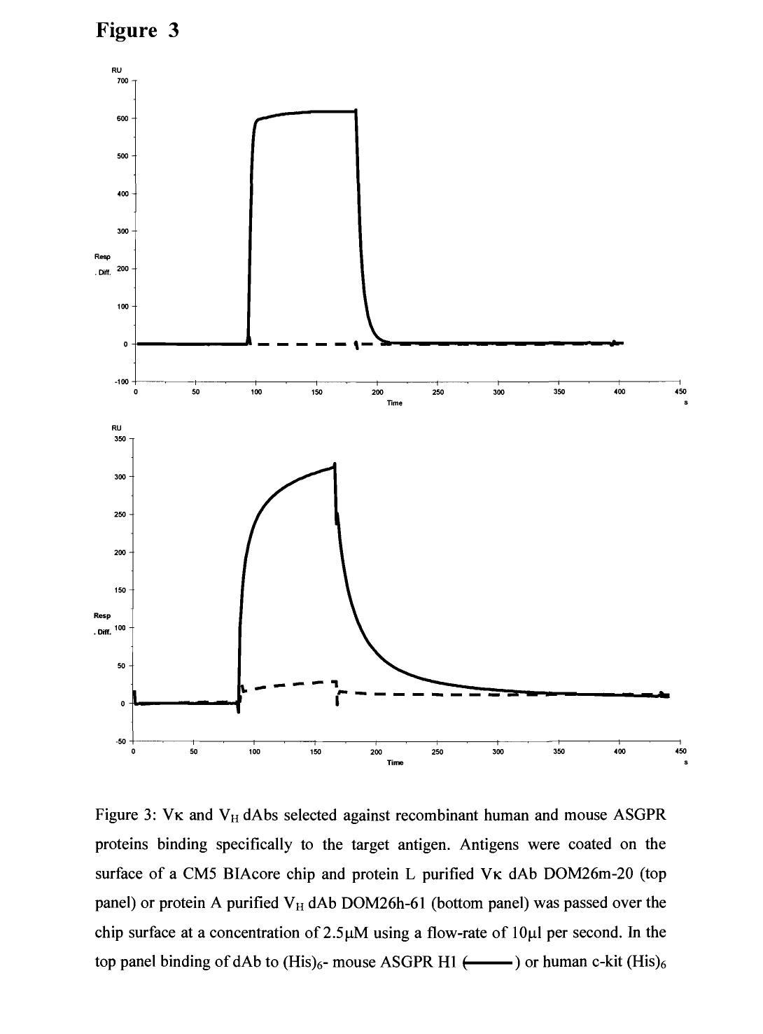

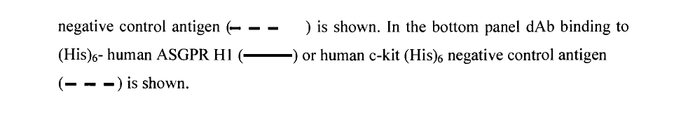

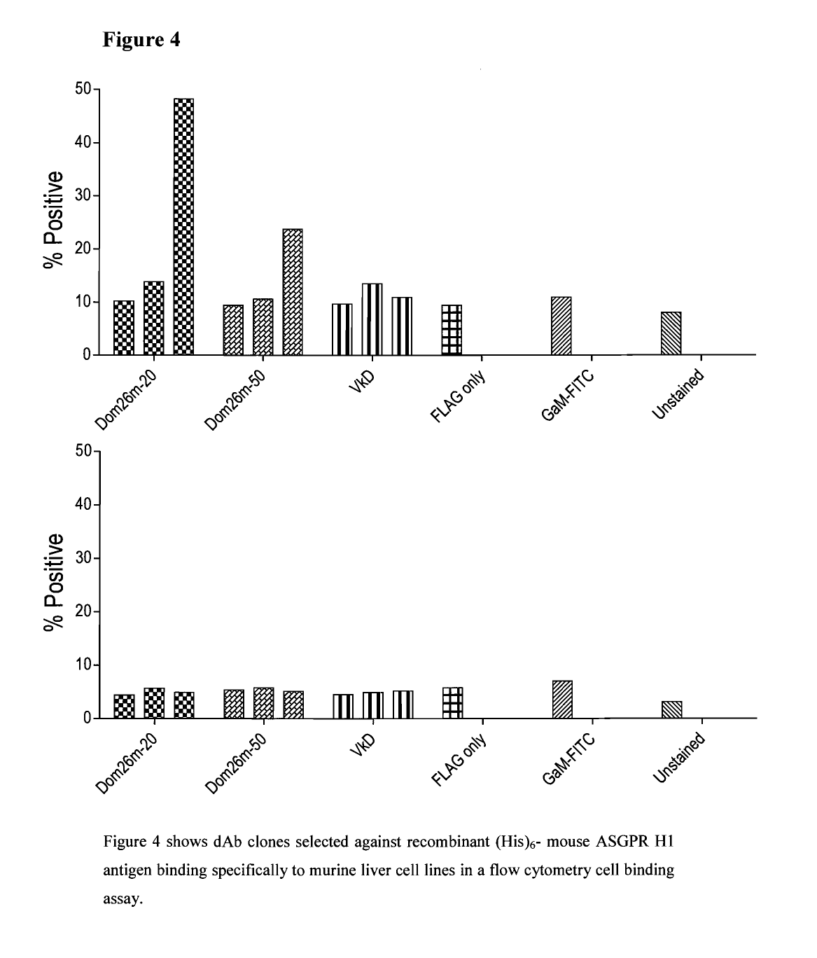

Those dAbs that showed specific binding to (His)₆ ASGPR H1 by ELISA or MSD assay were screened by BlAcore. Screening by BlAcore took place using dAb supernatant expressed as above diluted 1:2 with HBS-P BlAcore running buffer. Each dAb was then injected over a blank flow cell and a flow cell coated with human or mouse (His)₆-ASGPR H1 on a CM5 chip. Any dAb clone that showed specific binding to (His)₆-ASGPR H1 was streaked out and sequenced. All unique dAb clones were expressed in 50 ml cultures (OnEX plus carbenicillin) overnight at 37° C. and purified on protein A (V_{H} dAbs) or protein L (Vκ dAbs). Purified dAbs were passed over a CM5 BIAcore chip coated with either human or mouse (His)₆-ASGPR H1 at 20μg/ml (FIG. 3). Those dAbs that bound specifically to (His)₆-ASGPR H1 were then analysed in the flow cytometry cell binding assay (FIG. 4).

Two cell lines were used as human ASGPR positive lines (HepG2 and Hep3b) and one as a negative control human line (HeLa). Two cell lines were used as mouse ASGPR positive cell lines (Hepal cic7 and NMuLi) and one as a negative control mouse line (L929). The flow cytometry cell binding assay was carried out as follows. Cells were harvested, and washed in PBS supplemented with 5% FCS and 0.5% BSA (FACS buffer). Cells were divided between the appropriate number of wells at a concentration of 1×10⁵ cells per well and incubated for one hour at 4° C. The cells were then incubated for one hour with the appropriate concentration of dAb which had previously been cross-linked by incubation with 5 μg/ml anti-FLAG M2 (Sigma) for 30 minutes at 4° C. The cells were then washed with FACS buffer and incubated for one hour at 4° C. with Goat anti-mouse FITC (Sigma) diluted 1:100 in FACS buffer. The cells were then washed with FACs buffer and resuspended in 200 μl FACS buffer before analysis by flow cytometry (FACS Canto II, using FACS Diva software). CDR sequences (determined using the method of Kabat) of clones specific for the human liver cell line HepG2 are described in the Table 2 below:

TABLE 2

| dAb | CDR1 | CDR2 | CDR3 |
|---|---|---|---|
| DOM26h-25 | RASGDIGHALW | RGGSALQS | GQSHVRPFT |
| DOM26h-34 | QASKNIGERLV | GFASLLQS | GQYRWVPAT |
| DOM26h-61 | STYPMH | SISPSGDS | NALRFDY |
| DOM26h-99 | KPYAMH | SISSTGLS | DASRFRQPFDY |
| DOM26h-104 | PKYGMA | RIGATGSE | HRGTAHSSFFDY |
| DOM26h-110 | SANGMH | VISATGDQ | GYDRRHRKFDY |
| DOM26h-159 | ADYSMY | DISPSGSM | GLPGQNMHVGFDY |
| DOM26h-161 | RASQAIGRWLL | YAASRLQS | QQAYSLPPT |
| DOM26h-162 | RASMSIDESLW | RGGSGLQS | GQAARRPYT |
| DOM26h-163 | RASHYIGNELW | RRGSGLQS | GQARHRPYT |
| DOM26h-164 | RASSNIGRSLV | AGGSLLQS | GQYAEEPFT |
| DOM26h-166 | RASSYIGGELW | SGTSGLQS | GQAAKRPFT |
| DOM26h-165 | RASVKIGERLW | RDASLLQS | GQSWMRPYT |
| DOM26h-167 | RASSWINSDLV | AGGSLLQS | GQYLEEPYT |
| Seq ID No. s | 14-27 | 28-41 | 42-55 |

CDR sequences (determined using the method of Kabat) of clones specific for the mouse liver cell line Hepal clc7 are described in the Table 3 below:

TABLE 3

| dAb | CDR1 | CDR2 | CDR3 |
|---|---|---|---|
| DOM26m-7 | DDYEMG | LISAQGRV | NSPSYLLNFDY |
| DOM26m-20 | RASKYIGSDLY | GGGSRLQS | GQKWARPLT |
| DOM26m-29 | EDSGMI | GIASEGST | SGLSFDY |
| DOM26m-33 | AKYDMI | GINHSGSR | SGSSFDY |
| DOM26m-50 | RASISIYEHLN | WDSSGLQS | VQHHSHPPT |
| DOM26m-52 | REHPMS | SISKHGSE | SVREFDY |
| DOM26m-54 | RASLNIDTDLV | AGWSGLQS | GQFAREPFT |
| DOM26m-58 | RASQPIRNALT | YRTSHLQS | QQTWTMPLT |
| Seq ID No. s | 56-63 | 64-71 | 72-79 |

Lead dAbs were analysed by size exclusion chromatography with multi-angle LASER light scattering (SEC-MALLS) to determine whether they were monomeric or formed higher order oligomers in solution. SEC-MALLS was carried out as follows. Proteins (at a concentration of 1 mg/mL in Dulbecco's PBS or 0.1 M Tris-Glycine, pH 8.0) were separated according to their hydrodynamic properties by size exclusion chromatography (column: TSK3000; S200). Following separation, the propensity of the protein to scatter light is measured using a multi-angle LASER light scattering (MALLS) detector. The intensity of the scattered light while protein passes through the detector is measured as a function of angle. This measurement taken together with the protein concentration determined using the refractive index (RI) detector allows calculation of the molar mass using appropriate equations (integral part of the analysis software Astra v.5.3.4.12). Results are shown in Table 4 below.

TABLE 4

| | Mean Molar mass | | |
|---|---|---|---|
| Name | over main peak | | In-solution state |
| DOM26m-7 | 14.5 | kDa | monomer (65%) |
| | 28 | kDa | dimer (25%) |
| | 49 | kDa | tetramer (10%) |
| DOM26m-20 | 13 | kDa | monomer (100%) |
| *DOM26m-29 | ? | kDa | monomer/dimer (80%) |
| | 42 | kDa | trimer/tetramer (20% |
| DOM26m-33 | 15.6 | kDa | monomer (90%) |
| DOM26m-50 | 12 | kDa | monomer (100%) |
| DOM26m-52 | 29 | kDa | dimer (70%) |
| DOM26m-54 | Not determined | | Not determined |
| | (protein failed to | | (protein failed to elute) |
| | elute) | | |
| DOM26m-58 | 14 | kDa | monomer (100%) |
| DOM26h-25 | 13.8 | kDa | Monomer |
| DOM26h-34 | 12.6 | kDa | Monomer |
| DOM26h-61 | 31 | kDa | dimer (45%) |
| | 41 | kDa | tri/tetramer (35%) |
| | 100 | kDa | octamer (10%) |
| | HMWS | | soluble aggregate (5%) |
| DOM26h-99 | 22.2 | kDa | dimer (95%) |
| | 7 | kDa | contaminant(5%) |
| DOM26h-104 | 17 | kDa | monomer/dimer (80%) |
| DOM26h-110 | 20 | kDa | monomer/dimer (90%) |
| DOM26h-159 | 17.7 | kDa | monomer/dimer (90%) |
| DOM26h-161 | 12.6 | kDa | Monomer |
| DOM26h-162 | 12.3 | kDa | Monomer |
| DOM26h-163 | 18 | kDa | monomer/dimmer |
| DOM26h-164 | 17 | kDa | monomer |
| DOM26h-165 | 13.2 | kDa | Monomer |
| DOM26h-166 | 12.6 | kDa | Monomer |
| DOM26h-167 | 18 | kDa | Monomer |

*= main peak elutes at the buffer front, hence no Mw determination was possible

Lead dAbs were also analysed by differential scanning calorimetry (DSC) to determine the apparent melting temperature. DSC was carried out as follows. Protein was heated at a constant rate of 180° C./hrs (at 1 mg/mL in PBS) and a detectable heat change associated with thermal denaturation measured. The transition midpoint (appTm) is determined, which is described as the temperature where 50% of the protein is in its native conformation and the other 50% is denatured. Here, DSC determined the apparent transition midpoint (appTm) as most of the proteins examined do not fully refold. The higher the Tm, the more stable the molecule. The software package used was OriginR v7.0383. Results are shown in Table 5 below.

TABLE 5

| | App Tm | App Tm |
|---|---|---|
| Name | 1/° C. | 2/° C. |
| DOM26m-7 | 62.0 | 63.7 |
| DOM26m-20 | 63.3 | 63.2 |
| DOM26m-29 | 61.4 | — |
| DOM26m-33 | 60.9 | 60.8 |
| DOM26m-50 | 72.4 | — |
| DOM26m-52 | 61.0 | 64.9 |
| DOM26m-54 | 62.2 | 62.2 |
| DOM26m-58 | 62.9 | 62.7 |
| DOM26h-25 | 60.5 | 61.7 |
| DOM26h-34 | 57.1 | 60.2 |
| DOM26h-61 | 61.7 | 66.6 |
| DOM26h-99 | 57.0 | 60.0 |
| DOM26h-104 | 60.0 | 64.0 |
| DOM26h-110 | 57.8 | 59.6 |
| DOM26h-159 | 62.7 | 65.4 |
| DOM26h-161 | 64.9 | — |
| DOM26h-162 | 58.2 | 67.2 |
| DOM26h-163 | 58.2 | 66.6 |
| DOM26h-164 | 55.1 | 73.3 |
| DOM26h-165 | 64.3 | — |
| DOM26h-166 | 62.7 | — |
| DOM26h-167 | 63.4 | — |

In some cases App Tm 2 could not be determined due to insufficient refolding of protein after determination of App Tm 1 (DOM26m-29, DOM26m-50 and DOM26h-161 for example) or because the molecule unfolds via a single transition (as in the case of DOM26h-161, DOM26h-165, DOM26h-166 and DOM26h-167).

### Example 4

Analysis of ASGPR-Specific dAb Binding to Murine Liver Cell Lines by Immunofluorescence Confocal Microscopy

In order to study cell surface binding, internalisation and intracellular localisation of ASGPR specific dAbs confocal microscopy assays were developed. Briefly, cells were grown on glass chamber slides and incubated with 5 μM ASGPR specific dAbs with a c-terminal FLAG epitope tag at 37° C. for 45 minutes. Cells were then fixed with 2% formaldehyde at room temperature for 10 minutes. Following washing with 5%FCS/PBS the cells were then co-stained with and either a rabbit polyclonal antibody specific to early endosomal antigen 1 (EEA1) as an early endosomal marker or rabbit polyclonal specific to lysosomal associated membrane protein 1 (LAMP1) as a lysosomal marker. The antibodies were diluted in 5%FCS/PBS including Saponin at a final concentration of 0.2% and incubated at room temperature for 1 hour with the cells. Following washing steps, the dAbs and polyclonal antibodies were detected using an anti-FLAG M2-Cy3 conjugated monoclonal and anti-rabbit Alexa Fluor 488 antibody respectively. The cells were also co-stained with 4′,6-diamidino-2-phenylindole (DAPI) as a marker for DNA. The cells were prepared for imaging and visualised using confocal microscopy.

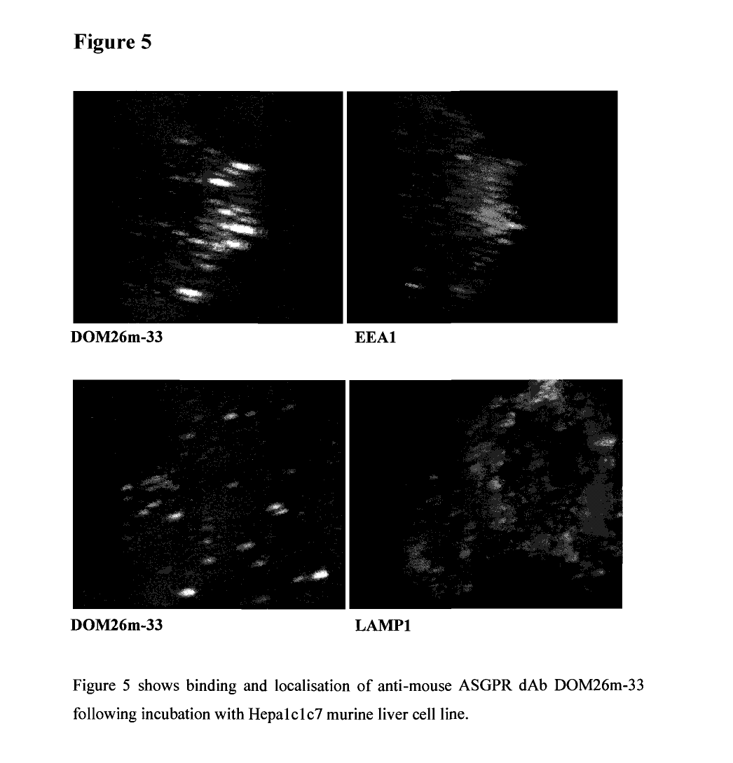

The results showed that the murine ASGPR specific dAb clone DOM26m-33 bound to the murine liver cell line Hepal clc7 and was internalised into early endosomes, as shown by partial co localisation of anti-FLAG and anti-EEA1 staining (FIG. 5). However, the staining pattern was predominantly cell surface indicating that no significant internalisation is occurring. Under no circumstances was co localisation of anti-FLAG and LAMP1 staining observed, therefore it seems likely that ASGPR specific dAb clone DOM26m-33 is not targeted for degradation in the lysosome.

No staining of L929 murine fibroblast negative control cells with DOM26m-33 was observed, demonstrating that the staining pattern observed with this dAb in experiments with the Hepal clc7 line was liver cell specific. Similarly no staining of Hepal clc7 with VH germline sequence VHD2 was observed.

### Example 5

Epitope Mapping by Surface Plasmon Resonance

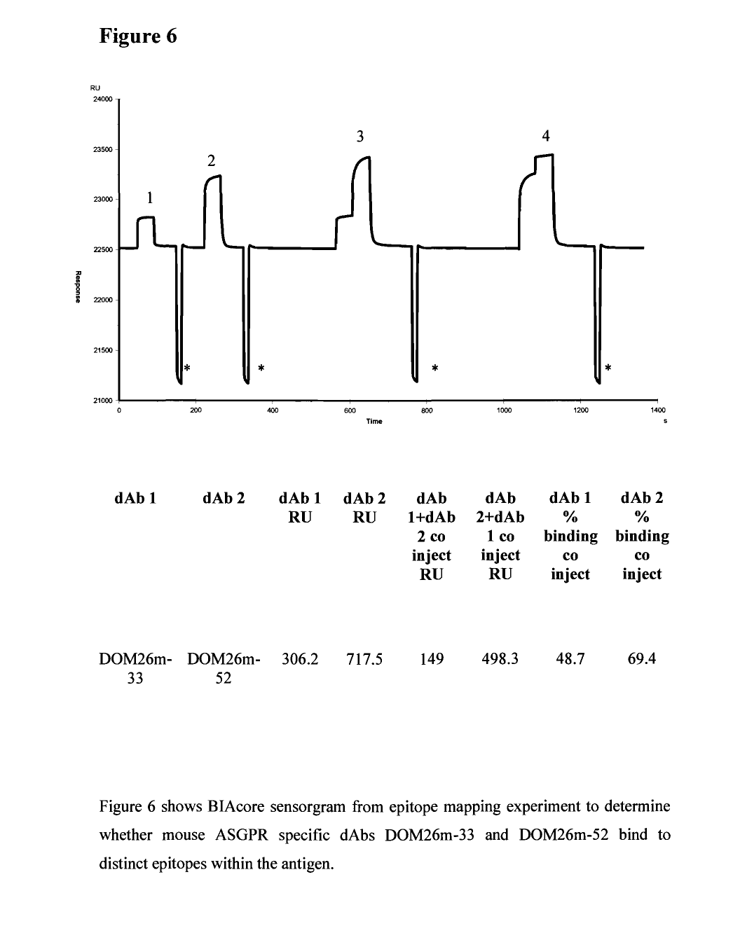

After coating a BlAcore CM5 chip with (His)₆ mouse ASGPR H1, protein A or protein L purified dAb proteins were injected one after the other and in combination over the same antigen surface. The resulting binding RUs were determined in order to see whether the maximal binding capacity of the chip by one dAb molecule can be exceeded by simultaneously injecting a second dAb onto the same antigen surface. If so, the second dAb clearly binds a different epitope compared to the first one. 1 μM concentrations of each dAb were injected at a flow rate of 10 μl per second, both in single injection and co-injection experiments. If injection of the second dAb in the presence of the first dAb reduced the observed binding to the chip surface by greater than 20% (in comparison to observed binding of the second dAb to the chip surface in the absence of the first dAb) both dAbs were assumed to bind overlapping epitopes within the antigen (FIG. 6). Based on results obtained in these experiments murine ASGPR specific dAb clones could be grouped according to epitopes bound within the antigen as shown in FIG. 12.

Epitope mapping by BlAcore shows that several distinct epitopes within the (His)₆ mouse ASGPR H1 antigen are bound by these 8 clones. Epitope mapping data also show that Vκ and V_{H} clones bind to overlapping epitopes in some cases, therefore all 8 clones were used to generate further libraries for affinity maturation.

### Example 6

Binding of ASPGR Specific dAbs to Murine Liver In Vivo

Anti-mouse ASGPR dAb DOM26m-33 and Vκ dummy/V_{H} dummy 2 germline control dAbs were used to generate point mutations such that the arginine residue at the C-terminus of Vκ clones and the serine residue at the C-terminus of V_{H} clones was mutated to cysteine. Therefore Vκ dummy carried the point mutation R108C, V_{H} dummy 2 carried the point mutation S127C and DOM26m-33 carried the point mutation S116C. dAbs were amplified from pDOM10 by PCR using primers DOM008 and PBS-ECVH2 for V_{H} dAbs and primers DOM008 and PBS-ECVK2 for Vκ clones. Oligonucleotide sequences are shown in Table 6 below.

TABLE 6

| | | |
|---|---|---|
| DOM008 | AGCGGATAAC AATTTCACAC AGGA | PUC reverse primer sequence complementary |
| | (Seq ID No. 80) | to region of pDOM10 vector upstream of |
| | | leader and dAb sequence. Adds a SalI site for |
| | | cloning into pDOM10. |
| PBS- | CTAGCGTTGGCTTTGCGGCCGCGGATCCTTA | 3′ reverse primer for VH domains. Changes |
| ECVH2 | TTAGCACGAGACGGTGAC | terminal serine to a cysteine. Also adds a NotI |
| | (Seq ID No. 81) | site for cloning into pDOM10. |
| PBS- | AGCCGGATCCGCGGCCGCTTATTAGCATTTG | 3′ reverse primer for V_{κ} domains. Changes |
| ECVK2 | ATTTCCACCTTGGTCCC | terminal arginine to cysteine. Also adds a NotI |
| | (Seq ID No. 82) | site for cloning into pDOM10. |

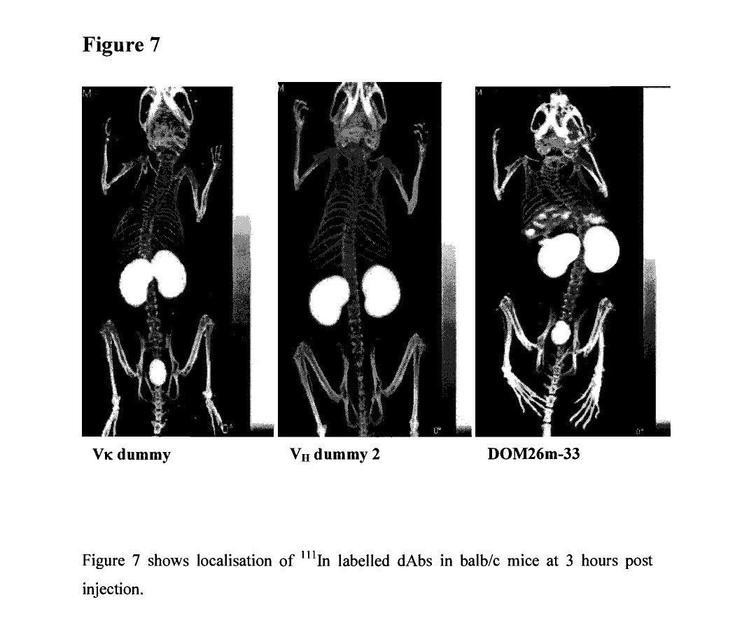

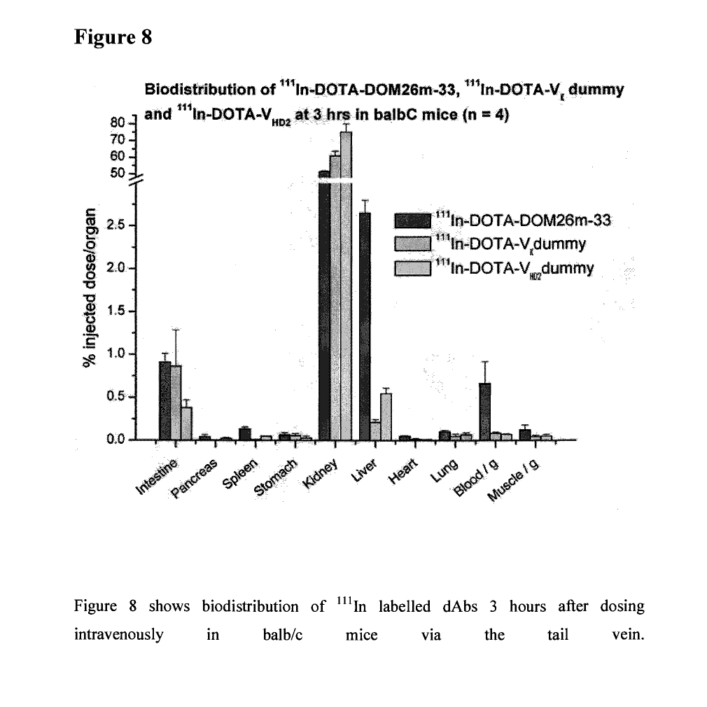

dAb inserts were then digested with SalI and NotI restriction enzymes and cloned into the corresponding sites in pDOM10. dAbs were expressed in 500 ml cultures (OnEX plus carbenicillin) for 3 days at 30° C. and purified on protein A (V_{H} dAbs) or protein L (Vκ dAbs). dAbs were then conjugated with DOTA-Maleimide and labeled with ¹¹¹In. Briefly, dAb solution (and all buffers used in the conjugation method) was passed through Chelex 100 resin to remove cations. Chelex treated dAb solution was then reduced by addition of 0.5M TCEP, 1% (v/v). After 30 minutes reducing agent was removed by size exclusion chromatography using a PD10 column. Conjugation was carried out overnight at room temperature by addition of 30 fold molar excess of DOTA-Maleimide dissoloved in 25 mM HEPES, pH 7. DOTA-Maleimide conjugated dAb was purified from the reaction mixture using protein A streamline resin and eluted in 0.1 M Glycine, pH2. Eluate was neutralized by addition of 1/10 volume 1 M Tris, pH 8.0. 1/3 volume 2 M ammonium actetate was then added to neutralized eluate to adjust pH to 5.5 and protein concentration calculated by measuring absorbance at 280 nm. The degree of conjugation was determined by mass spectrometric analysis. Purified DOTA-Maleimide conjugated dAb solution was then radiolabeled in 35 μl reaction volumes by addition of 5-20 μl ¹¹¹InCl₃ (dissolved in 0.05 M HCl) and 1-4 μl of 1 M ammonium acetate, pH 5.5 to 25 μg DOTA-Maleimide conjugated dAb. Reaction was allowed to proceed at 37° C. for 1-3 hours before radiolabelling efficiency was analysed using thin layer chromatography. Following successful radiolabelling reaction mixture was quenched using 0.001% (v/v) 0.1 M EDTA. Approximately 12 MBq radiolabeled dAb was injected into isofluorane anaesthetized balb/c mice intravenously via the tail vein before imaging over a 7 day time course using the Nanospect/CT preclinical in vivo imaging system. Analysis of images showed that in mice injected with ¹¹¹In labeled DOM26m-33 signal was observed in the kidney, bladder and liver after 3 hours (FIG. 7). However in Mice injected with ¹¹¹In labeled Vκ dummy or V_{H} dummy 2 no signal was observed in the liver over 7 days post injection, therefore liver specific binding of DOM26m-33 in vivo is a direct consequence of ASGPR binding. Signal was observed in the kidney and bladder in all cases due to excretion via this route. In order to quantitatively determine the in vivo distribution of ¹¹¹In labeled dAbs whole body autoradiography experiments were carried out. Balb/c mice were dosed with approximately 0.5 MBq of radiolabelled dAb as above. Mice were then sacrificed 3 hours after injection before removing organs and counting in a gamma counter. Counts detected in various organs were expressed as percent injected dose. Results of these experiments show that counts in the liver of mice injected with DOM26m-33 were 12.4 times higher compared to counts in the liver of mice injected with Vκ dummy and 4.9 times higher compared to counts in the liver of mice injected with V_{H} dummy 2 (FIG. 8).

### Example 7

Cloning and Expression of Murine Interferon Alpha Fused to ASGPR Specific dAbs

Mouse Interferon-alpha 2 cDNA was custom synthesised by DNA2.0. DNA encoding the full length protein (without the signal peptide sequence) with a partial linker sequence (described below) and an AvrII restriction site appended to the c-terminus, was amplified by PCR using primers DX132 and DX133. Oligonucleotide sequences are shown in Table 7 below.

TABLE 7

| | | |
|---|---|---|
| DX132 | GGATCCACCGGCTGCGATCTGCCTCACACT | Addition of 5′ BamHI to Mouse |
| | TA (Seq ID No. 83) | IFNa2 for cloning into pDOM50 |
| DX133 | CCTAGGAGCGGCGACGGTCTCCTTCTCTTC | Addition of 3′ TVAAPS and AvrII |
| | ACTCAGTCT (Seq ID No. 84) | site to Mouse IFNa2 for cloning |
| | | into pDOM50 |

PCR fragments were inserted into holding vector pCR-Zero Blunt (Invitrogen) by Topoisomerase cloning and sequenced to obtain error-free clones using M13 forward amd M13 reverse primers. Mouse IFNa2 encoding DNA was obtained by gel purification following BamHI/AvrII digestion of pCR-Zero Blunt containing the insert and inserts ligated into the corresponding sites in pDOM50 to produce the vector pDOM38mIFN-N1.

Anti-mouse ASGPR dAbs (or germline control dAbs Vκ dummy and V_{H} dummy 2) were then cloned into pDOM38mIFN-N1 to produce Mouse IFNa2 fused at the C-terminus to dAb sequence with the intervening linker sequence TVAAPS as described below:

Following PCR amplification of dAb nucleotide sequence with primers DX008 and DX018 for Vκ clones or DX009 and DX019 for V_{H} clones PCR fragments were inserted into holding vector and sequenced to obtain error-free clones as above. DNA encoding dAb sequence was obtained by gel purification following NheI/HindIII digestion of pCR-Zero Blunt containing the insert and inserts ligated into pDOM38mIFN-N1 digested with AvrII/HindIII.

Constructs with c-terminal residue of dAb mutated to cysteine was also produced as above, except antisense primers used in place of DX018 and DX019 were DLT048 for Vκ clones and DLT049 for V_{H} clones. Oligonucleotide sequences are shown in Table 8 below.

TABLE 8

| | | |
|---|---|---|
| DX008 | GCTAGCGACATCCAGATGACCCAG | Addition of 5′ NheI to V_{κ} for cloning into |
| | TCTCCAT (Seq ID No. 85) | pDOM38mIFN-N1 |
| DX009 | GCTAGCGAGGTGCAGCTGTTGGA | Addition of 5′ NheI to V_{H} for cloning into |
| | GTCTGGG (Seq ID No. 86) | pDOM38mIFN-N1 |
| DX018 | AAGCTTTTATTACCGTTTGATTTCC | Addition of 3′ 2× STOP and HindIII to V_{κ} for |
| | ACCTTGGTCCC (Seq ID No. 87) | cloning into pDOM38mIFN-N1 |
| DX019 | AAGCTTTTATTAGCTCGAGACGGT | Addition of 3′ 2× STOP and HindIII to V_{H} for |
| | GACCAGGGTTCCC | cloning into pDOM387h-14-N1 |
| | (Seq ID No. 88) | |
| DLT048 | AAGCTTTTATTAGCATTTGATTTCC | Addition of 3′ 2× STOP and HindIII to V_{κ} for |
| | ACCTTGGTCCC | cloning into pDOM38mIFN-N1. Also mutates |
| | (Seq ID No. 89) | C-terminal serine to cysteine. |
| DLT049 | AAGCTTTTATTAGCACGAGACGGT | Addition of 3′ 2× STOP and HindIII to V_{H} for |
| | GACCAGGGTTCC | cloning into pDOM38mIFN-N1. Also mutates |
| | (Seq ID No. 90) | C-terminal serine to cysteine. |

Linker Sequence (amino acid):
(Seq ID No. 91)
TVAAPS
Linker Sequence (nucleotide):
(Seq ID No. 92)
ACCGTCGCCGCTCCTAGC

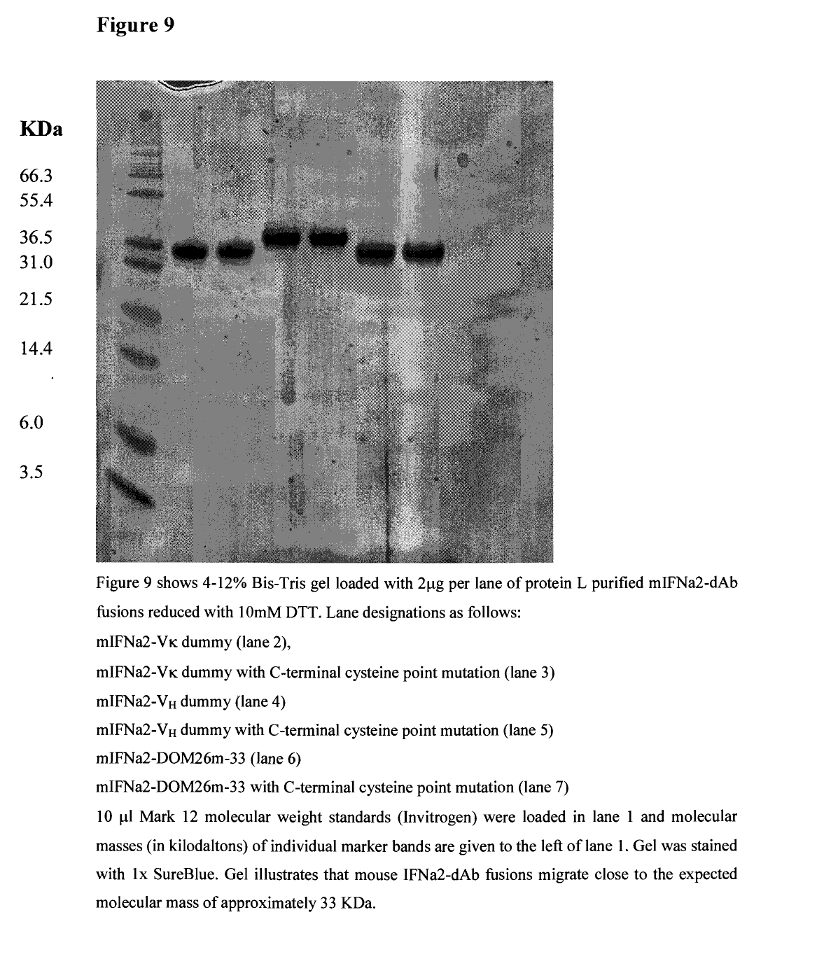

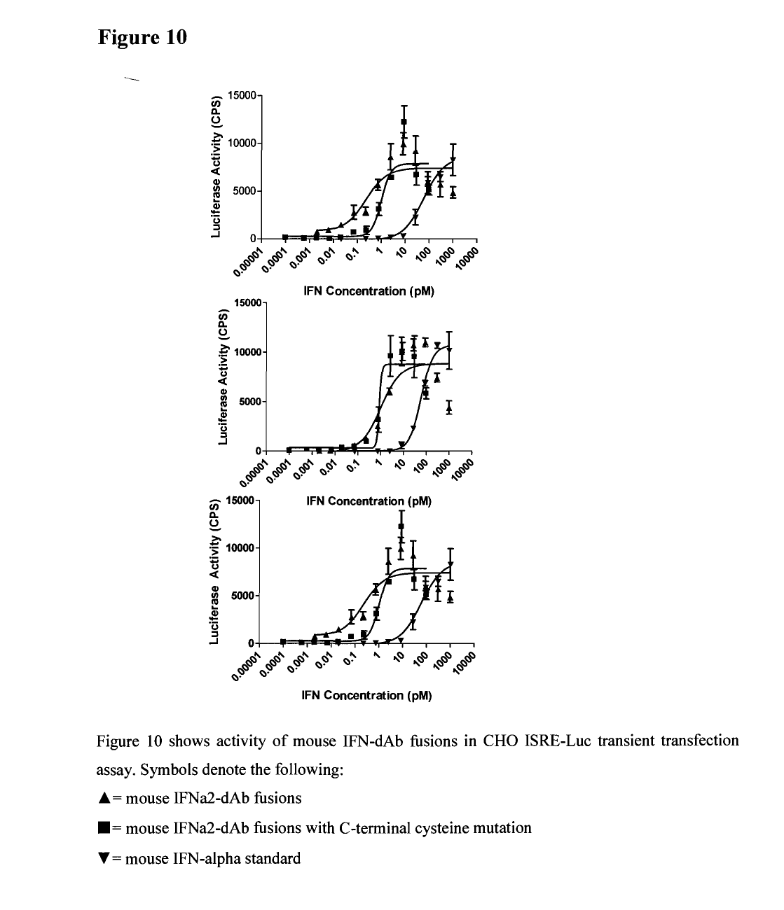

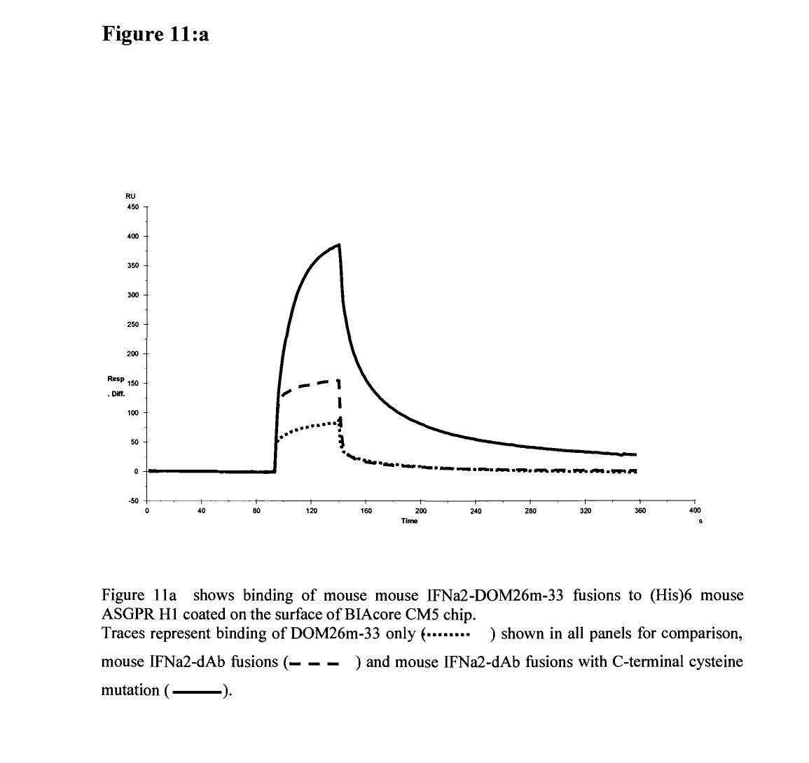

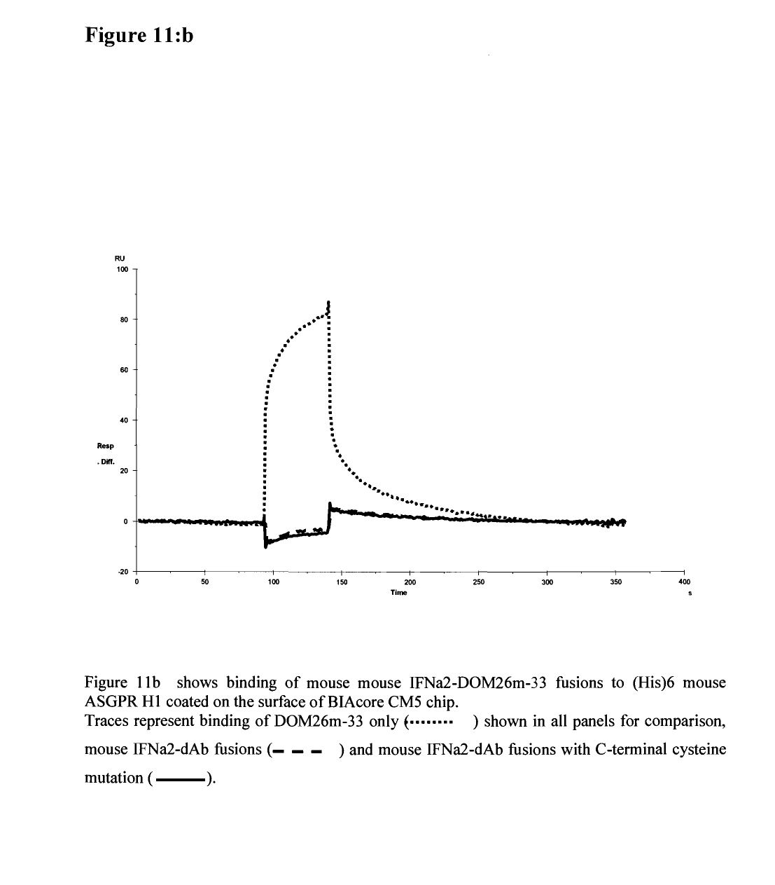

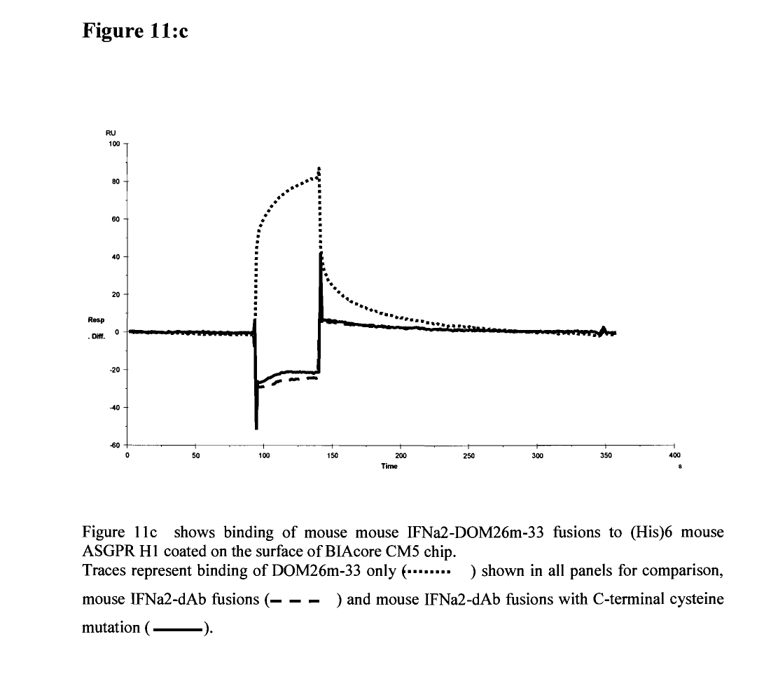

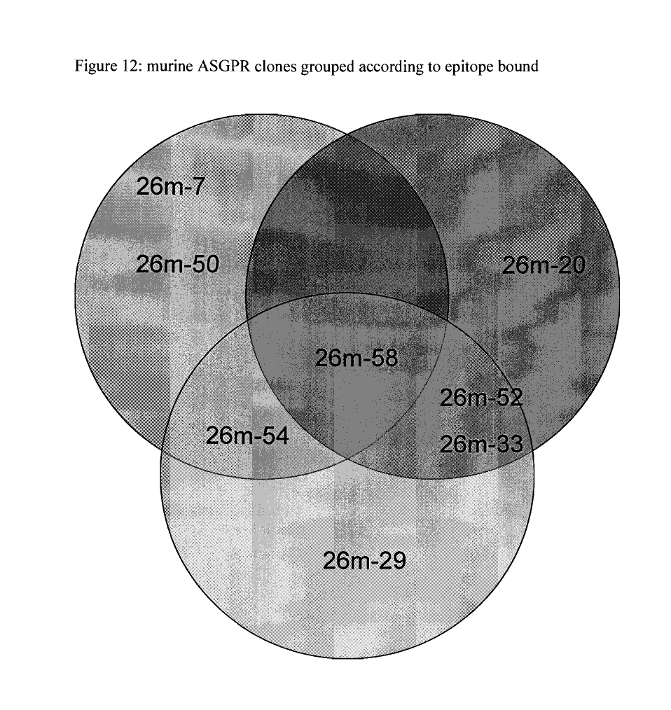

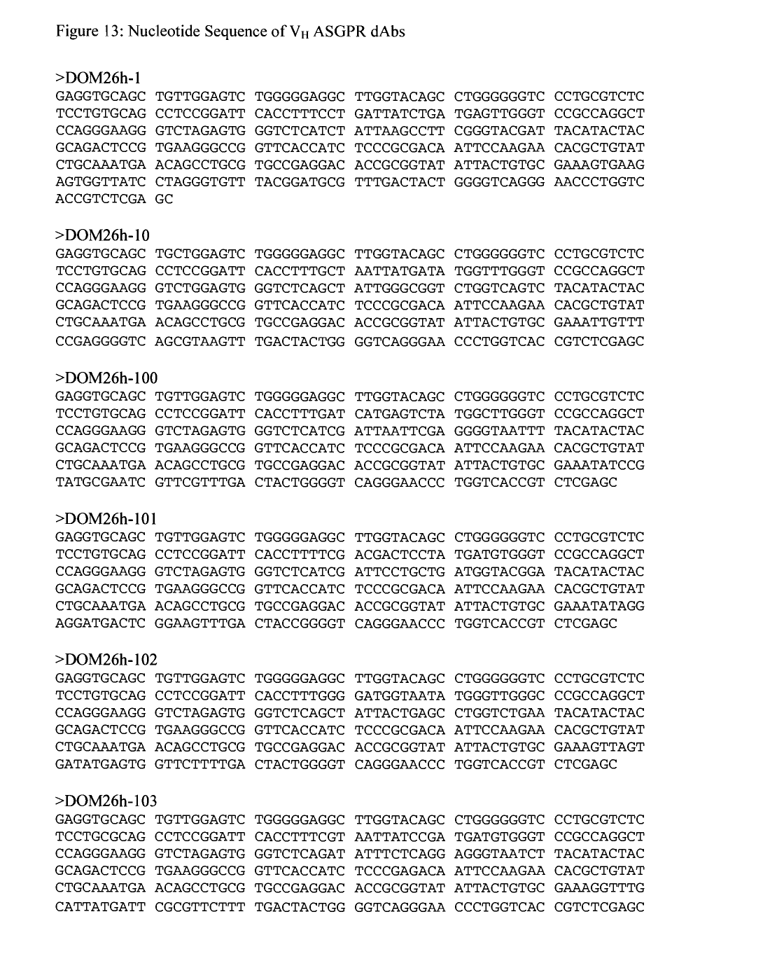

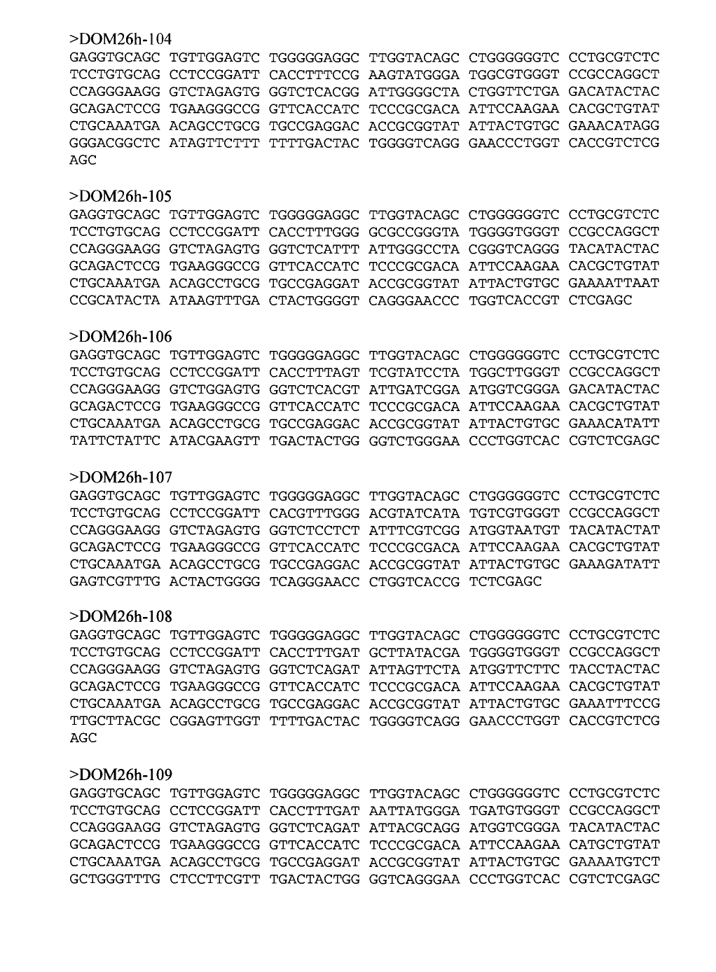

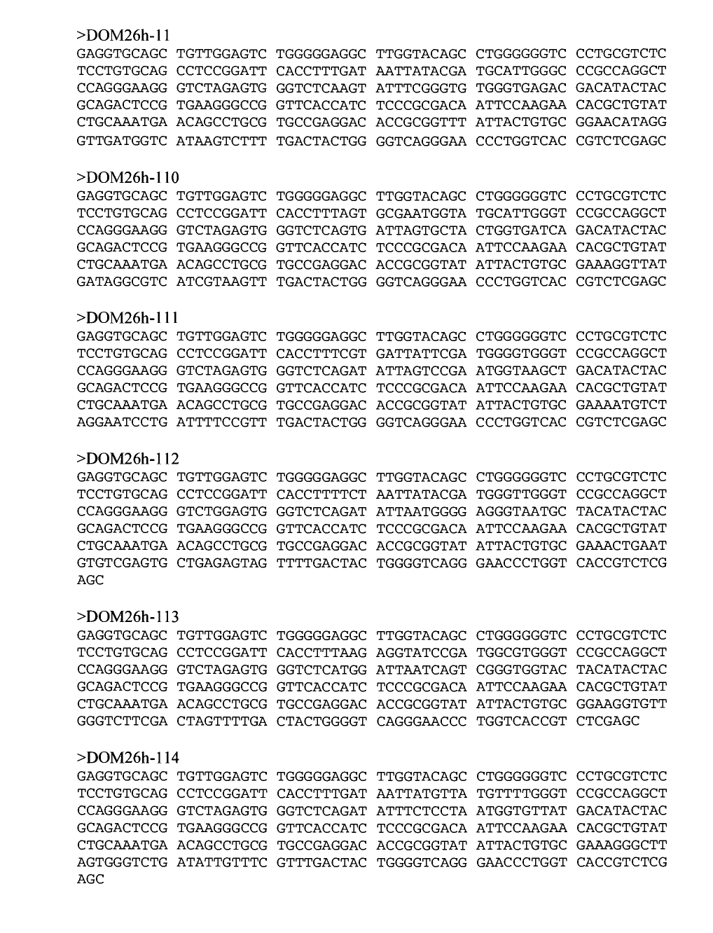

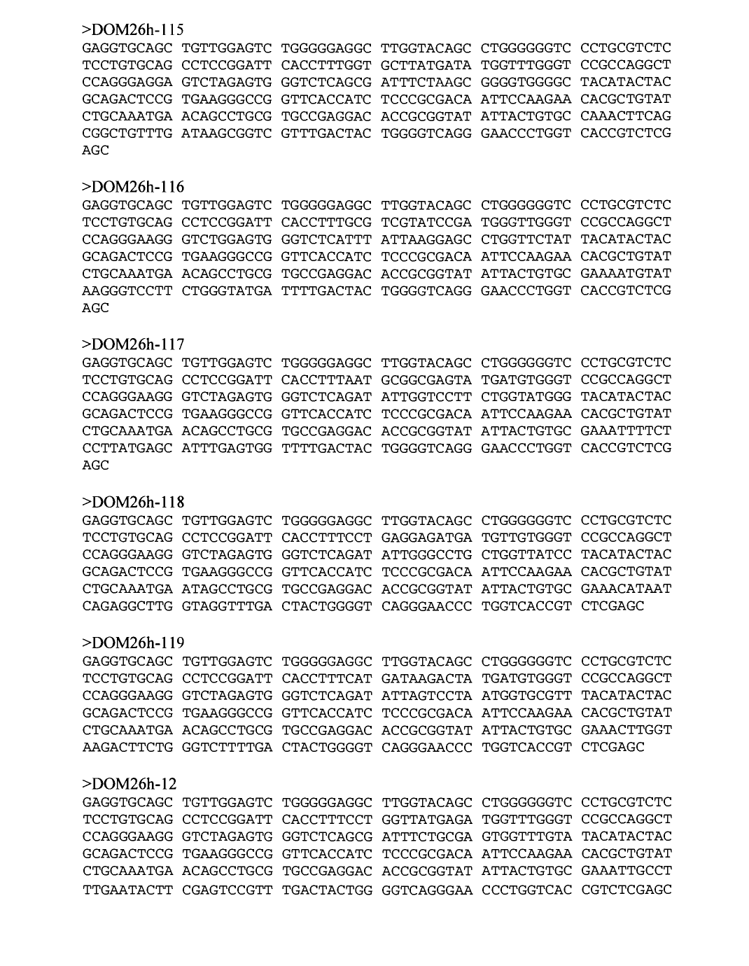

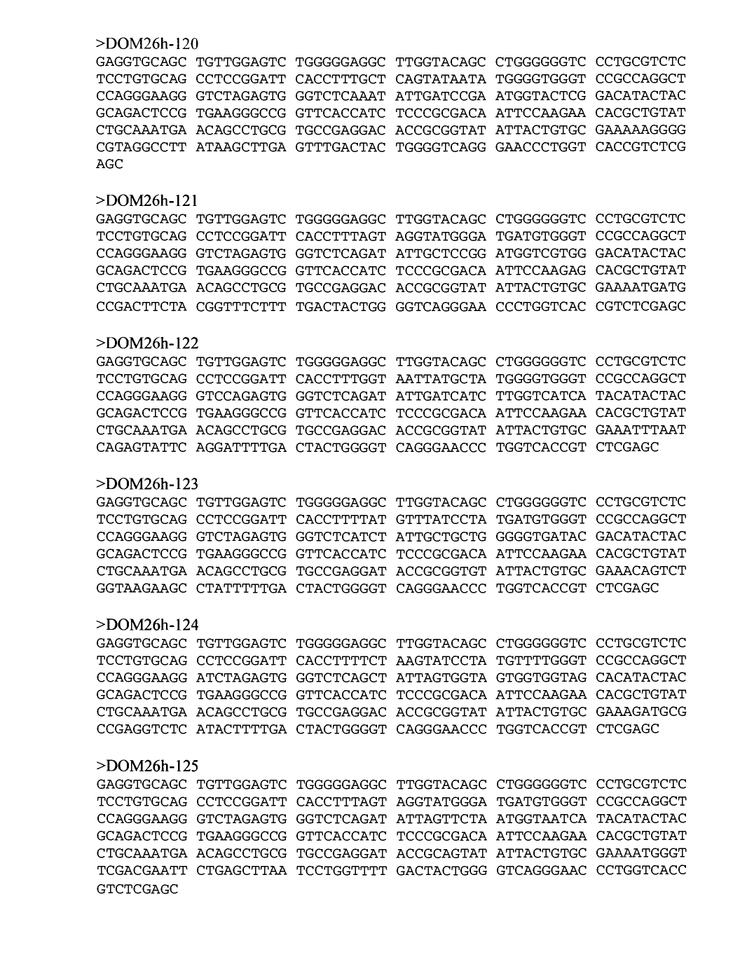

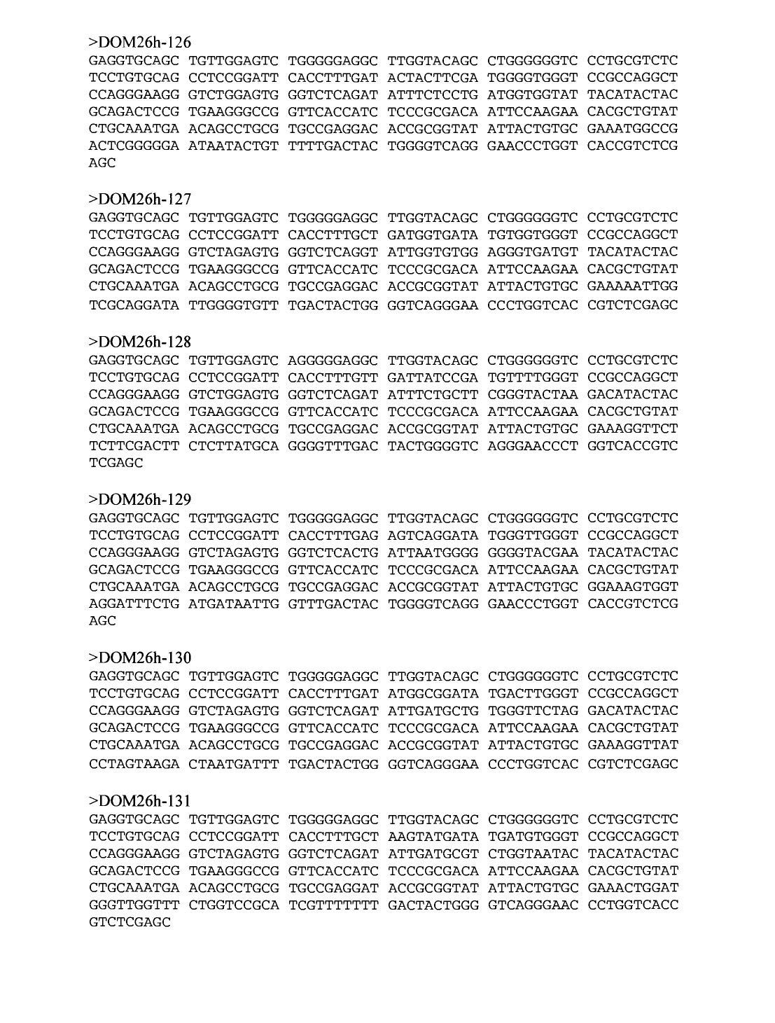

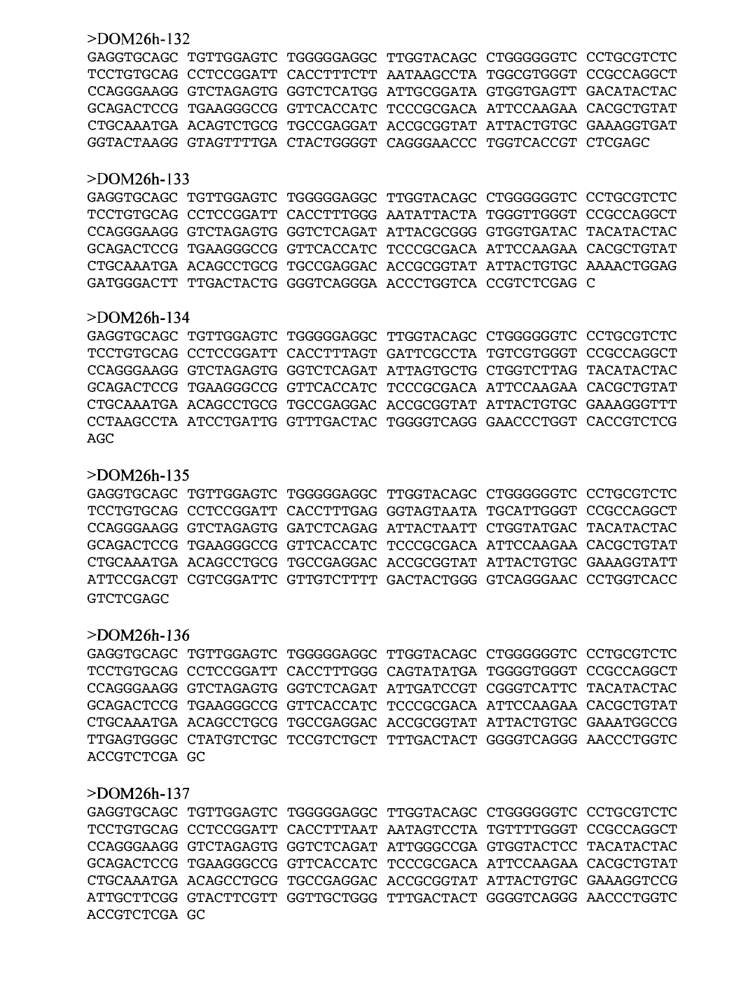

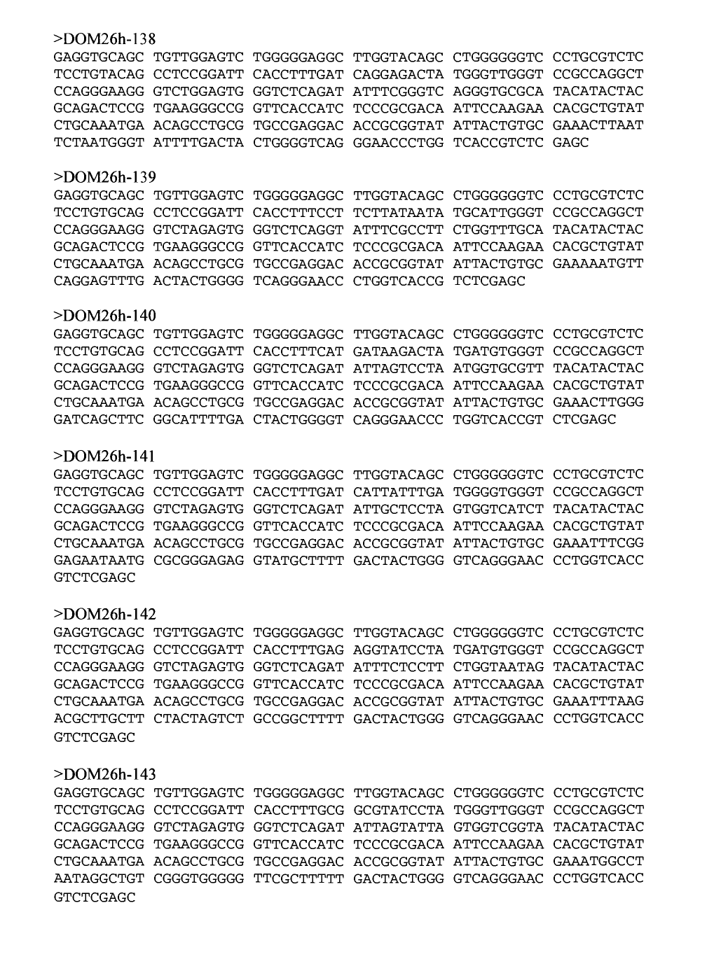

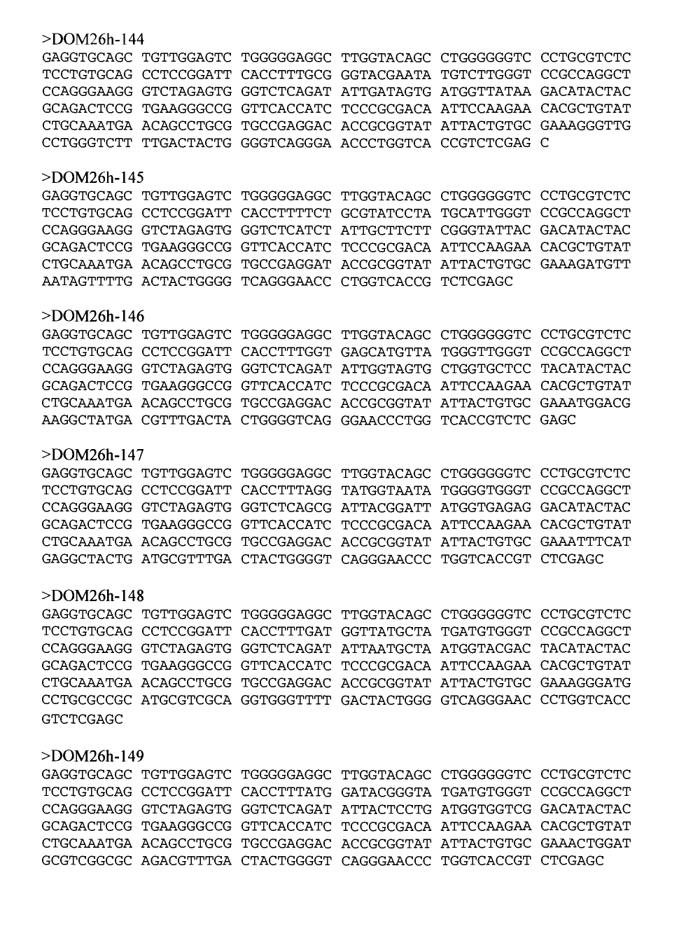

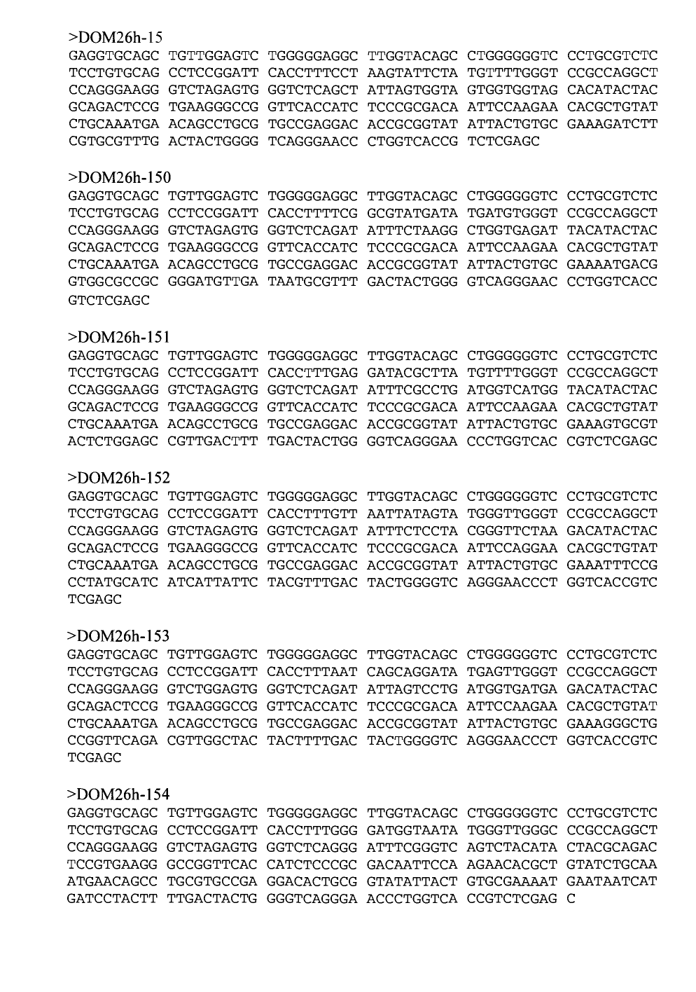

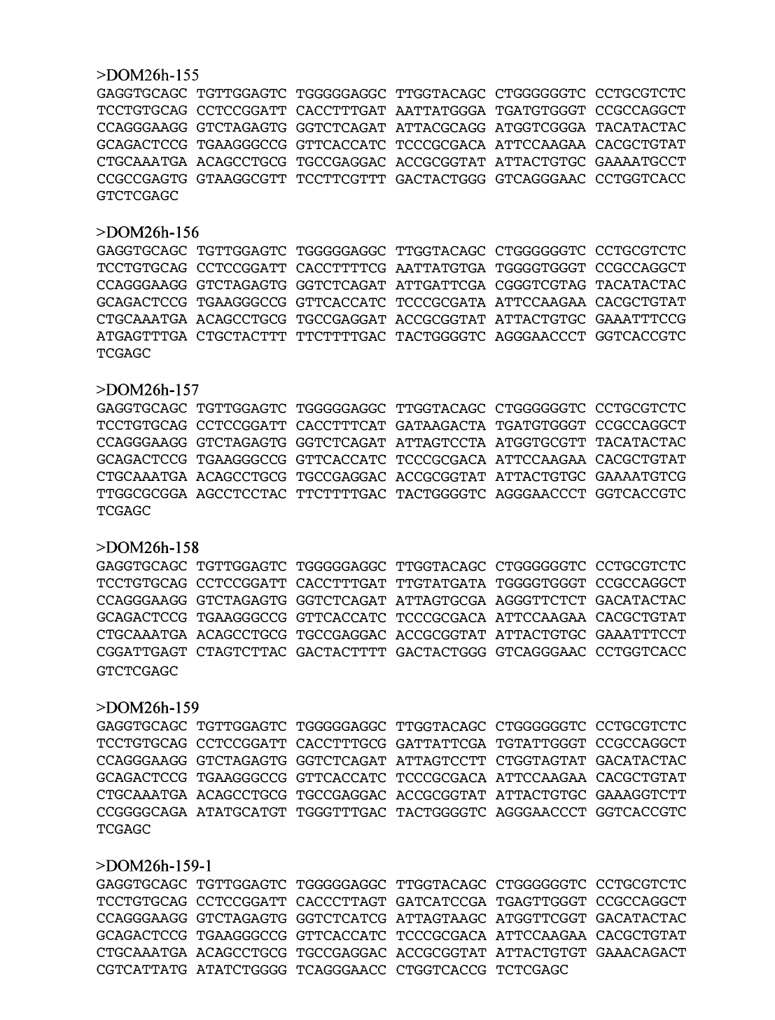

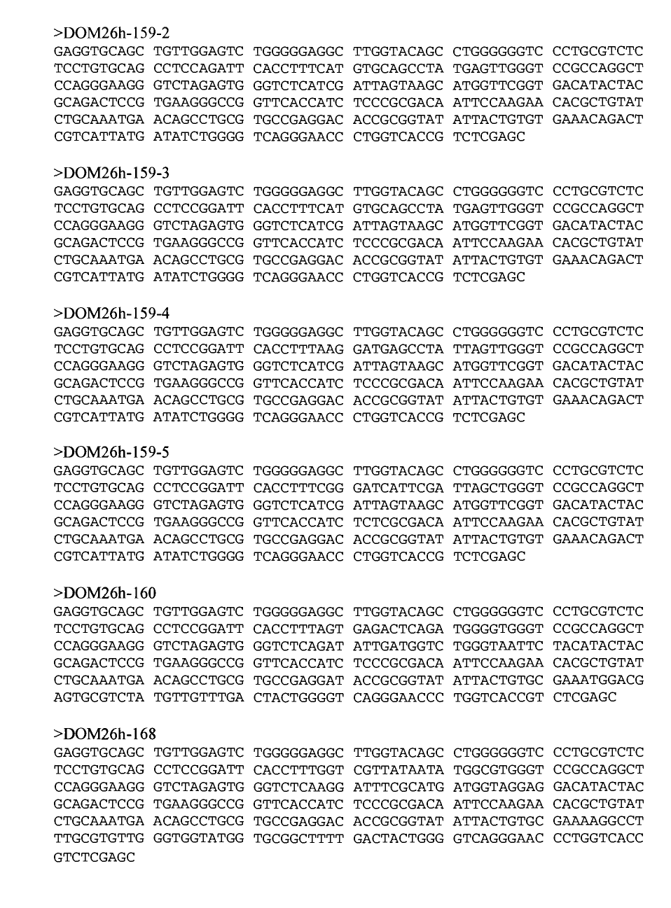

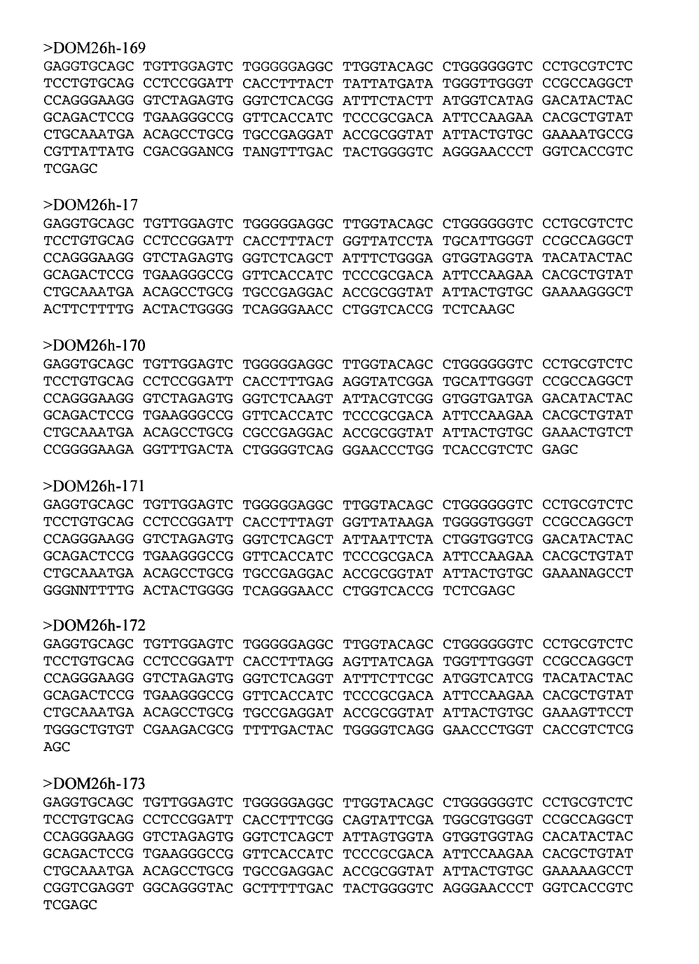

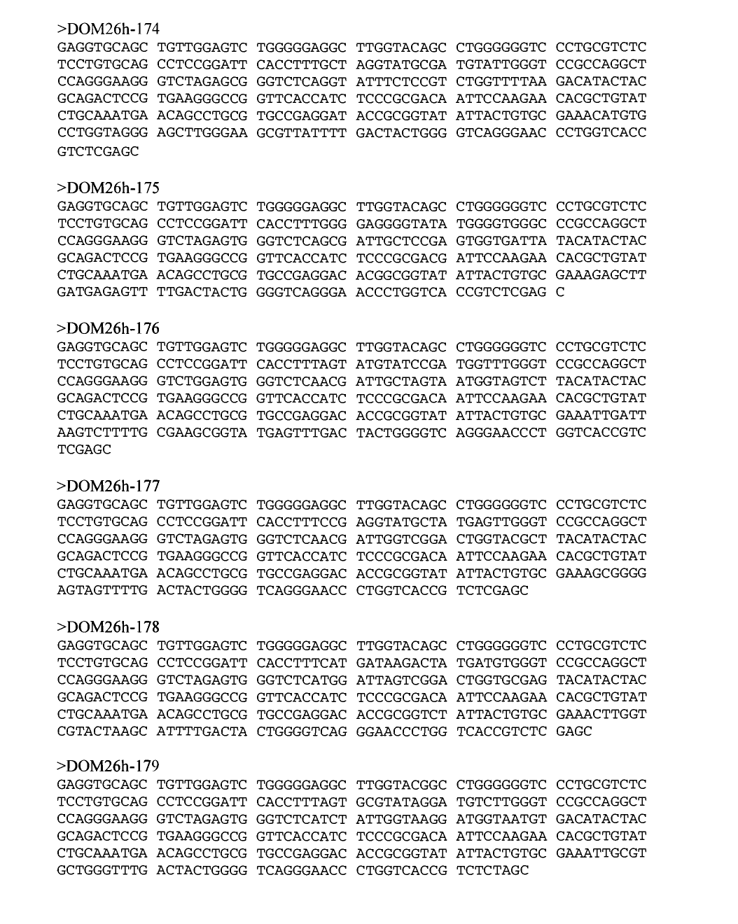

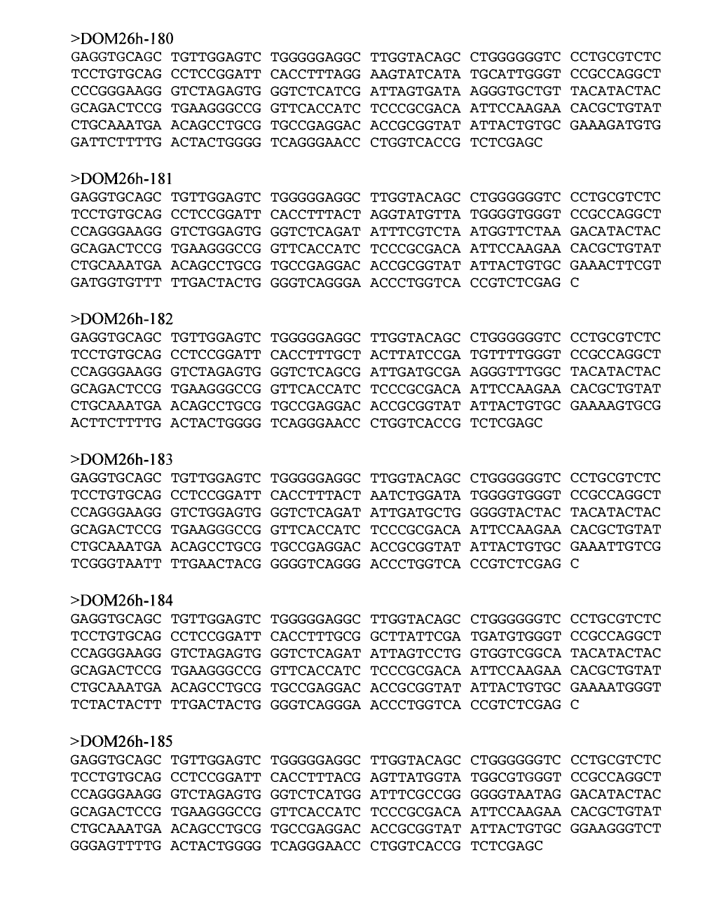

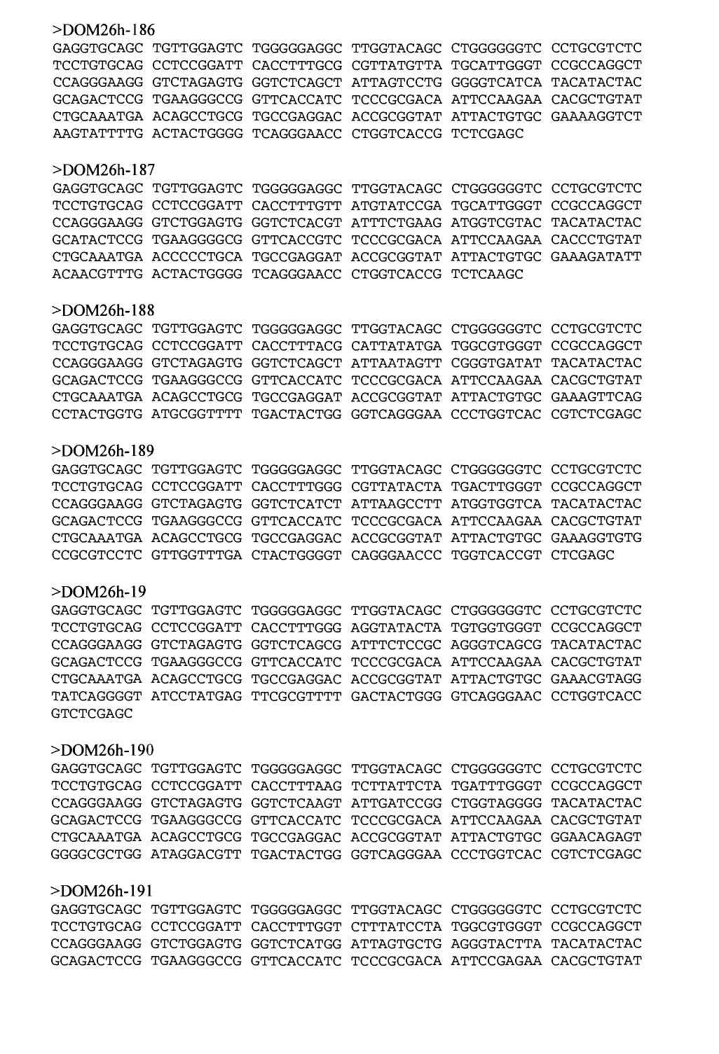

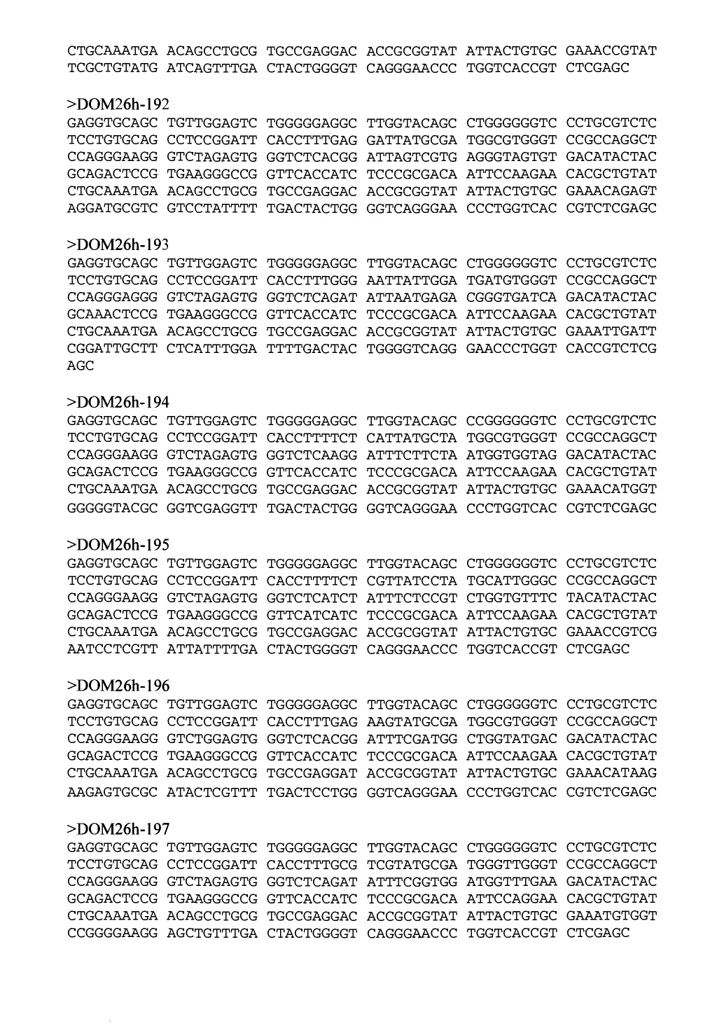

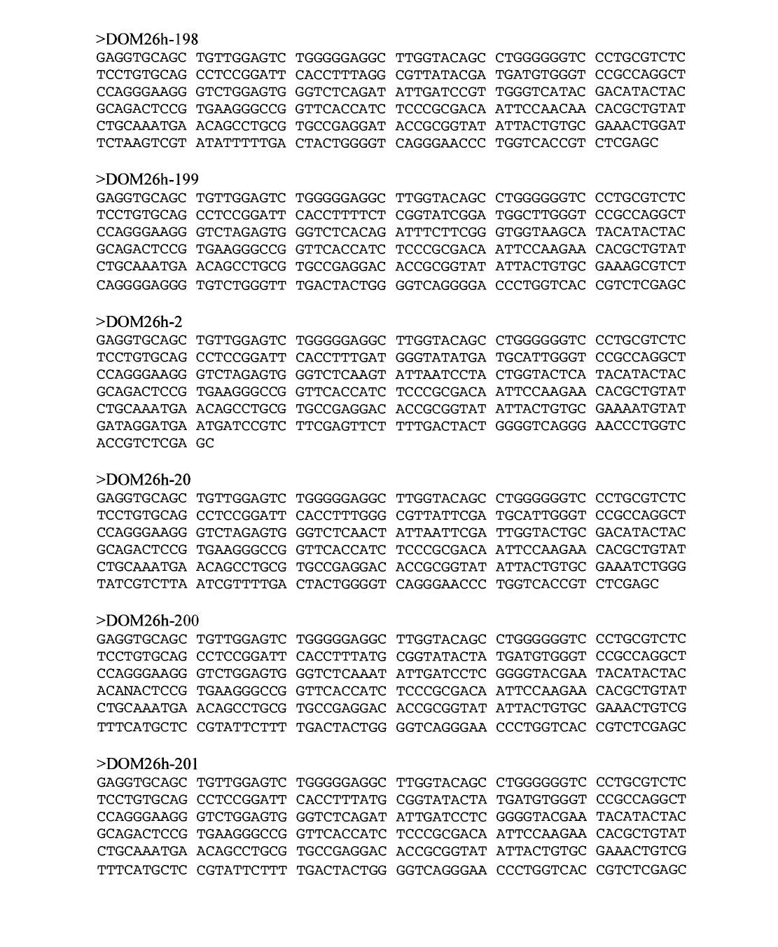

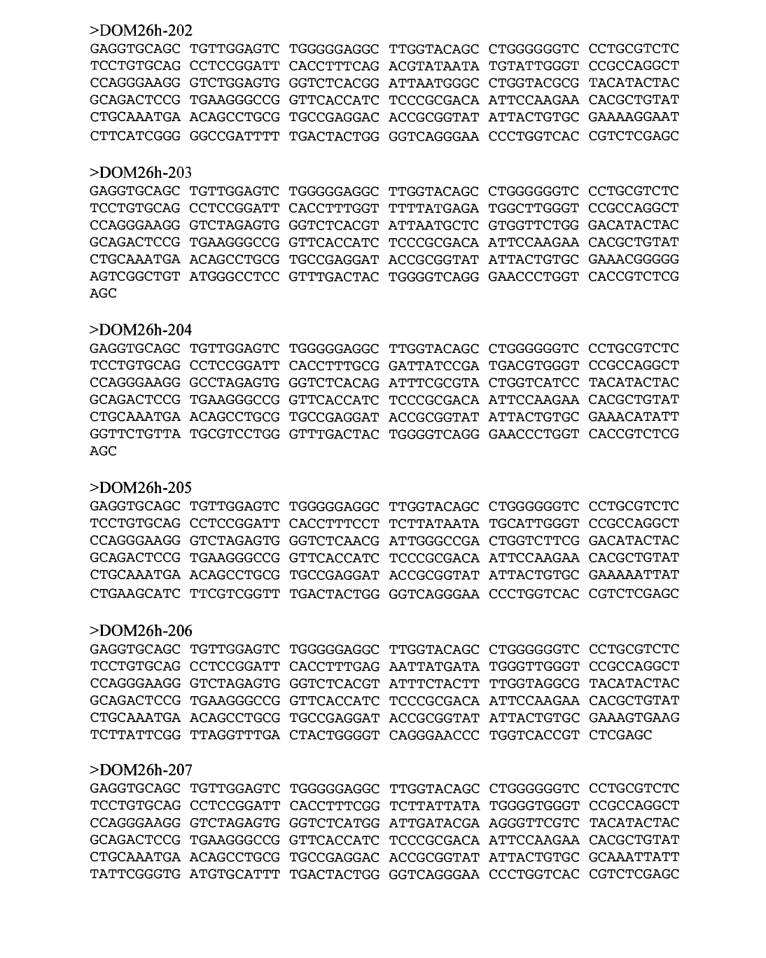

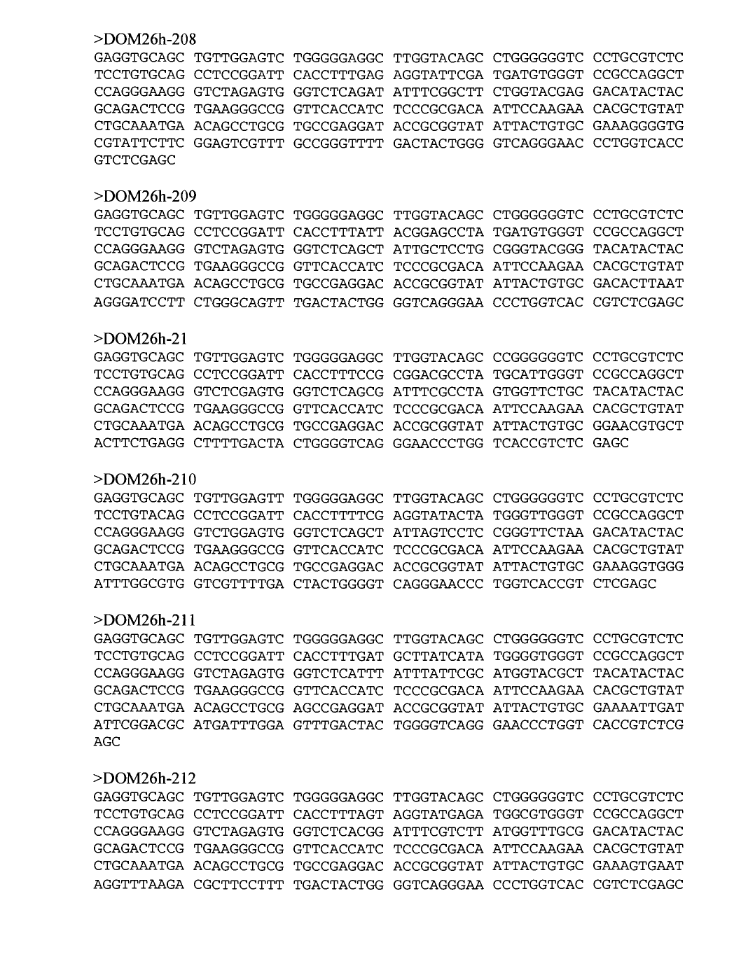

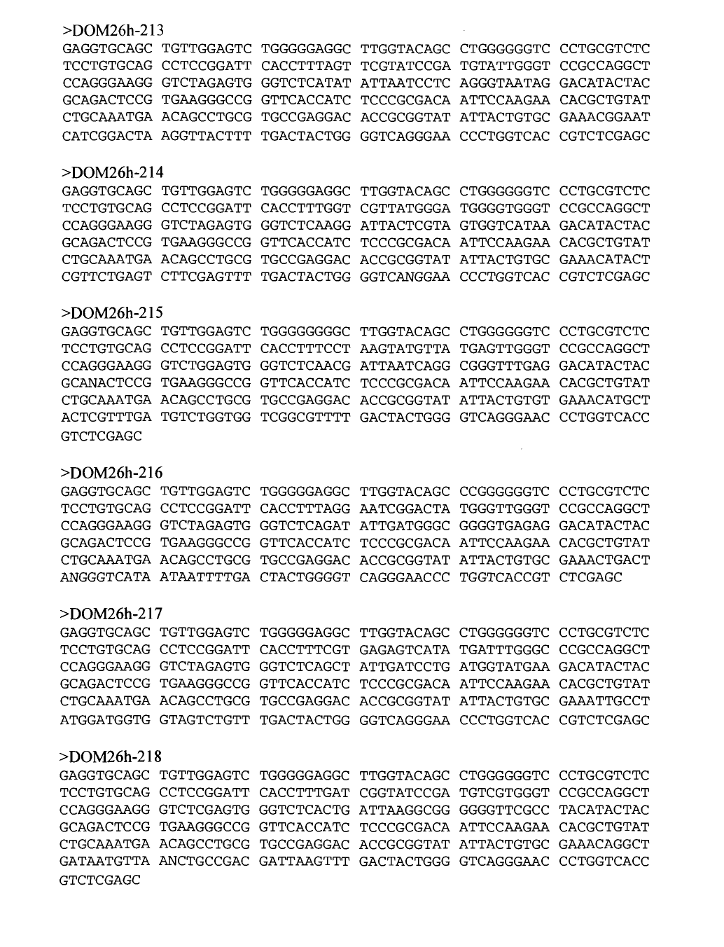

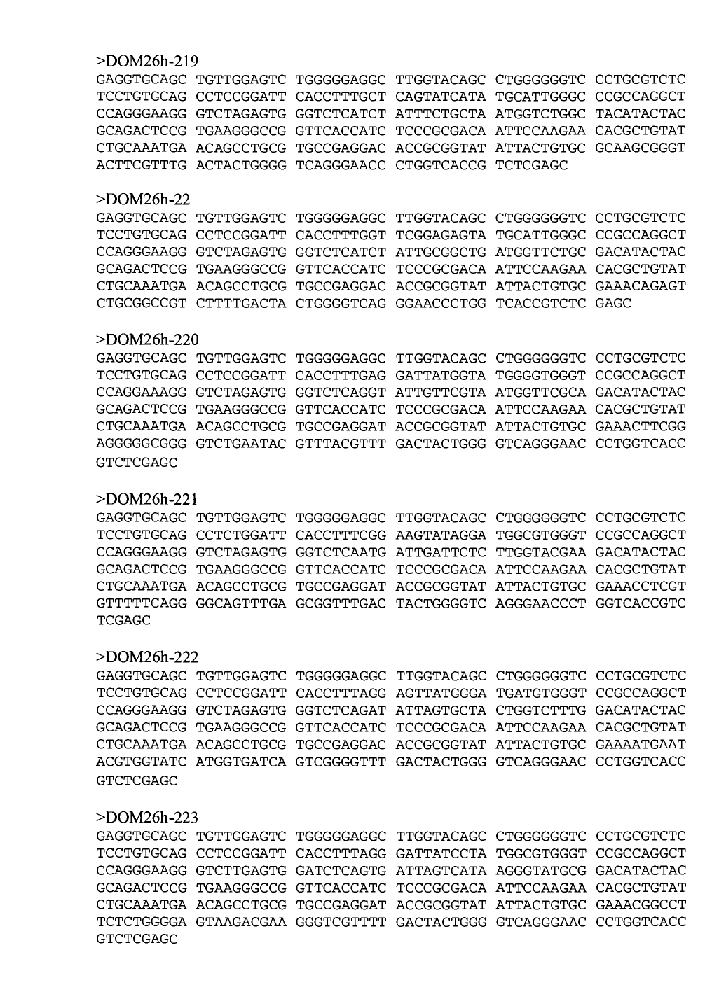

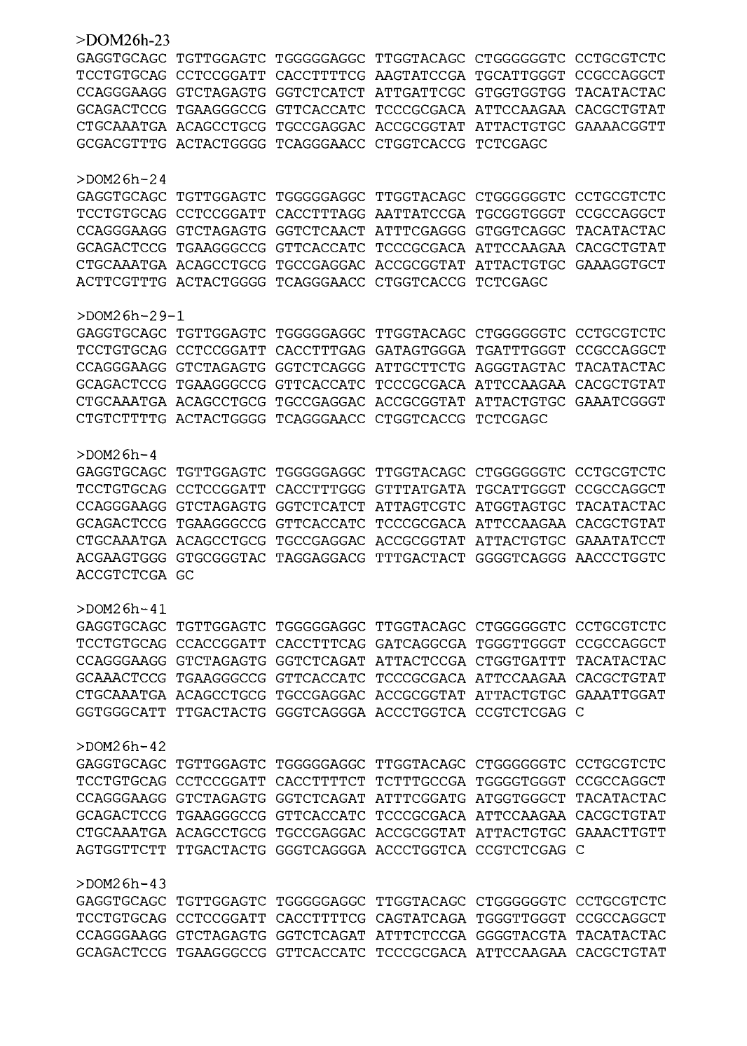

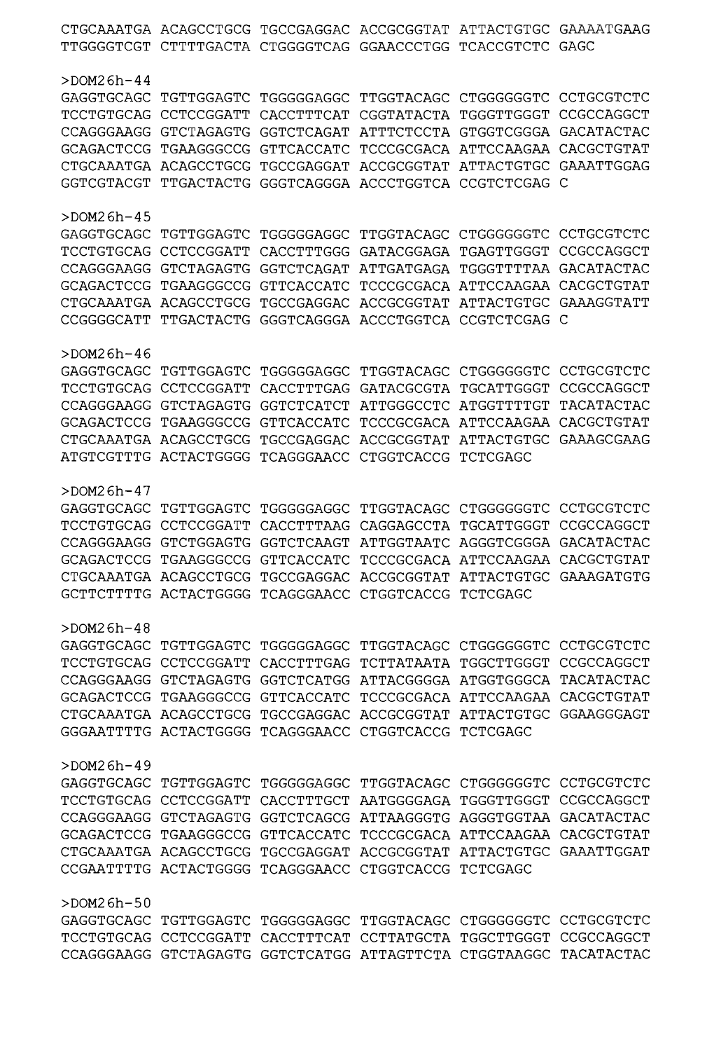

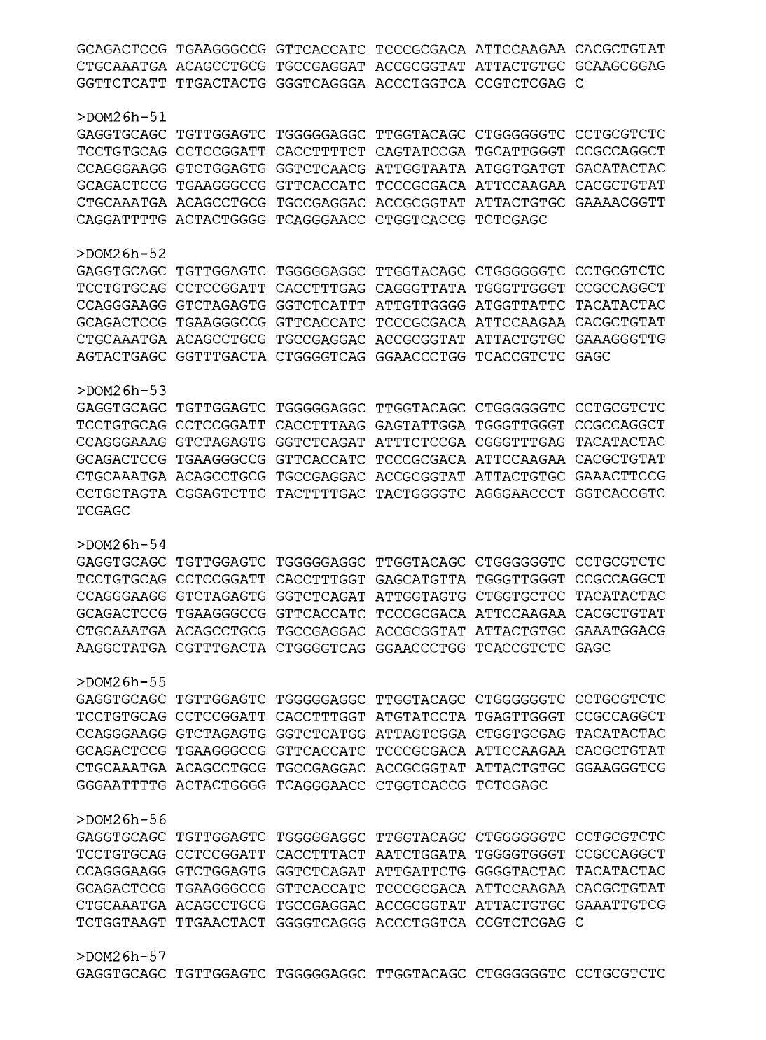

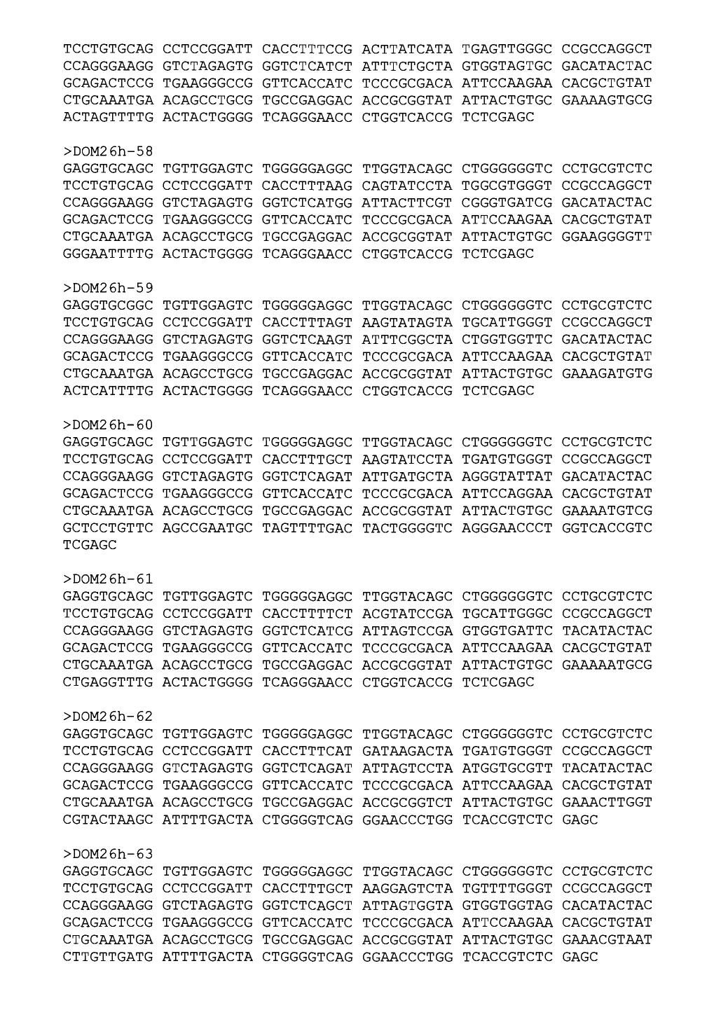

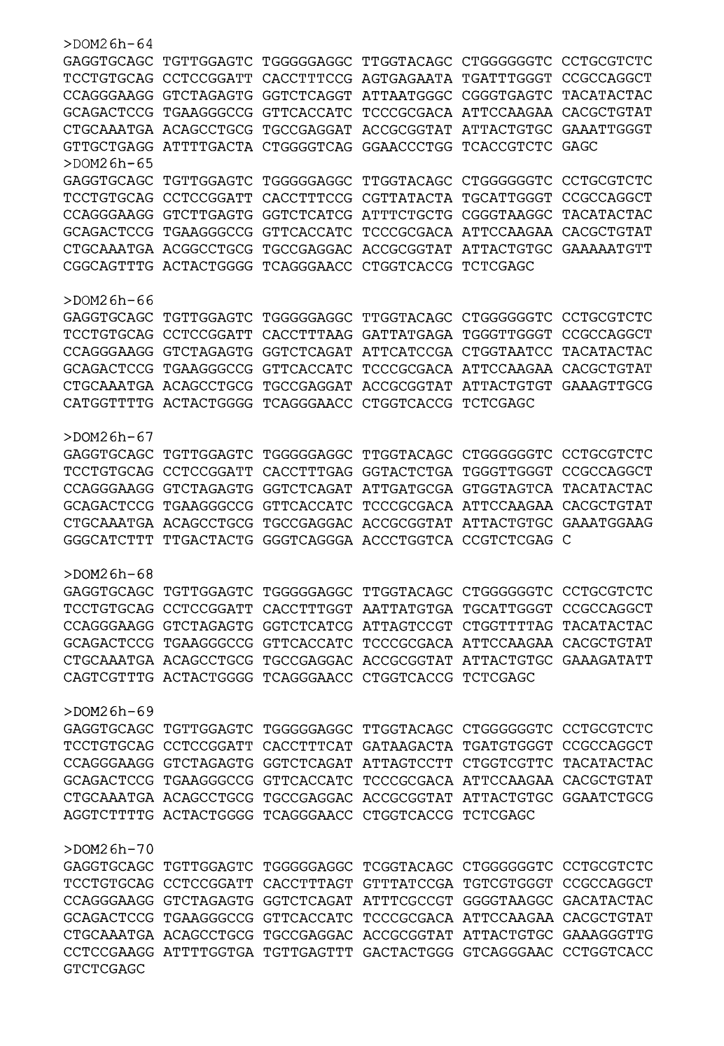

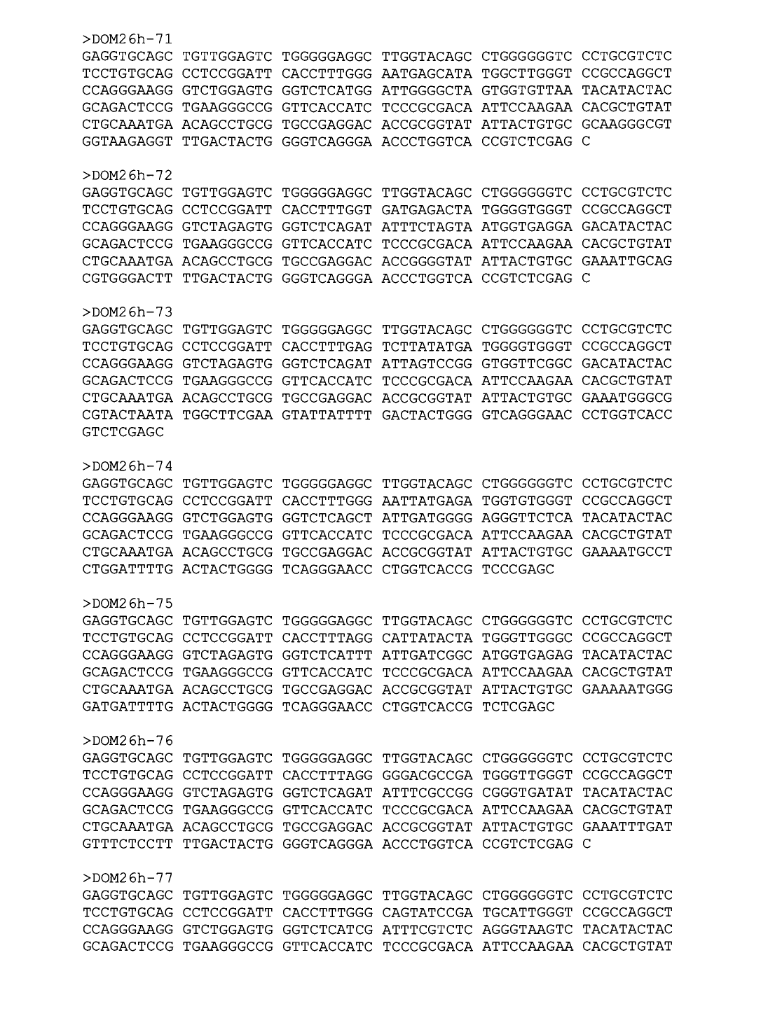

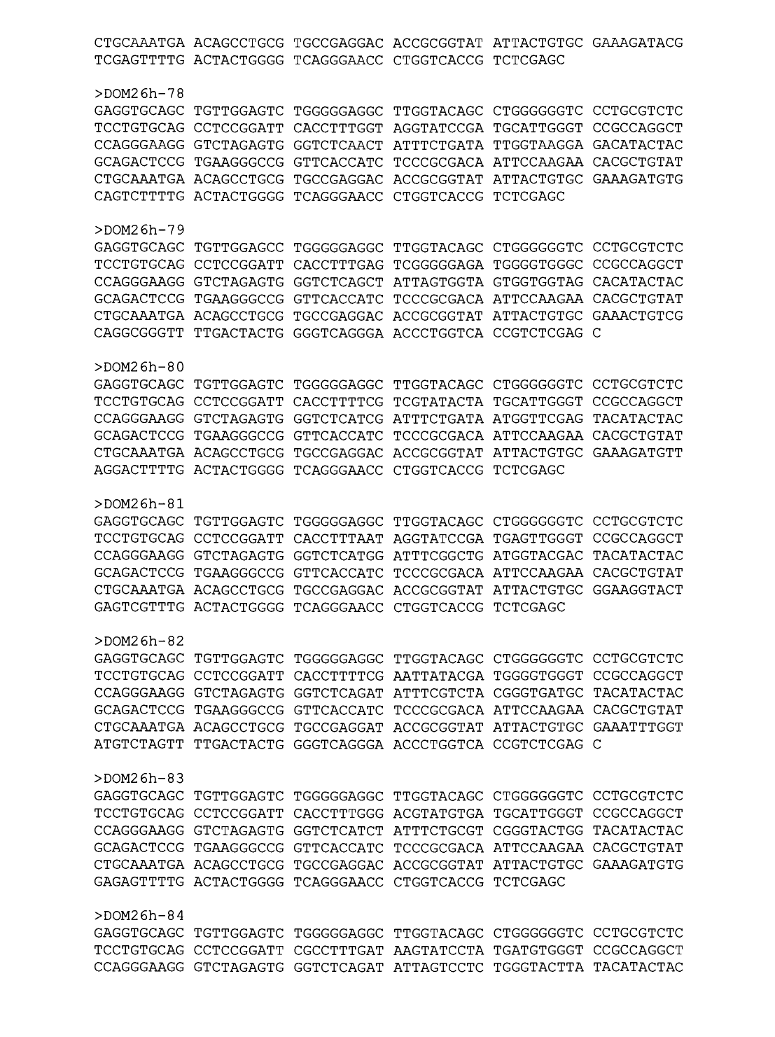

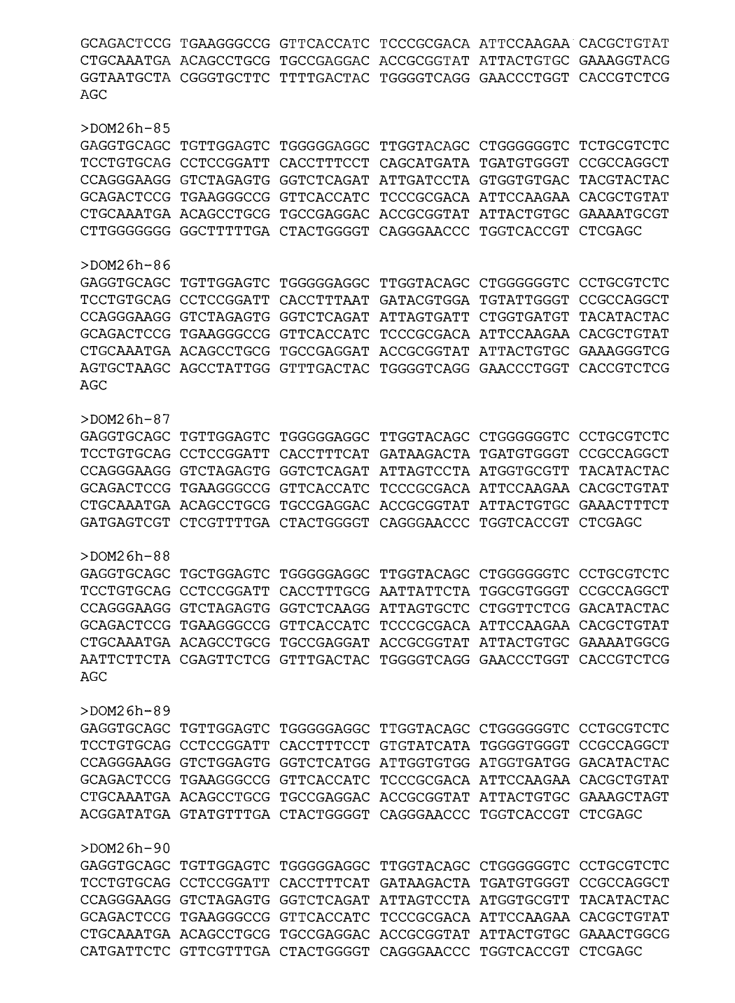

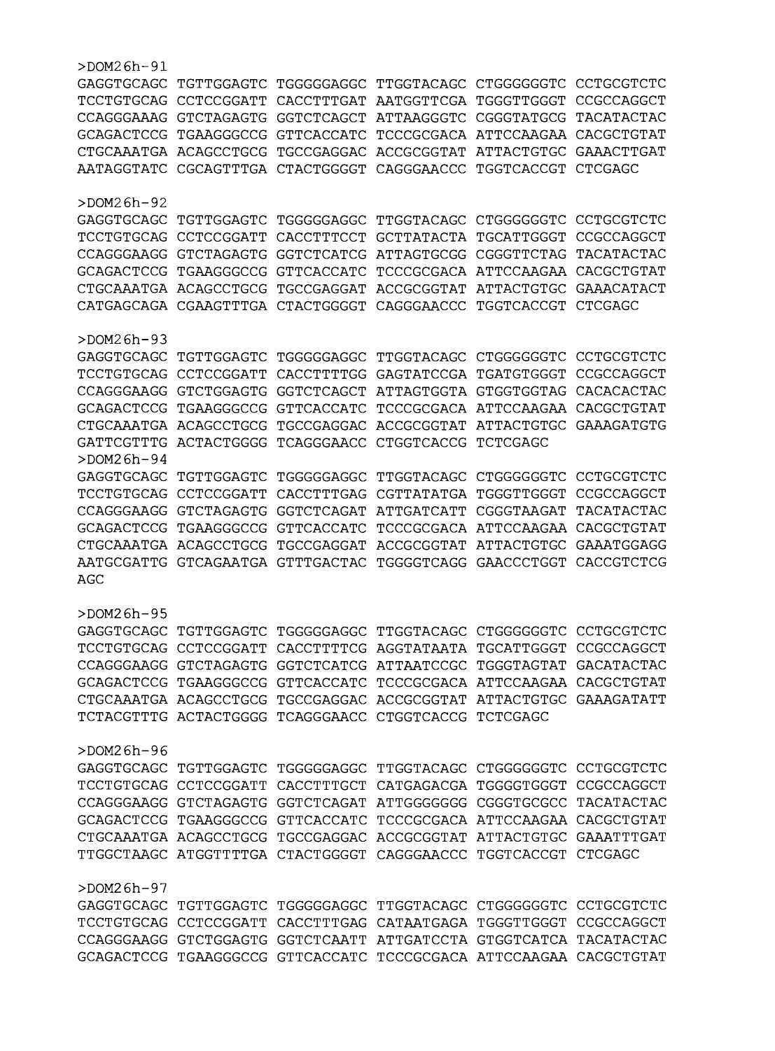

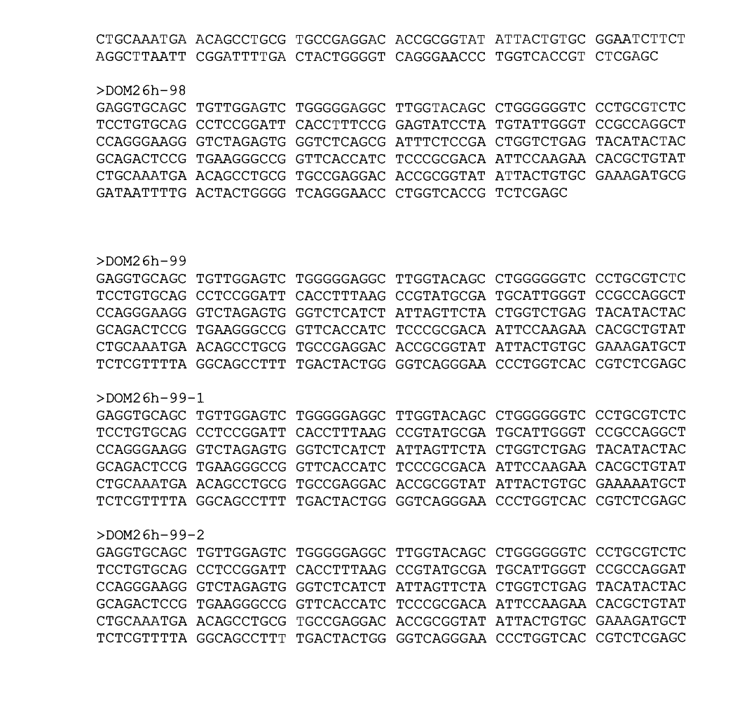

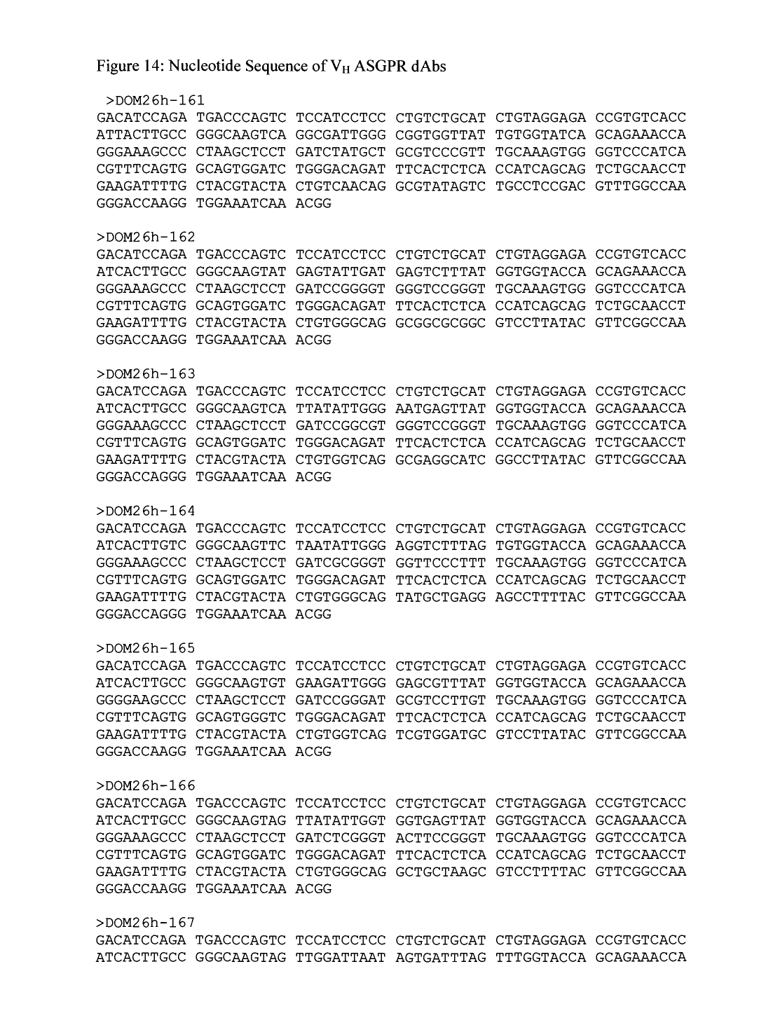

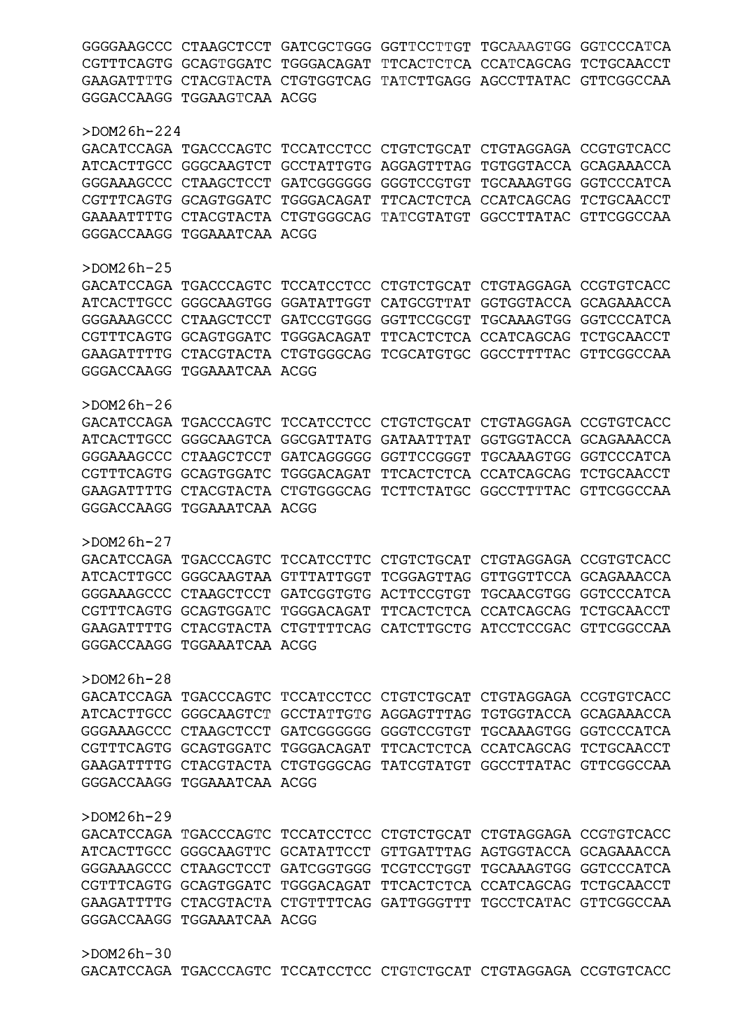

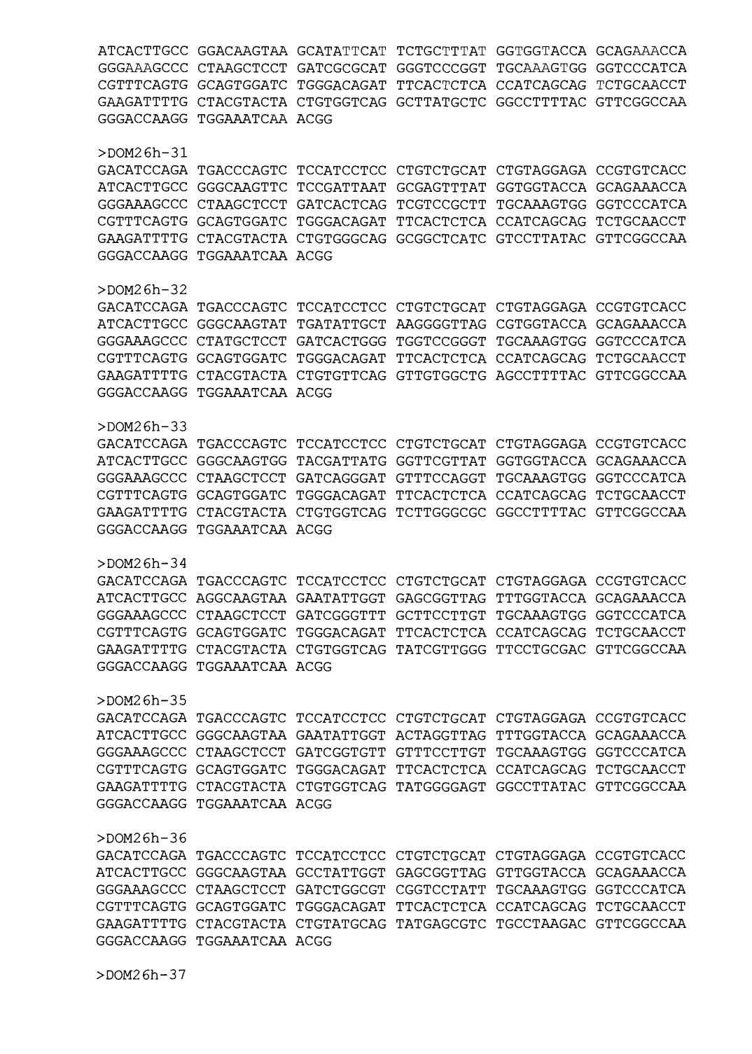

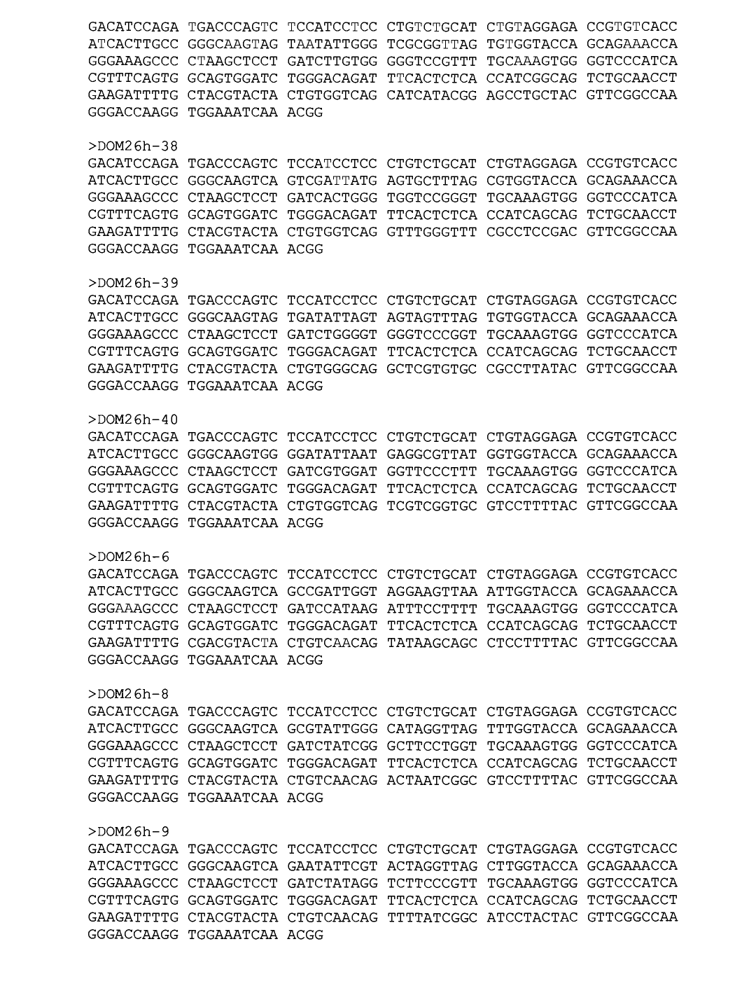

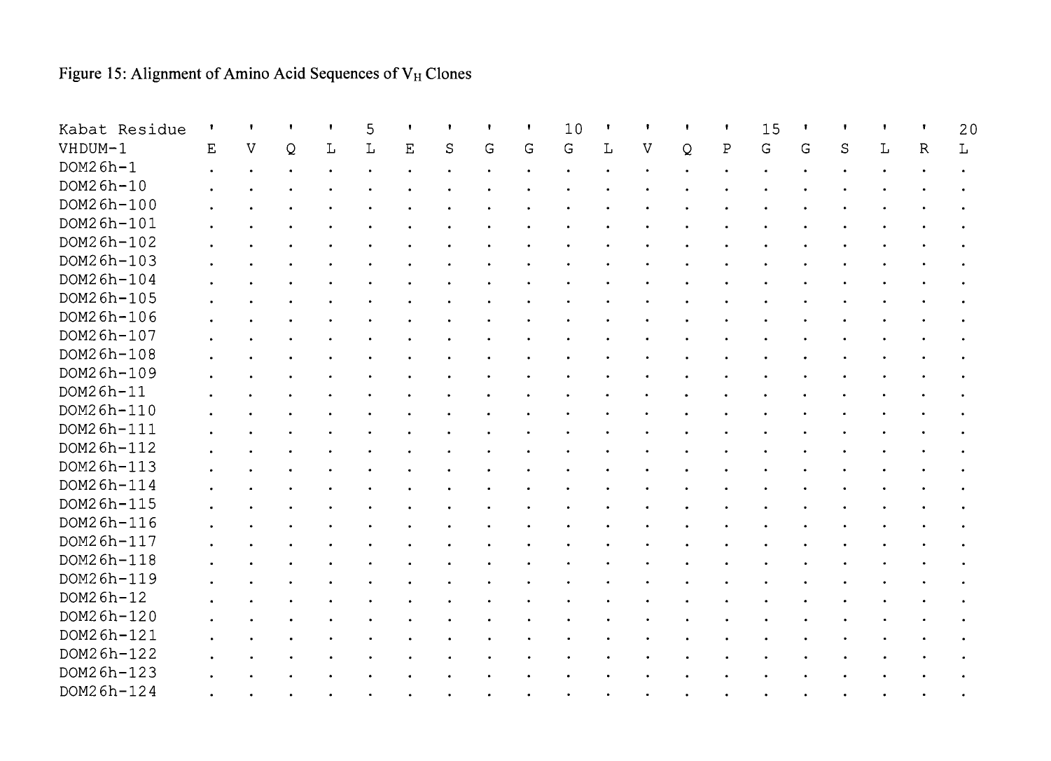

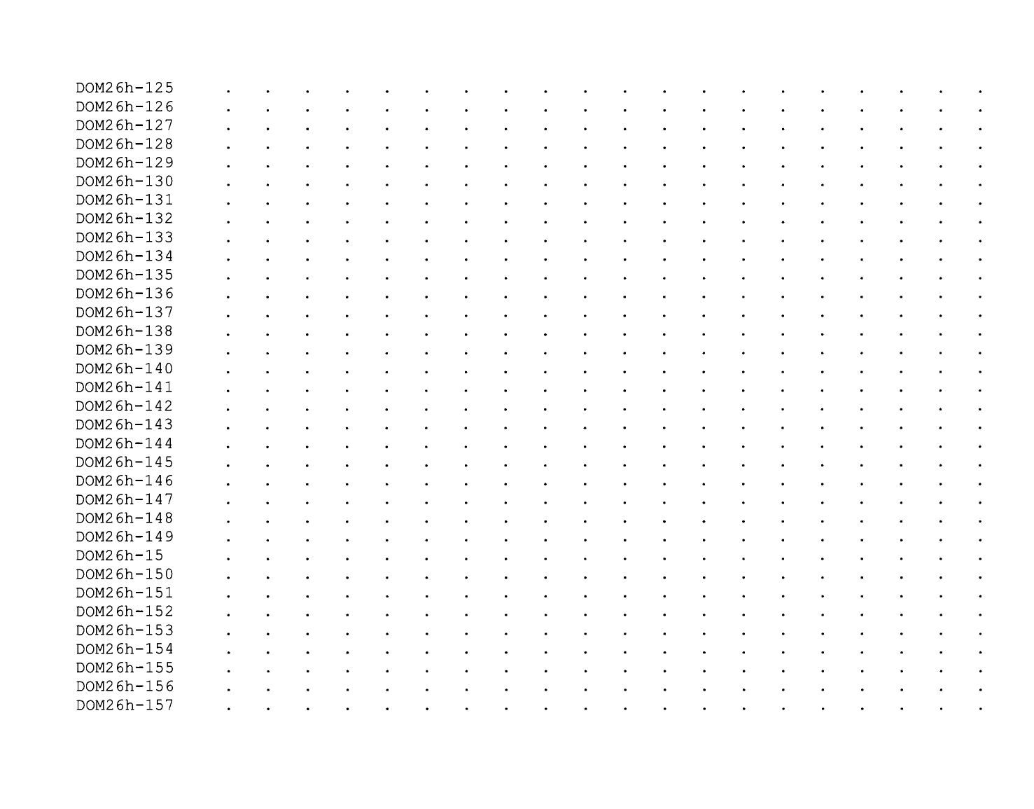

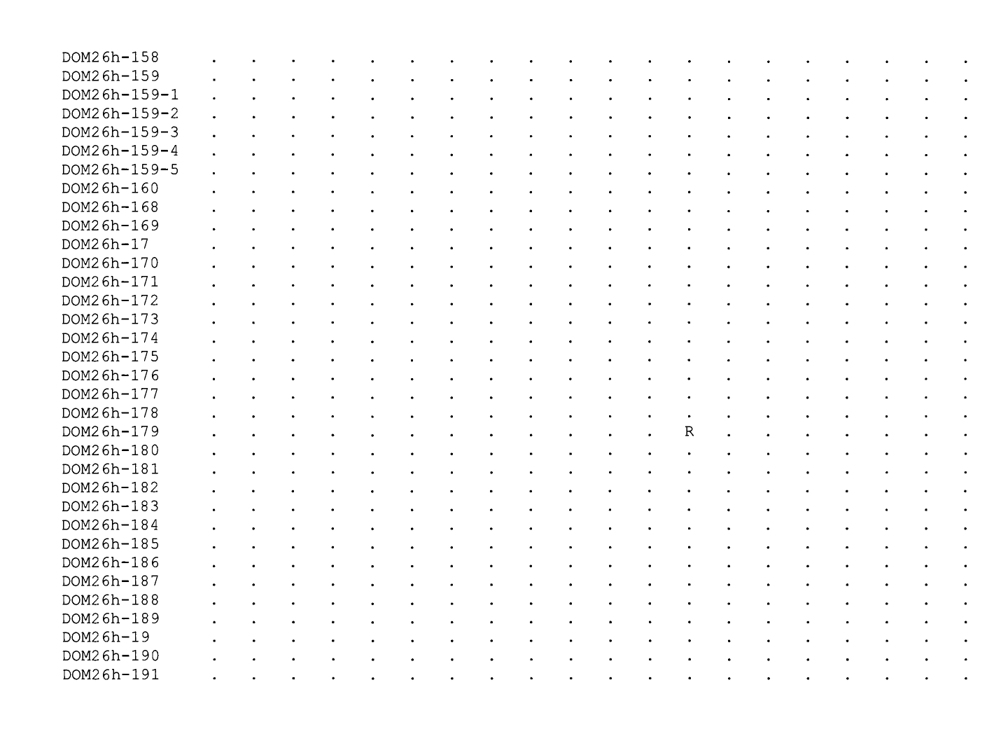

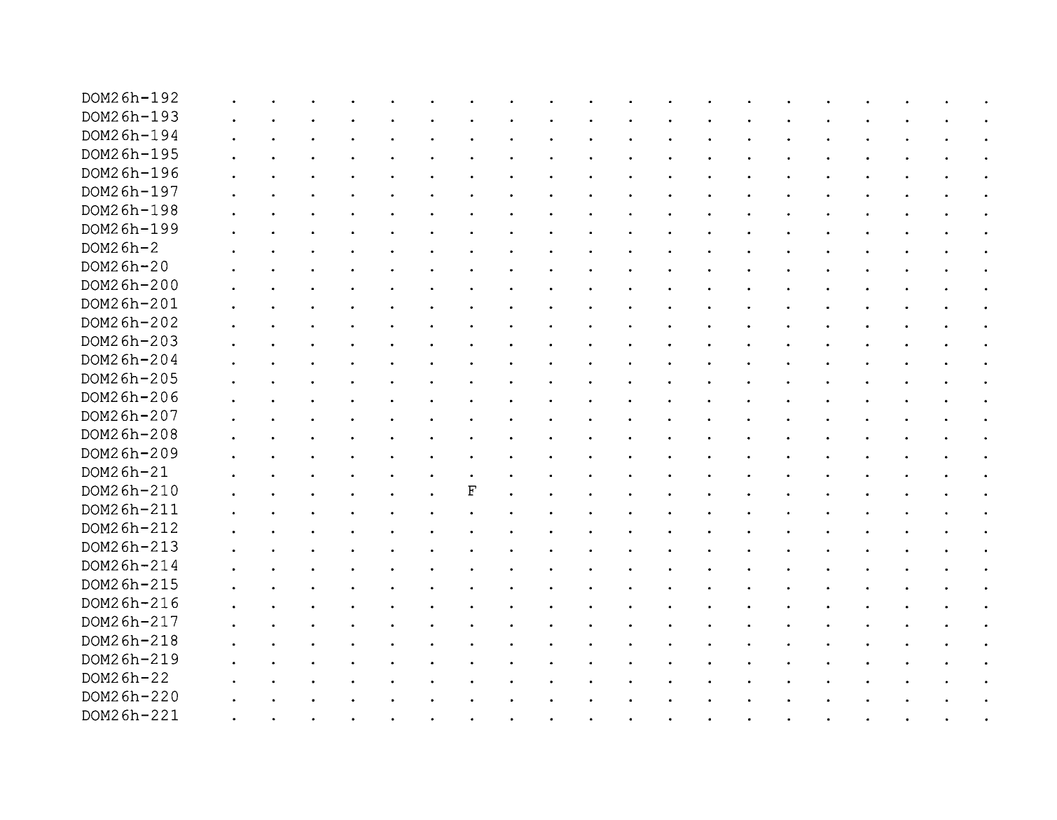

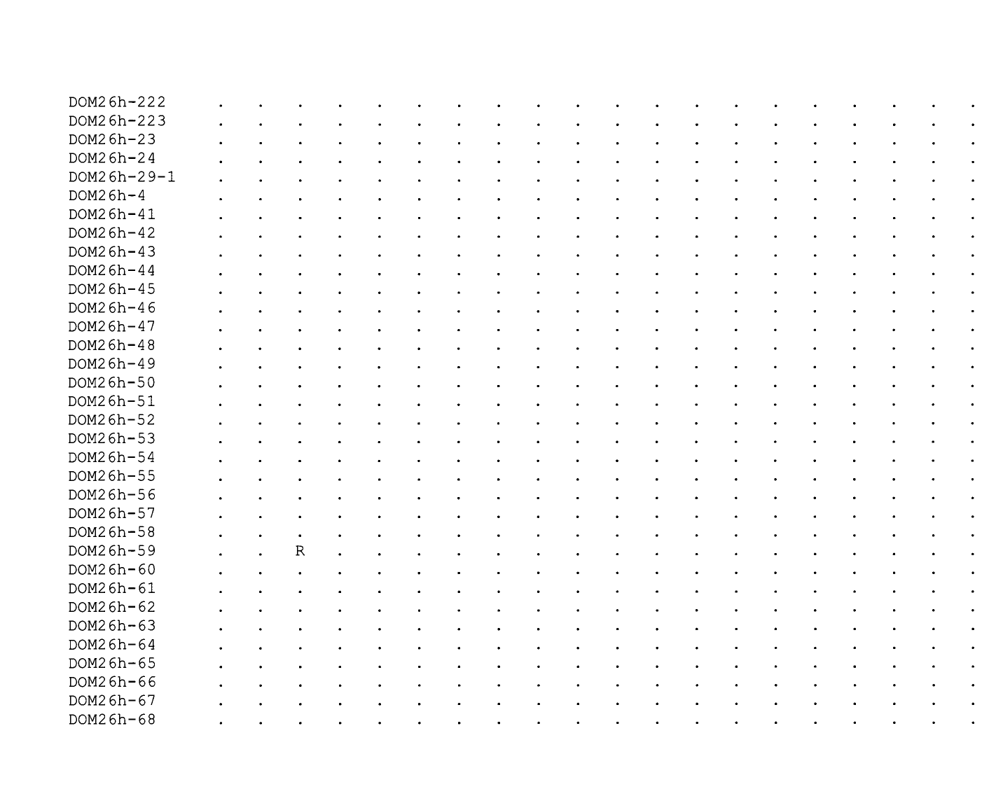

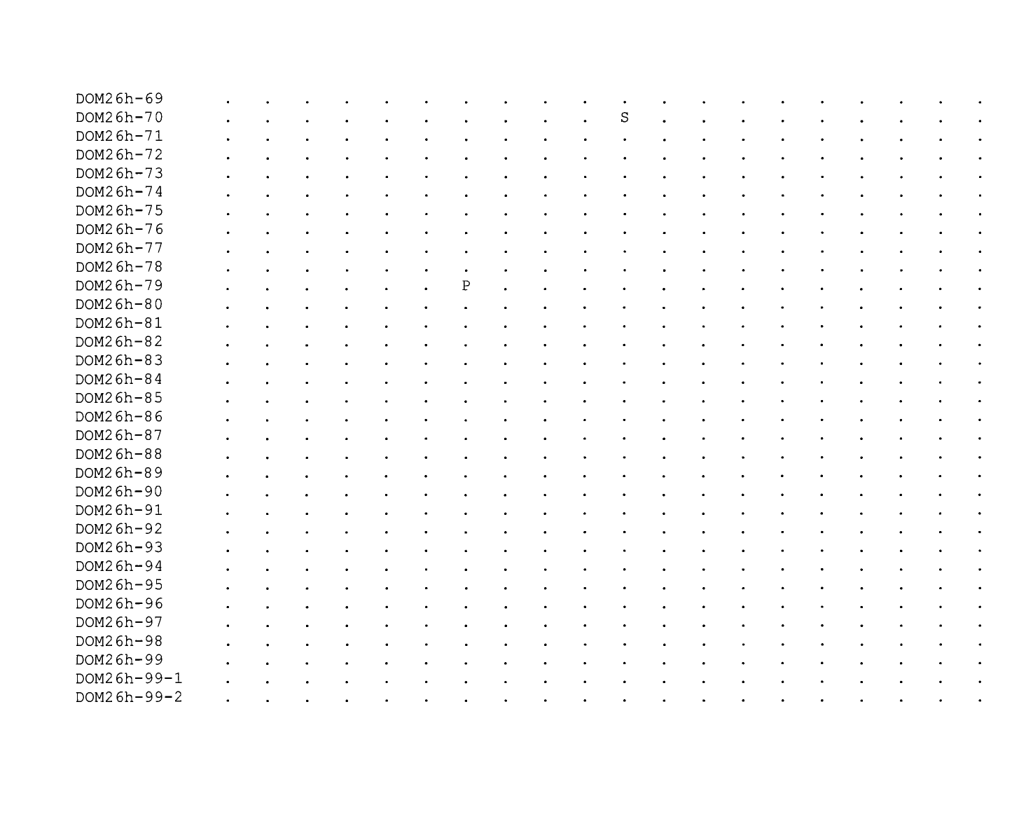

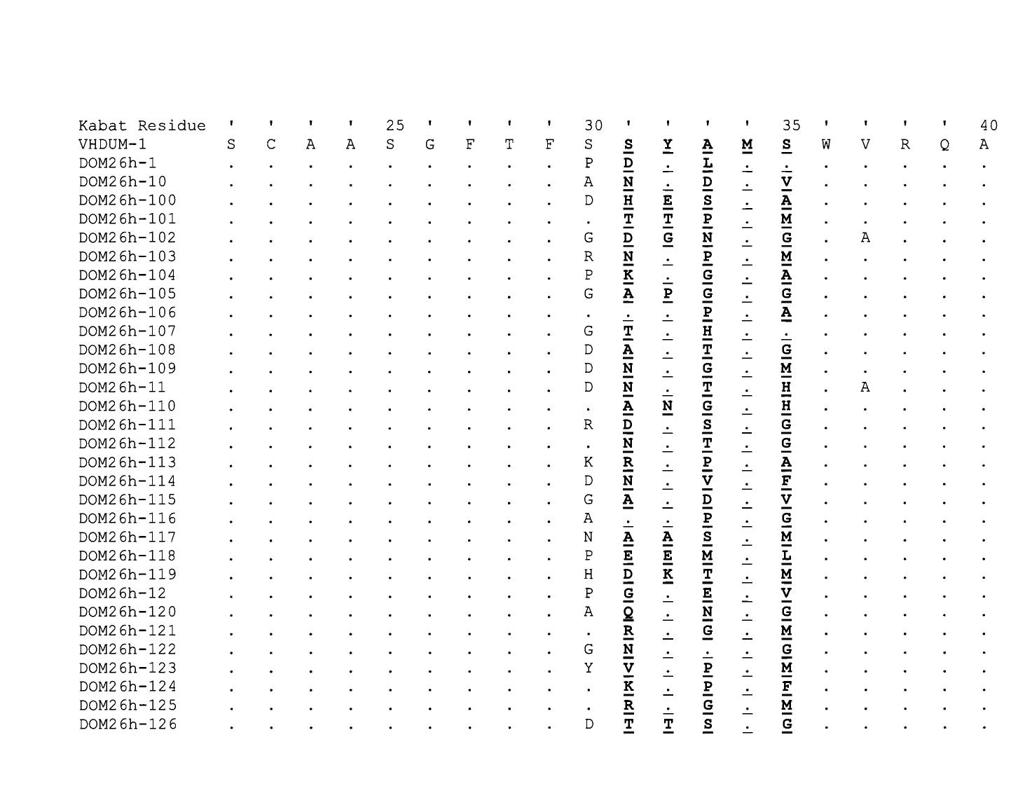

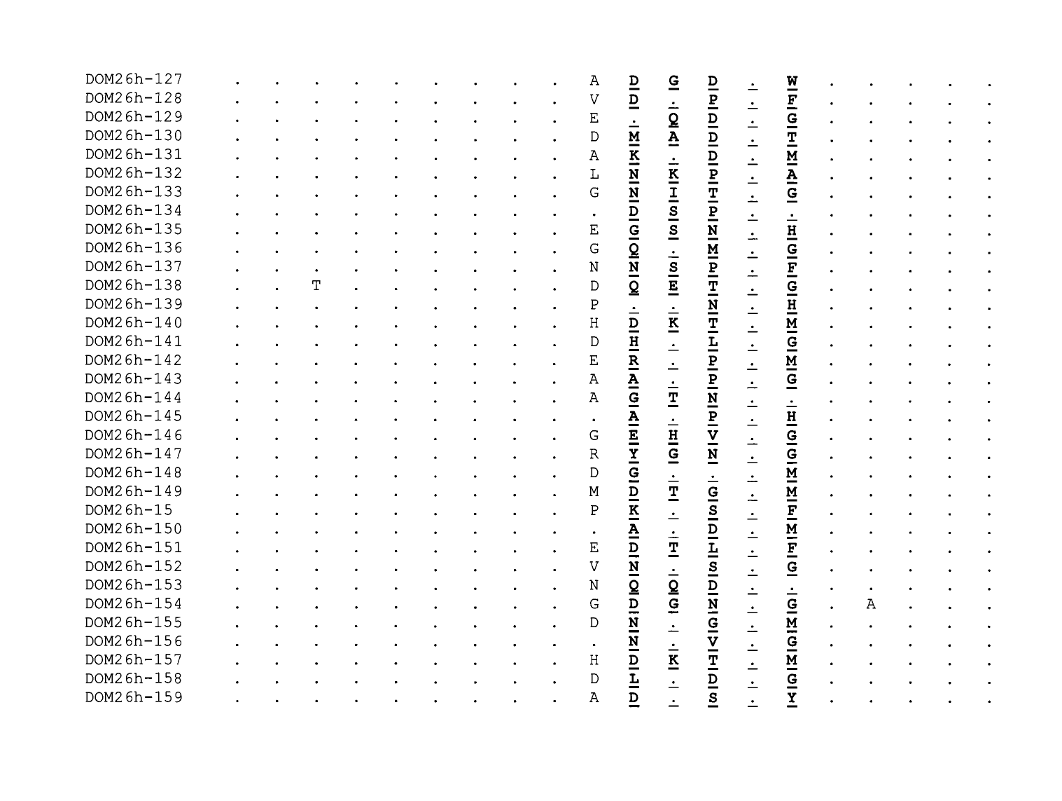

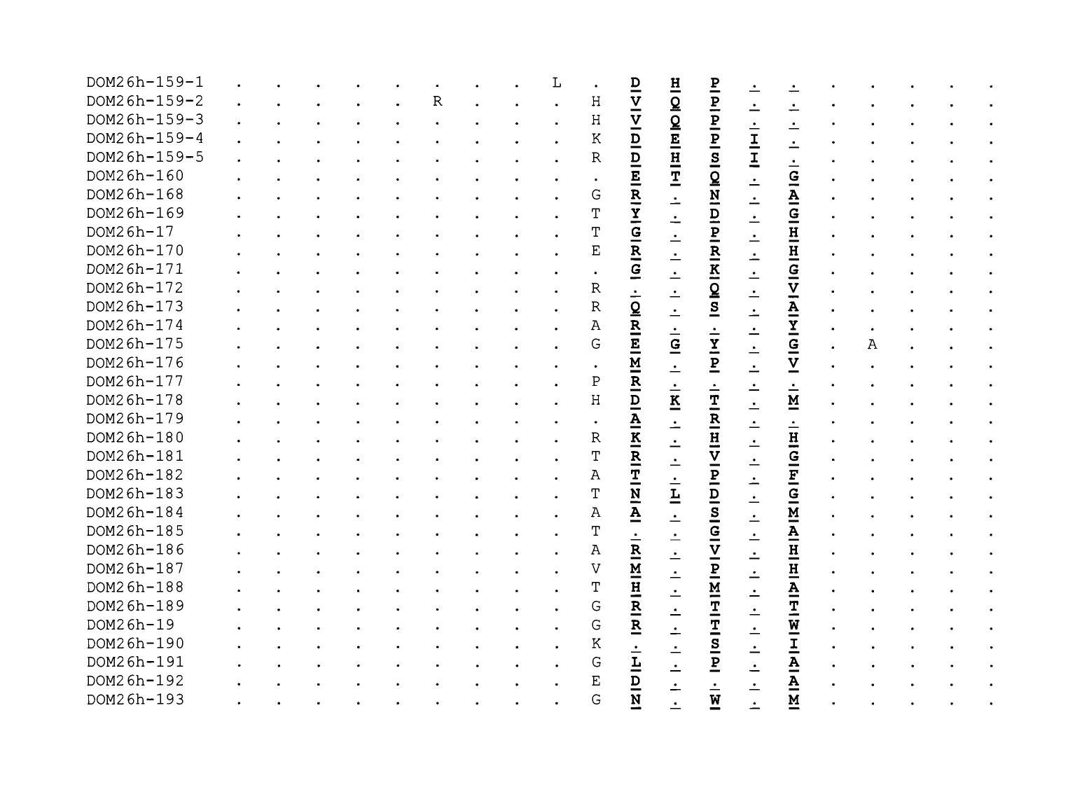

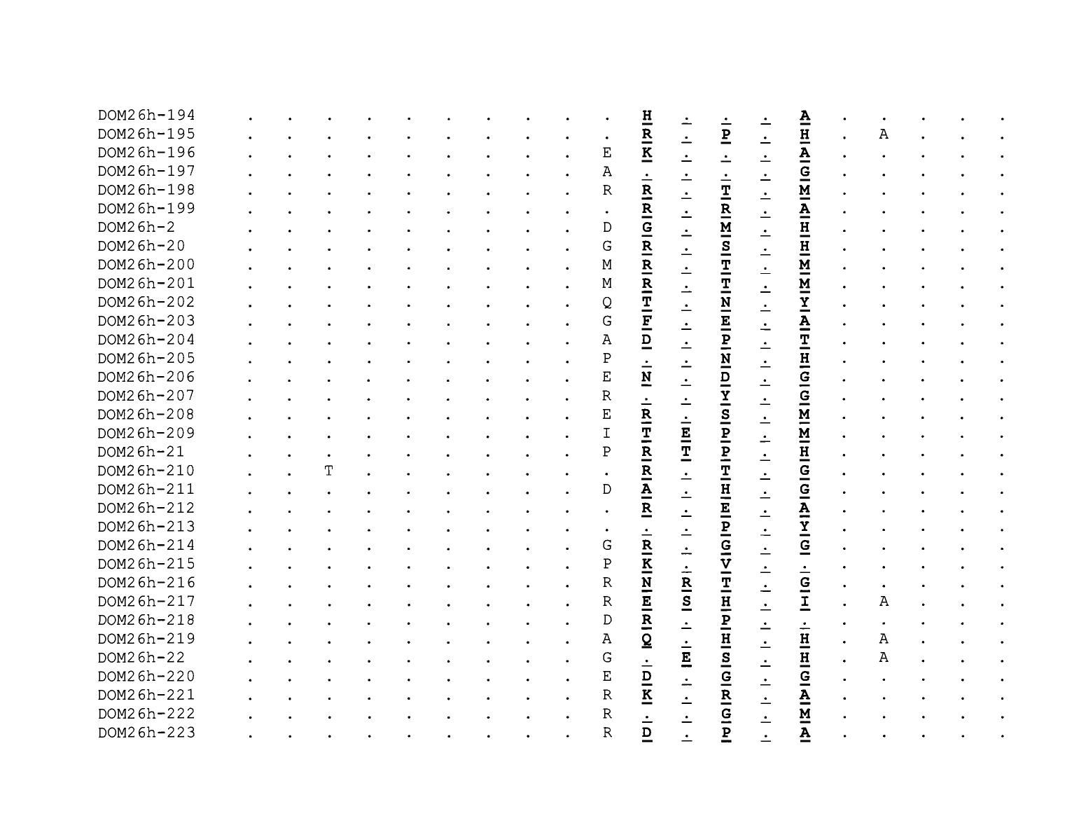

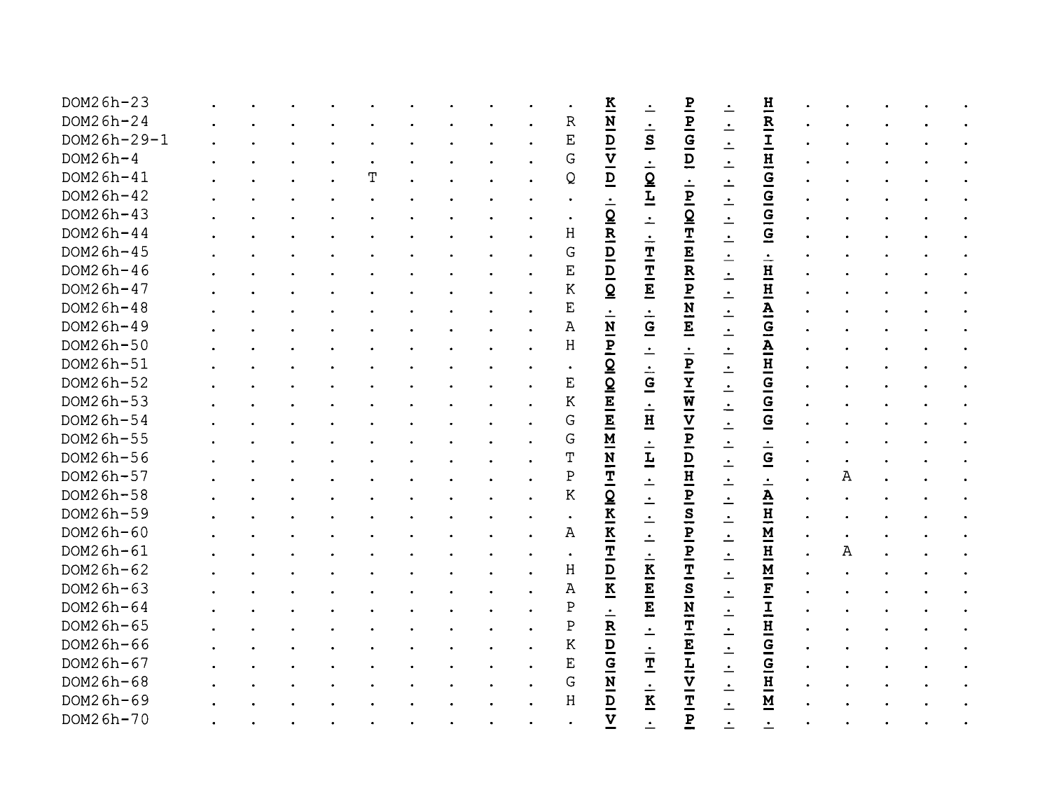

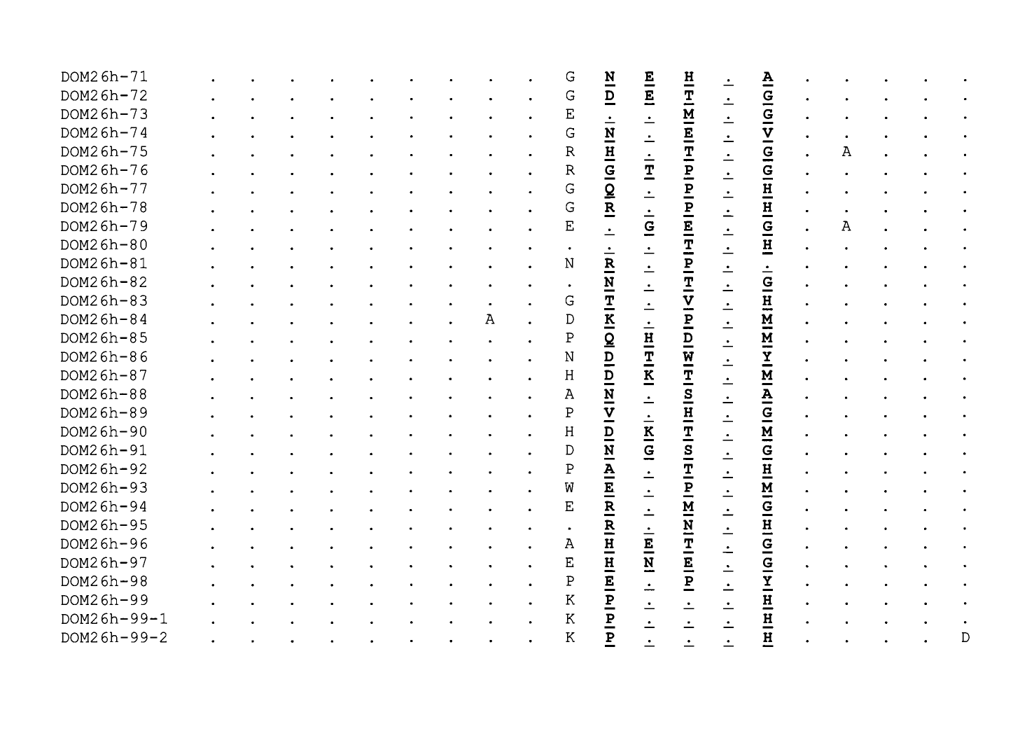

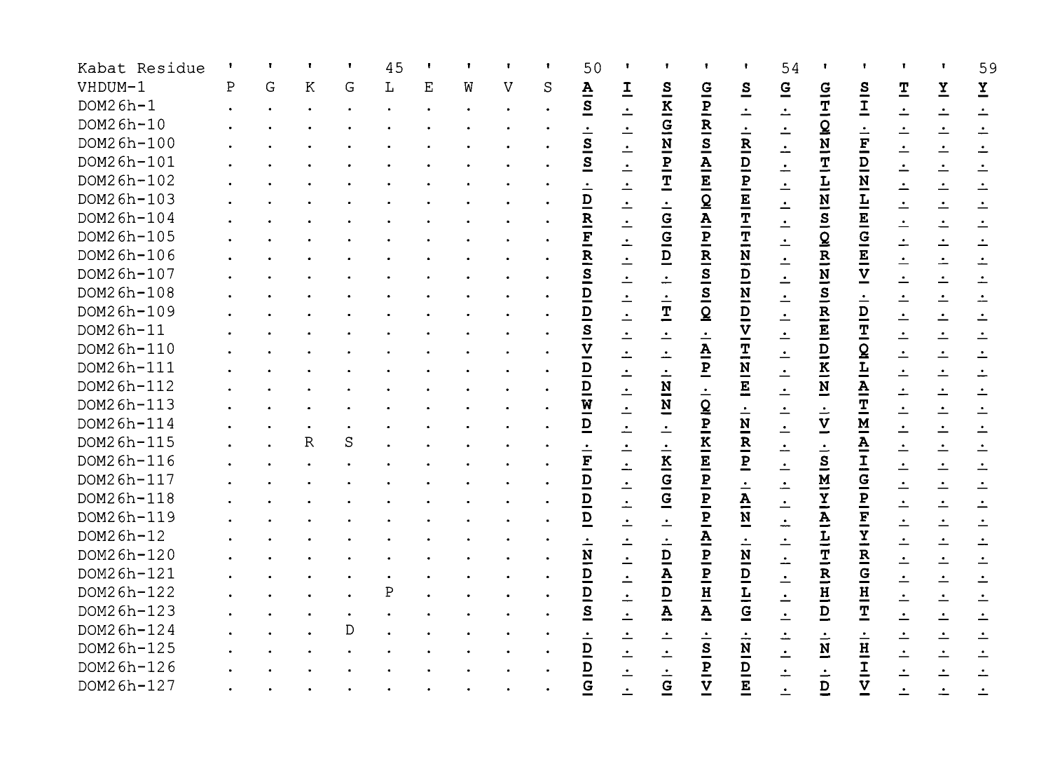

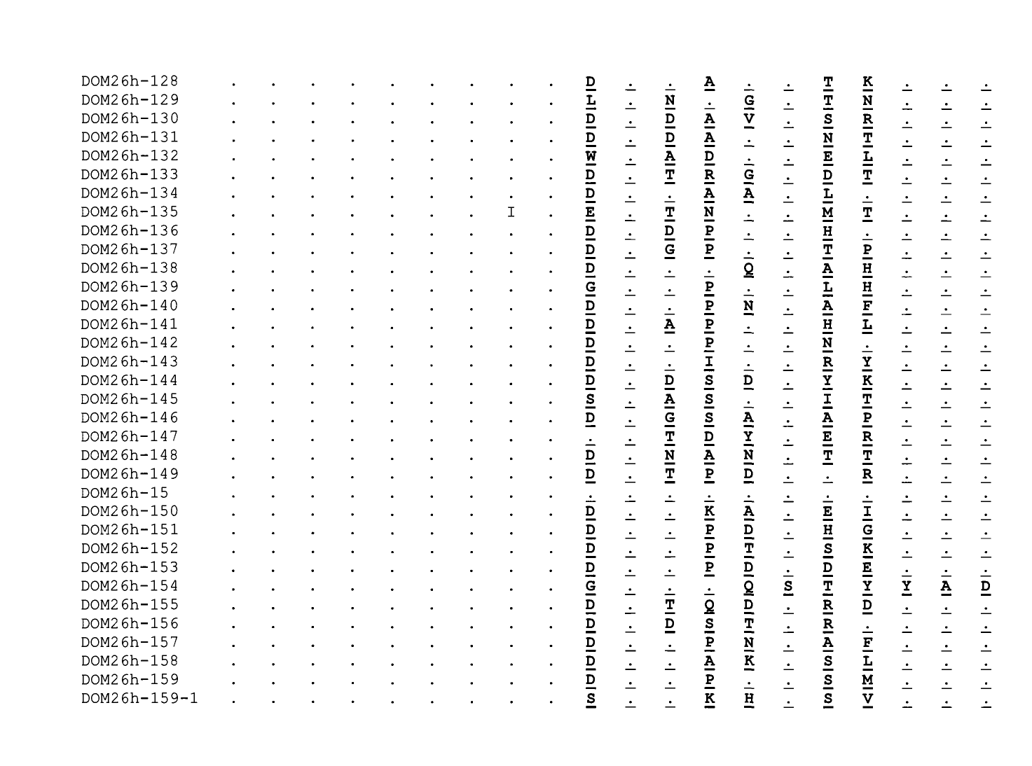

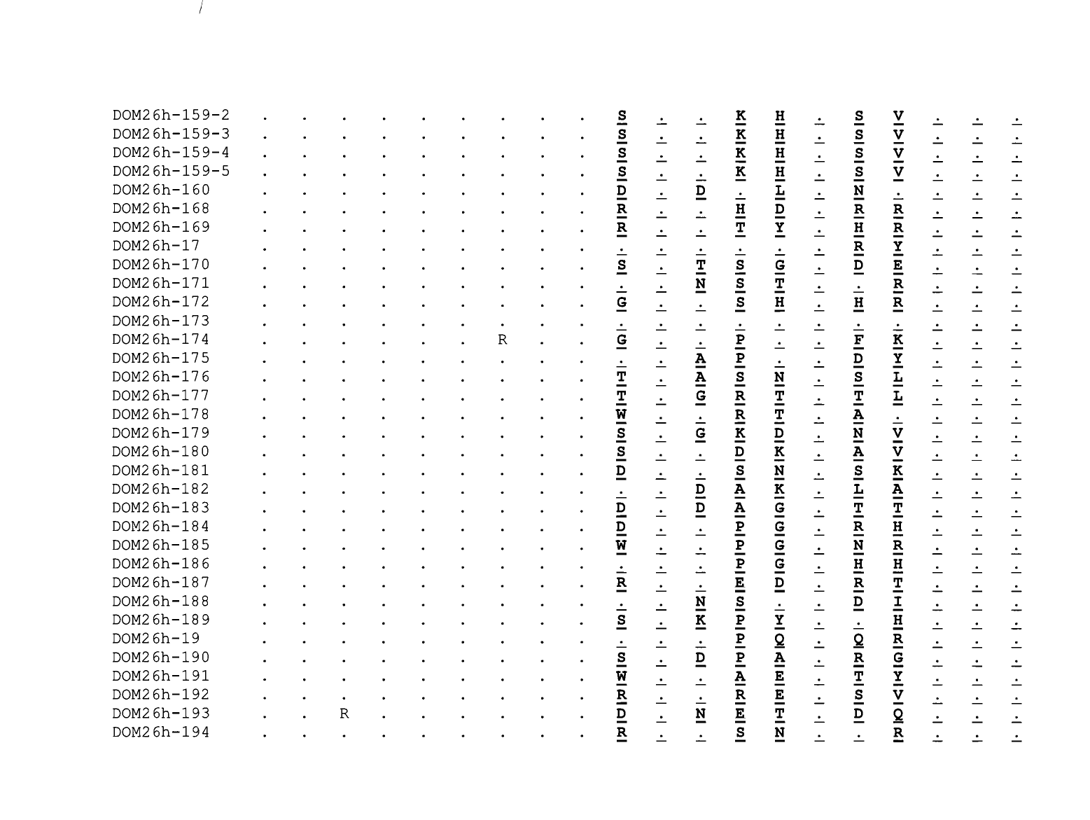

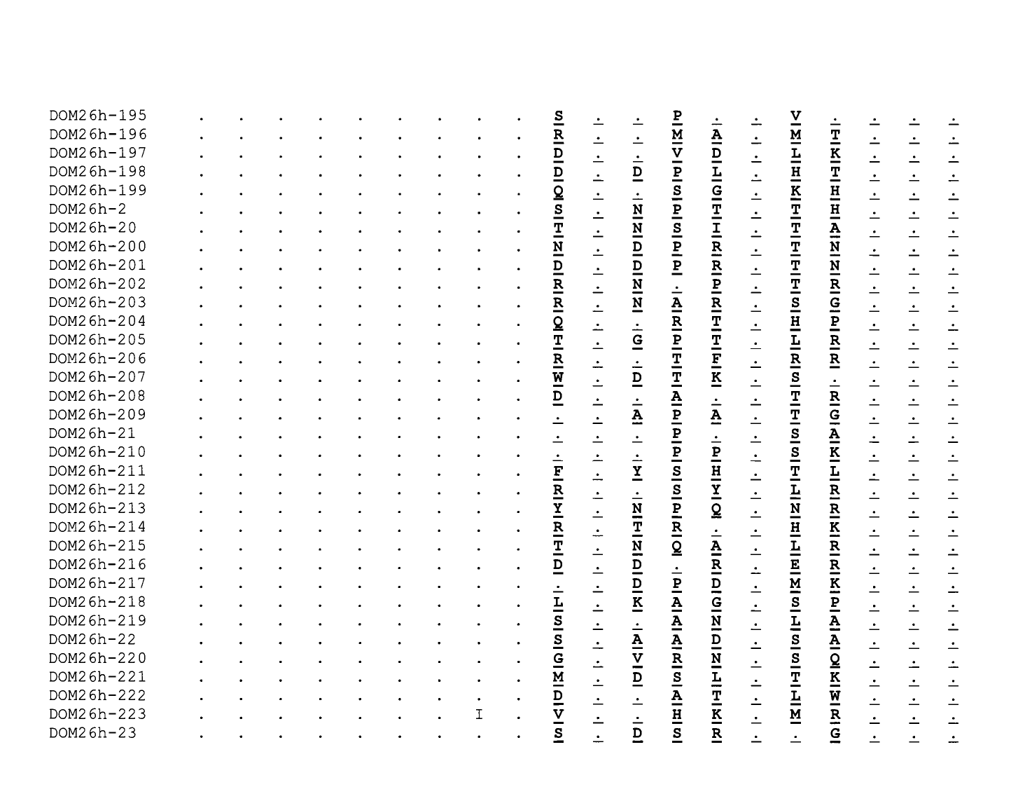

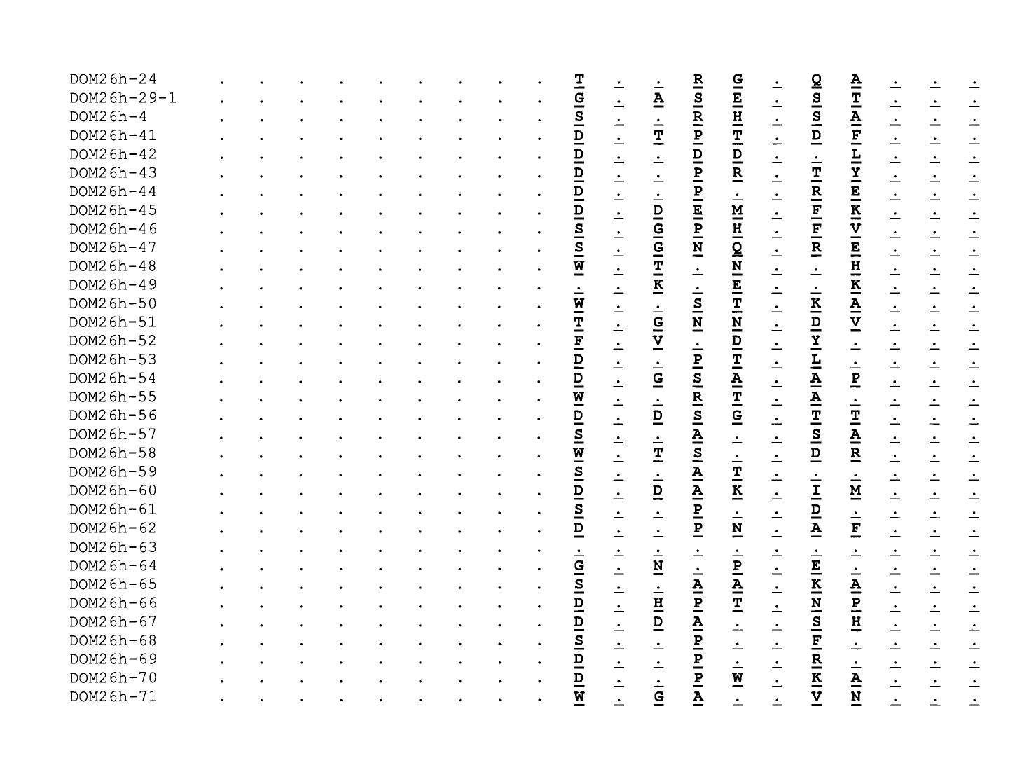

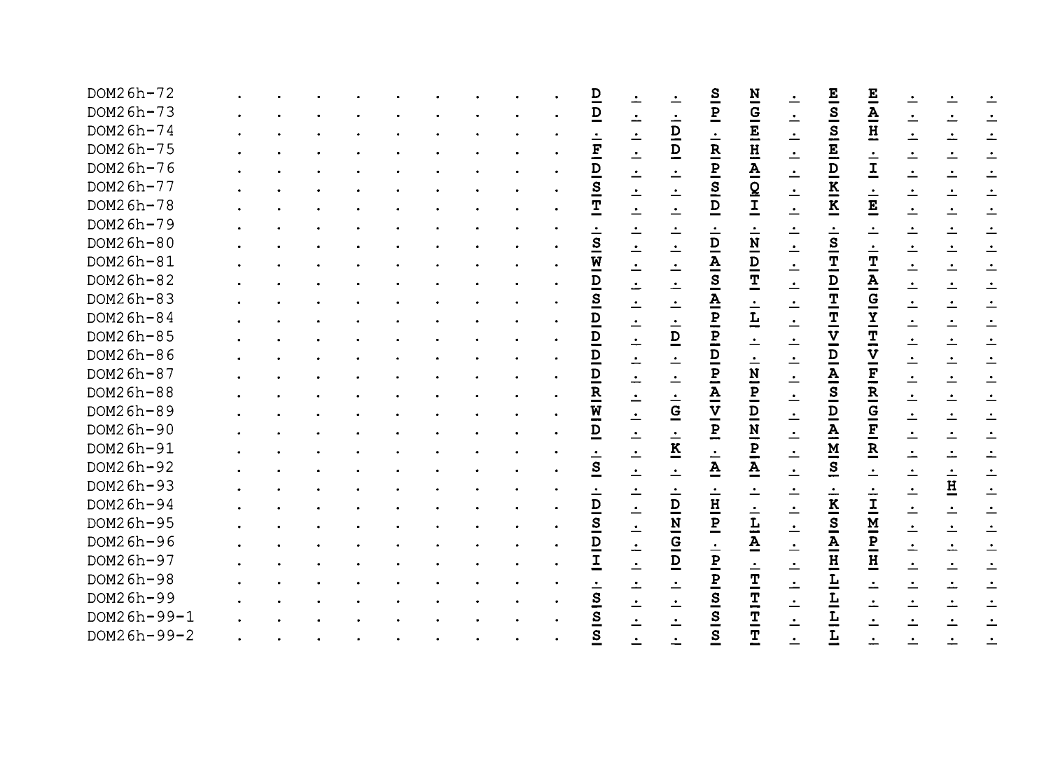

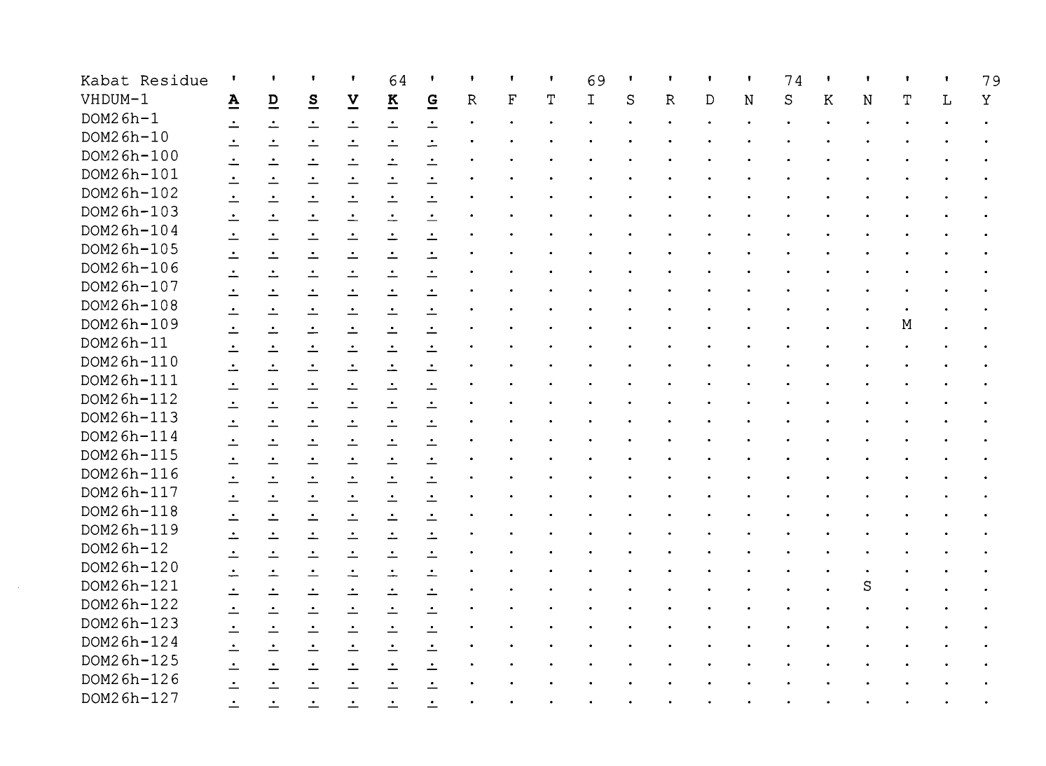

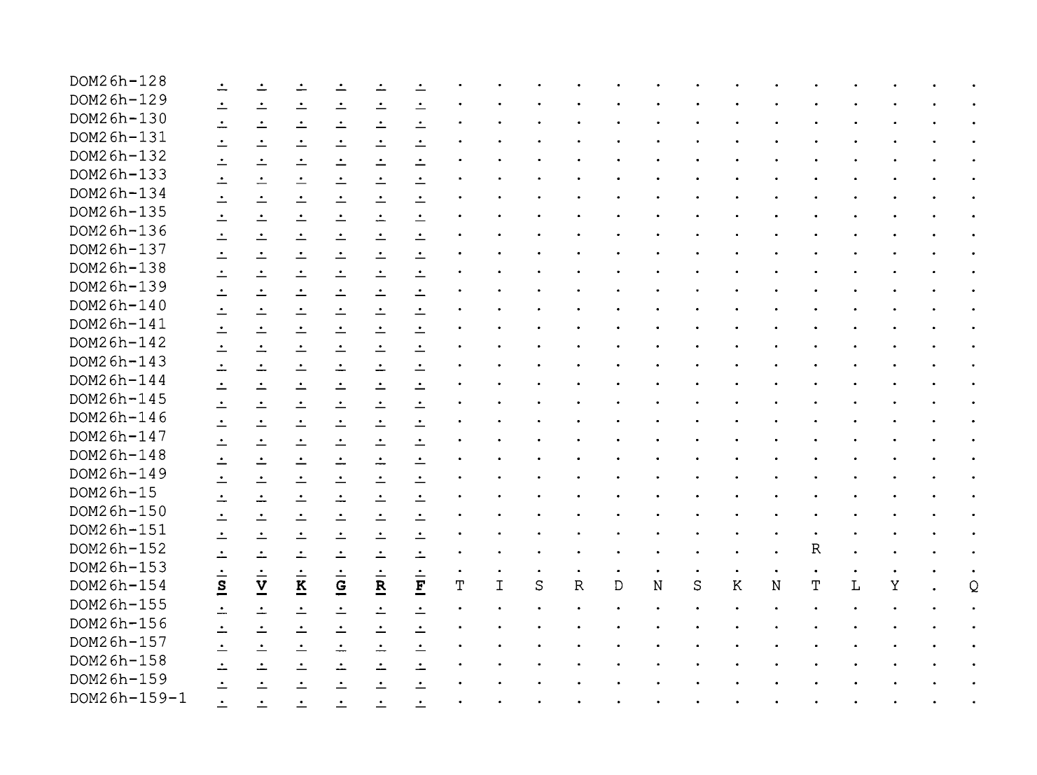

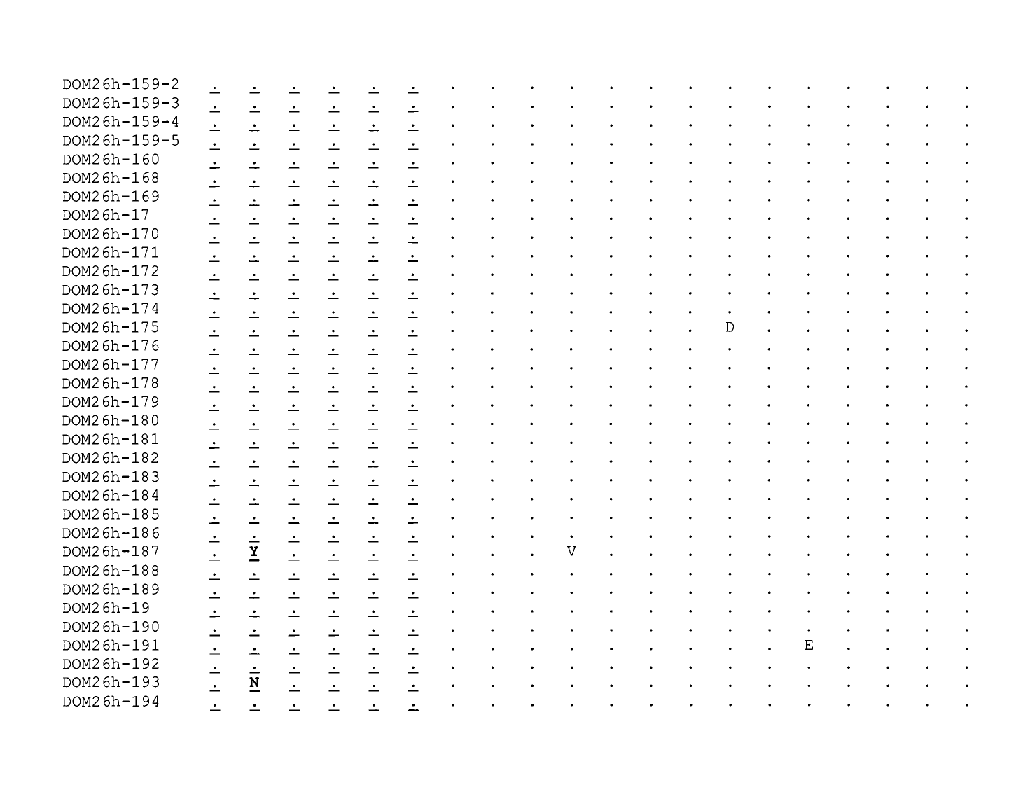

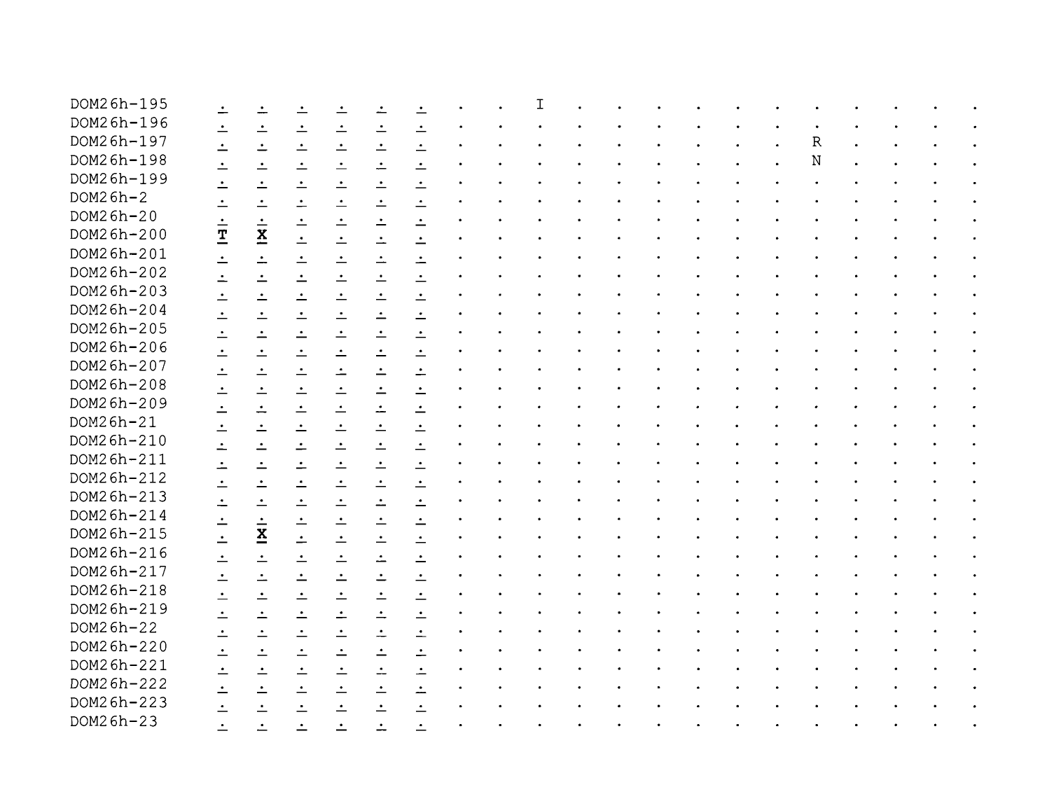

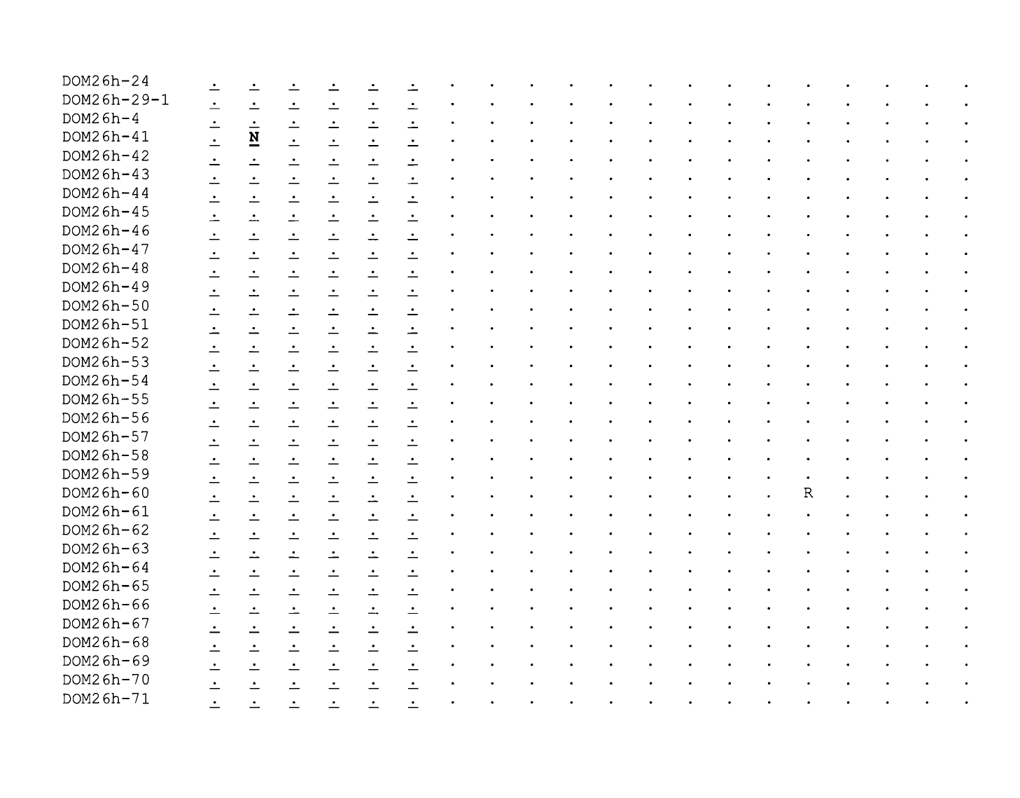

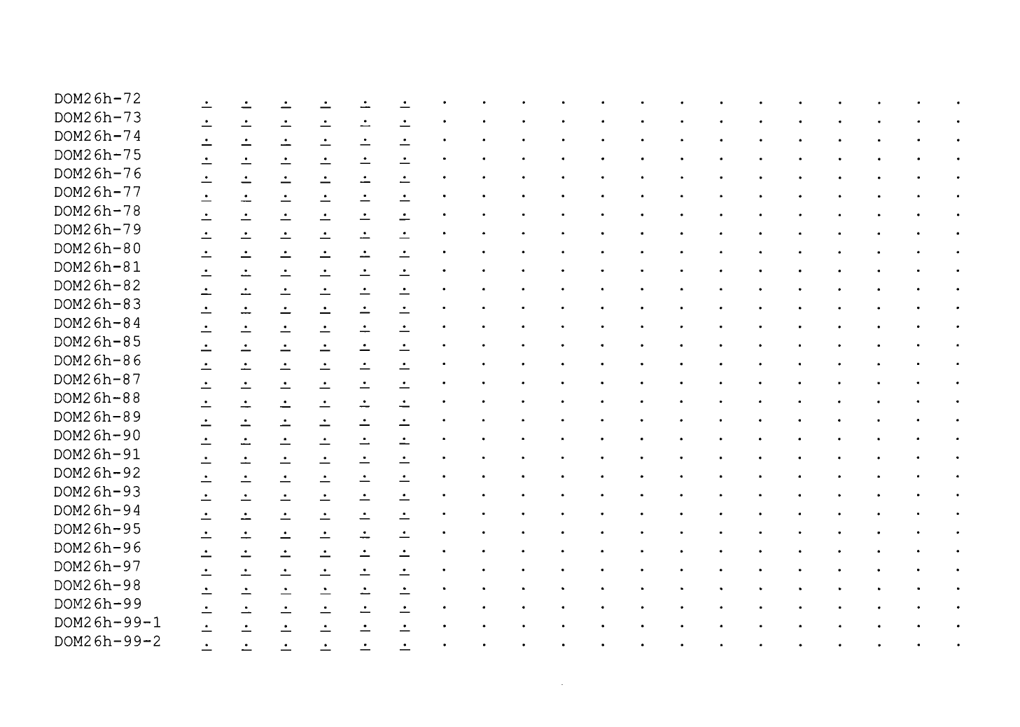

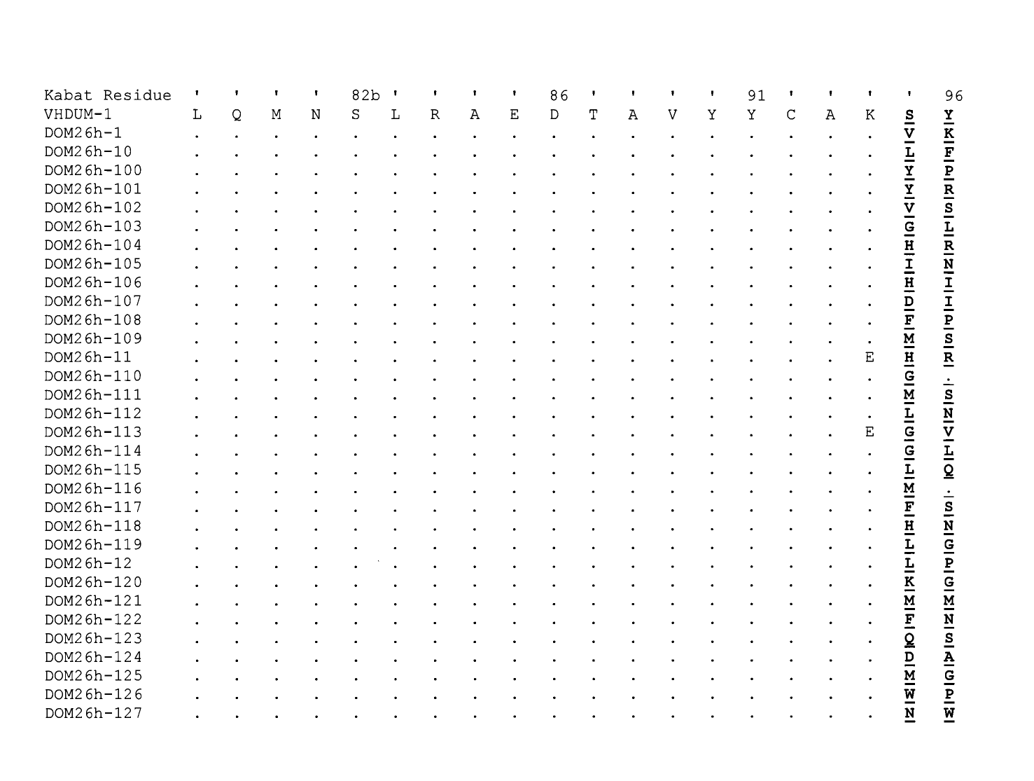

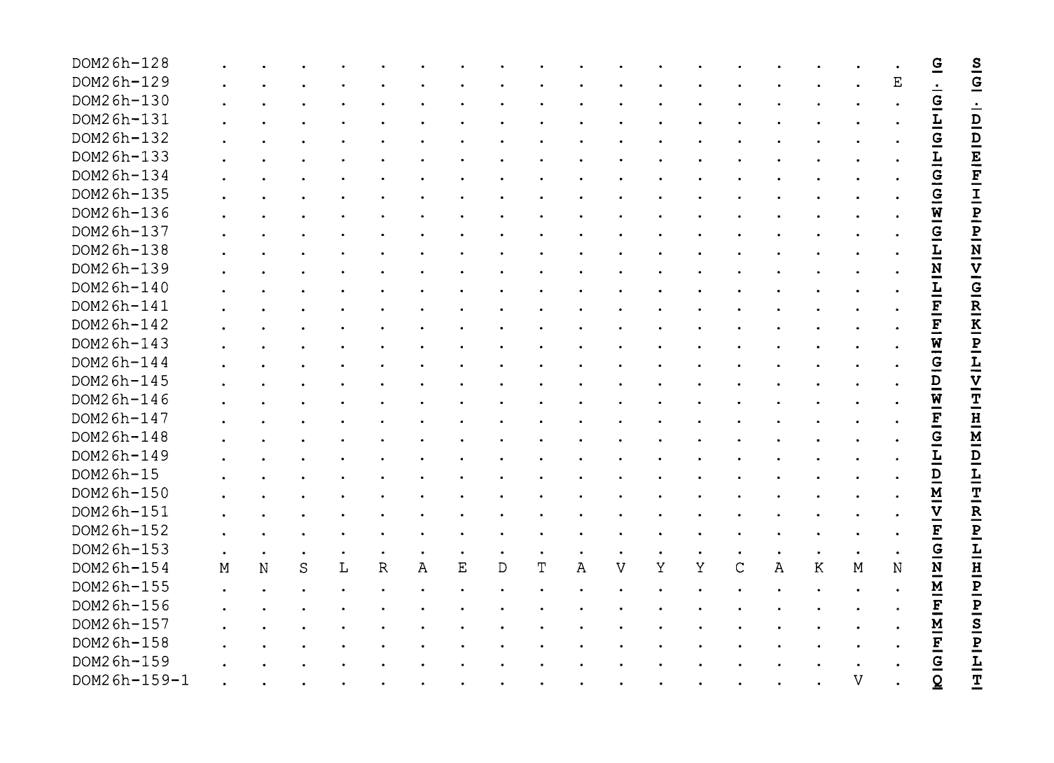

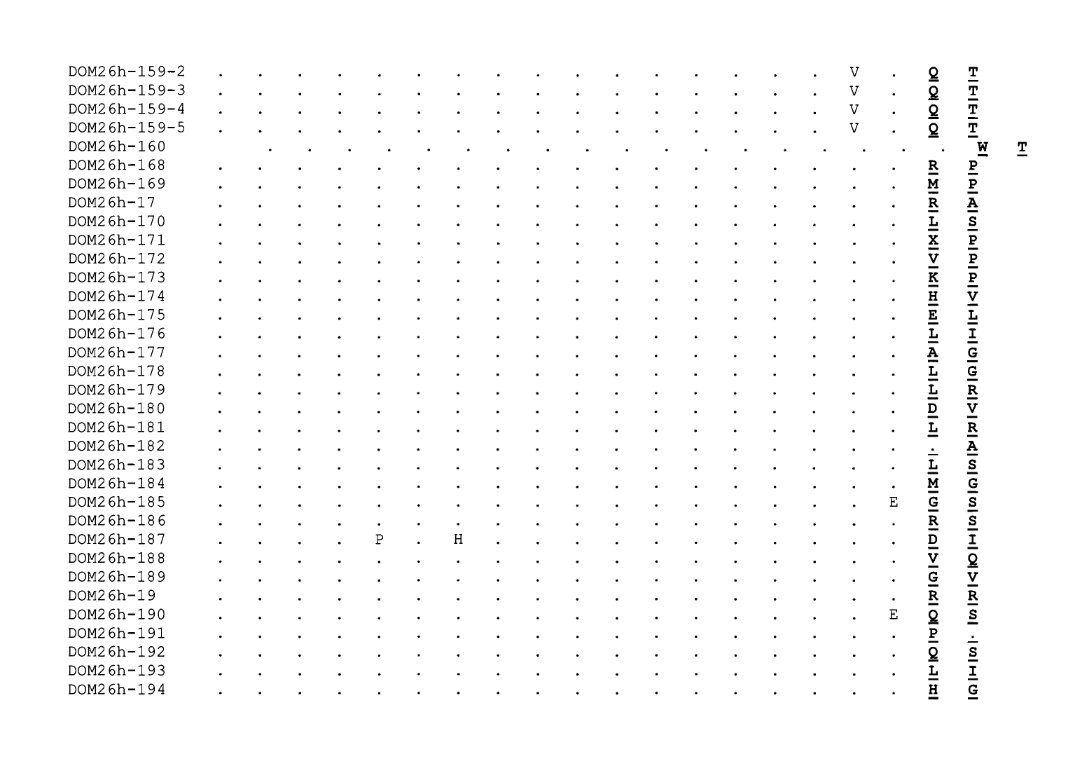

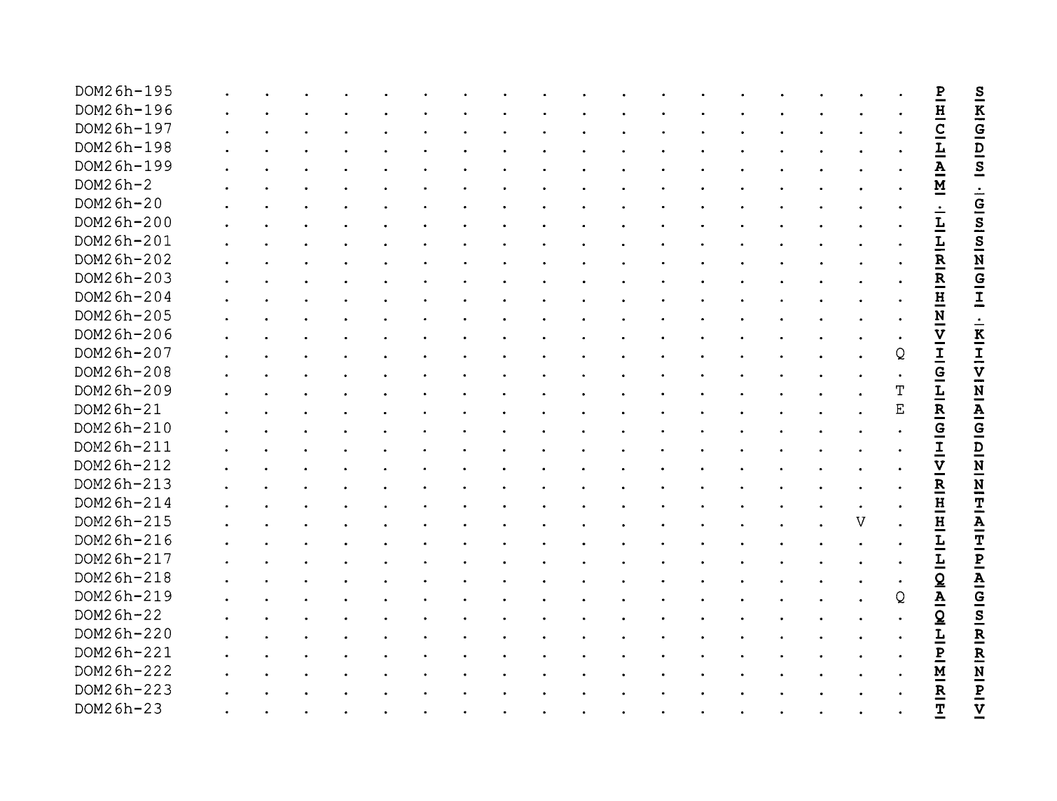

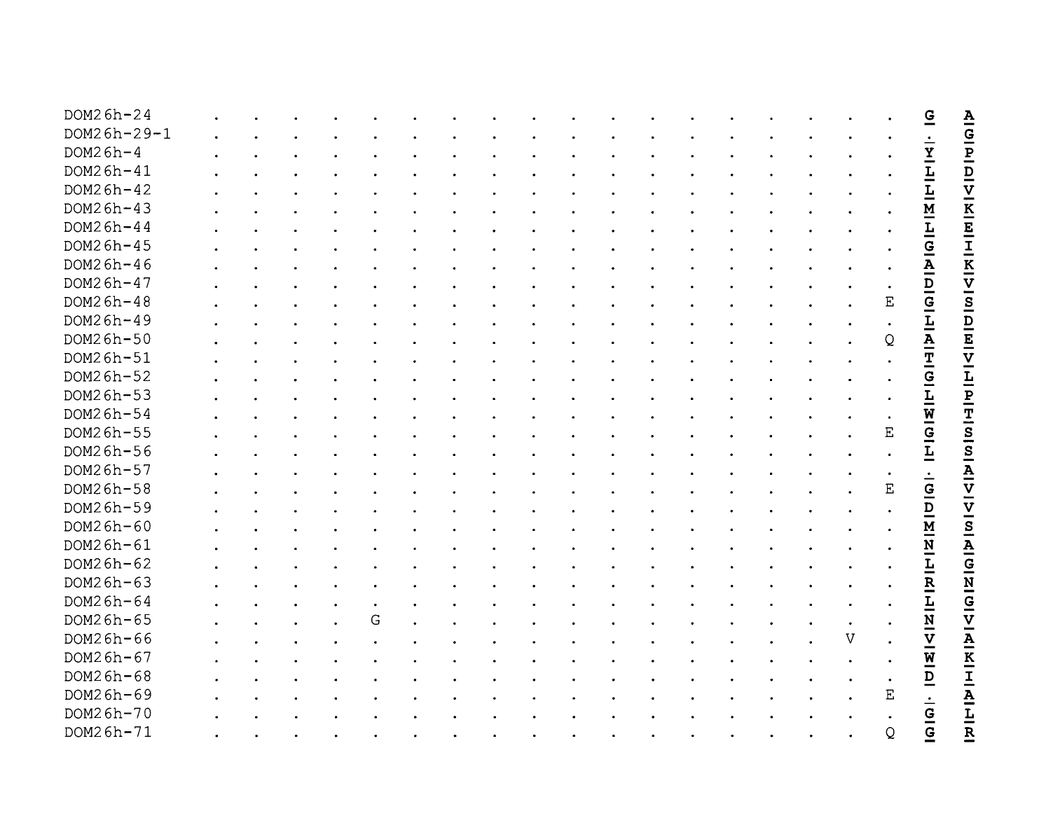

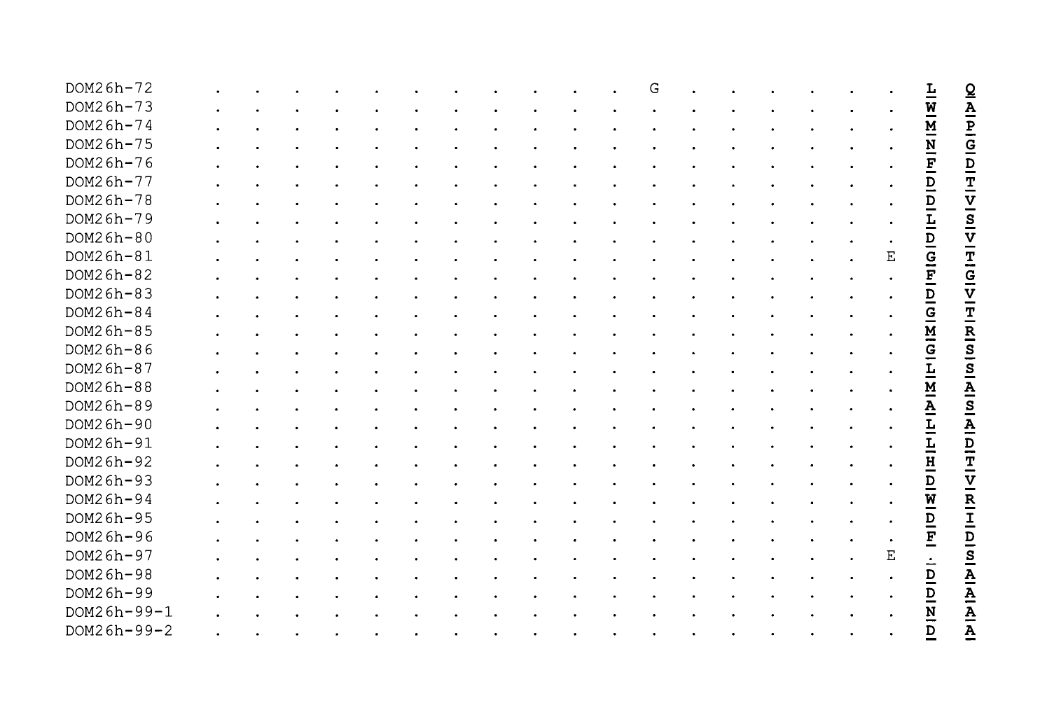

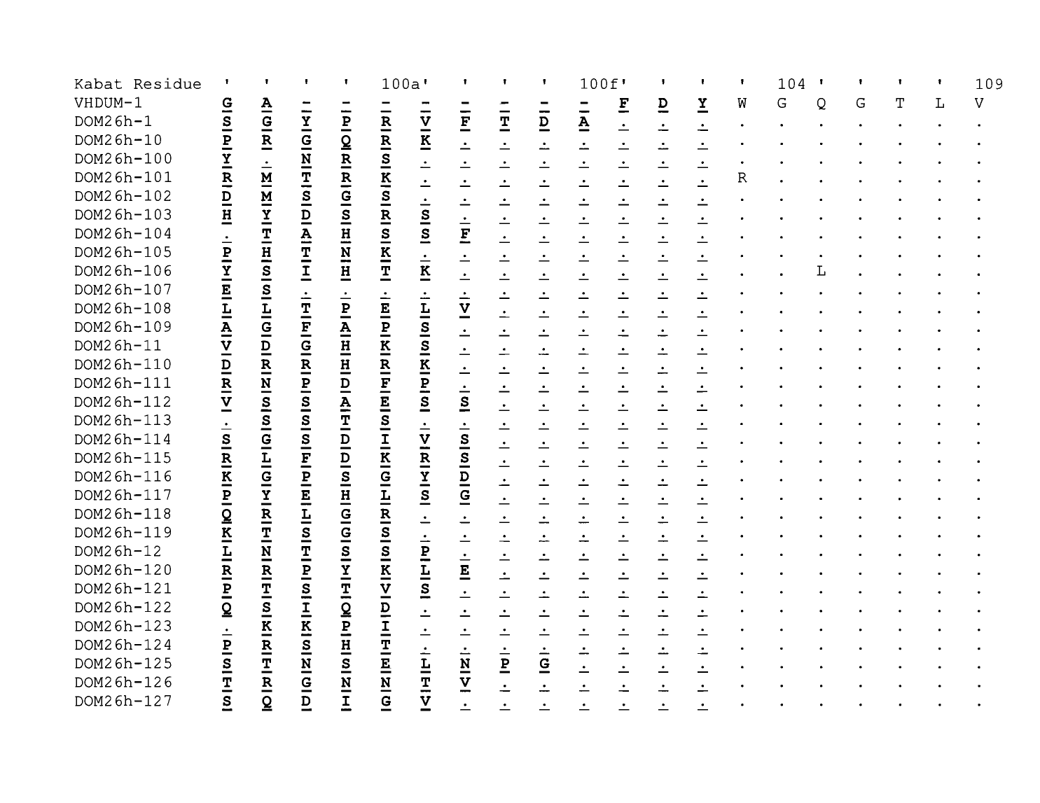

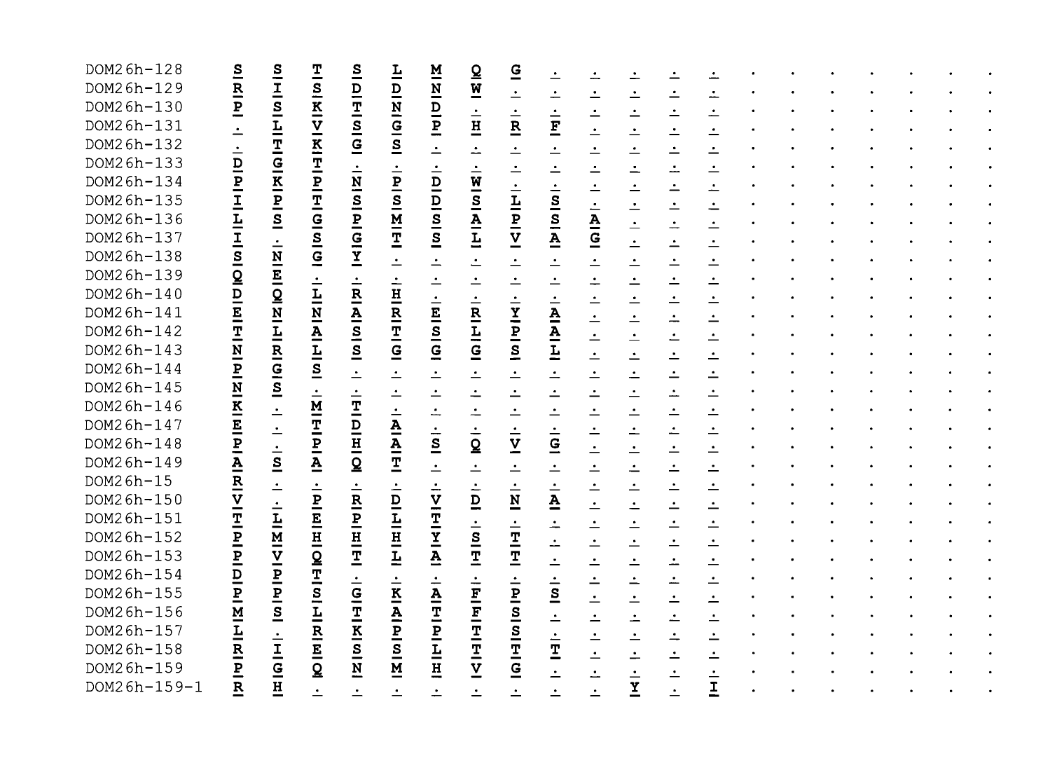

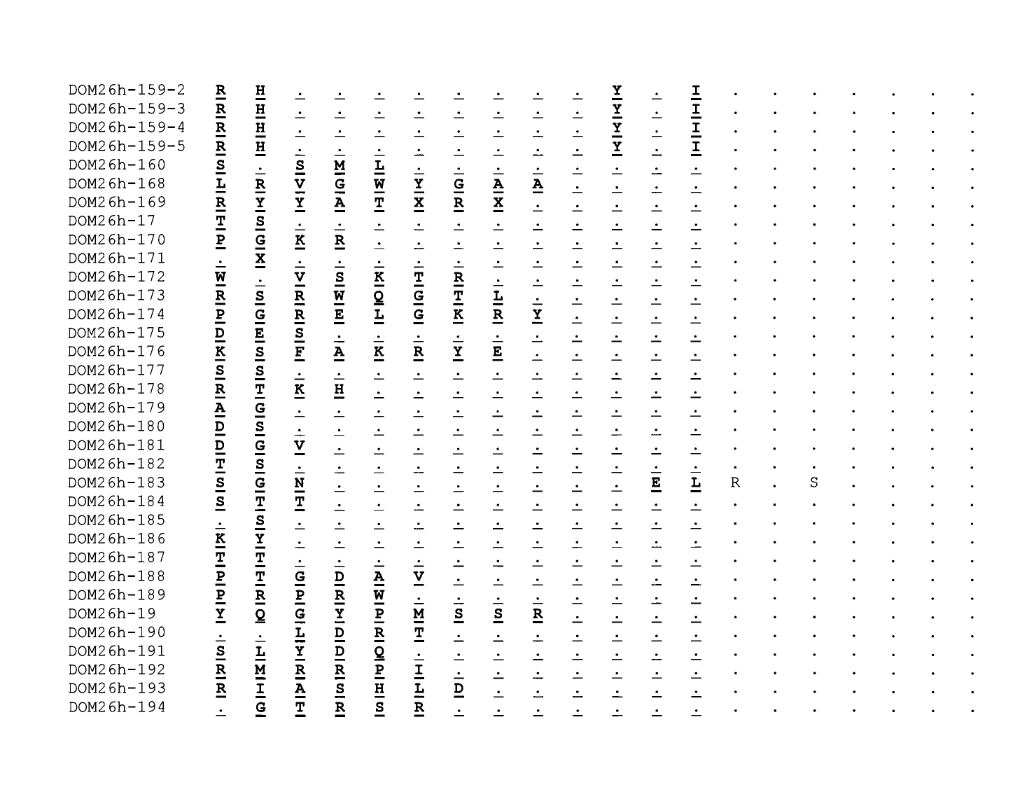

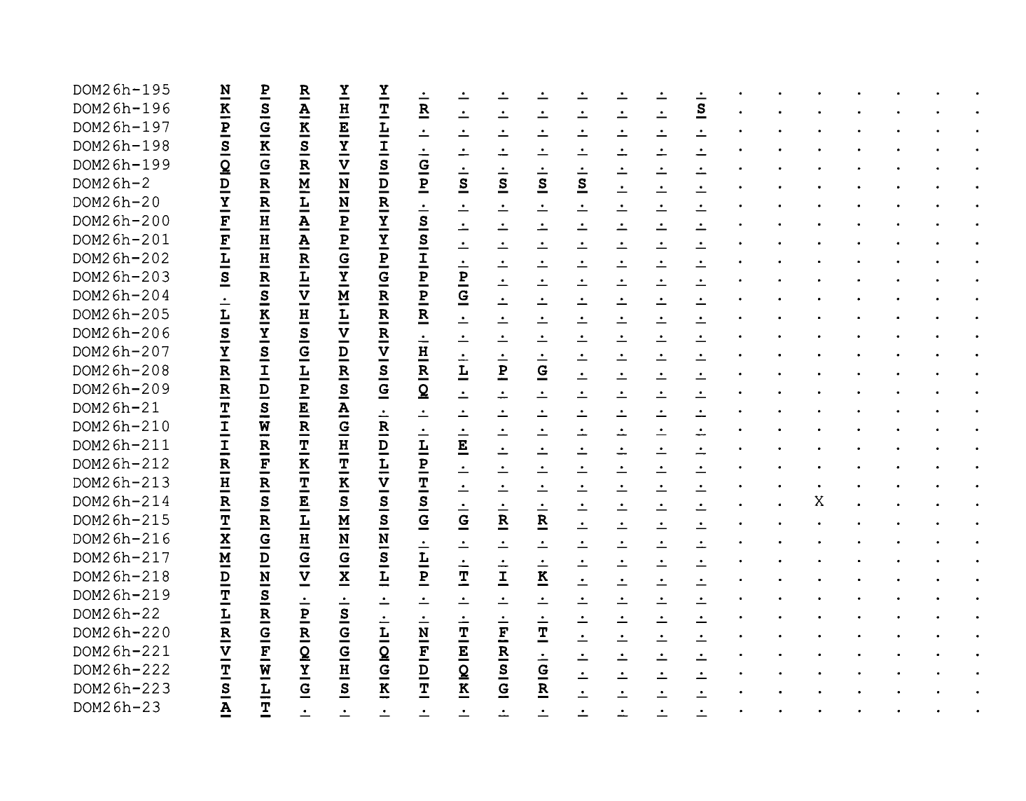

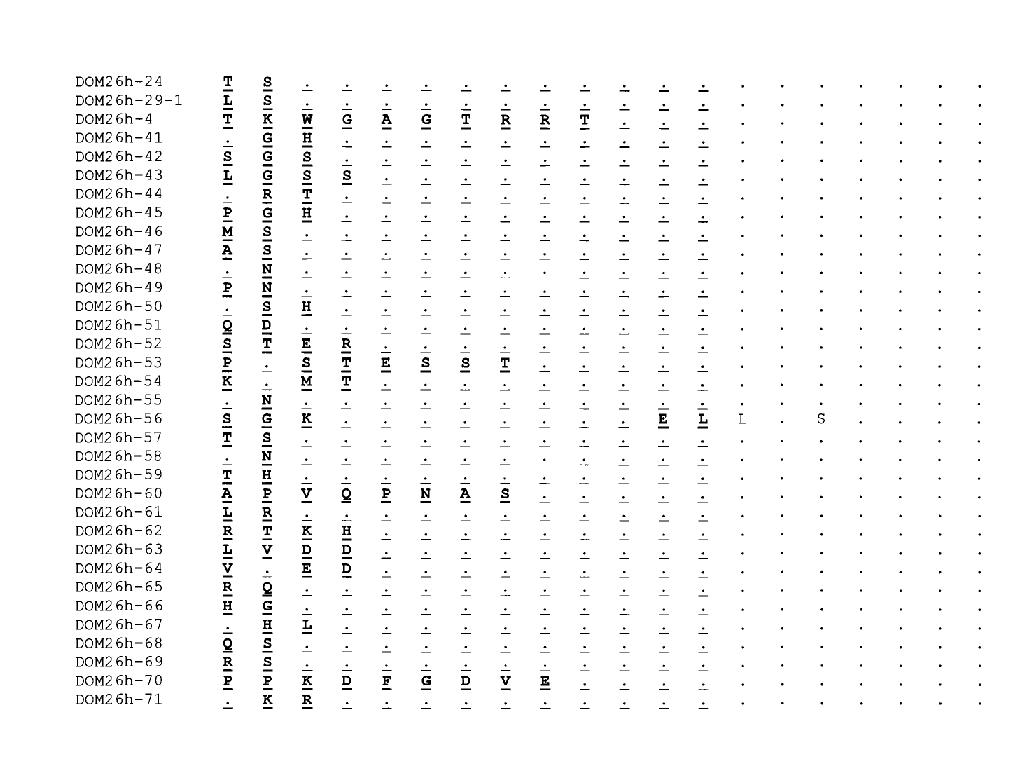

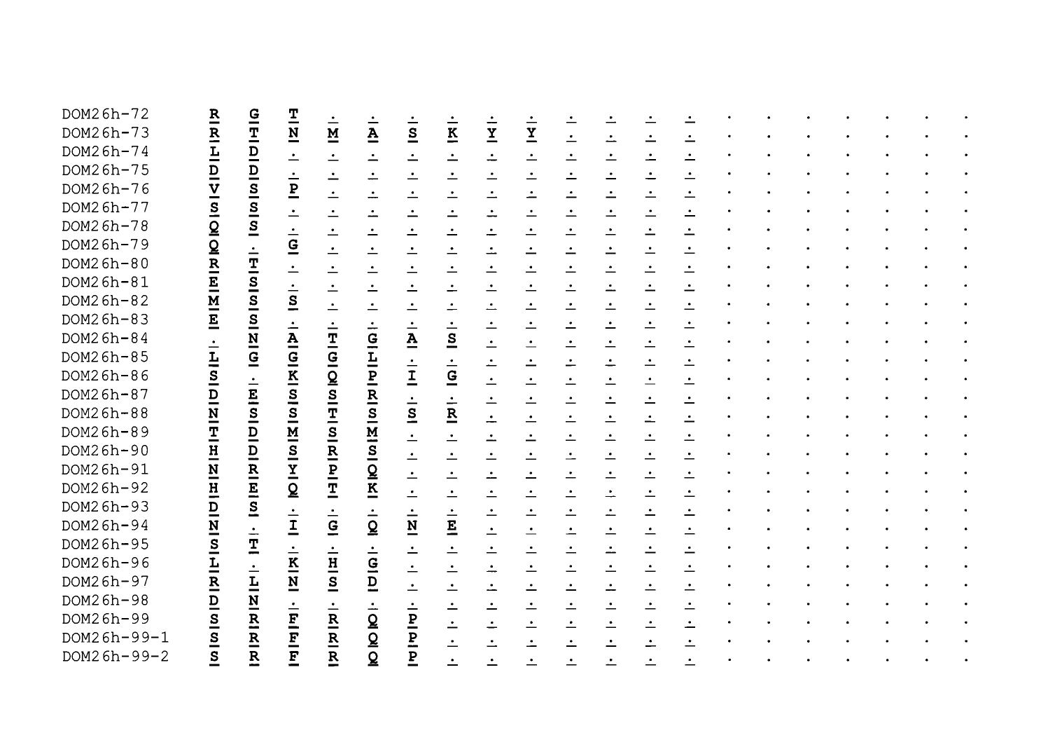

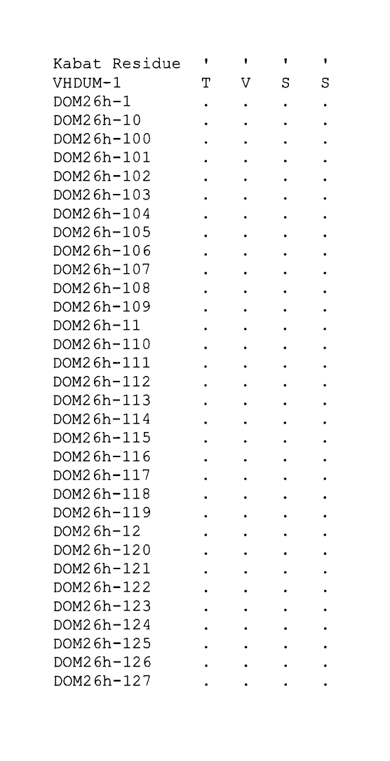

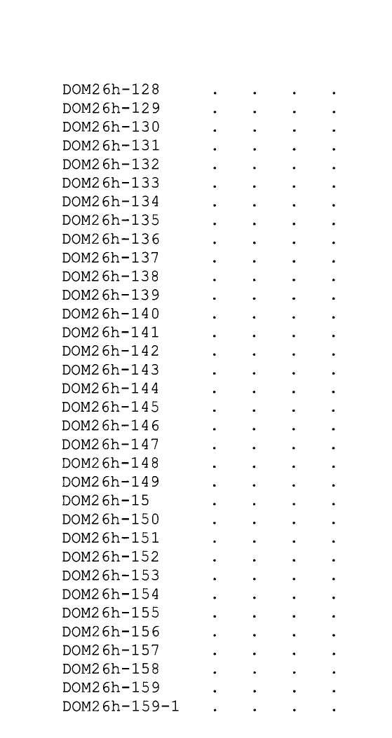

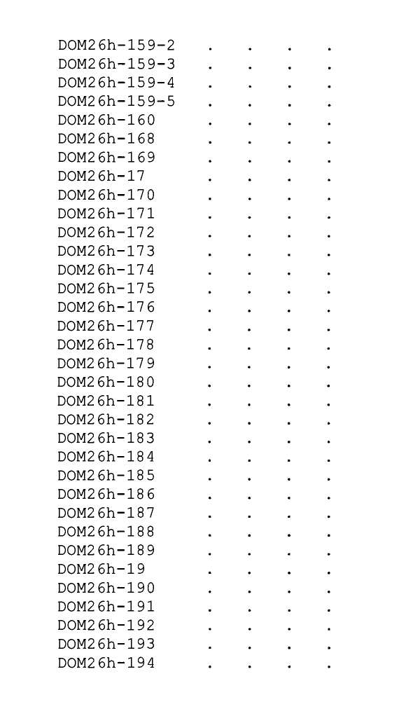

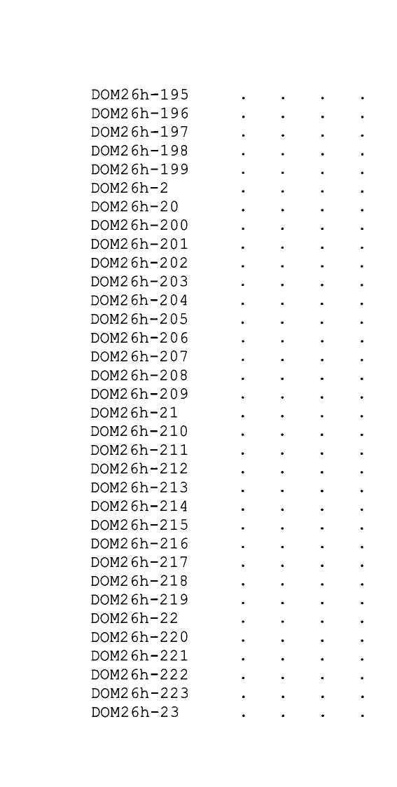

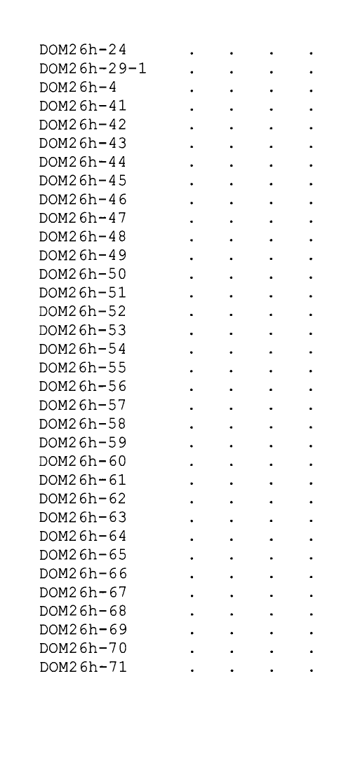

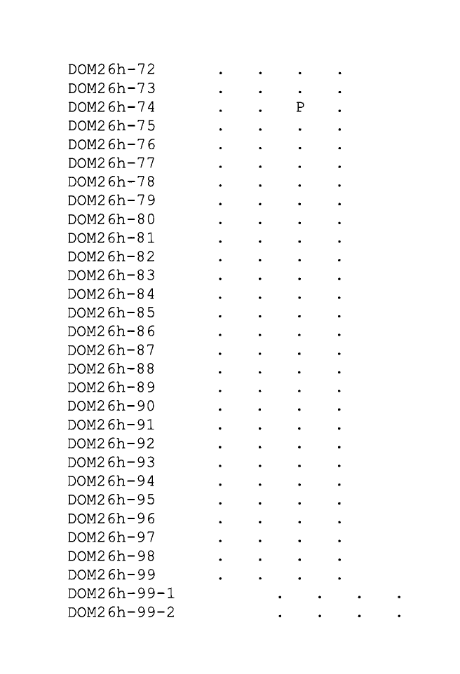

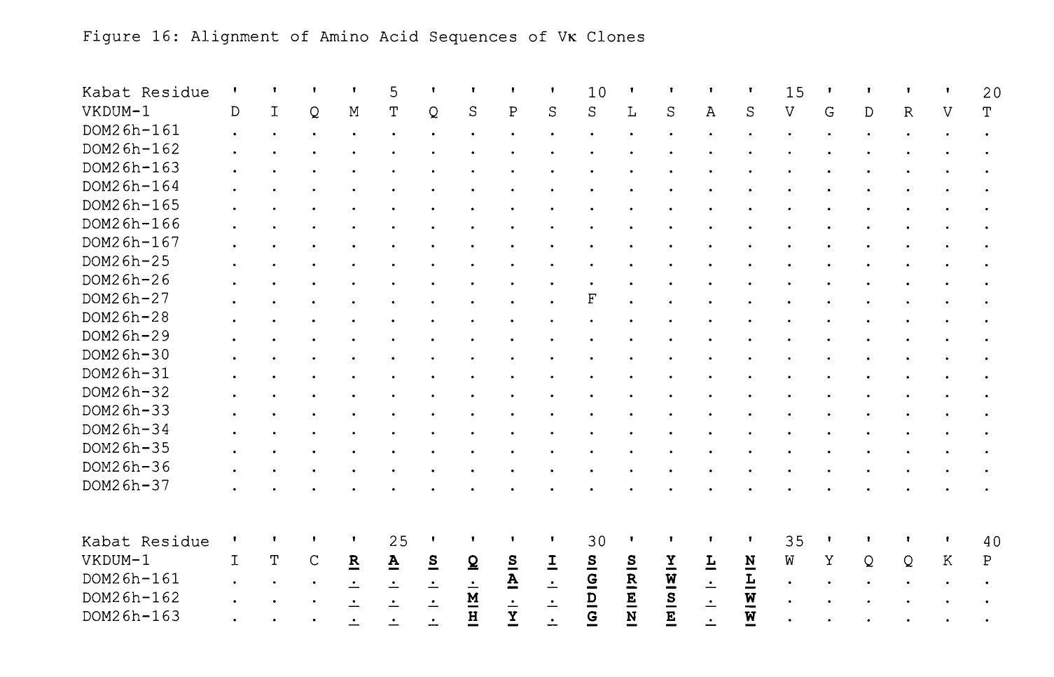

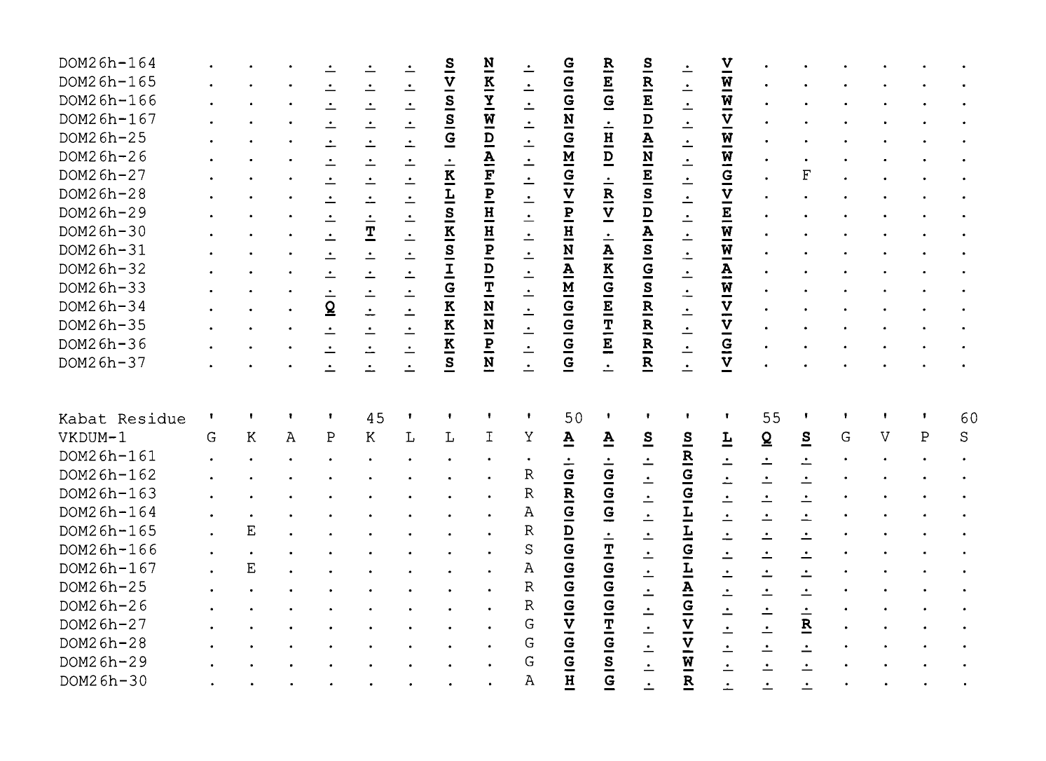

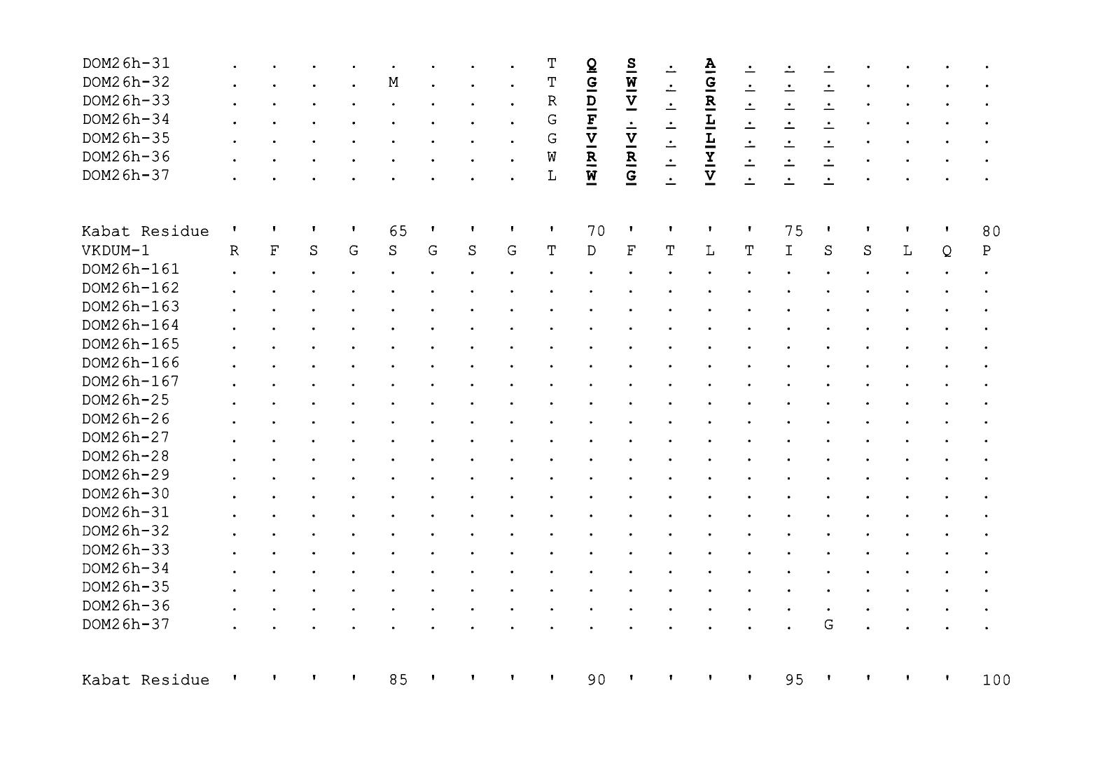

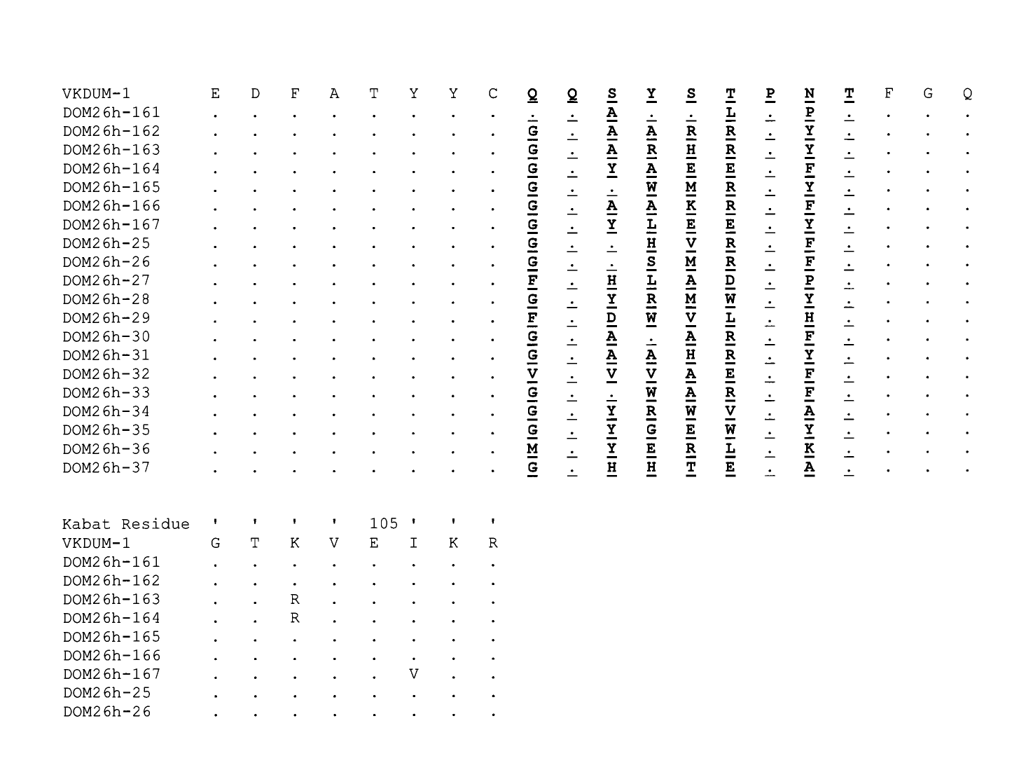

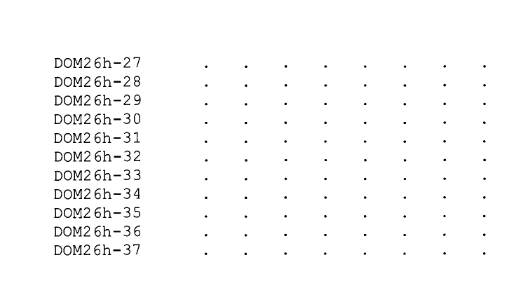

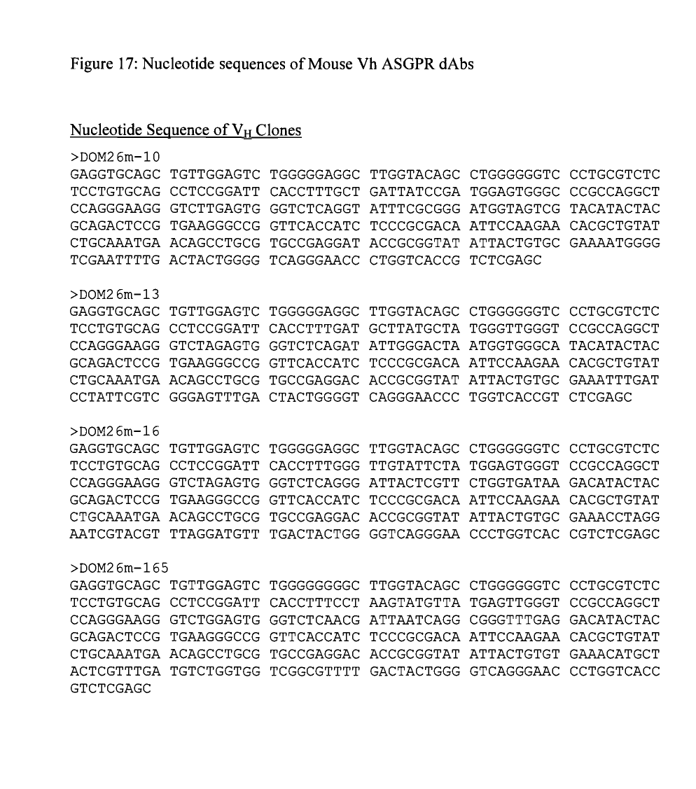

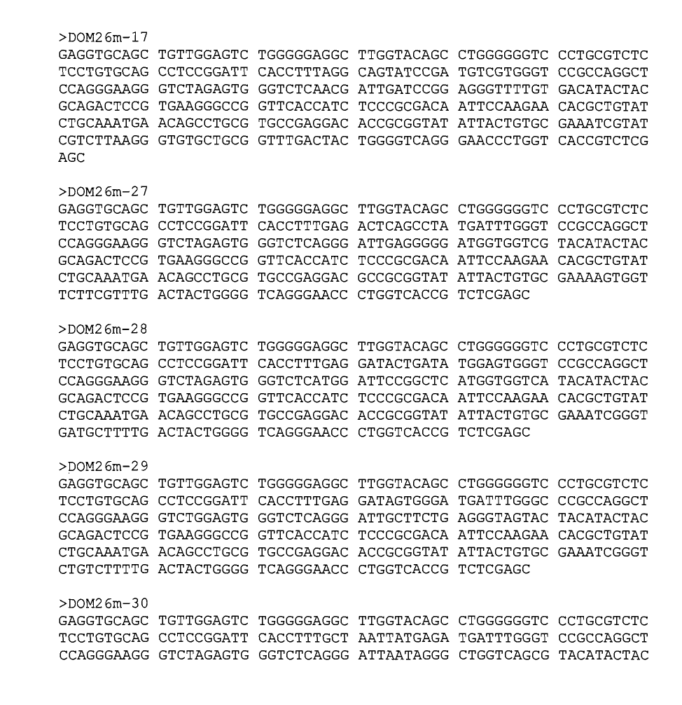

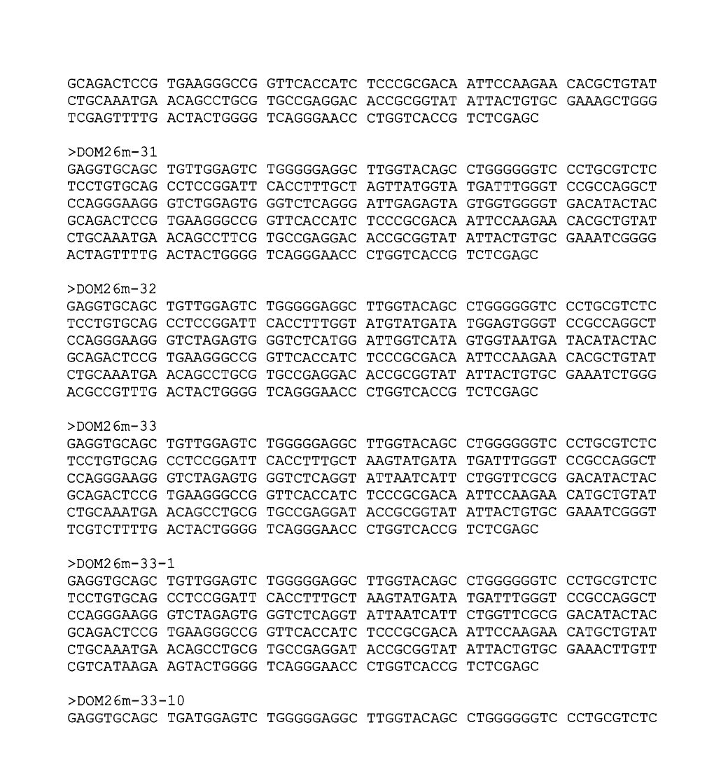

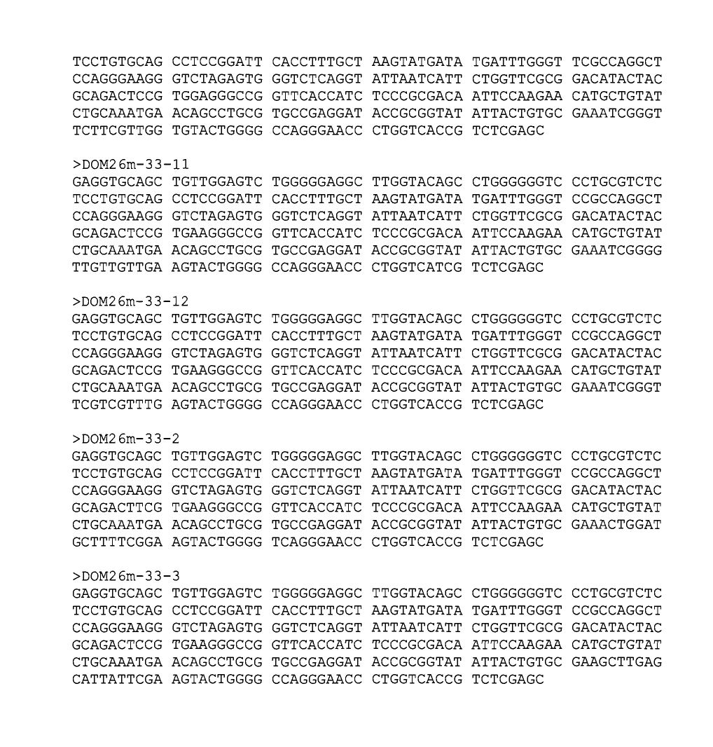

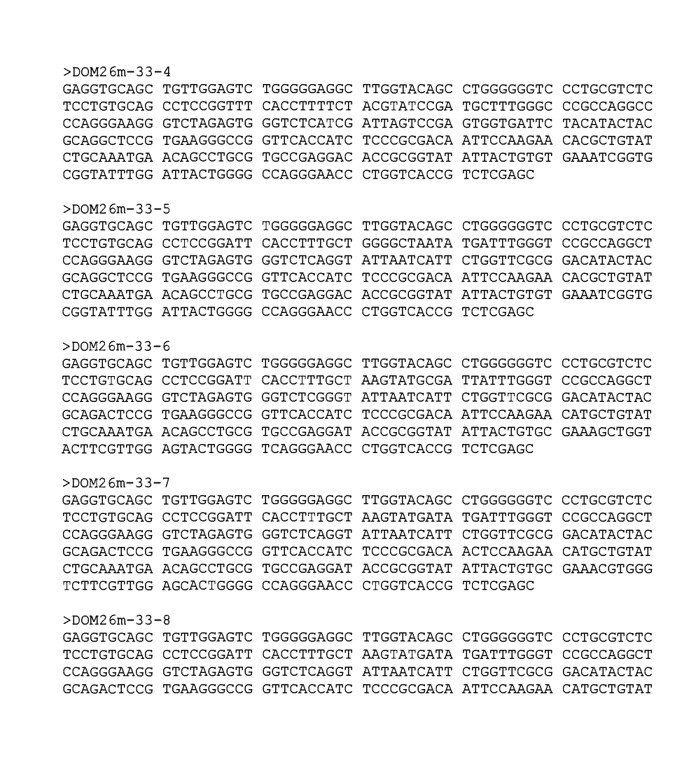

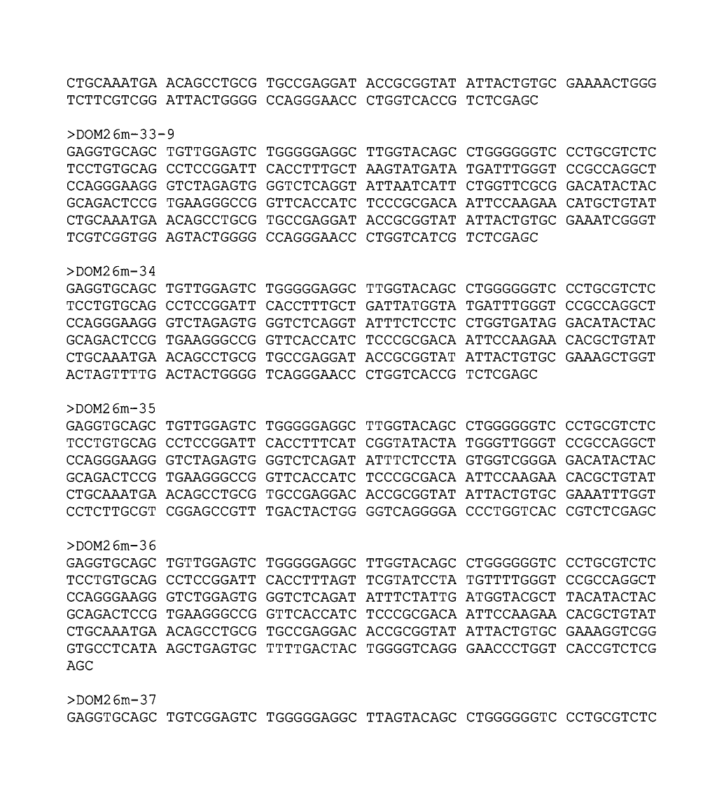

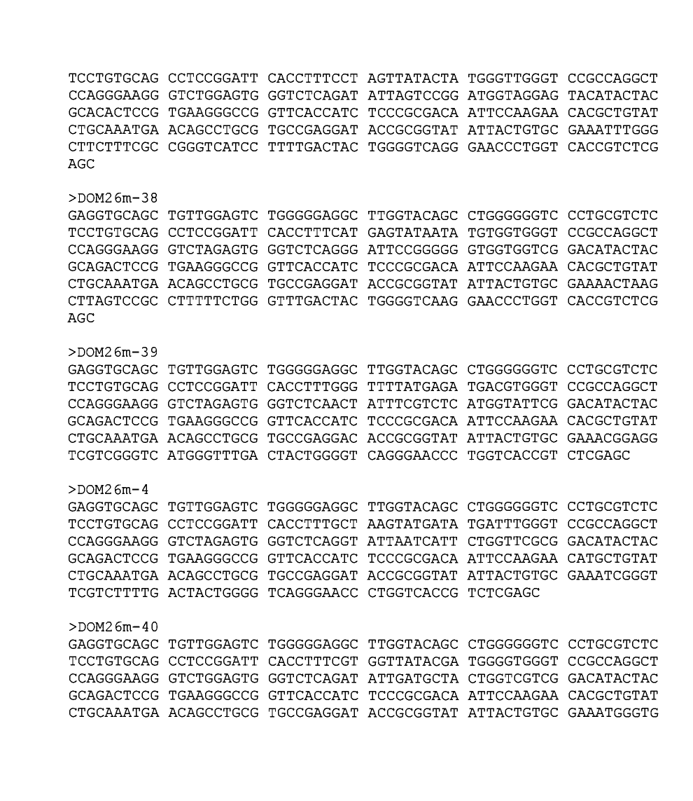

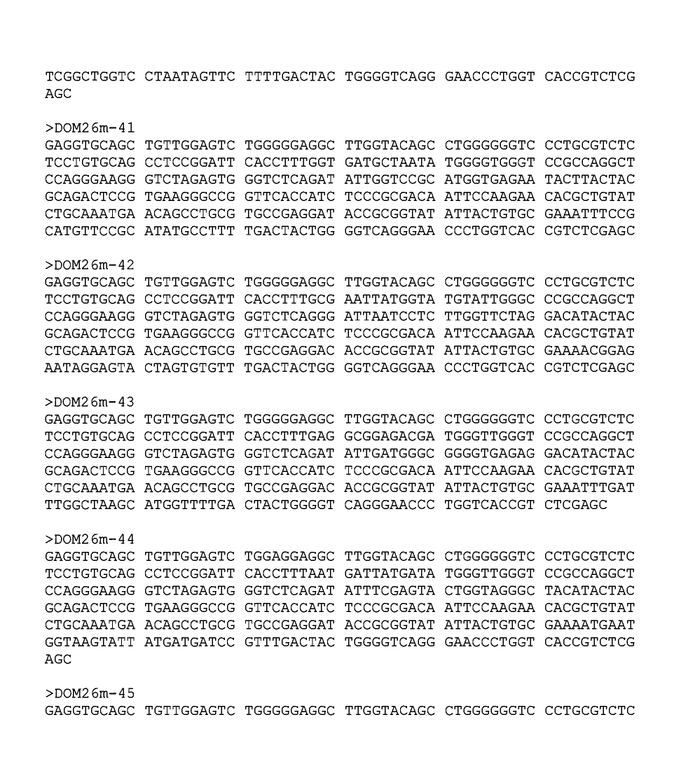

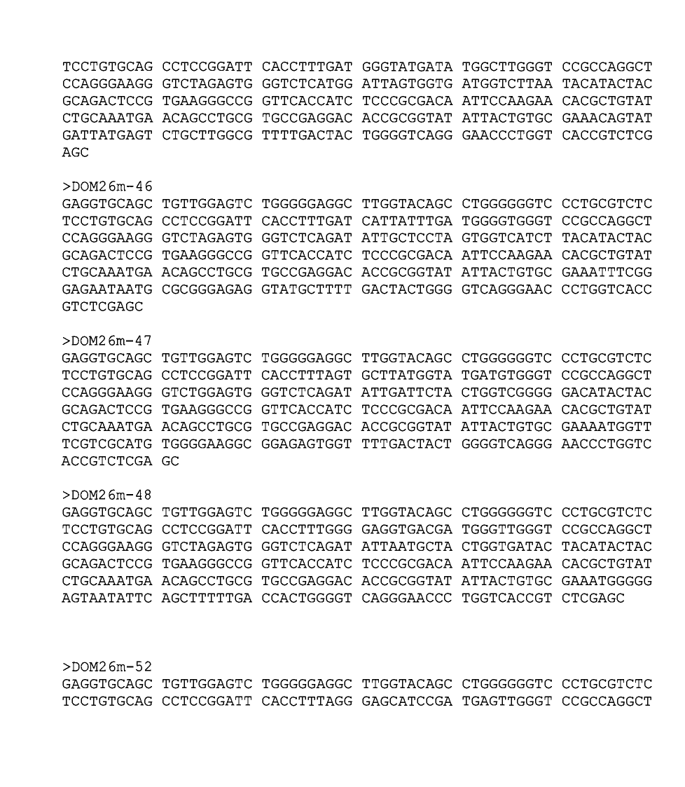

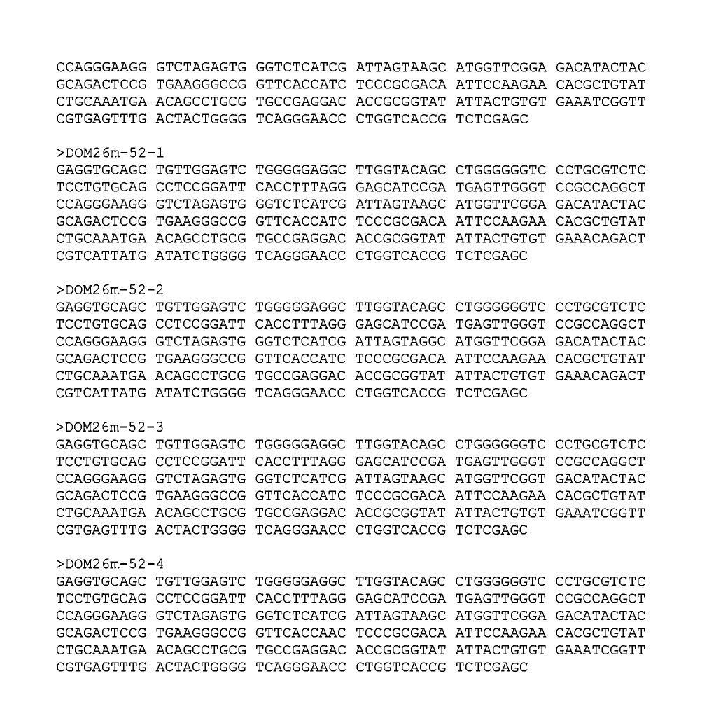

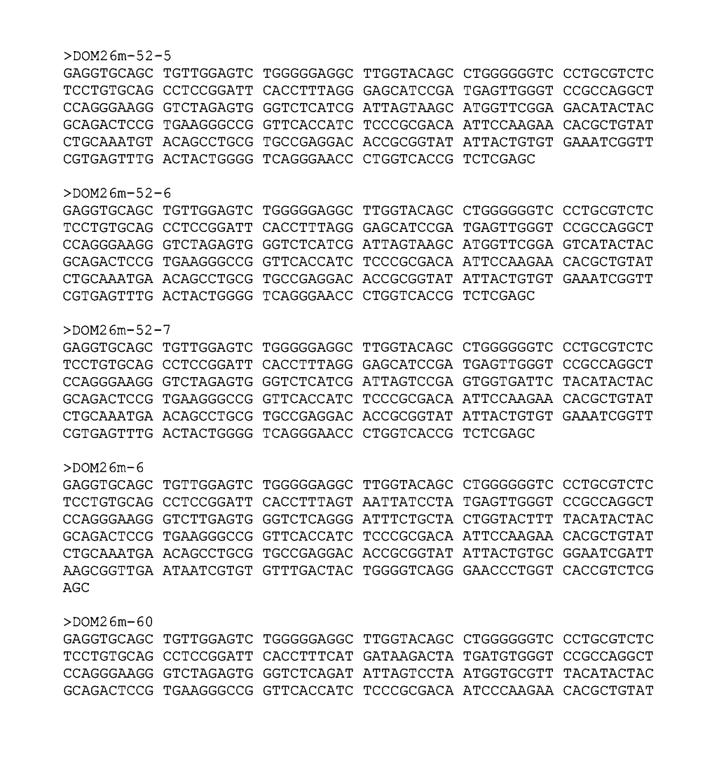

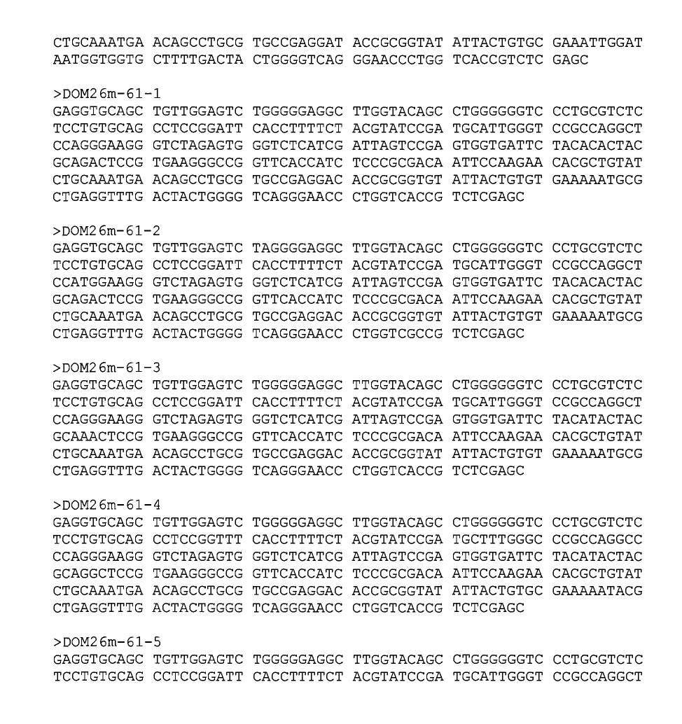

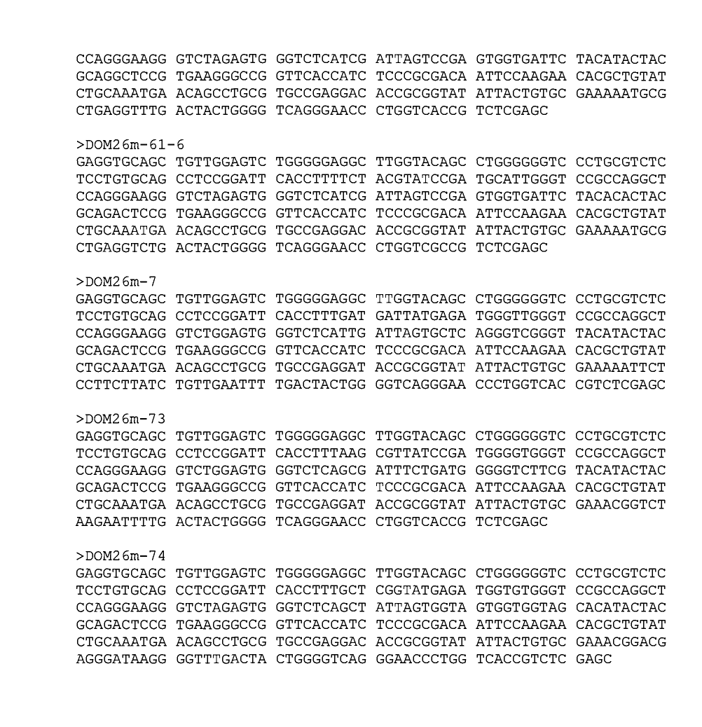

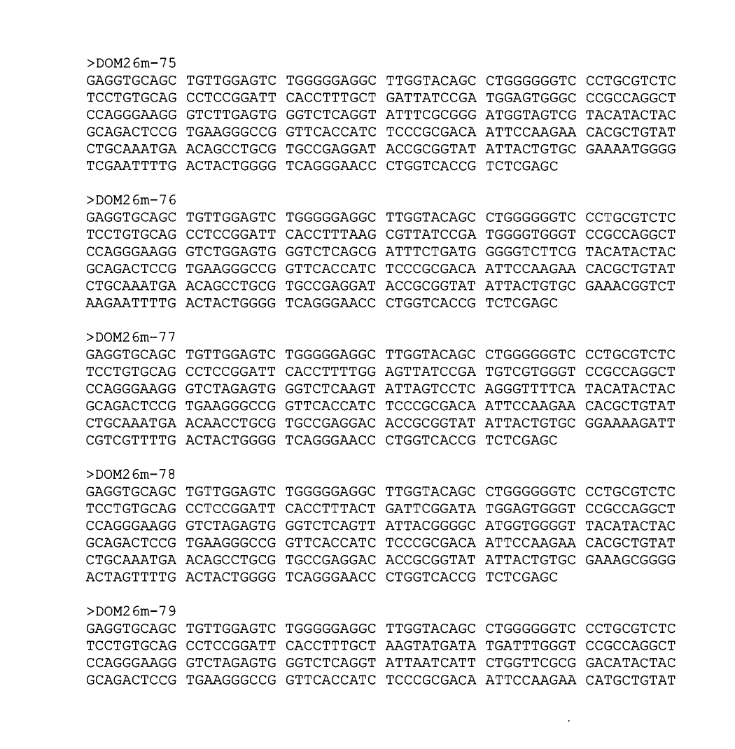

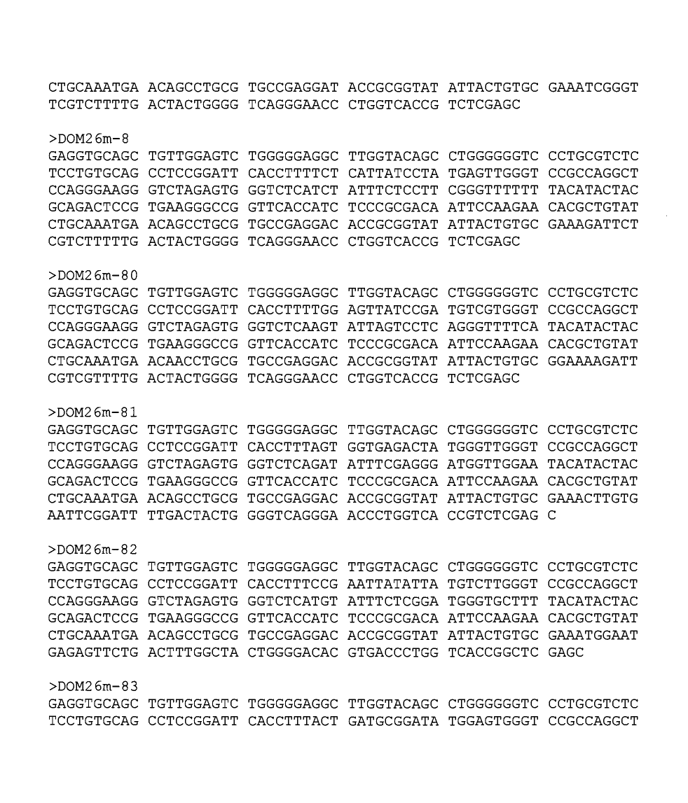

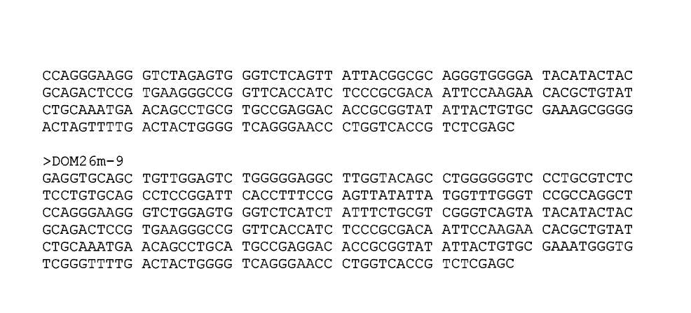

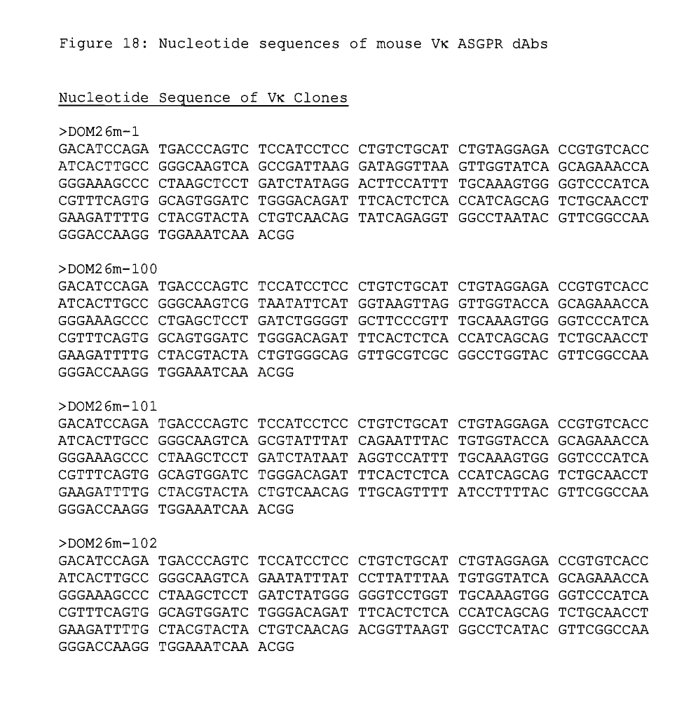

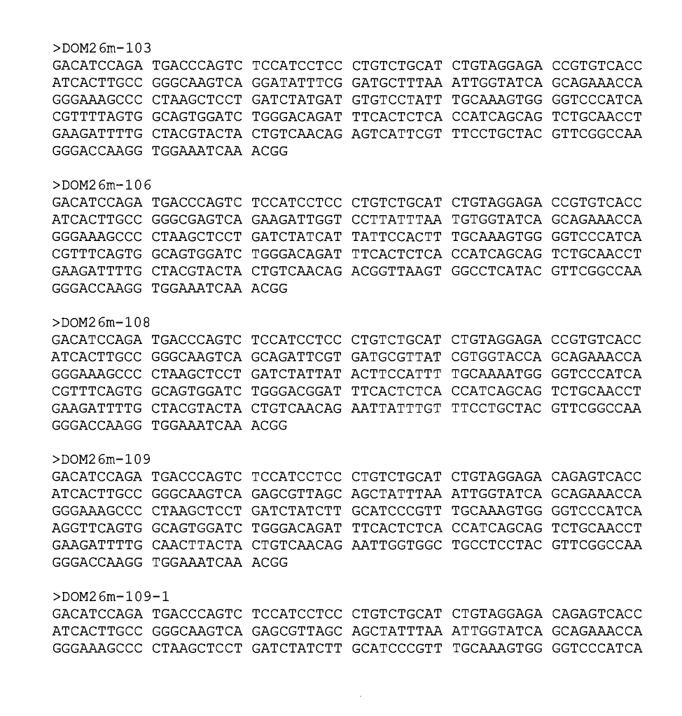

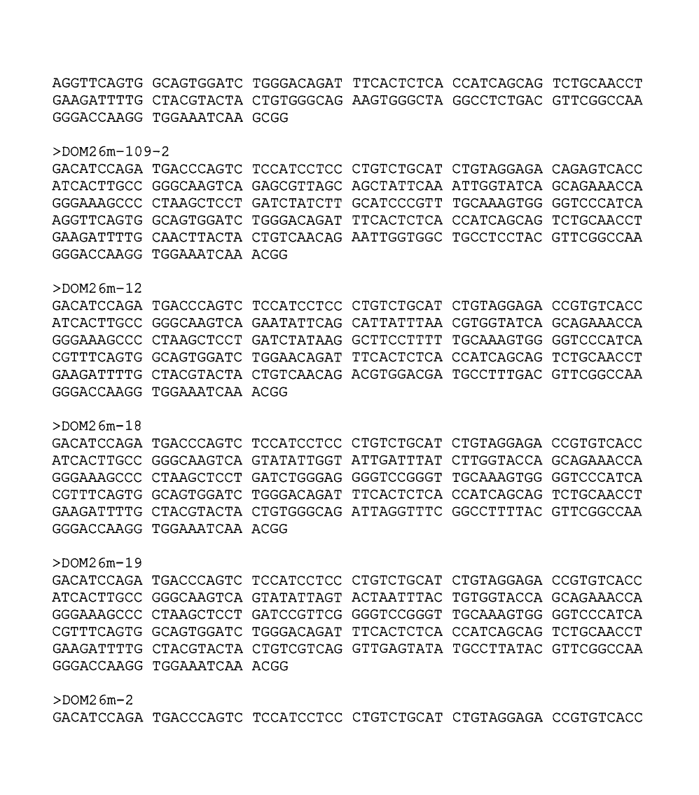

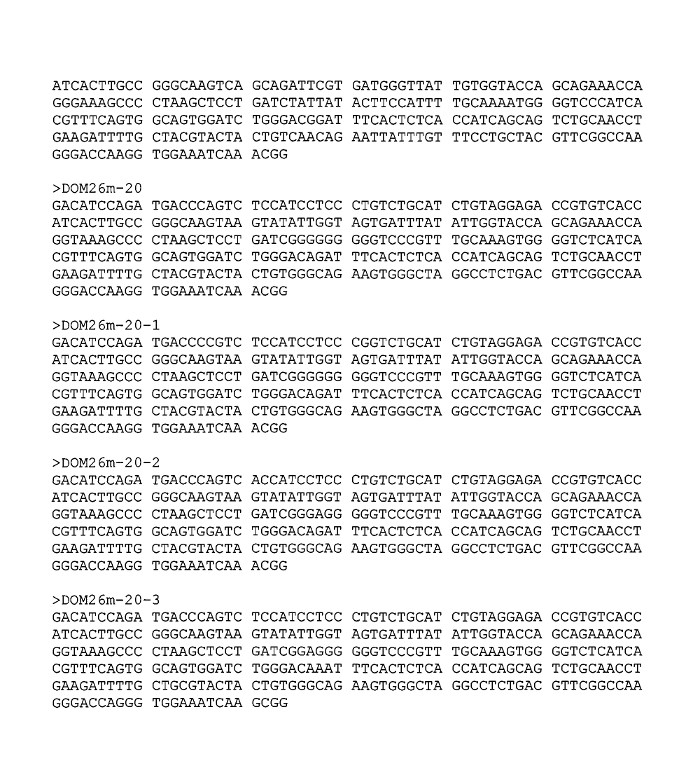

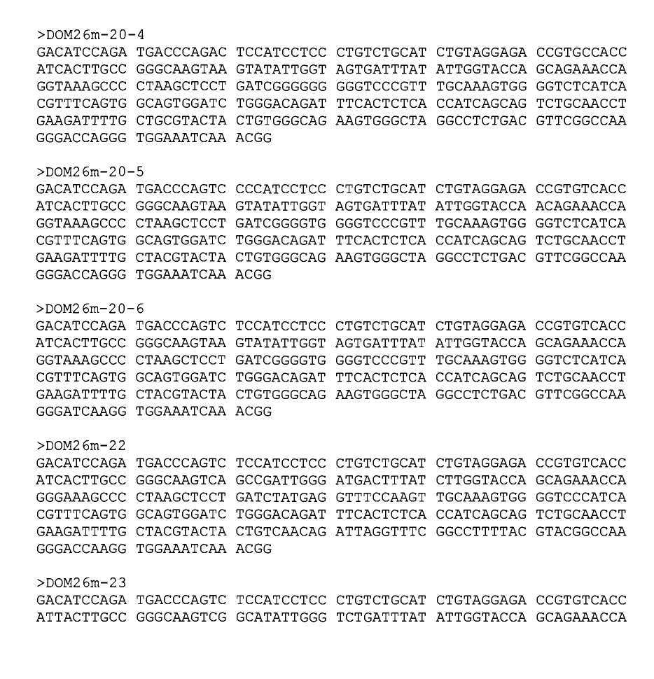

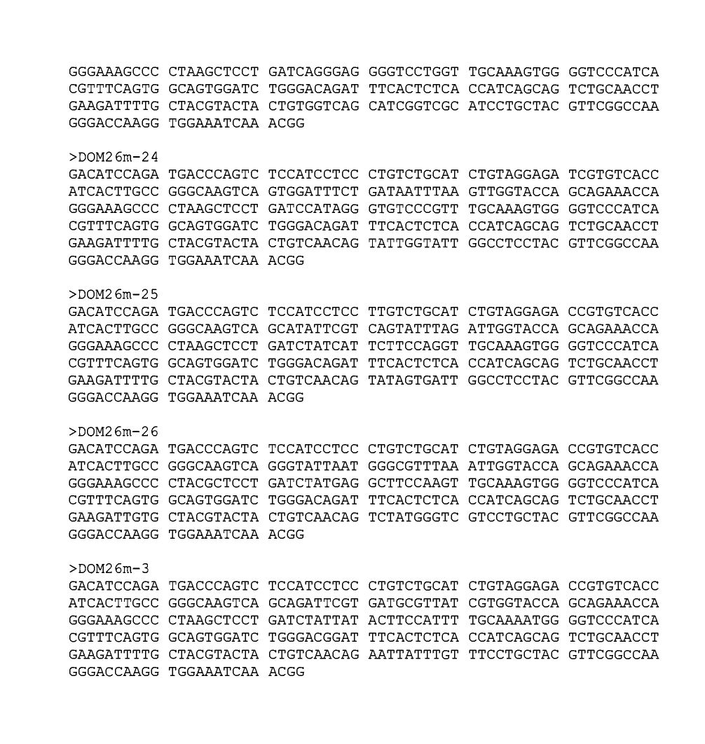

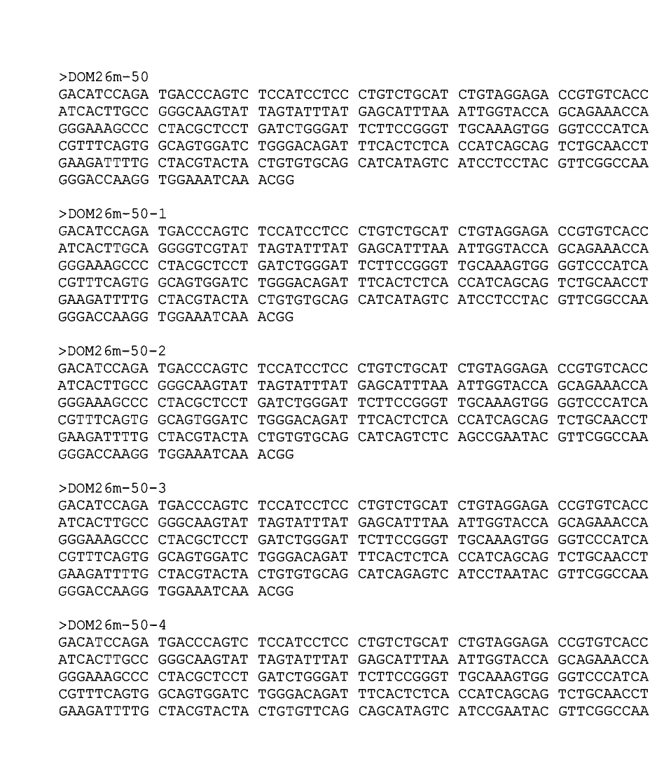

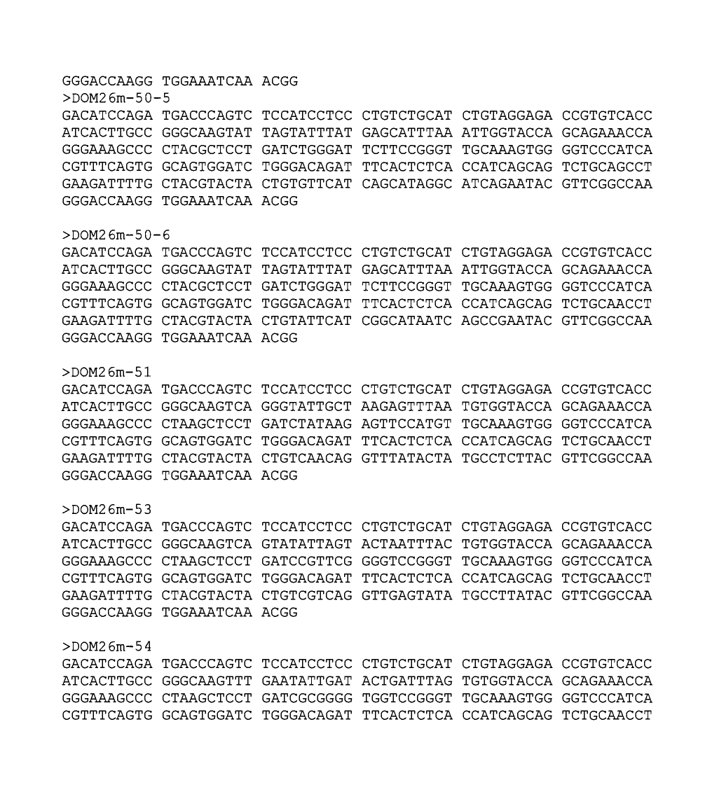

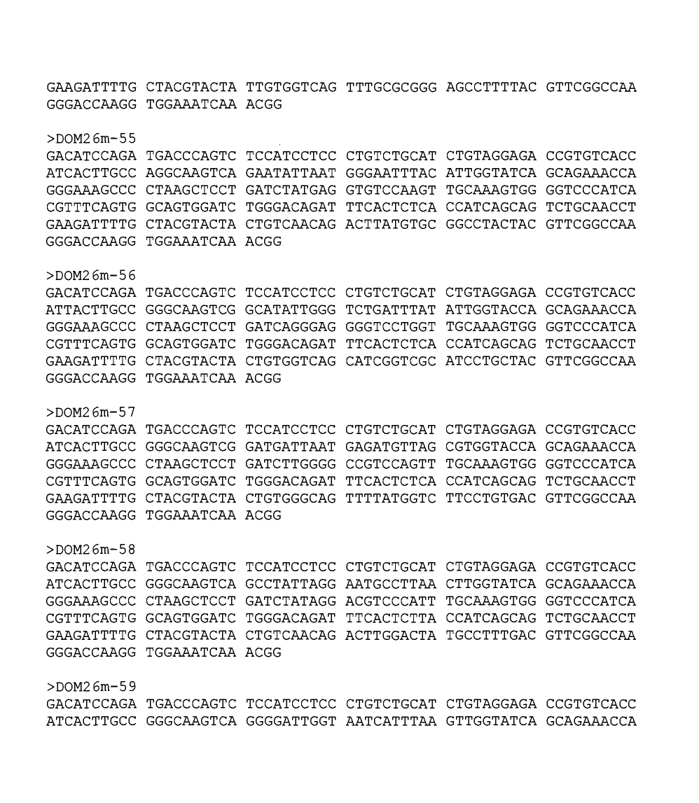

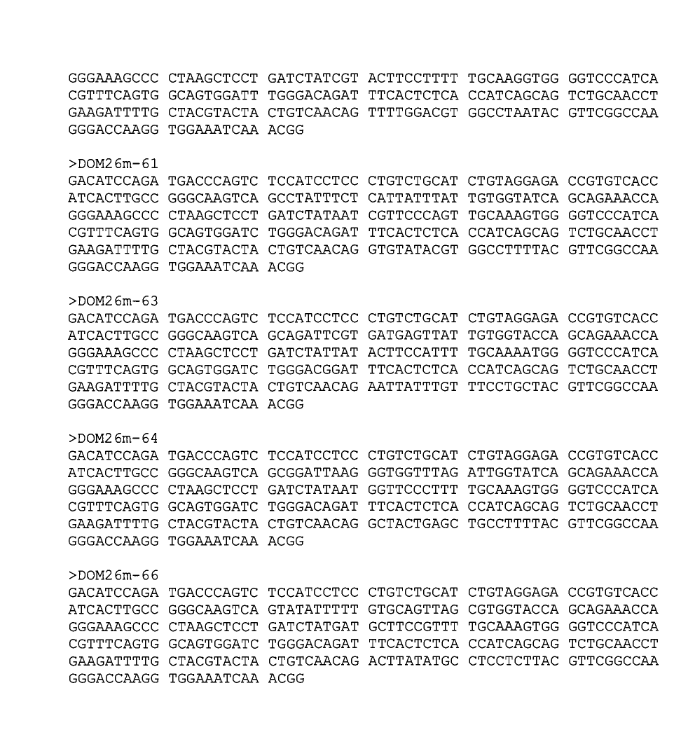

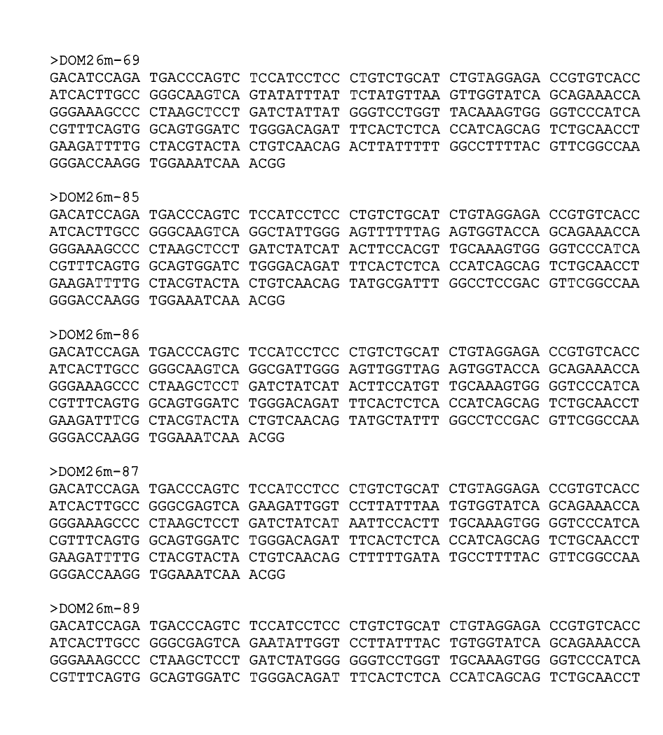

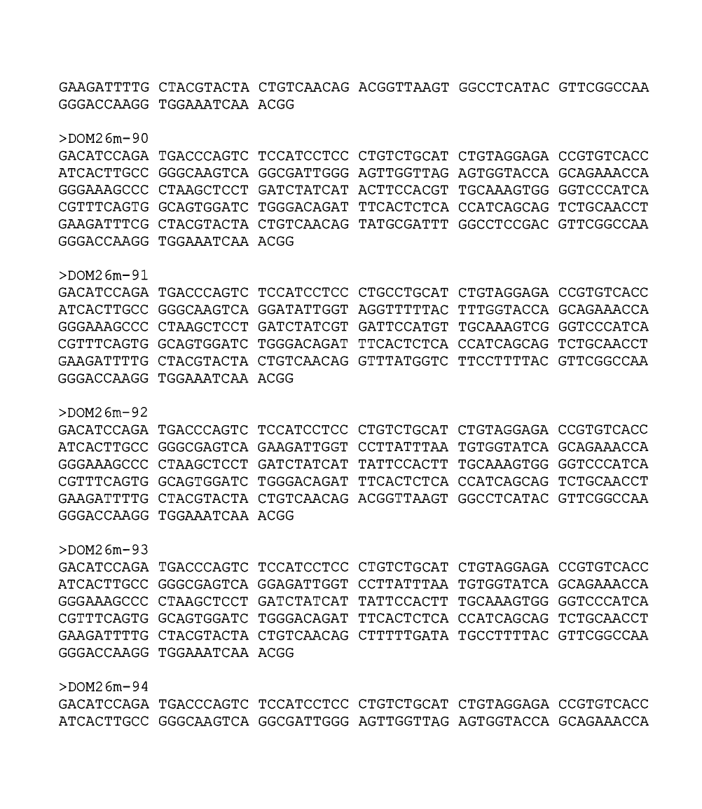

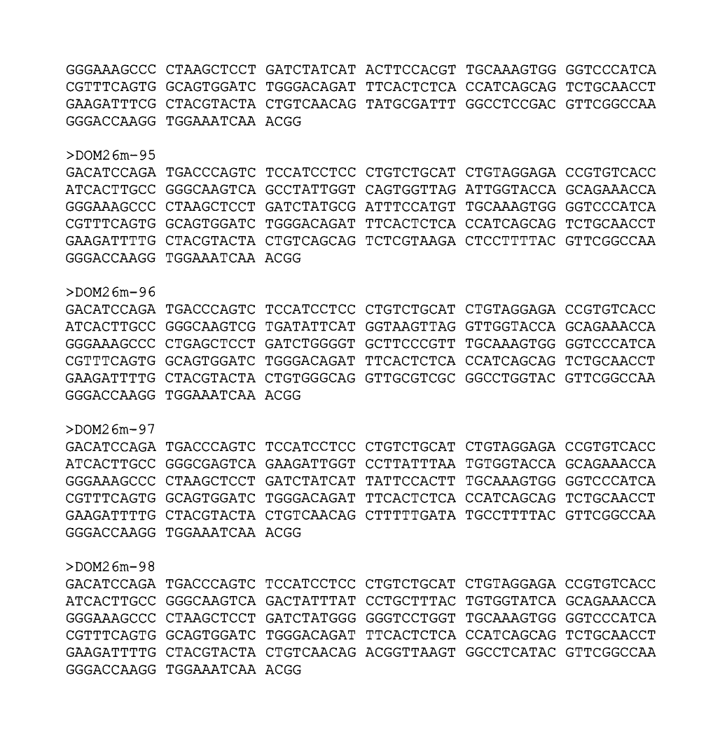

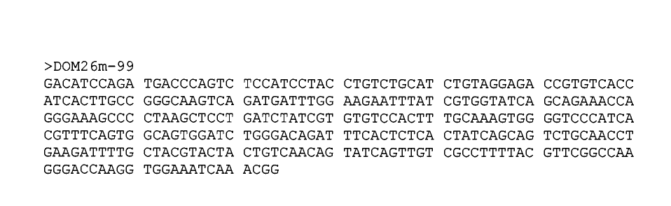

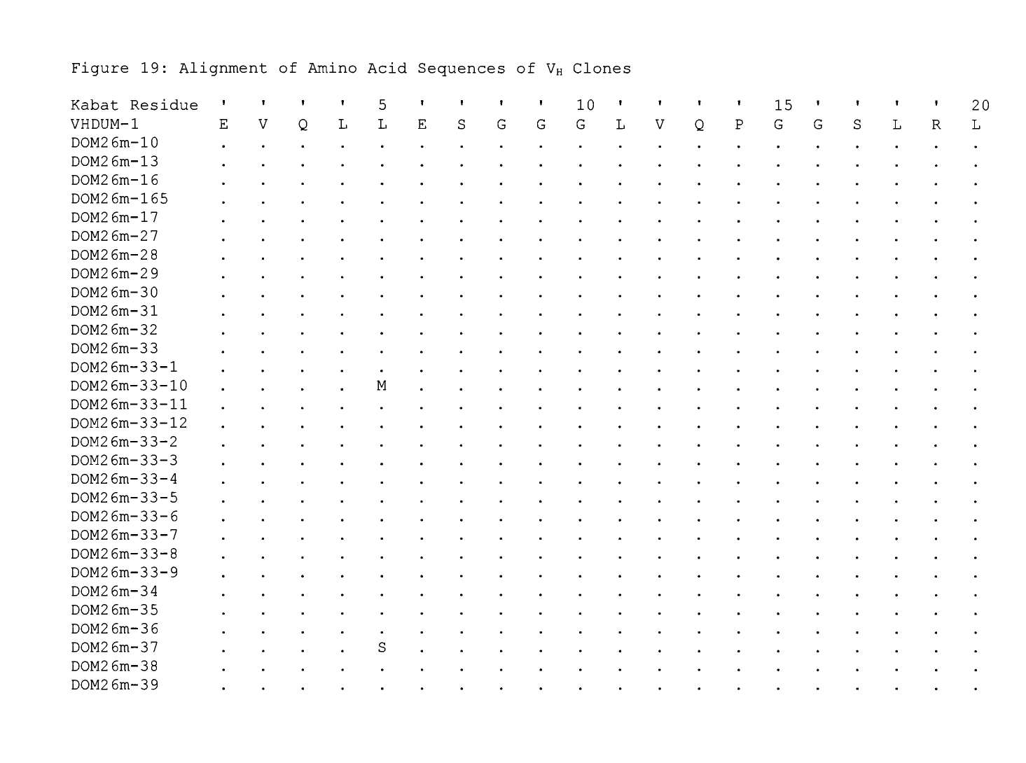

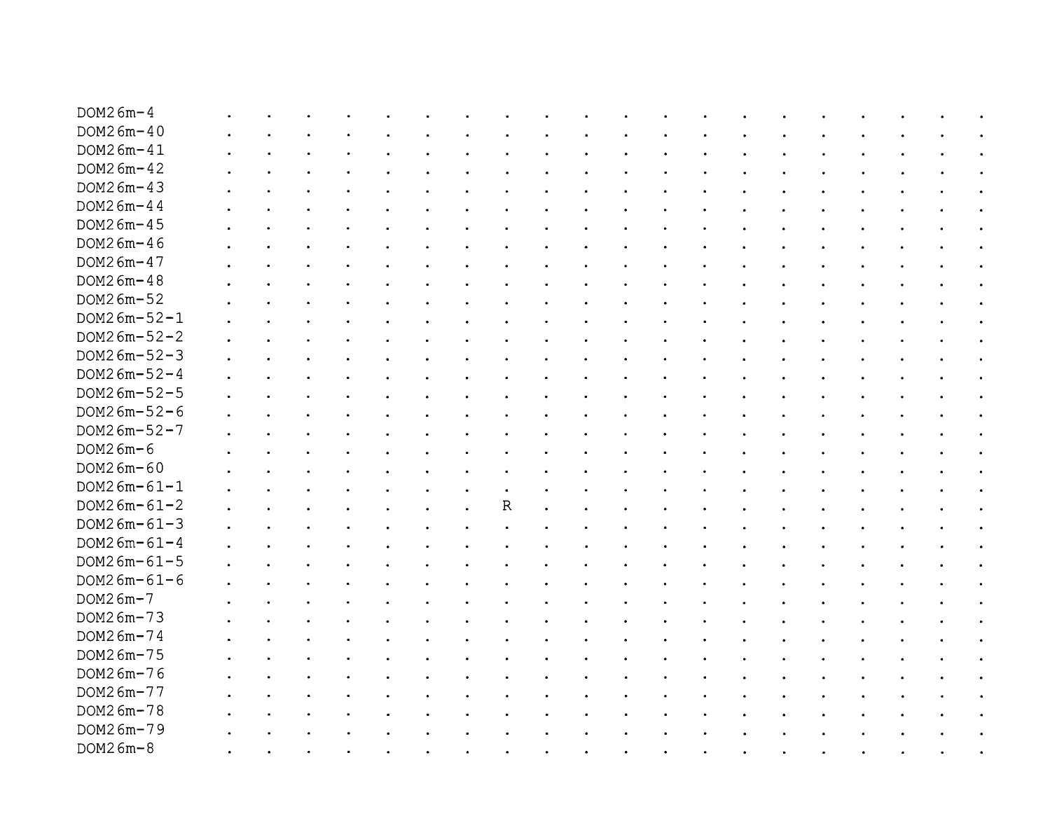

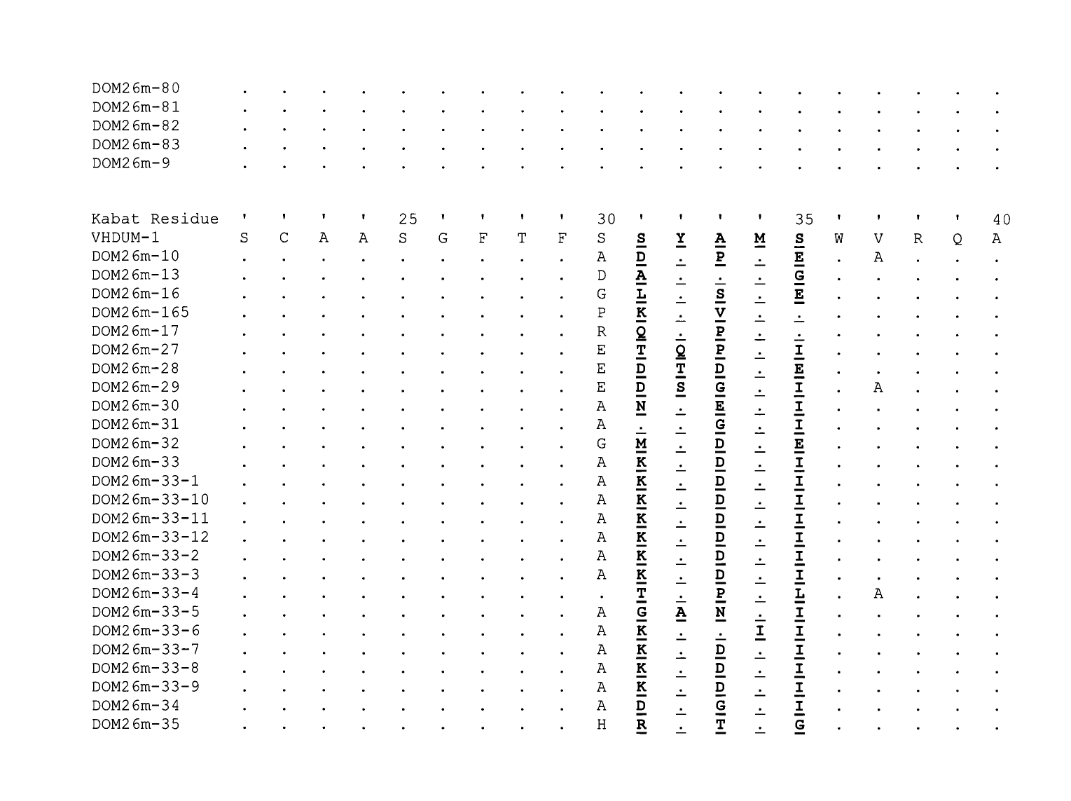

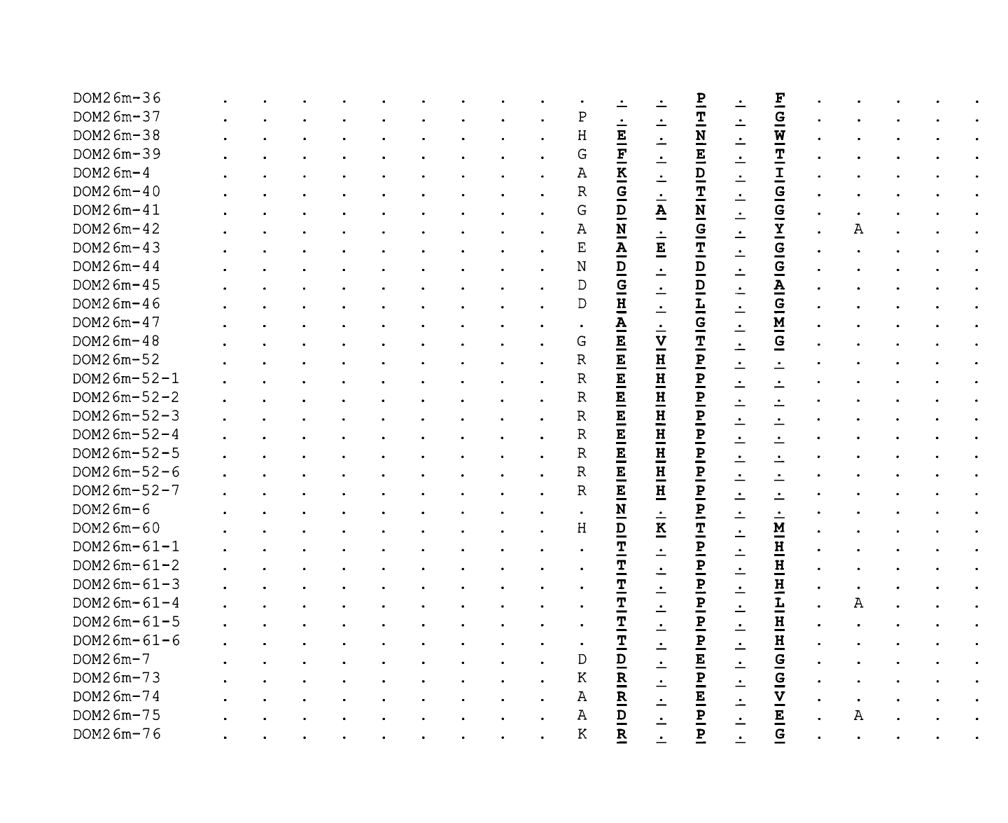

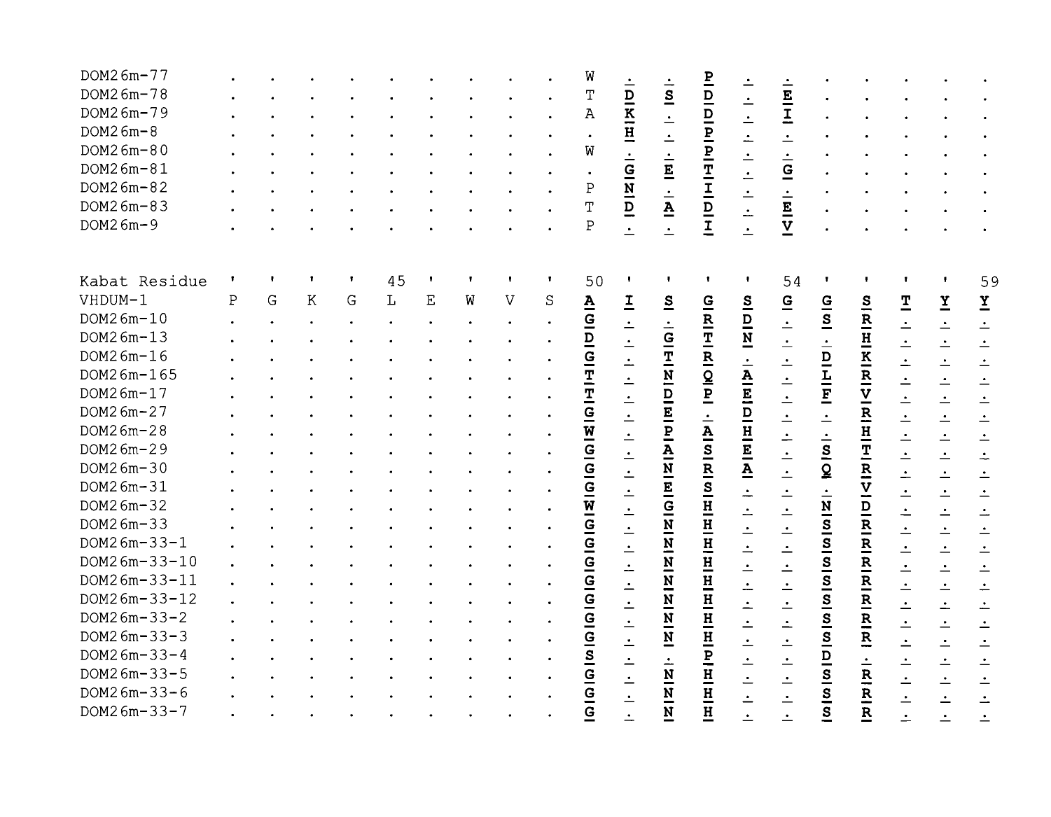

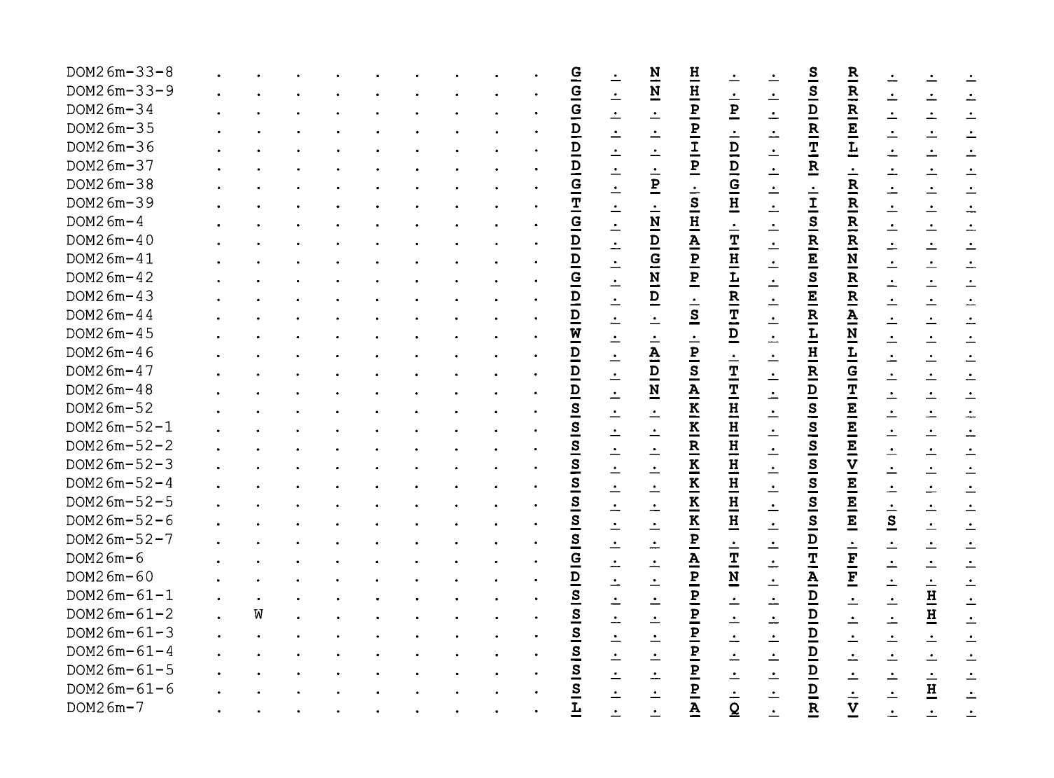

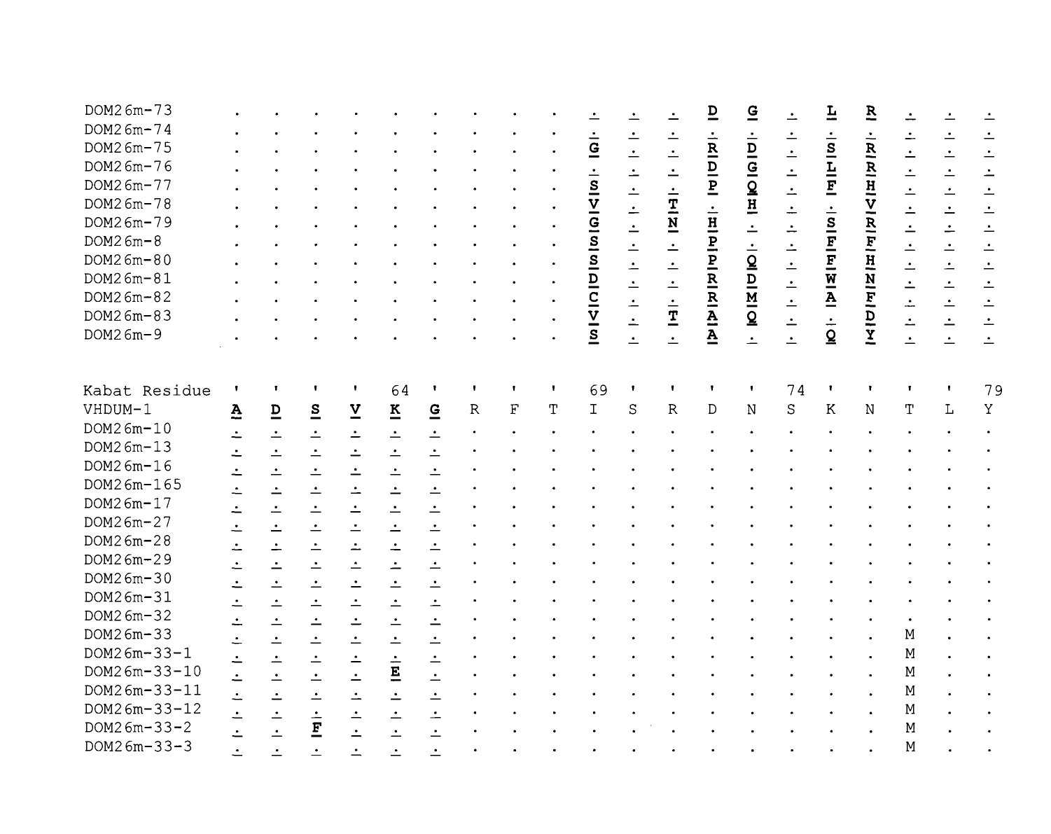

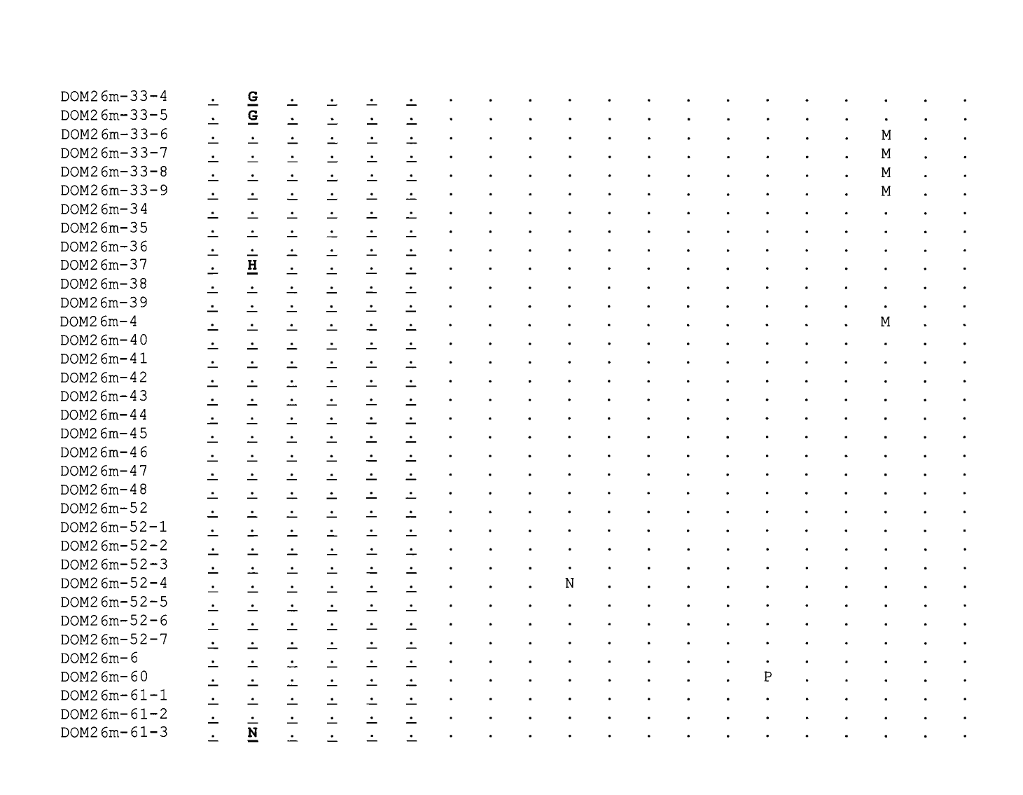

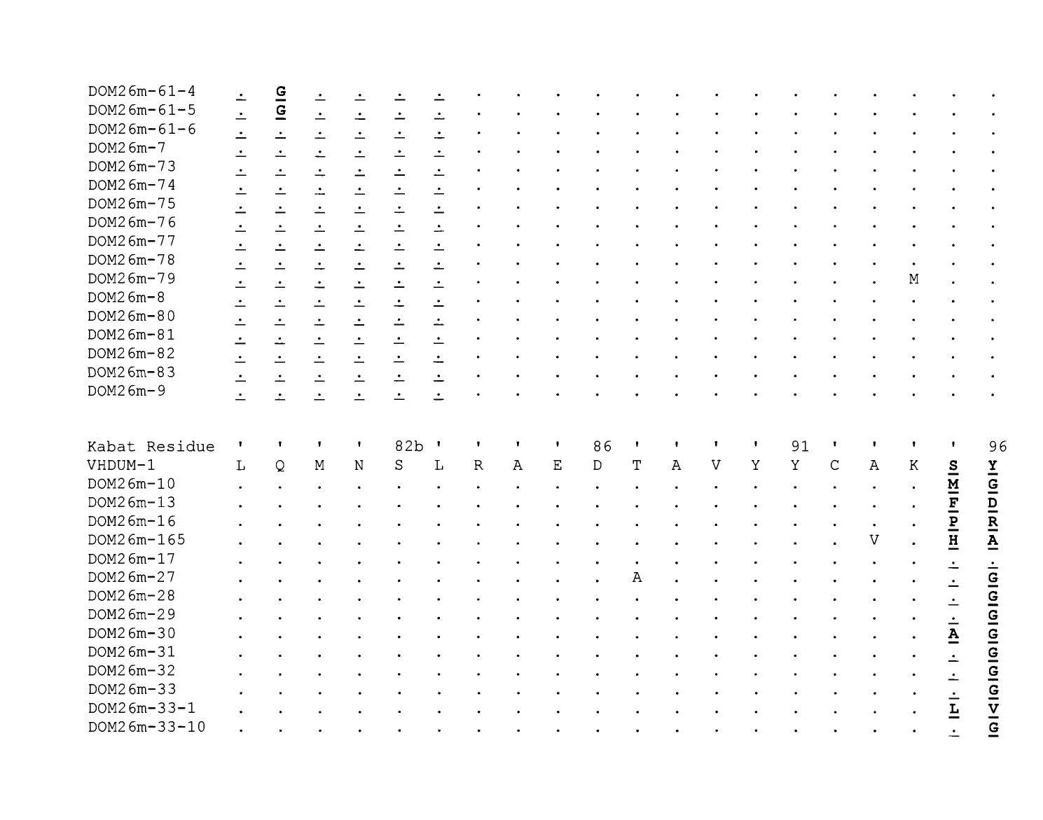

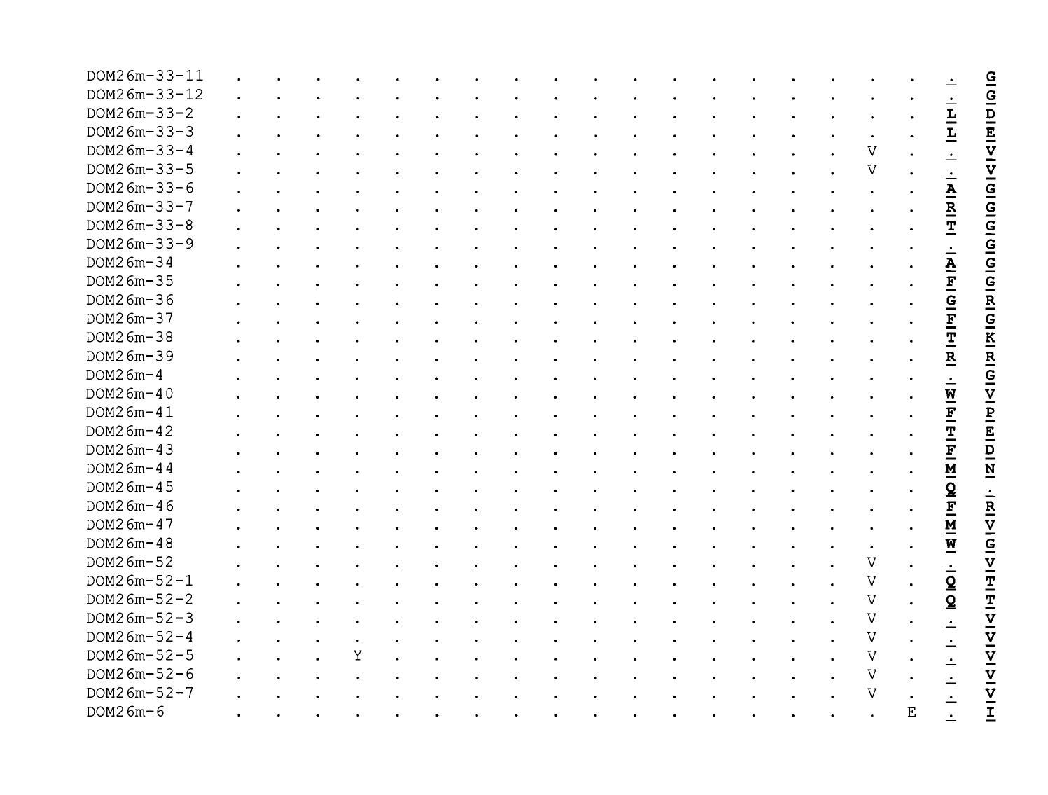

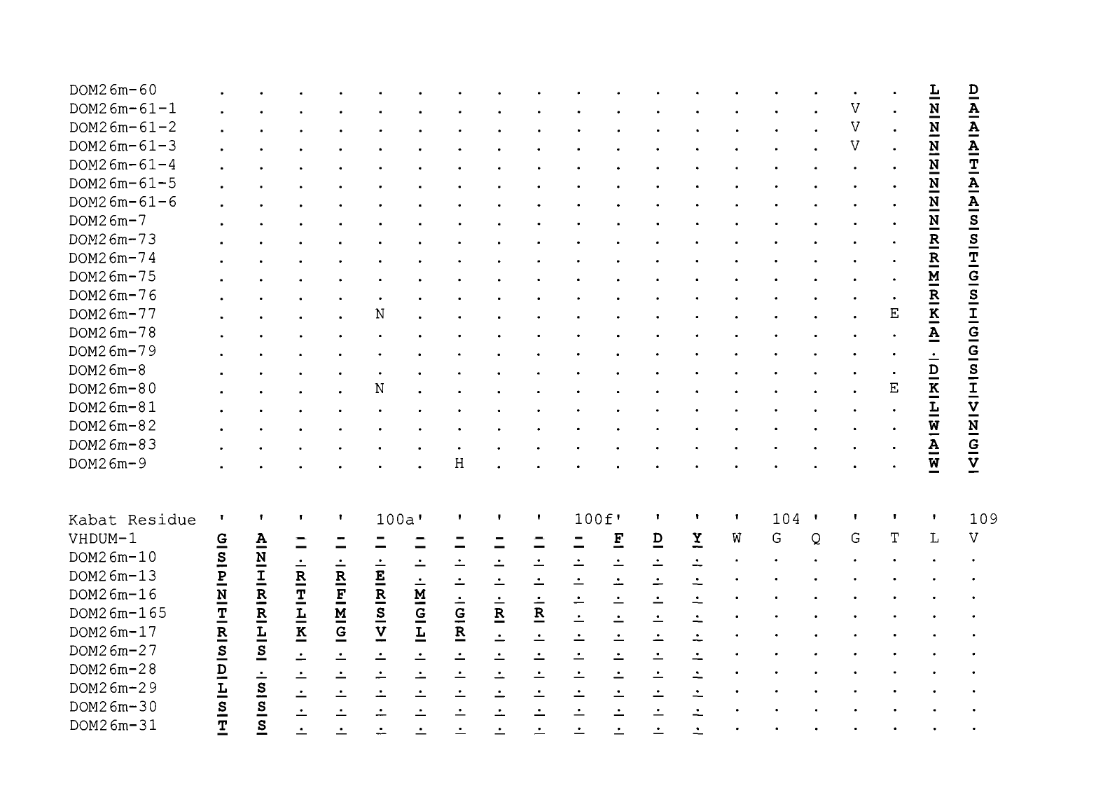

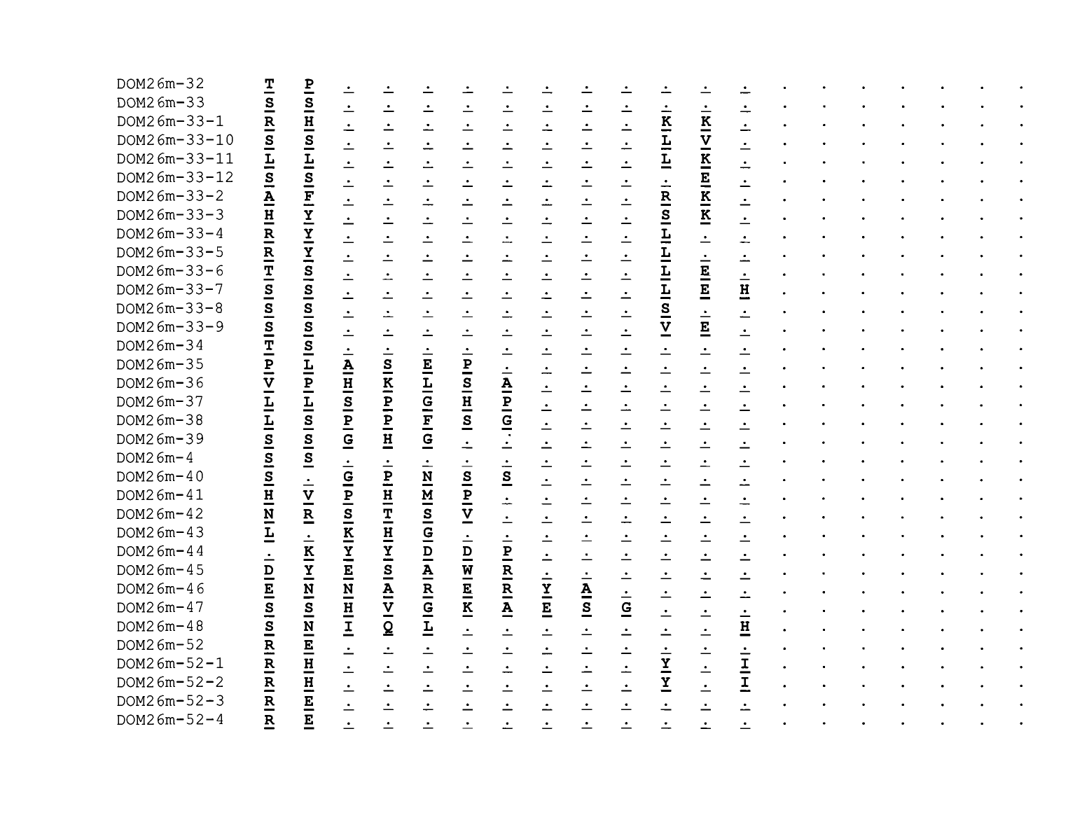

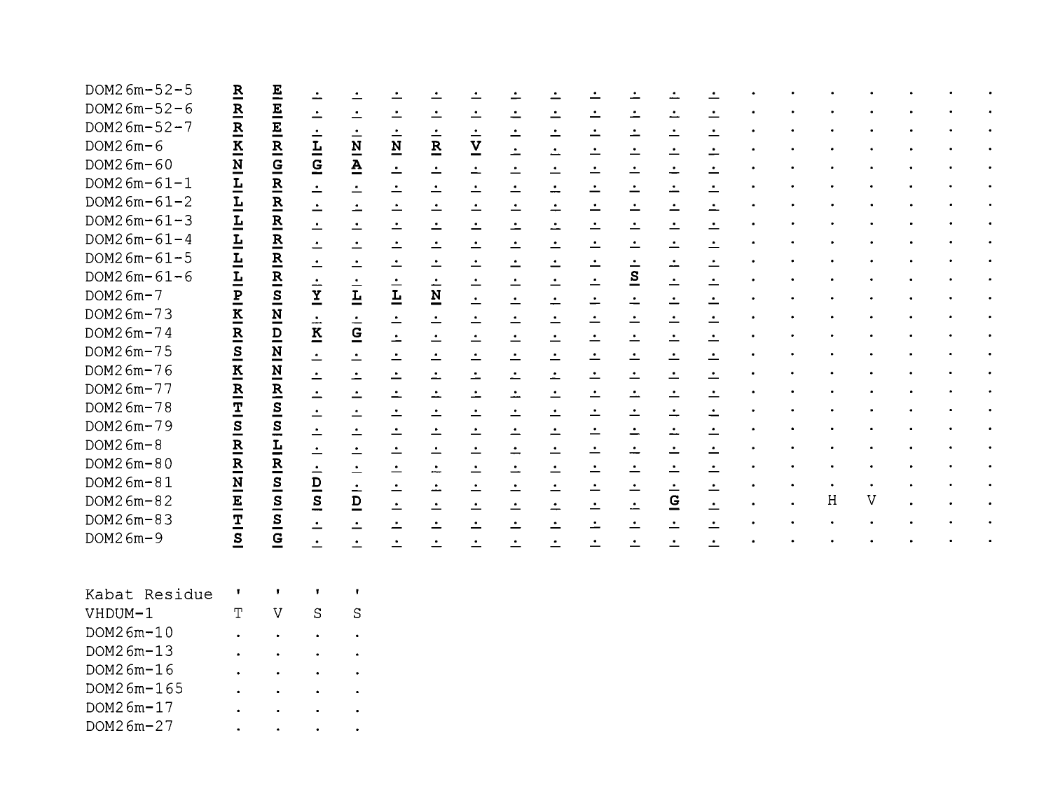

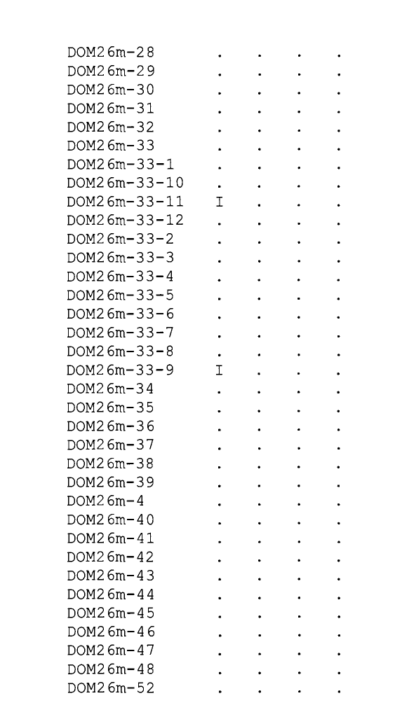

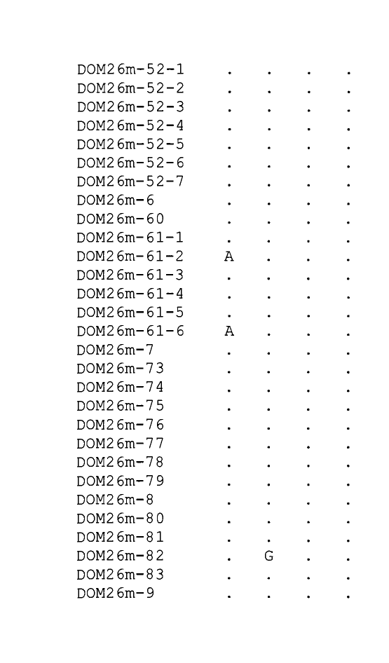

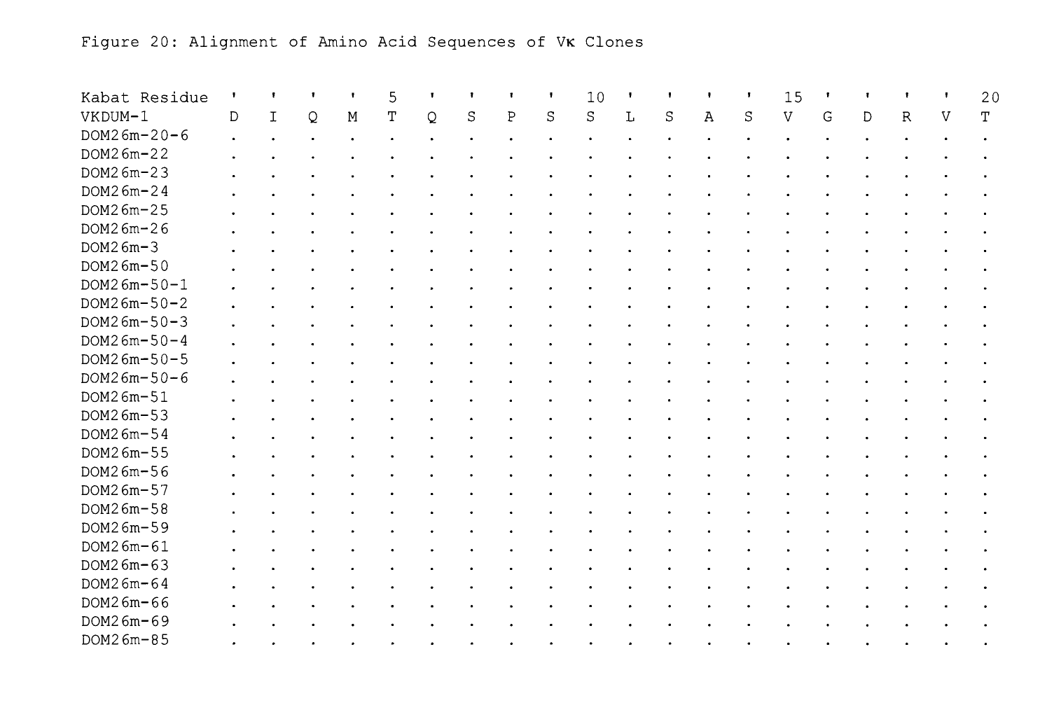

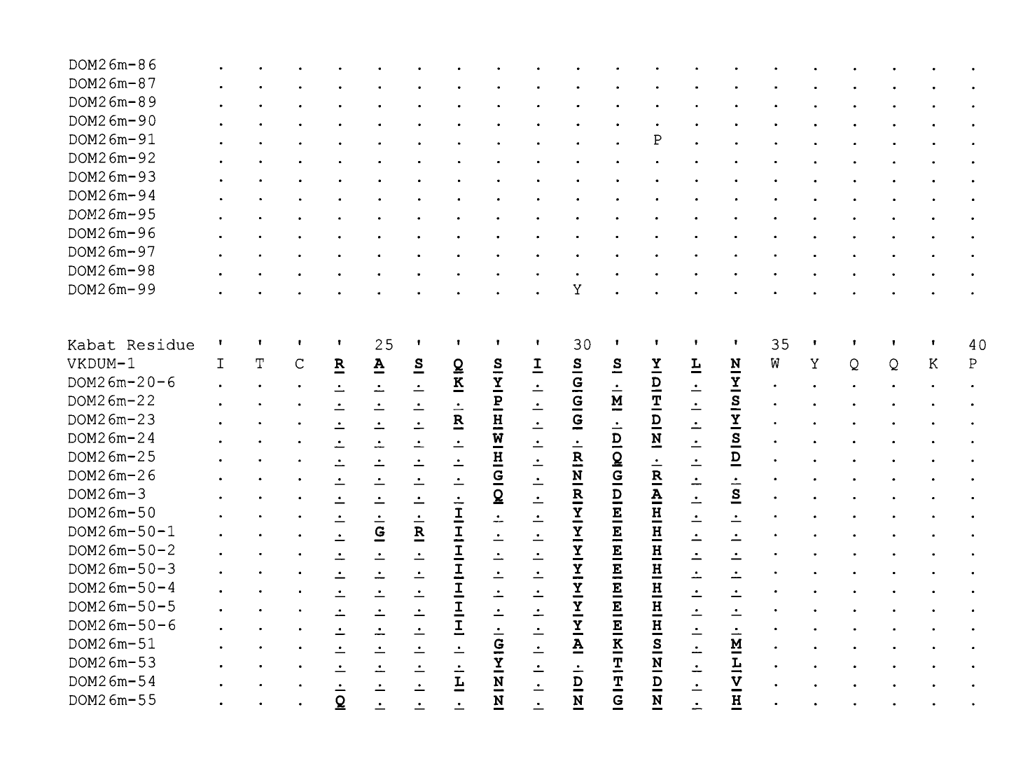

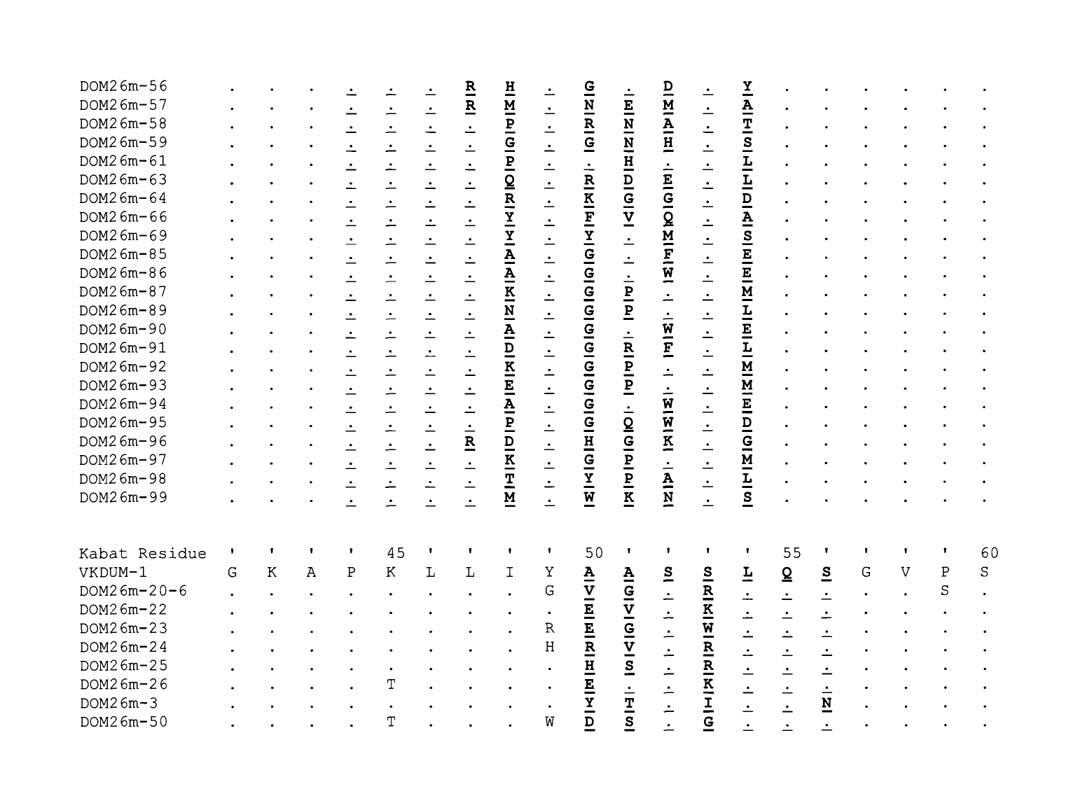

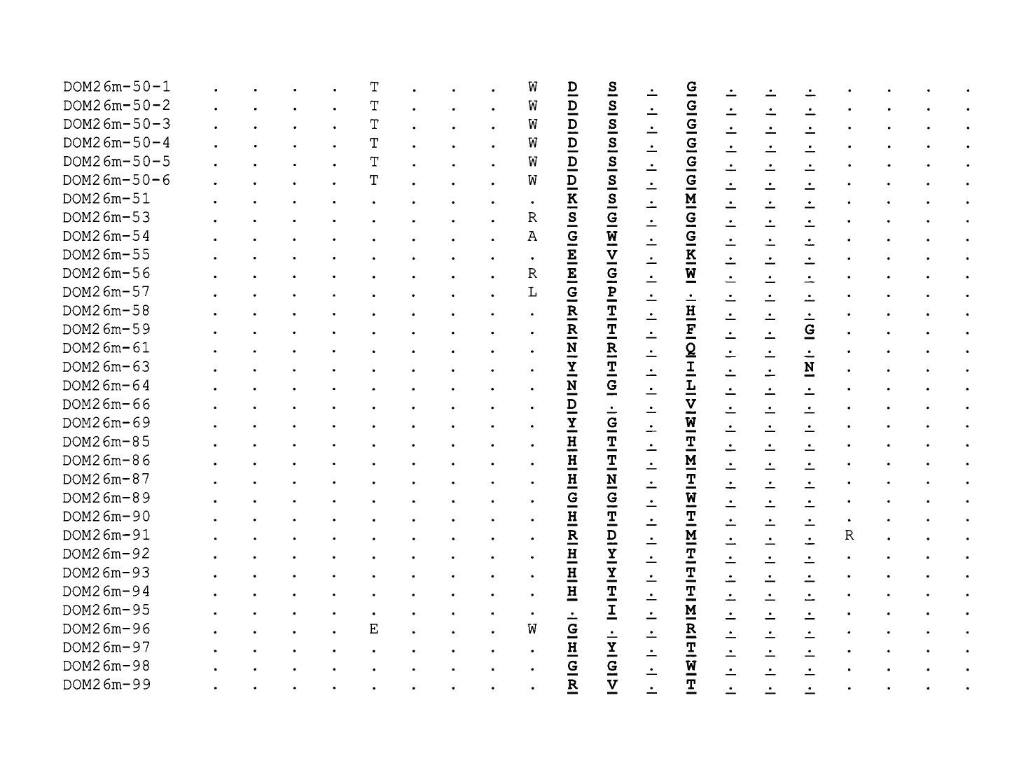

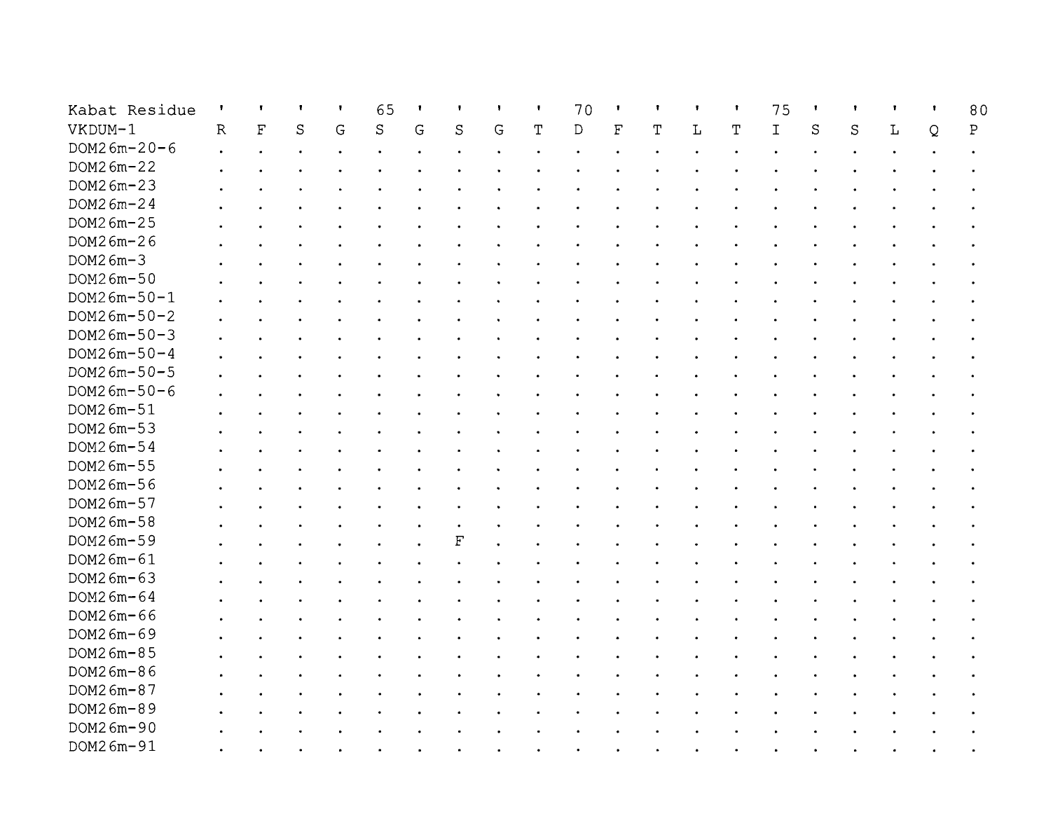

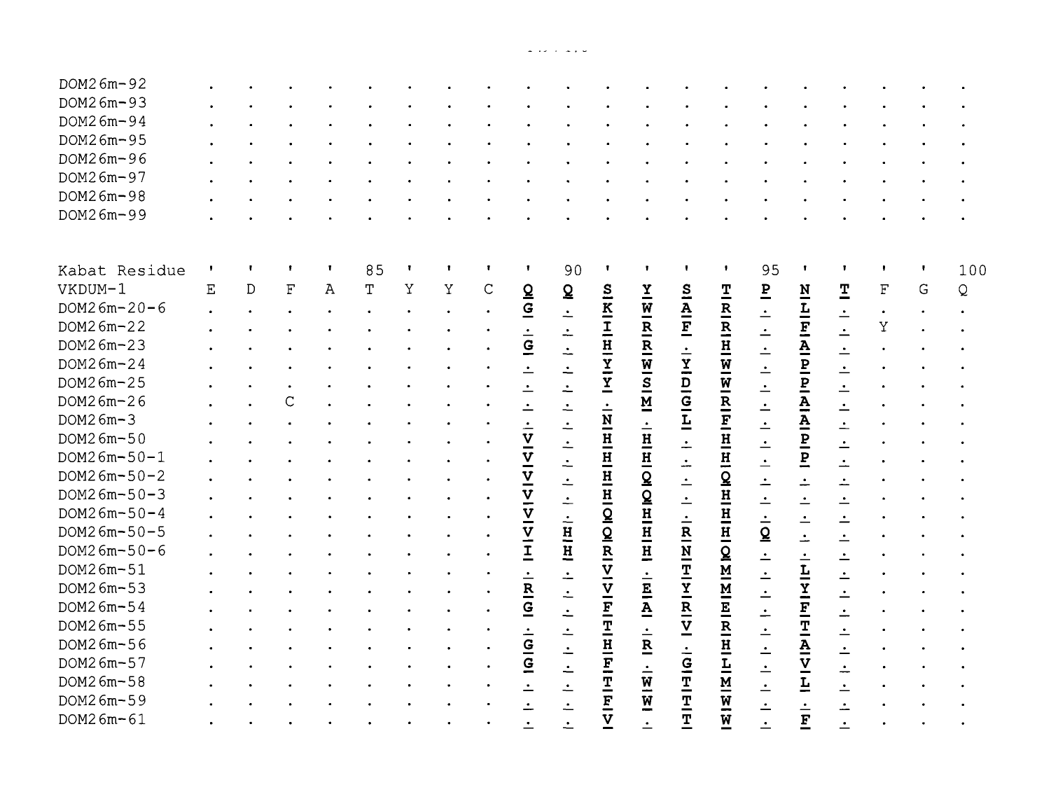

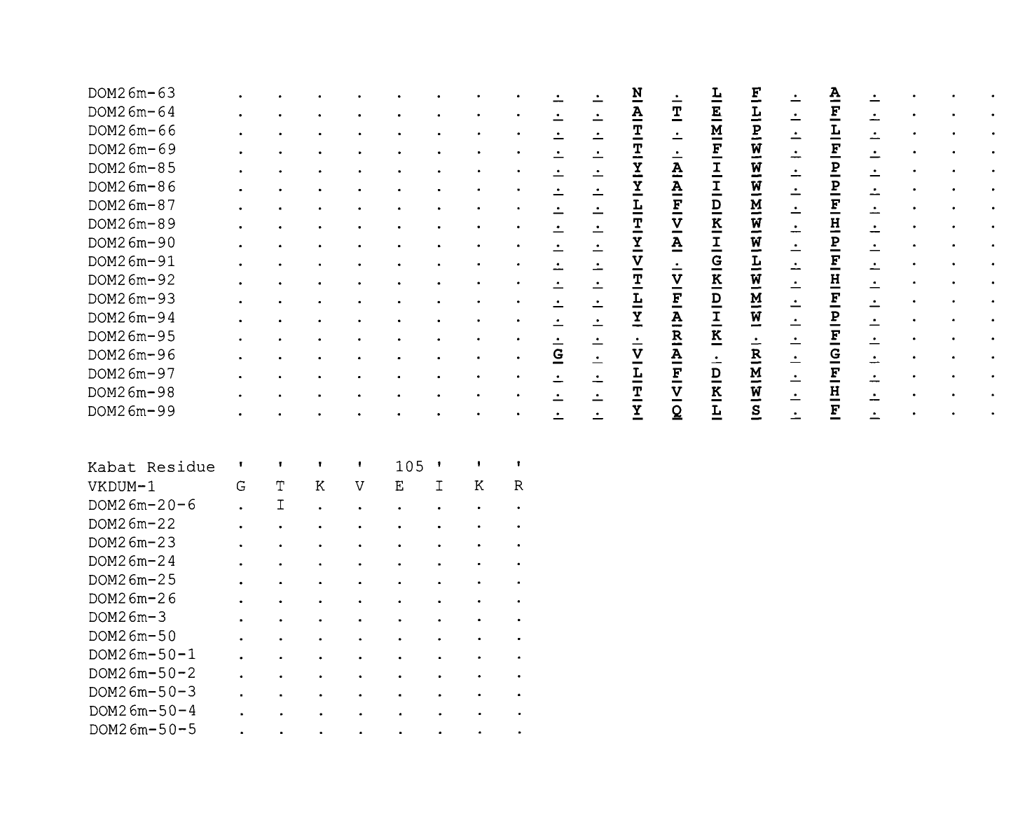

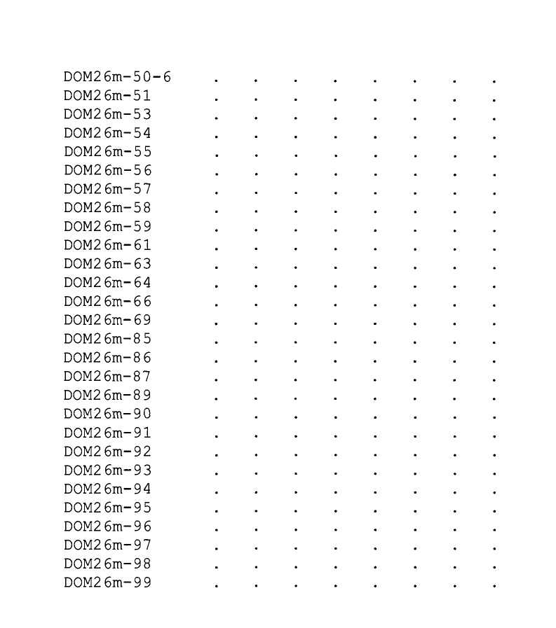

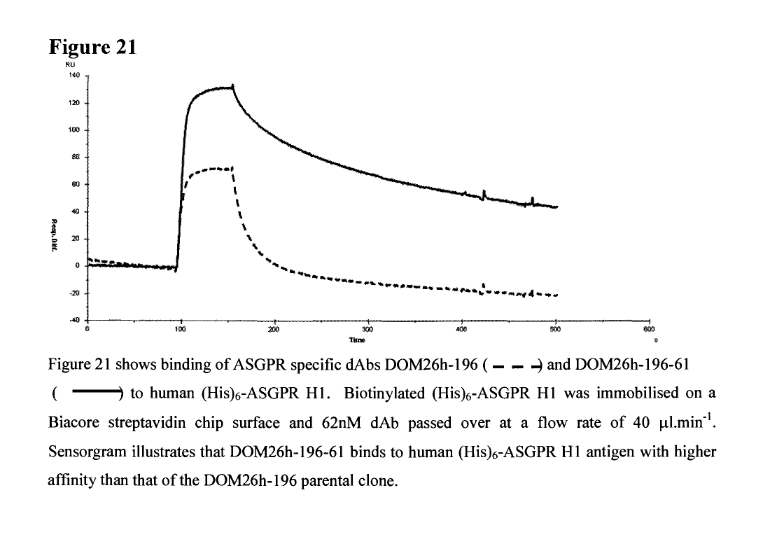

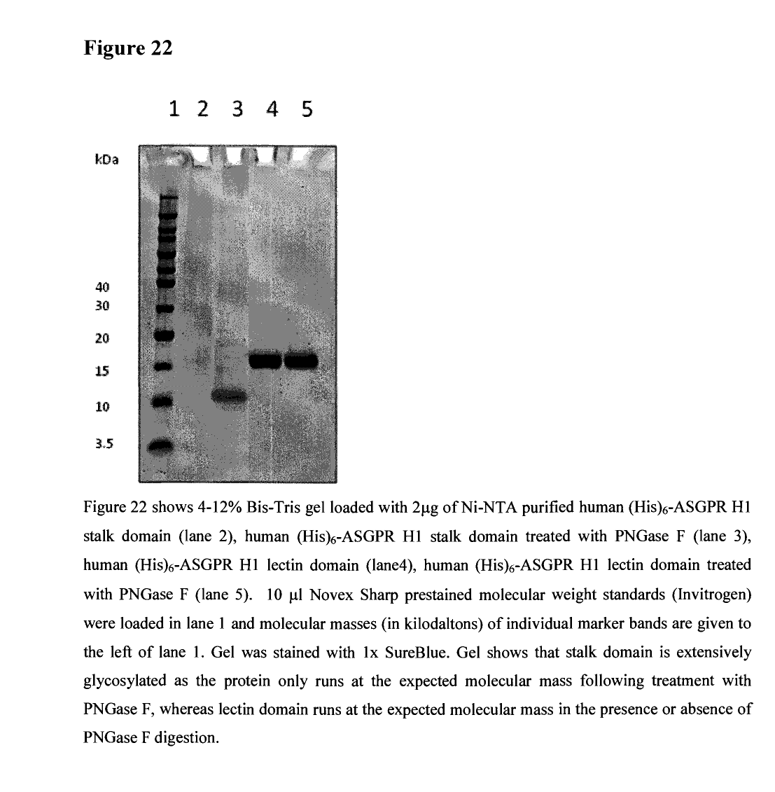

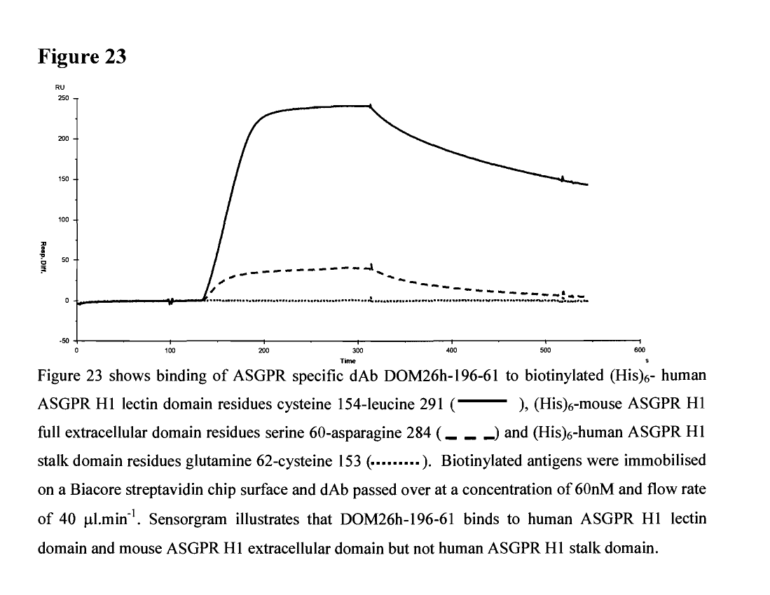

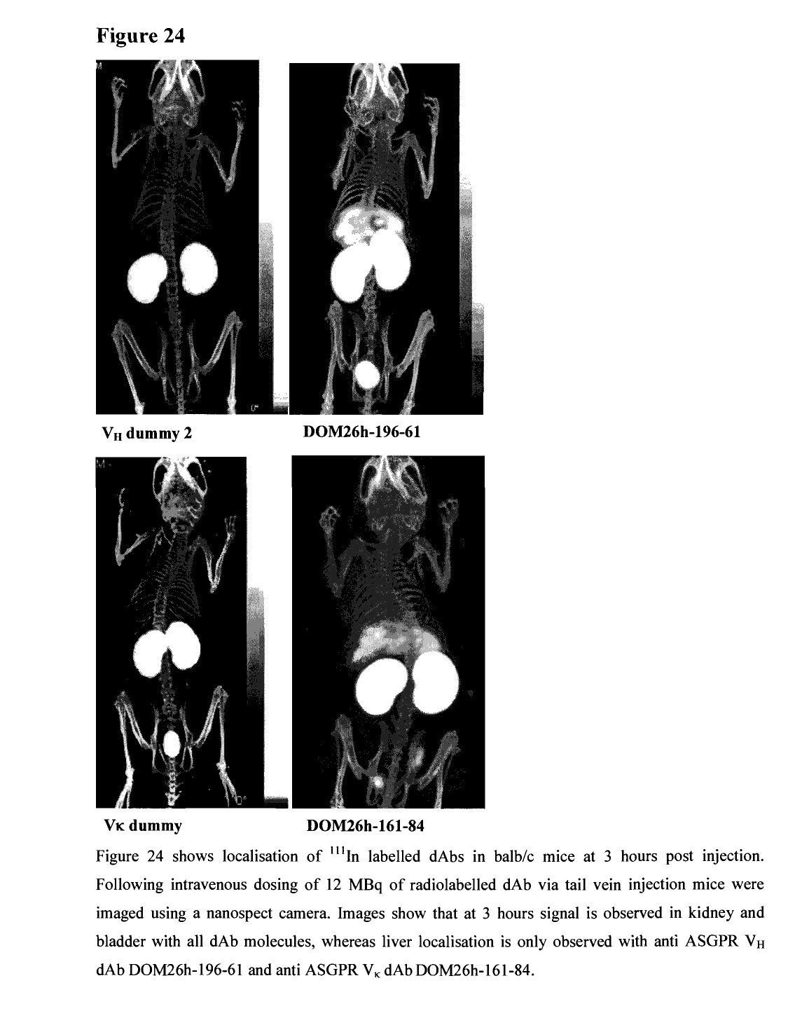

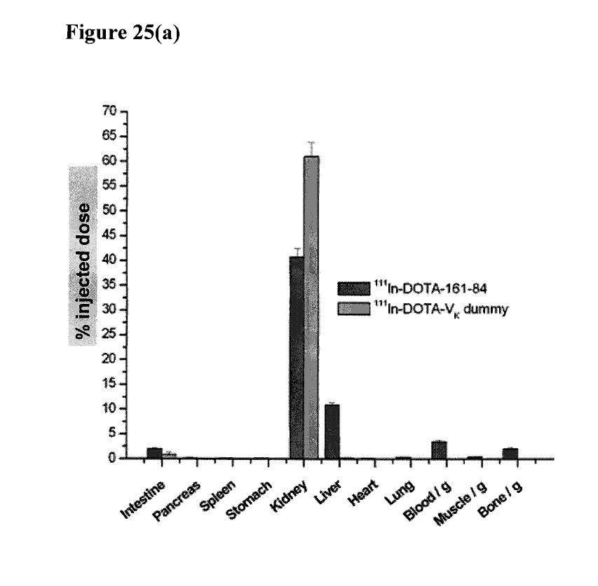

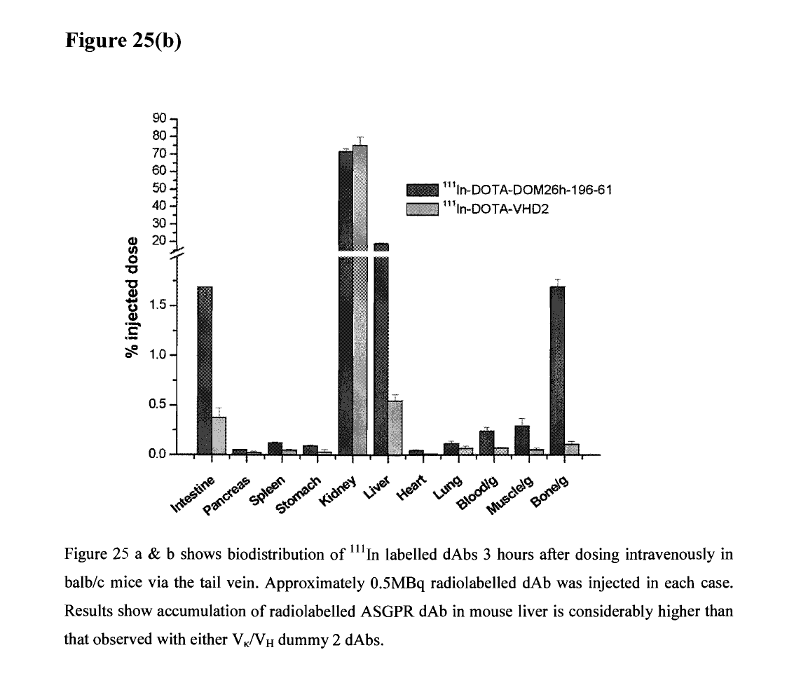

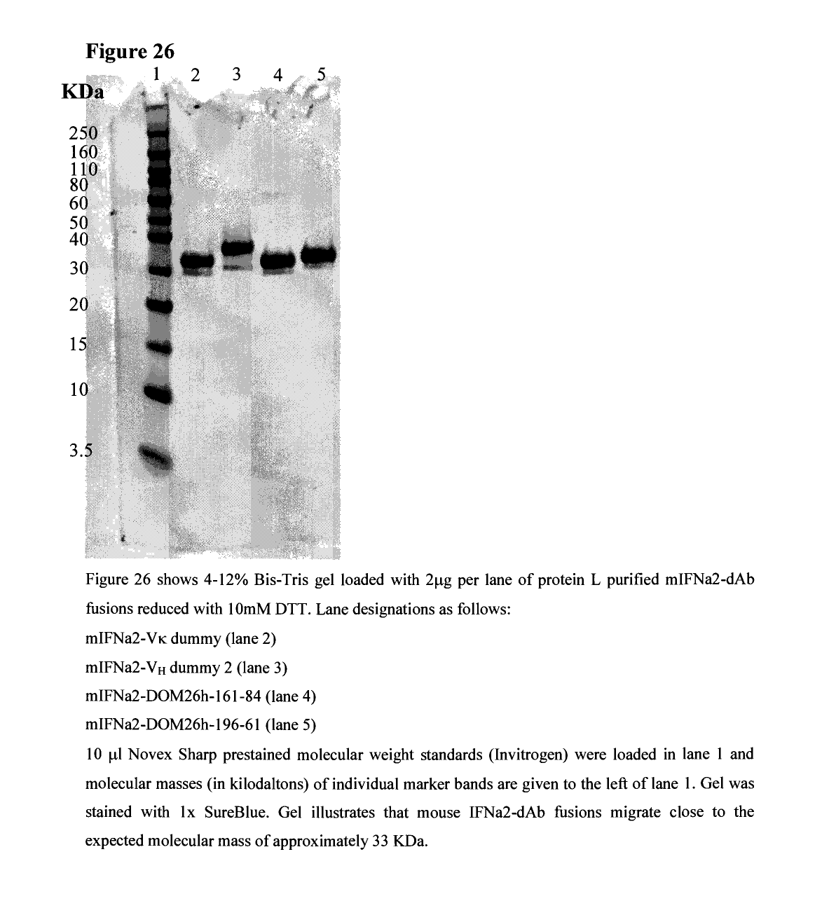

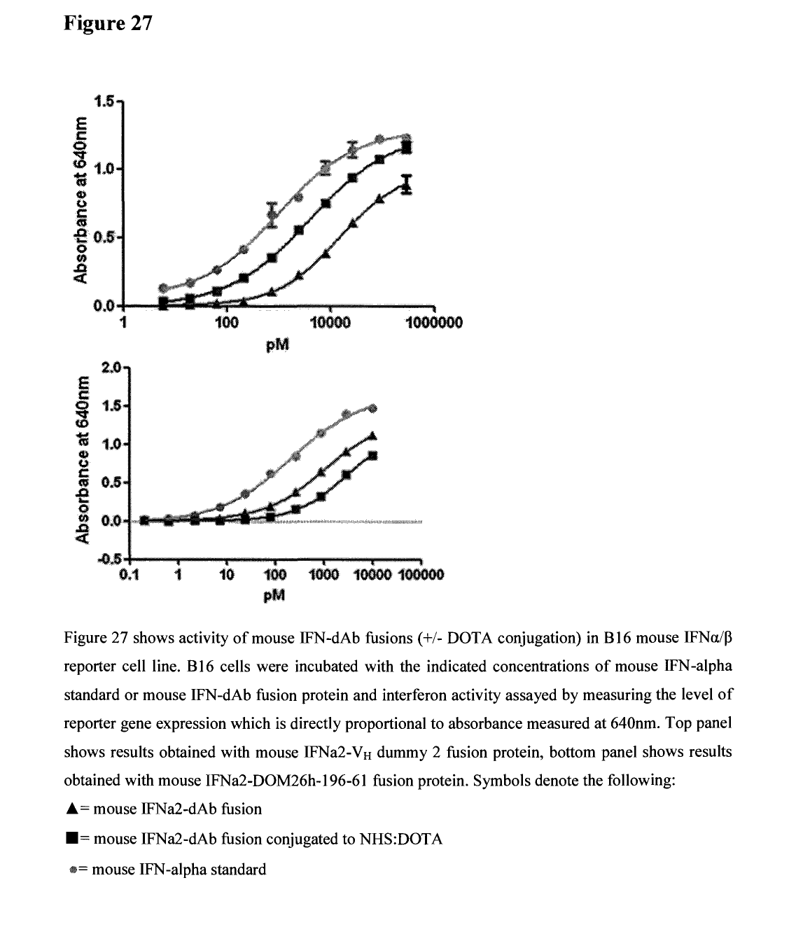

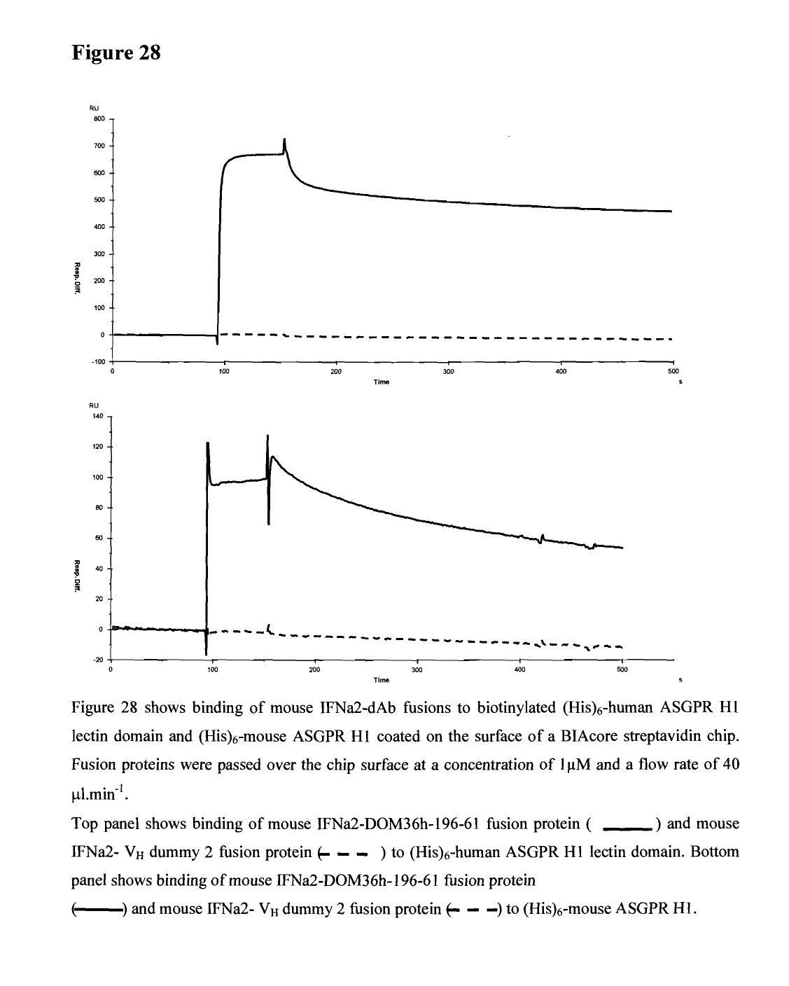

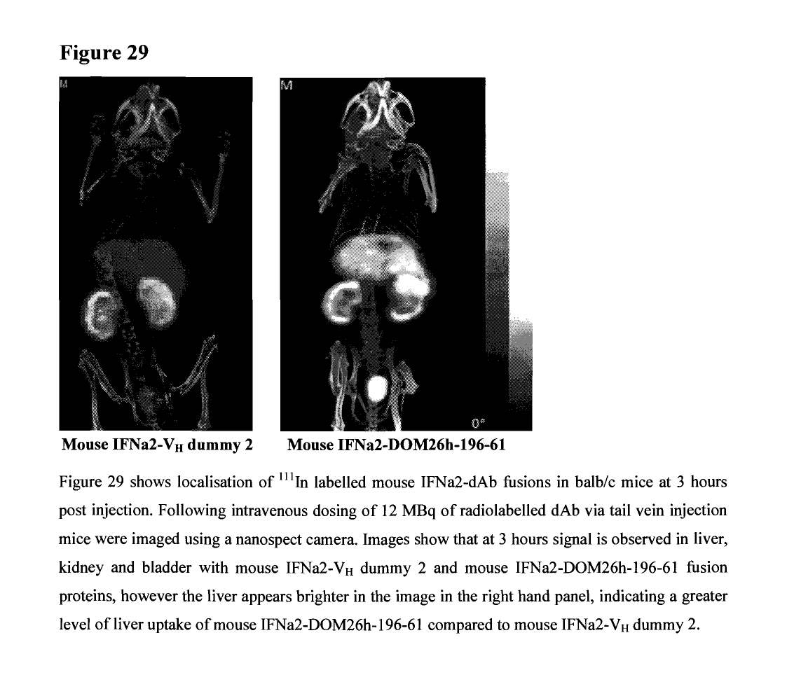

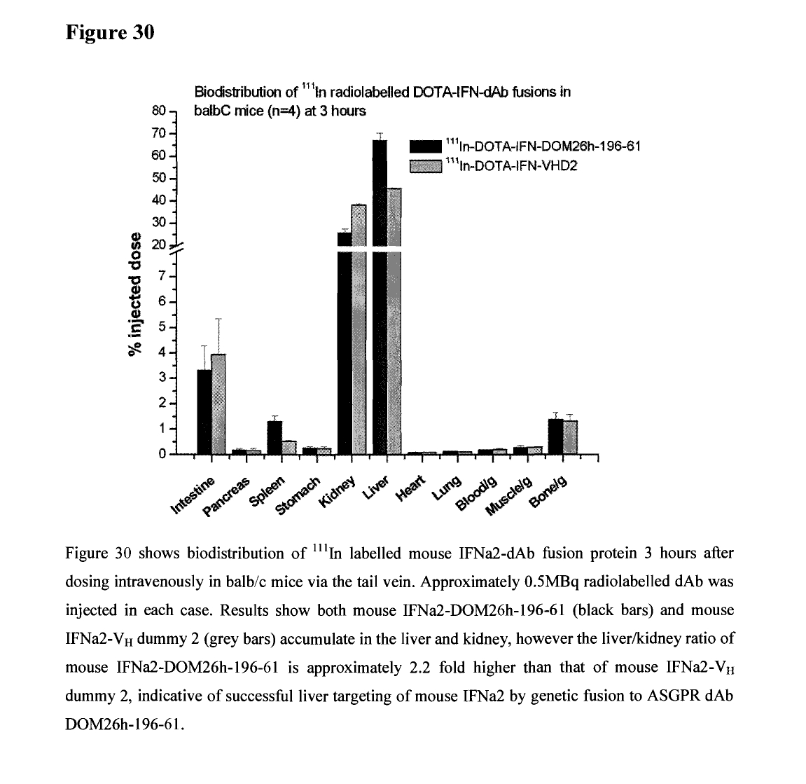

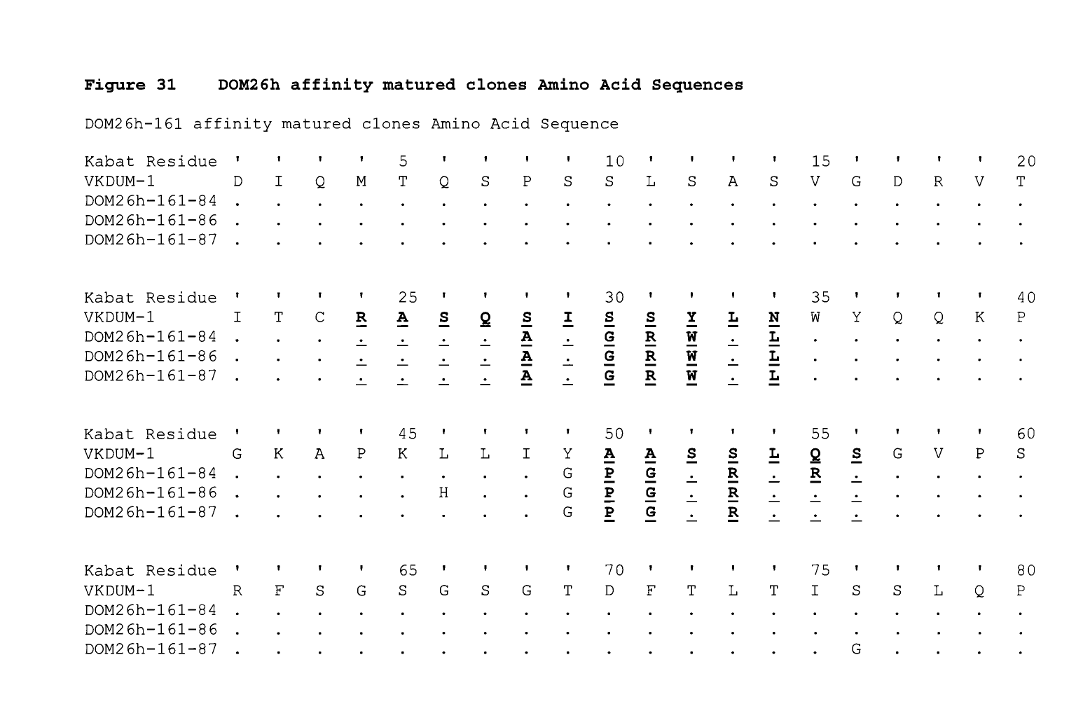

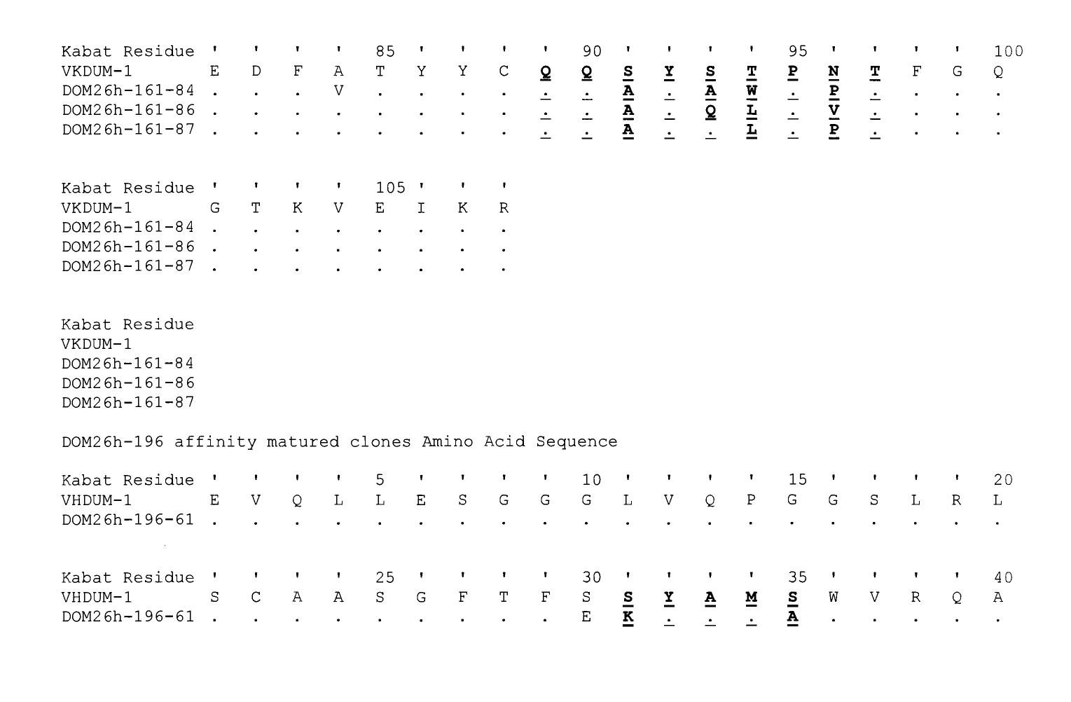

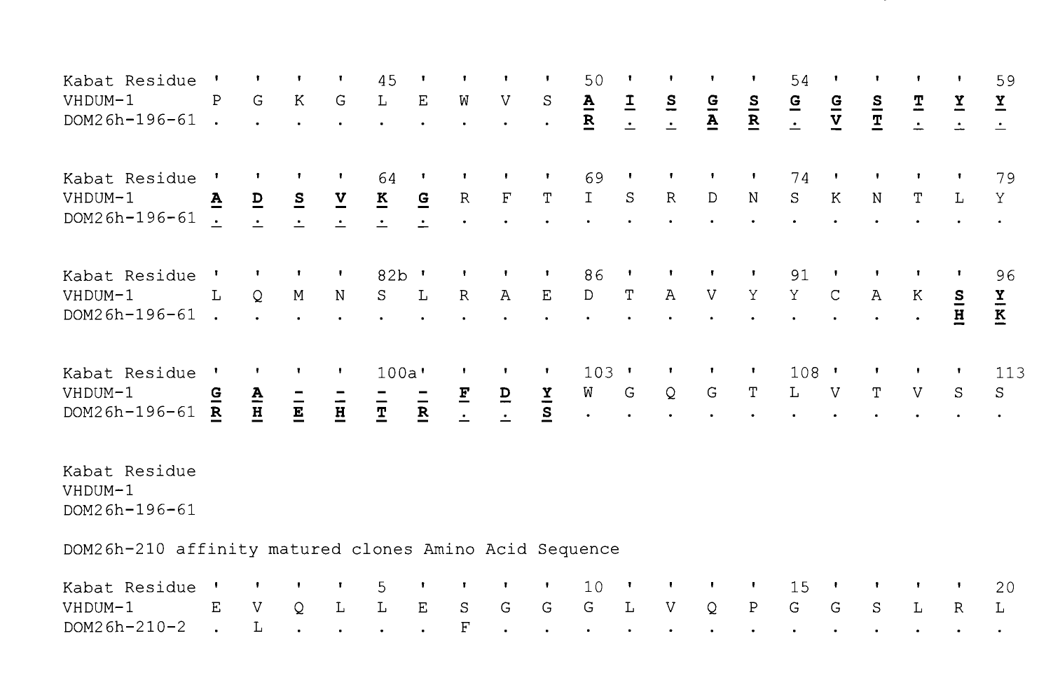

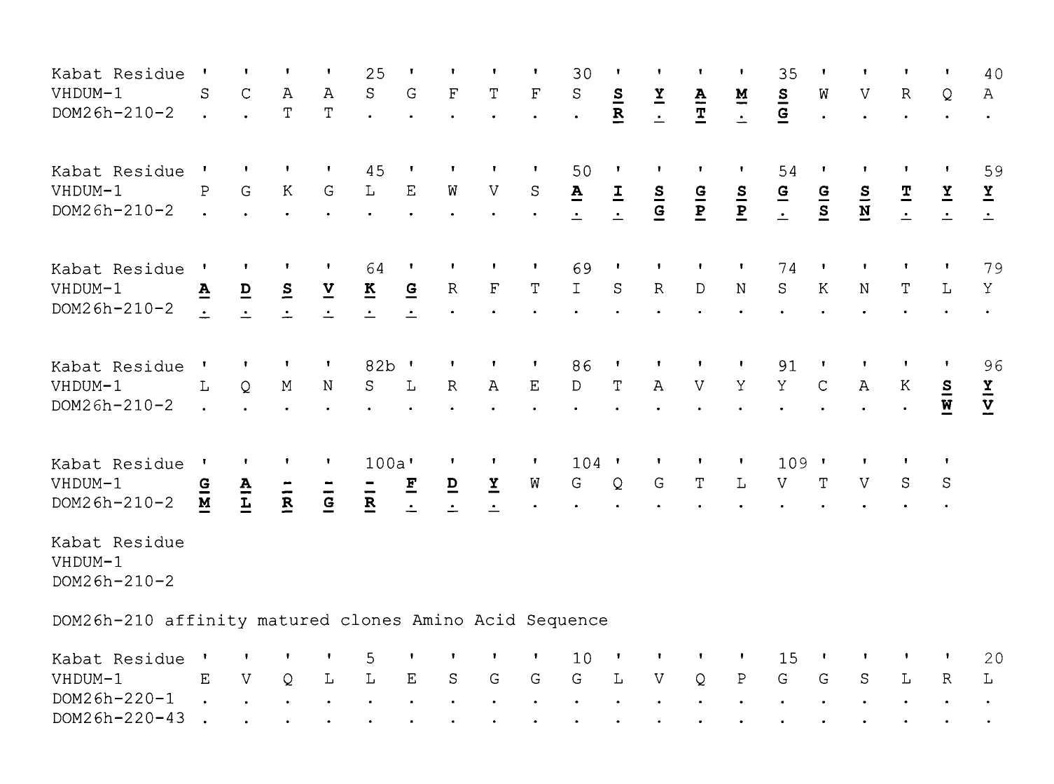

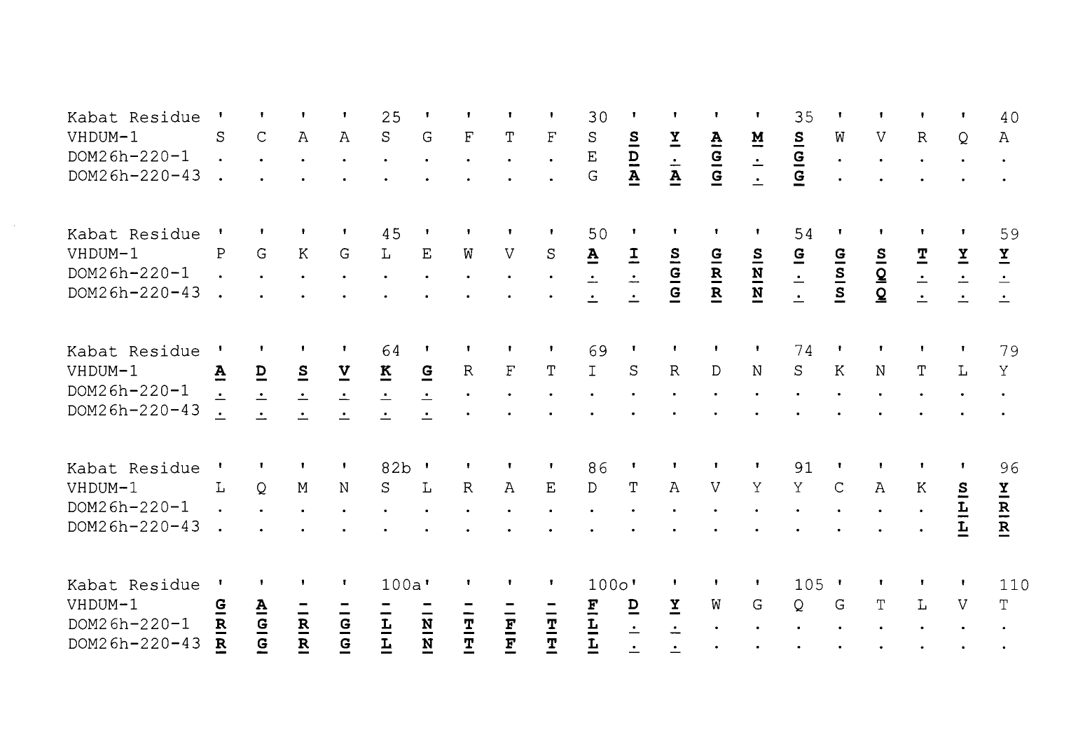

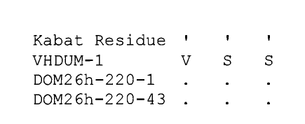

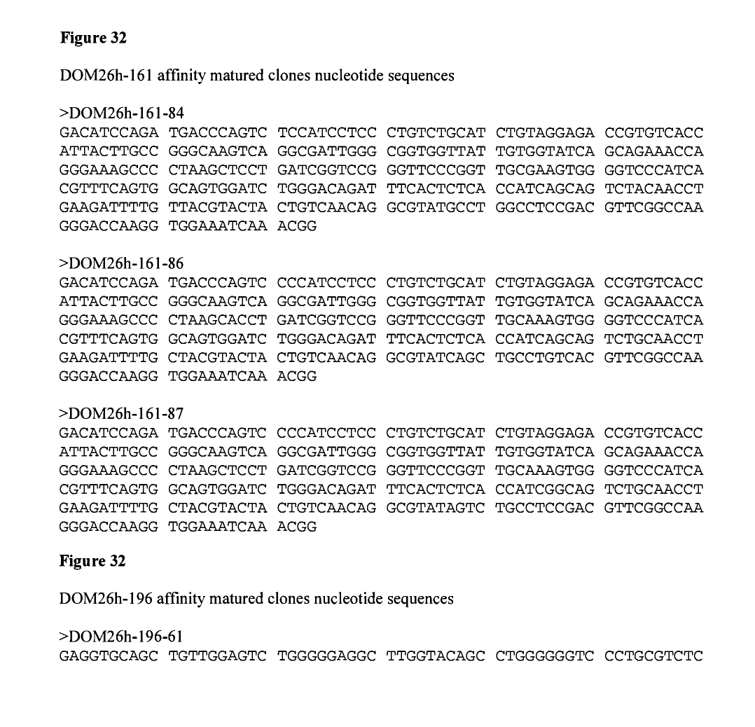

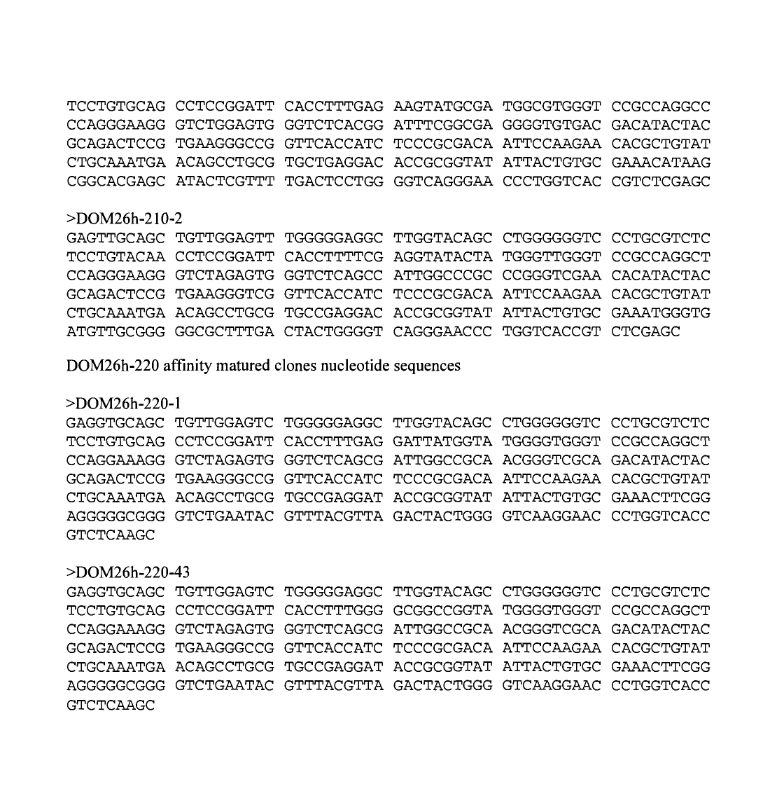

Plasmid DNA was prepared using QIAfilter megaprep (Qiagen). 1 μg DNA/ml was transfected with 293-Fectin into HEK293E cells and grown in serum free media. The protein is expressed in culture for 5 days and purified from culture supernatant using protein L streamline resin, eluted with 0.1 M glycine pH 2.0 and neutralised with 1 M Tris pH 8.0. The proteins were buffer exchanged into PBS. Purity was assessed by reducing SDS-PAGE as above (FIG. 9).

Interferon activity of mouse IFNa2-dAb fusions was assayed using a luciferase reporter assay (CHO-ISRE Luc assay). CHO-Kl cells were transiently transfected with the luciferase reporter construct pISRE-Luc (Clontech; http://www.clontech.com/images/pt/PT3372-5.pdf). Following overnight incubation transfected cells were plated onto 96 well microtitre plates and incubated for 4 hours at 37° C. before treatment with mouse IFNa2-dAb fusions for one hour. IFN-stimulated cells were then treated with Bright-Glo Luciferase reagent (http://www.promega.com/tbs/tm052/trn052.pdf) and read on a Wallac microplate reader. Recombinant mouse Interferon-alpha expressed in E coli (PBL Biomedical Laboratories) was used as a standard. Results show that mouse IFNa2-dAb fusions are active in this assay (FIG. 10).

ASGPR binding activity of mouse IFNa2-dAb fusions was also tested by biacore as above. DOM26m-33 binding activity was retained in the context of an in-line fusion to mouse IFNa2 (FIG. 11).

Sequences:

Mouse IFNa2
(Seq ID No. 93)
CDLPHTYNLRNKRALKVLAQMRRLPFLSCLKDRQDFGFPLEKVDNQQIQK
AQAIPVLRDLTQQTLNLFTSKASSAAWNTTLLDSFCNDLHQQLNDLQTCL
MQQVGVQEPPLTQEDALLAVRKYFHRITVYLREKKHSPCAWEVVRAEVWR
ALSSSVNLLPRLSEEKE
(Seq ID No. 94)
TGCGATCTGCCTCACACTTATAACCTCAGGAACAAGAGGGCCTTGAAGGT
CCTGGCACAGATGAGGAGGCTCCCCTTTCTCTCCTGCCTGAAGGACAGGC
AGGACTTTGGATTCCCCCTGGAGAAGGTGGATAACCAGCAGATCCAGAAG
GCTCAAGCCATCCCTGTGCTGCGAGATCTTACTCAGCAGACCTTGAACCT
CTTCACATCAAAGGCTTCATCTGCTGCTTGGAATACAACCCTCCTAGACT
CATTCTGCAATGACCTCCACCAGCAGCTCAATGACCTGCAAACCTGTCTG
ATGCAGCAGGTGGGGGTGCAGGAACCTCCTCTGACCCAGGAAGACGCCCT
GCTGGCTGTGAGGAAATATTTCCACAGGATCACTGTGTACCTGAGAGAGA
AGAAACACAGCCCCTGTGCCTGGGAGGTGGTCAGAGCAGAAGTCTGGAGA
GCCCTGTCTTCCTCAGTCAACTTGCTGCCAAGACTGAGTGAAGAGAAGGA
G

### Example 8

Affinity Maturation of DOM26m and DOM26h Leads

Error-prone PCR libraries were assembled for clones DOM26m-20, -50, -29, -33, -52 and DOM26h-61, -99, -104, -110 and -159. The parent clones in pDOM5 vector were subjected to two rounds of error-prone PCR using GeneMorph II kit (Stratagene). In the PCR reaction 0.75 μg of vector was amplified for 30 cycles using primers AS9 and AS339, according to manufacturer's protocol. In the second round of amplification 0.1 μl of the first amplification reaction product was reamplified in 100 ill volume for 35 cycles using primers AS639 and AS65. The reaction product was purified by electrophoresis using 2% E-Gels (Invitrogen) and Qiagen Gel Purification kit (Qiagen). The purified reaction product was cut with 200 units of Sal I (High concentration, NEB) and 100 units Not I (High concentration, NEB) in 100 μl volume at 37C for 18 hours. The digested DOM26m and DOM26h inserts were gel purified using 2% E-gels and eluted into 20 μl of water.

Each library insert was ligated into 1 μl of 30 nM pIE2a²A vector (see WO2006018650) using T4 DNA Ligase (NEB) in an overnight reaction at 160C in 25 μl volume. An aliquot of 0.1 μl of the ligated library was used to quantify the number of ligated vector molecules. The reaction yield in the form of circularized vectors was measured by qPCR (Mini-Opticon, iQ SYBR Green pre-mix, Bio-Rad cat no. 170-8880) using primers AS79 and AS80 (p174, R17058). Amplification cycles were: 2 min 94° C., followed by 40 cycles of 15 sec 94° C., 30 sec 60° C. and 30 sec 72° C. . The amount of DNA was quantified on a BioRad MiniOpticon Real-Time PCR Machine (Bio-Rad Laboratories, Hercules Calif.) and analysed using Opticon Monitor version 3.1.32 (2005) software provided by Bio-Rad Laboratories. Standard curve from a sample of known DNA concentration covered the range from 500 to 5×10⁸ molecules per reaction.Typical reaction yield (of independent ligations that equals to library diversity) varied between 2×10⁸ and 2×10⁹ circularized copies of vector per reaction.

0.5 μl of the ligation mix was also used to transform a 10 μl of XL10-Gold cells (Stratagene). The inserts from the colonies were amplified using primers AS79 and AS80, SuperTaq DNA polymerase. The reaction products were purified using Millipore Multiscreen plates and 8 clones were sequenced for each library using T7 primer. On average, the libraries contained 1.8-2.8 amino acid mutations per gene (p179, R17058).

The rest of the ligation mix was PCR amplified in 15 μl volume using SuperTaq DNA polymerase with primers AS 11 and AS 17 to generate the PCR fragments required for the selection.

### Selections

Nine rounds of selection were carried out in total, whilst keeping all the libraries separate and using a series of nested primer sets AS12+AS18, AS13+AS19, AS14+AS20, AS15+AS21, AS16+AS22, AS29+AS153, AS106+AS154, AS109+AS155 and AS98+AS156, according to the method described in WO2006018650, except that KOD Hot-Start DNA polymerase (Merck) was used throughout the process. In the first round of selection 5×10⁹ molecules of library were emulsified in 1 ml of emulsion, whereas in the subsequent eight rounds 5×10⁸ molecules per reaction were used. Affinity capture of protein DNA complexes was carried out using mouse ASGPR biotinylated with NHS-LC-biotin (Pierce, according to manufacturer's protocol). M280 Streptavidin Dynabeads at 3×10⁷ beads per reaction (Invitrogen) were used throughout to capture ligand-dAb-DNA complexes. 4-6 fmol of mouse ASGPR was pre-coated onto beads (in round 1) or used in solution 200 μl volume during the capture phase (rounds 2-9).

Following the final round of selection, the amplified DNA was cut with SalI/NotI enzymes and the dAb insert gel purified on 2% E-Gel. The purified insert was cloned into SalI/NotI-cut pDOM10 vector and transformed into Machl Chemically competent cells (Invitrogen). 96 colonies were picked for each library. The bacterial colonies were used to run PCR reactions and to inoculate 100 μl stock LB and 600 μl TB/OnEx (Merck) cultures. The TB/OnEx cultures were used for autoinduction expression during 72 h incubation at 300 C, 750 RPM in 2.2 ml DeepWell plates. The expression products were screened on BlAcore using HBS-P buffer and SA chips (all BIAcore) coated with biotinylated proteins, human ASGPR in channel 2, mouse ASGPR in channel 3 and either protein A or protein L in channel 4. Channel 1 was left uncoated. The colony PCR was performed using SuperTaq with primers AS9 and AS65. The PCR reaction products were purified using Multiscreen plates (Millipore) and sequenced using M13 reverse primer.

Results

A number of clones were identified by sequence enrichment (DOM26m-20 and DOM26h-61 libraries) or BlAcore screening of supernatants (DOM26m-52 library). No improved clones or sequence enrichments were observed for the rest of the libraries.

Further affinitiy maturation of DOM26m and DOM26h leads was carried out using doped libraries. Libraries were assembled by PCR using SuperTaq DNA polymerase and targetd dAb genes in pDOM5 vector. The doped oligonucleotides consisted of fixed positions (indicated by a capital letter and in which case 100% of oligonucleotides have the indicated nucleotide at that position) and mixed nucleotide composition, indicated by lower case in which case 85% of oligonucleotides will have the dominant nucleotide at this position and 15% will have an equal split between the remaining three nucleotides.

DOM26m-20: In the first reaction CDR1 of DOM26m-20 was randomized using oligonucleotides AS9 and AS1253, while CDR2 was randomized using oligonucleotides AS1257 and AS339. The reaction products were gel purified, mixed and spliced by SOE-PCR (Horton et al. Gene, 77, p61 (1989)) using primers AS65 and AS639 as secondary nested primers, providing a library with both CDR1 and CDR2 randomisation. CDR3 was randomized using primersAS9 and AS 1259.

DOM26m-50: In the first reaction CDR1 of DOM26m-20 was randomized using oligonucleotides AS9 and AS1254, while CDR2 was randomized using oligonucleotides AS1258 and AS339. The reaction products were gel purified, mixed and spliced by SOE-PCR using primers AS65 and AS639 as secondary nested primers, providing a library with both CDR1 and CDR2 randomisation. CDR3 was randomized using primersAS9 and AS 1260.

DOM26m-29: In the first reaction CDR1 of DOM26m-20 was randomized using oligonucleotides AS9 and AS1261, while CDR2 was randomized using oligonucleotides AS1267 and AS339. The reaction products were gel purified, mixed and spliced by SOE-PCR using primers AS65 and AS639 as secondary nested primers, providing a library with both CDR1 and CDR2 randomisztion. CDR3 was randomized using primersAS9 and AS 1270.

DOM26m-33: In the first reaction CDR1 of DOM26m-20 was randomized using oligonucleotides AS9 and AS1262, while CDR2 was randomized using oligonucleotides AS1268 and AS339. The reaction products were gel purified, mixed and spliced by SOE-PCR using primers AS65 and AS639 as secondary nested primers, providing a library with both CDR1 and CDR2 randomisation. CDR3 was randomized using primersAS9 and AS 1271.

DOM26h-99: Separate libraries for each CDR was assembled by SOE-PCR. CDR1: the first amplifications with primer pairs AS1290+AS339 and AS9+AS1310 for CDR1, AS 1294+AS339 and AS9+AS1278 for CDR2 and AS1298+AS339 and AS9+AS1304 for CDR3. The amplification products for individual CDRs were mixed, spliced by SOE PCR and reamplified using primers AS639 and AS65.

DOM26h-159: Separate libraries for each CDR was assembled by SOE-PCR. CDR1: the first amplifications with primer pairs AS1322+AS339 and AS9+AS1310 for CDR1, AS 1323+AS339 and AS9+AS1278 for CDR2 and AS1324+AS339 and AS9+AS1304 for CDR3. The amplification products for individual CDRs were mixed, spliced by SOE PCR and reamplified using primers AS639 and AS65.

DOM26m-52-3: The first amplifications were carried out with primer pairs AS1287+AS339 and AS9+AS1263 for CDR1, AS1325+AS339 and AS9+AS1327 for CDR2 (first library), AS1326+AS339 and AS9+AS1327 for CDR2 (second library), and AS9+AS1272 for CDR3. The amplification products for individual CDRs1-2 were mixed, spliced by SOE PCR and reamplified using primers AS639 and AS65.

All assembled library fragments were gel purifed, SalI/NotI cut and ligated into pIE2a²A vector as described above, with ligation yields exceeding 10⁹ independent ligations per reaction, as measured by qPCR and described above. (23, 27, 28 R17479)

Selections

Nine rounds of selection were carried out in total, whilst keeping all the libraries separate and using a series of nested primer sets AS12+AS18, AS13+AS19, AS14+AS20, AS15+AS21, AS16+AS22, AS29+AS153, AS106+AS154, AS109+AS155 and AS98+AS156, as described above. In the first round of selection 2.5×10⁹ molecules of library were emulsified in 1 ml of emulsion, whereas in the subsequent eight rounds 5×10⁸ molecules per reaction were used. Affinity capture of protein DNA complexes was carried out using mouse or human ASGPR biotinylated with NHS-LC-biotin (Pierce, according to manufacturer's protocol). M280 Streptavidin Dynabeads at 3×10⁷ beads per reaction (Invitrogen) were used throughout to capture ligand-dAb-DNA complexes. 2-6 fmol of mouse ASGPR was pre-coated onto beads (in round 1) or used in solution 200 μl volume during the capture phase (rounds 2-9).

Following the final round of selection, the amplified DNA was cut with SalI/NotI enzymes and the dAb insert gel purified on 2% E-Gel. The purified insert was cloned into SalI/NotI-cut pDOM10 vector and transformed into Machl Chemically competent cells (Invitrogen). 96 colonies were picked for each library and processed as described above for the error-prone PCR library.

Results

A number of clones were identified by sequence enrichment (DOM26m-20 and DOM26h-61 libraries) or BlAcore screening of supernatants (DOM26m-52 library). No improved clones or sequence enrichments were observed for the rest of the libraries.

Oligonucleotide sequences are shown in Table 9 below:

TABLE 9

| | |
|---|---|
| AS9 | CAGGAAACAGCTATGACCATG Seq ID No. 95 |
| AS11 | TTCGCTATTACGCCAGCTGG Seq ID No. 96 |
| AS12 | AAAGGGGGATGTGCTGCAAG Seq ID No. 97 |
| AS13 | AAGGCGATTAAGTTGGGTAAC Seq ID No. 98 |
| AS14 | CCAGGGTTTTCCCAGTCAC Seq ID No. 99 |
| AS15 | GAGATGGCGCCCAACAGTC Seq ID No. 100 |
| AS16 | CTGCCACCATACCCACGCC Seq ID No. 101 |
| AS17 | CAGTCAGGCACCGTGTATG Seq ID No. 102 |
| AS18 | AACAATGCGCTCATCGTCATC Seq ID No. 103 |
| AS19 | TCGGCACCGTCACCCTGG Seq ID No. 104 |
| AS20 | TGCTGTAGGCATAGGCTTGG Seq ID No. 105 |
| AS21 | CCTCTTGCGGGATATCGTC Seq ID No. 106 |
| AS22 | TCCATTCCGACAGCATCGC Seq ID No. 107 |
| AS29 | GAAACAAGCGCTCATGAGCC Seq ID No. 108 |
| AS65 | TTGTAAAACGACGGCCAGTG Seq ID No. 109 |
| AS79 | GGCGTAGAGGATCGAGATC Seq ID No. 110 |
| AS80 | TTGTTACCGGATCTCTCGAG Seq ID No. 111 |
| AS98 | CCAGCAACCGCACCTGTG Seq ID No. 112 |
| AS106 | AGTGGCGAGCCCGATCTTC Seq ID No. 113 |
| AS109 | CGATATAGGCGCCAGCAACC Seq ID No. 114 |
| AS153 | CAGTCACTATGGCGTGCTGC Seq ID No. 115 |
| AS154 | TAGCGCTATATGCGTTGATGC Seq ID No. 116 |
| AS155 | TTCTATGCGCACCCGTTCTC Seq ID No. 117 |
| AS156 | AGCACTGTCCGACCGCTTTG Seq ID No. 118 |
| AS339 | TTCAGGCTGCGCAACTGTTG Seq ID No. 119 |
| AS639 | CGCCAAGCTTGCATGCAAATTC Seq ID No. 120 |
| AS1253 | GGCTTTACCTGGTTTCTGCTGGTACCAATAMAAMTCMCTMCCMA |
| | TATAMTTMCTMGCMCGGCAAGTGATGGTGACACGG |
| | Seq ID No. 121 |
| AS1257 | TATTGGTACCAGCAGAAACCAGGTAAAGCCCCTAAGCTCCTGAT |
| | KGGKGGKGGKTCKCGKTTKCAKAGTGGGGTCTCATC |
| | Seq ID No. 122 |
| AS1259 | TGTGTGTGGCGGCCGCCCGTTTGATTTCCACCTTGGTCCCTTGG |
| | CCGAACGTMAGMGGMCTMGCMCAMTTMTGMCCMCAGTAGTACG |
| | TAGC Seq ID No. 123 |
| AS1254 | GGCTTTCCCTGGTTTCTGCTGGTACCAATTMAAMTGMTCATAMA |
| | TMCTMATMCTMGCMCGGCAAGTGATGGTGACACGG |
| | Seq ID No. 124 |
| AS1258 | AATTGGTACCAGCAGAAACCAGGGAAAGCCCCTACGCTCCTGAT |
| | KTGKGAKTCKTCKGGKTTKCAKAGTGGGGTCCCATC |
| | Seq ID No. 125 |
| AS1260 | TGTGTGTGGCGGCCGCCCGTTTGATTTCCACCTTGGTCCCTTGG |
| | CCGAACGTMGGMGGMTGMCTMTGMTGMTGMACMCAGTAGTACGT |
| | AGC Seq ID No. 126 |
| AS1261 | TGAGACCCACTCCAGACCCTTCCCTGGAGCCTGGCGGGCCCAMA |
| | TMATMCCMCTMTCMTCAAAGGTGAATCCGGAG |
| | Seq ID No. 127 |
| AS1262 | TGAGACCCACTCTAGACCCTTCCCTGGAGCCTGGCGGACCCAMA |
| | TMATMTCATAMTTMGCAAAGGTGAATCCGGAG |
| | Seq ID No. 128 |
| AS1263 | TGAGACCCACTCTAGACCCTTCCCTGGAGCCTGGCGGACCCAMC |
| | TMATMGGMTGMTCMCTAAAGGTGAATCCGGAG |
| | Seq ID No. 129 |
| AS1267 | CTCCAGGGAAGGGTCTGGAGTGGGTCTCAGGKATKGCKTCKGAK |
| | GGKAGKACKACKTACTACGCKGAKTCKGTKAAKGGKCGGTTCAC |
| | CATC Seq ID No. 130 |
| AS1268 | CTCCAGGGAAGGGTCTAGAGTGGGTCTCAGGKATKAAKCAKTCK |
| | GGKTCKCGKACKTACTACGCKGAKTCKGTKAAKGGKCGGTTCAC |
| | CATC Seq ID No. 131 |
| AS1270 | TGTGTGTGGCGGCCGCGCTCGAGACGGTGACCAGGGTTCCCTGA |
| | CCCCAGTAMTCMAAMGAMAGMCCMGATTTCGCACAGTAATA |
| | Seq ID No. 132 |
| AS1271 | TGTGTGTGGCGGCCGCGCTCGAGACGGTGACCAGGGTTCCCTGA |
| | CCCCAGTAMTCMAAMGAMGAMCCMGATTTCGCACAGTAATA |
| | Seq ID No. 133 |
| AS1272 | TGTGTGTGGCGGCCGCGCTCGAGACGGTGACCAGGGTTCCCTGA |
| | CCCCAGTAMTCMAAMTCMCGMACMGATTTCACACAGTAATA |
| | Seq ID No. 134 |
| AS1278 | TGAGACCCACTCTAGACCCTTCCCTGGAGCCTGGCGGACCCA |
| | Seq ID No. 135 |
| AS1287 | TGGGTCCGCCAGGCTCCAGGGAAGGGTCTAGAGTGGGTCTCA |
| | Seq ID No. 136 |
| AS1294 | GAAGGGTCTAGAGTGGGTCTCATCKATTAGKTCKACKGGKCTKA |
| | GKACKTACTACGCKGAKTCKGTGAAKGGKCGGTTCACCATCTCC |
| | CG Seq ID No. 137 |
| AS1298 | CGGTATATTACTGTGCGAAAGAKGCKTCKCGKTTKAGKCAKCCK |
| | TTKGAKTACTGGGGTCAGGGAACCCTGGTC |
| | Seq ID No. 138 |
| AS1304 | TTTCGCACAGTAATATACCGC Seq ID No. 139 |
| AS1310 | AAAGGTGAATCCGGAGGCTGCACAGGAGAGACGCAG |
| | Seq ID No. 140 |
| AS1322 | GCCTCCGGATTCACCTTTGCKGAKTATTCKATGTATTGGGTCCG |
| | CCAGGCTCCAGG Seq ID No. 141 |
| AS1323 | GAAGGGTCTAGAGTGGGTCTCAGAKATKAGKCCKTCKGGKAGKA |
| | TKACKTACTACGCKGAKTCKGTKAAKGGKCGGTTCACCATCTCC |
| | CGTGACAATTC Seq ID No. 142 |
| AS1324 | CGGTATATTACTGTGCGAAAGGKCTKCCKGGKCAKAAKATKCAK |
| | GTKGGKTTKGAKTACTGGGGTCAGGGAACCCTGGTC |
| | Seq ID No. 143 |
| AS1325 | GGGTCTCATCGATTAGTAAGCATGGTNNKNNKNNKTACTACGCA |
| | GACTCCGTG Seq ID No. 144 |
| AS1326 | GGGTCTCATCGATTAGTAAGNNKNNKNNKGTGACATACTACGCA |
| | GAC Seq ID No. 145 |
| AS1327 | CTTACTAATCGATGAGACCC Seq ID No. 146 |

### Example 9

Cloning and Expression of Human Asialoglycoprotein H1 Receptor Lectin and Stalk Domains

Full length human asialoglycoprotein receptor H1 subunit (ASGPR H1) cDNA was synthesised by DNA2.0 (see example 1). DNA encoding the stalk domain (Q62-C153) with an N-terminal (His)₆ tag was generated by site directed mutagenesis of Human (His)₆ ASGPR H1 Q62-L291 in pDOM50 expression vector (see example 1) using the Quikchange site directed mutagenesis kit (Stratagene) according to manufacturer's instructions. Primers LT020 and LT021 were used to introduce a double stop codon in this construct such that translation of Human (His)₆ ASGPR H1 Q62-L291 in pDOM50 terminates immediately after residue C153. DNA encoding the lectin domain (C154-L291) with an N-terminal (His)₆ tag was amplified by PCR using primers LT013 and LT014.

| | | |
|---|---|---|
| LT020 | CCGAGAGAACTTGCTAATAATGCCCCGTCAATTGGG | Human (His)₆ ASGPR H1 stalk |
| | (Seq ID No. 147) | domain 5′ primer |
| LT021 | CCCAATTGACGGGGCATTATTAGCAAGTTCTCTCGG | Human (His)₆ ASGPR H1 stalk |
| | (Seq ID No. 148) | domain 3′ primer |
| LT022 | GCCCGGATCCACCGGCCATCATCATCATCATCACGGG | Human (His)₆ ASGPR H1 |
| | TCGTGCCCCGTCAATTGGGTG | lectin domain 5′ primer |
| | (Seq ID No. 149) | |
| LT013 | GGGTGCCCGGATCCACCGGCCATCATCATCATCATCA | Human (His)₆ ASGPR H1 |
| | CGGGTCGCACGAGCGGTCTTGTTATTGGAGC | lectin domain 3′ primer |
| | (Seq ID No. 150) | |

PCR fragment was digested with BamHI/HindIII, gel purified and ligated into the corresponding sites in pDOM50 (see example 1).

Leader sequence (amino acid):
(Seq ID No. 5)
METDTLLLWVLLLWVPGSTG
Leader sequence (nucleotide):
(Seq ID No. 6)
ATGGAGACCGACACCCTGCTGCTGTGGGTGCTGCTGCTGTGGGTGCCCGG
ATCCACCGGGC

Plasmid DNA was prepared using QIAfilter megaprep (Qiagen). 1 μg DNA/ml was transfected with 293-Fectin into HEK293E cells and grown in serum free media. The protein was expressed in culture for 5 days and purified from culture supernatant using Ni-NTA resin and eluted with PBS+0.5 M Imidazole. The proteins were buffer exchanged into PBS.

Purity of lectin and stalk domains eluted from Ni-NTA was analysed by non-reducing SDS-PAGE (FIG. 14). SDS-PAGE analysis shows that human (His)₆-ASGPR H1 stalk domain migrates close to the expected molecular mass (10 KDa based on amino acid sequence) only when treated with 500 units of PNGase F (New England Biolabs) for 2 hours at 37° C., consistent with N-linked glycosylation of residues in the stalk domain. Human (His)₆-ASGPR H1 lectin domain migrates close to the expected molecular mass of 17.2 KDa irrespective of PNGase F treatment, indicating that the lectin domain of human ASGPR Hlis not extensively modified by N-linked glycosylation.

Sequences:

(His)₆-Human ASGPR H1 Stalk Domain
(Seq ID No. 151)
HHHHHHQNSQLQEELRGLRETFSNFTASTEAQVKGLSTQGGNVGRKMKSL
ESQLEKQQKDLSEDHSSLLLHVKQFVSDLRSLSCQMAALQGNGSERTC
(Seq ID No. 152)
CATCATCATCATCATCACCAGAACTCCCAACTCCAGGAAGAACTTCGAGG
ACTGAGGGAGACTTTCTCCAATTTCACCGCAAGCACGGAGGCTCAAGTGA
AGGGCCTCAGCACCCAGGGCGGGAATGTGGGCAGGAAAATGAAATCCCT
GGAGAGCCAGCTCGAAAAGCAGCAGAAAGATCTGTCCGAGGACCACTCT
AGCCTGTTGTTGCACGTGAAACAGTTTGTTTCCGACCTTAGGAGTCTTTC
TTGCCAAATGGCCGCCCTCCAGGGAAACGGGTCCGAGAGAACTTGC
(His)₆-Human ASGPR H1 Lectin Domain
(Seq ID No. 153)
HHHHHHGSCPVNWVEHERSCYWFSRSGKAWADADNYCRLEDAHLVVVTS
WEEQKFVQHHIGPVNTWMGLHDQNGPWKWVDGTDYETGFKNWRPEQPDD
WYGHGLGGGEDCAHFTDDGRWNDDVCQRPYRWVCETELDKASQEPPLL
(Seq ID No. 154)
CATCATCATCATCATCACGGGTCGTGCCCCGTCAATTGGGTGGAGCACGA
GCGGTCTTGTTATTGGTTTAGCCGAAGCGGAAAAGCCTGGGCCGATGCAG
ATAACTACTGCCGGCTTGAGGACGCCCATCTGGTCGTGGTGACCAGTTGG
GAGGAACAGAAATTCGTACAGCATCATATCGGGCCTGTTAACACATGGAT
GGGCCTTCATGACCAGAATGGTCCTTGGAAGTGGGTTGACGGAACCGATT
ACGAAACCGGATTCAAGAACTGGCGGCCTGAACAGCCAGACGACTGGTAT
GGACACGGCCTCGGAGGCGGGGAGGACTGCGCGCATTTCACAGACGATG
GCCGGTGGAATGATGATGTGTGCCAAAGGCCTTACAGATGGGTCTGCGAG
ACAGAGCTGGATAAGGCTTCACAAGAGCCTCCACTCCTG

### EXAMPLE 10

Surface Plasmon Resonance to Determine Binding of ASGPR dAbs to Human ASGPR Stalk Domain, Human ASGPR Lectin Domain and Mouse ASGPR Extracellular Domain

To assay for potential dAb binding activity human (His)₆-ASGPR H1 stalk domain, human (His)₆-ASGPR H1 lectin domain and mouse (His)₆-ASGPR H1 extracellular domain were biotinylated and immobilised on a biacore Streptavidin chip surface. ASGPR dAbs DOM26h-161-84, DOM26h-210-2, DOM26h-220-1 and DOM26h-196-61 with C-terminal FLAG epitope tags (expressed and purified from pDOM10 as in example 6) were passed over the chip surface at a flow rate of 40 μl.min⁻¹ and shown to bind human (His)₆-ASGPR H1 lectin domain and mouse (His)₆-ASGPR H1 extracellular domain. No binding to human (His)₆-ASGPR H1 stalk domain was observed with any of these clones (FIG. 15 shows an example of DOM26h-196-61 binding to (His)₆-ASGPR H1 stalk domain, human (His)₆-ASGPR H1 lectin domain and mouse (His)₆-ASGPR H1 extracellular domain).

### Example 11

Binding of ASPGR Lectin Domain Specific dAbs to Murine Liver In Vivo

ASGPR dAbs were expressed in 500 ml cultures (OnEX plus carbenicillin) for 3 days at 30° C. and purified on protein A (V_{H} dAbs) or protein L (Vκ dAbs). dAbs were then conjugated with DOTA-NHS and labelled with ¹¹¹In. Briefly, dAb solution (and all buffers used in the conjugation method) was passed through Chelex 100 resin to remove cations. Conjugation was carried out overnight at room temperature by addition of 4 fold molar excess of DOTA-NHS dissoloved to 20 mM in 1xPBS. DOTA-NHS conjugated dAb was purified from the reaction mixture using protein A (V_{H} dAbs) or protein L (Vκ dAbs) streamline resin and eluted in 0.1 M Glycine, pH2. Eluate was neutralized by addition of 1/10 volume 1 M Tris, pH 8.0. ⅓ volume 2 M ammonium actetate was then added to neutralized eluate to adjust pH to 5.5 and protein concentration calculated by measuring absorbance at 280 nm. The degree of conjugation was determined by mass spectrometric analysis. Purified DOTA-NHS conjugated dAb solution was then radiolabeled in 35 μl reaction volumes by addition of 5-20 μl ¹¹¹InCl₃ (dissolved in 0.05 M HCl) and 1-4 μl of 1 M ammonium acetate, pH 5.5 to 25 μg DOTA-NHS conjugated dAb. Reaction was allowed to proceed at 37° C. for 1-3 hours before radiolabelling efficiency was analysed using thin layer chromatography. Following successful radiolabelling reaction mixture was quenched using 0.001% (v/v) 0.1M EDTA.

Approximately 12 MBq radiolabelled dAb was injected into isofluorane anaesthetized balb/c mice intravenously via the tail vein before imaging over a 72 hour time course using the Nanospect/CT preclinical in vivo imaging system. Analysis of images showed that in mice injected with ¹¹¹In labeled DOM26h-161-84 and DOM26h-196-61 signal was observed in the kidney, bladder and liver after 3 hours (FIG. 16). In comparison mice injected with ¹¹¹In labeled Vκ dummy or V_{H} dummy 2 no signal was observed in the liver over 7 days post injection (FIG. 8), therefore liver specific binding of DOM26h-161-84 and DOM26h-196-61 in vivo is a direct consequence of ASGPR lectin domain binding. Signal was observed in the kidney and bladder in all cases due to excretion via this route. In order to quantitatively determine the in vivo distribution of ¹¹¹In labelled ASGPR lectin domain specific dAbs whole body autoradiography experiments were carried out. Balb/c mice were injected with approximately 0.5 MBq of radiolabelled dAb as above. Mice were then sacrificed 3 hours after injection before removing organs and counting in a gamma counter. Counts detected in various organs were expressed as percentage of injected dose. Results of these experiments show that counts in the liver of mice injected with DOM26h-196-61 were approximately 35 times higher compared to counts in the liver of mice injected with VH dummy 2. Similarly counts in the liver of mice injected with DOM26h-161-84 were 46 times higher compared to counts in the liver of mice injected with Vκ dummy (FIG. 17).

### Example 12

Cloning and Expression of Murine Interferon Alpha Fused to ASGPR Lectin Domain Specific dAbs

ASGPR lectin domain specific dAbs DOM26h-161-84 and DOM26h-196-61 were cloned into vector pDOM38mIFNa2-N1 as described in example 7. Plasmid DNA was prepared using QIAfilter megaprep (Qiagen). 1 μg DNA/ml was transfected with 293-Fectin into HEK293E cells and grown in serum free media. The protein is expressed in culture for 5 days and purified from culture supernatant using protein A or protein L streamline resin, eluted with 25 mM Na Acetate pH 3.0, neutralised with 1 M Na Acetate pH 6.0 and NaCl added to a final concentration of 150 mM. Purity was assessed by SDS-PAGE (FIG. 18).

Interferon activity of mouse IFNa2-dAb fusions was assayed using a reporter cell assay consisting of B16 murine hepatoma cells stably transfected with an alkaline phosphatase reporter gene under the control of an interferon inducible element (hereafter referred to as the B16-Blue™ assay, supplied by Invivogen). Mouse IFNa2-dAb fusions were diluted in growth media (RPMI supplemented with 10% (v/v) fetal bovine serum, 50 U/ml penicillin, 50 μg/ml streptomycin, 100 μg/ml Normocin, 100 μg/ml Zeocin and 2 mM L-Glutamine) and 20 μl volumes added to each well of a 96 well microtitre plate. Cells were suspended in growth medium at a concentration of 420,000 cells/ml and 180 μl per well added to the diluted mouse IFNa2-dAb fusions before incubation for 24 hours at 37° C./5% CO₂.Quanti-Blue detection substrate was suspended according to manufacturer's instructions and 180 μl per well added to fresh microtitre plates. 20 μl per well of supernatant from cells incubated with mouse IFNa2-dAb fusions was then added and plates incubated for 1-5 hours before measuring absorbance at 640 nm in an M5e plate reader (Molecular Technologies). Recombinant mouse Interferon-alpha expressed in E coli (PBL Biomedical Laboratories) was used as a standard. Results show that mouse IFNa2-dAb fusions are active in this assay (FIG. 19).

Binding of mouse IFNa2-dAb fusions to human (His)₆ lectin domain and mouse (His)₆ extracellular domain was tested by BlAcore (method described in example 10). Binding of DOM26h-161-84, DOM26h-196-61 and DOM26h-210-2 to human (His)₆ lectin domain and mouse (His)₆ extracellular domain was retained in the context of an in-line fusion to mouse IFNa2 (an example of mouse IFNa2 fused to DOM26h-196-61 binding to human (His)₆ lectin domain and mouse (His)₆ extracellular domain is shown in FIG. 20).

Mouse IFNa2-dAb fusions were analysed by size exclusion chromatography with multi-angle LASER light scattering (SEC-MALLS) to determine whether they were monomeric or formed higher order oligomers in solution. SEC-MALLS was carried out as follows. Proteins (at a concentration of 1 mg/mL in 25mM NaAcetate, 150mM NaCl, pH5.5) were separated according to their hydrodynamic properties by size exclusion chromatography (column: TSK3000). Following separation, the propensity of the protein to scatter light is measured using a multi-angle LASER light scattering (MALLS) detector. The intensity of the scattered light while protein passes through the detector is measured as a function of angle. This measurement taken together with the protein concentration determined using the refractive index (RI) detector allows calculation of the molar mass using appropriate equations (integral part of the analysis software Astra v.5.3.4.12).

| | Mean Molar mass over | |
|---|---|---|
| Name | main peak | In-solution state |
| mIFNa2-V_{κ} dummy | 34.1 KDa | Monomer |
| mIFNa2-V_{H} dummy 2 | 35.4 KDa | Monomer |
| mIFNa2-DOM26h-161-84 | 64.3 KDa | Dimer |
| mIFNa2-DOM26h-196-61 | 35.2 KDa | Monomer |
| mIFNa2-DOM26h-210-2 | 35.3 KDa | Monomer |

Lead dAbs were also analysed by differential scanning calorimetry (DSC) to determine the apparent melting temperature. DSC was carried out as follows. Protein was heated at a constant rate of 180° C./hrs (at lmg/mL in Na Acetate, 150mM NaC1, pH5.5) and a detectable heat change associated with thermal denaturation measured. The transition midpoint (appTm) is determined, which is described as the temperature where 50% of the protein is in its native conformation and the other 50% is denatured. Here, DSC determined the apparent transition midpoint (appTm) as most of the proteins examined do not fully refold. The higher the Tm, the more stable the molecule. The software package used was OriginR v7.0383.

| | App Tm | App Tm |
|---|---|---|
| Name | 1/° C. | 2/° C. |
| mIFNa2-V_{κ} dummy | 64.63 | 75.63 |
| mIFNa2-V_{H} dummy 2 | 60.99 | 76.73 |
| mIFNa2-DOM26h-161-84 | 69.9 | — |
| mIFNa2-DOM26h-196-61 | 62.0 | 71.0 |
| mIFNa2-DOM26h-210-2 | 61.5 | 71.0 |

### Example 13

Binding of Mouse ASGPR-dAb Fusion Proteins to Murine Liver In Vivo

Fusion proteins consisting of mouse IFNa2 fused to either V_{H} dummy 2 or DOM26h-196-61 (described in example 12) were labelled with ¹¹¹In as described in examplel 1. NHS:DOTA conjugation protocol was modified slightly by replacing 1xPBS at all steps with 25 mM Na Acetate, 150 mM NaCl, pH5.5. Approximately 12 MBq radiolabelled IFN-dAb fusion was injected into isofluorane anaesthetized balb/c mice intravenously via the tail vein before imaging over a 72 hour time course using the Nanospect/CT preclinical in vivo imaging system. Analysis of images showed that in mice injected with ¹¹¹In labelled mouse IFNa2 fused to either V_{H} dummy 2 or DOM26h-196-61 signal was observed in the kidney, bladder and liver after 3 hours (FIG. 21). However the images collected from mice injected with both types of fusion protein show that the extent of uptake in liver and kidney appears to be equal in mice injected with mouse IFNa2 fused to DOM26h-196-61. Whilst some liver uptake is also observed in mice injected with mouse IFNa2 fused to V_{H} dummy 2 the majority of the signal was observed in the kidney (FIG. 21). These images show that a greater level of liver uptake is observed in mice injected with mouse IFNa2 fused to DOM26h-196-61 compared to mice injected with mouse IFNa2 fused to V_{H} dummy 2, however in order to quantitatively determine the in vivo distribution of ¹¹¹In labelled mouse IFNa2-dAb fusions whole body autoradiography experiments were carried out. Balb/c mice were injected with approximately 0.5 MBq of radiolabelled protein as above. Mice were then sacrificed 3 hours after injection before removing organs and counting in a gamma counter. Counts detected in various organs were expressed as percent injected dose. Results of these experiments show that counts in the liver of mice injected with mouse IFNa2 fused to DOM26h-196-61 were approximately 1.5 times higher compared to counts in the liver of mice injected with mouse IFNa2 fused to VH dummy 2 (FIG. 22). Comparison of the ratio of uptake in liver vs kidney also revealed differences in the two dose groups. In mice injected with mouse IFNa2 fused to V_{H} dummy 2 the ratio was calculated at 1.2, however in the mice injected with mouse IFNa2 fused to DOM26h-196-61 this ratio was increased to 2.6, further evidence of the increased liver uptake of mouse IFNa2 due to fusion to the N-terminus of the ASGPR lectin domain specific dAb DOM26h-196-61 .

1. A liver targeting composition that comprises (a) a protein ligand that binds to liver hepatocytes and (b) at least one therapeutic molecule for delivery to the liver.

2. The composition according to claim 1, wherein said protein ligand (a) and said at least one therapeutic molecule (b) are present together as a single fusion or conjugate.

3. The composition according to claim 1, wherein said protein ligand (a) binds to the ASGPR receptor on hepatocytes.

4. The composition according to claim 1, wherein said protein ligand (a) is an antibody or an antibody fragment.

5. The composition according to claim 4, wherein the antibody fragment is a single immunoglobulin variable domain (dAb).

6. The composition according to claim 5, wherein the dAb is selected from: a human Vh sequence, and a human V kappa sequence.

7. The composition according to claim 1, wherein the dAb can bind to at least one ASGPR receptor chosen from: the human and/or mouse ASGPR receptor.

8. The composition according to claim 1, wherein (b) said at least one therapeutic molecule for delivery to the liver comprises a protein or peptide molecule.

9. The composition according to claim 8, wherein (b) said at least one therapeutic molecule for delivery to the liver comprises an interferon molecule or a mutant, analogue or derivative thereof, which retains interferon activity.

10. The composition according to claim 9, wherein said interferon molecule is selected from the group consisting of: interferon alpha 2, interferon alpha 5, interferon alpha 6, and consensus interferon.

11. The composition according to claim 1 wherein said dAb binds to the human and/or mouse ASGPR receptor with an affinity measured by Biacore between 1 pM and about 10 nM.

12. The composition according to claim 11, wherein the affinity of said dAb is between 1 pM and about 1 nM.

13. The composition according to any preceding claim 1, wherein said protein ligand (a) comprises a dAb amino acid sequence that binds to human ASGPR and which dAb amino acid sequence is selected from a sequence that is 100%, 95%, 90%, 85% or 80% identical to any one of the amino acid sequences identified as:
DOM26h-1 (Seq ID No: 155); DOM26h-10 (Seq ID No: 157); DOM26h-100 (Seq ID No: 159); DOM26h-101 (Seq ID No: 161); DOM26h-102 (Seq ID No: 163); DOM26h-103 (Seq ID No: 165); DOM26h-104 (Seq ID No: 167); DOM26h-105 (Seq ID No: 169); DOM26h-106 (Seq ID No: 171); DOM26h-107 (Seq ID No: 173); DOM26h-108 (Seq ID No: 175); DOM26h-109 (Seq ID No: 177); DOM26h-11 (Seq ID No: 179); DOM26h-110 (Seq ID No: 181); DOM26h-111 (Seq ID No: 183); DOM26h-112 (Seq ID No: 185); DOM26h-113 (Seq ID No: 187); DOM26h-114 (Seq ID No: 189); DOM26h-115 (Seq ID No: 191); DOM26h-116 (Seq ID No: 193); DOM26h-117 (Seq ID No: 195); DOM26h-118 (Seq ID No: 197); DOM26h-119 (Seq ID No: 199); DOM26h-12; (Seq ID No: 201) DOM26h-120 (Seq ID No: 203); DOM26h-121 (Seq ID No: 205); DOM26h-122 (Seq ID No: 207); DOM26h-123 (Seq ID No: 209); DOM26h-124; (Seq ID No: 211); DOM26h-125 (Seq ID No: 213); DOM26h-126 (Seq ID No: 215); DOM26h-127 (Seq ID No: 217); DOM26h-128 (Seq ID No: 219); DOM26h-129 (Seq ID No: 221); DOM26h-130 (Seq ID No: 223); DOM26h-131 (Seq ID No: 225); DOM26h-132 (Seq ID No: 227); DOM26h-133 (Seq ID No: 229); DOM26h-134 (Seq ID No: 231); DOM26h-135 (Seq ID No: 233); DOM26h-136 (Seq ID No: 235); DOM26h-137 (Seq ID No: 237); DOM26h-138 (Seq ID No: 239); DOM26h-139 (Seq ID No: 241); DOM26h-140 (Seq ID No: 243); DOM26h-141 (Seq ID No: 245); DOM26h-142 (Seq ID No: 247); DOM26h-143 (Seq ID No: 249); DOM26h-144 (Seq ID No: 251); DOM26h-145 (Seq ID No: 253); DOM26h-146 (Seq ID No: 255); DOM26h-147 (Seq ID No: 257); DOM26h-148 (Seq ID No: 259); DOM26h-149 (Seq ID No: 261); DOM26h-15 (Seq ID No: 263); DOM26h-150 (Seq ID No: 265); DOM26h-151 (Seq ID No: 267); DOM26h-152 (Seq ID No: 269); DOM26h-153 (Seq ID No: 271); DOM26h-154 (Seq ID No: 273); DOM26h-155 (Seq ID No: 275); DOM26h-156 (Seq ID No: 277); DOM26h-157 (Seq ID No: 279); DOM26h-158 (Seq ID No: 281); DOM26h-159 (Seq ID No: 283); DOM26h-159-1 (Seq ID No: 285); DOM26h-159-2 (Seq ID No: 287); DOM26h-159-3 (Seq ID No: 289); DOM26h-159-4 (Seq ID No: 291); DOM26h-159-5 (Seq ID No: 293); DOM26h-160 (Seq ID No: 295); DOM26h-168 (Seq ID No: 297); DOM26h-169 (Seq ID No: 299); DOM26h-17 (Seq ID No: 301); DOM26h-170 (Seq ID No: 303); DOM26h-171 (Seq ID No: 305); DOM26h-172 (Seq ID No: 307); DOM26h-173 (Seq ID No: 309); DOM26h-174 (Seq ID No: 311); DOM26h-175 (Seq ID No: 313); DOM26h-176 (Seq ID No: 315); DOM26h-177 (Seq ID No: 317); DOM26h-178 (Seq ID No: 319); DOM26h-179 (Seq ID No: 321); DOM26h-180 (Seq ID No: 323); DOM26h-181 (Seq ID No: 325); DOM26h-182 (Seq ID No: 327); DOM26h-183 (Seq ID No: 329); DOM26h-184 (Seq ID No: 331); DOM26h-185 (Seq ID No: 333); DOM26h-186 (Seq ID No: 335); DOM26h-187 (Seq ID No: 337); DOM26h-188 (Seq ID No: 339); DOM26h-189 (Seq ID No: 341); DOM26h-19 (Seq ID No: 343); DOM26h-190 (Seq ID No: 345); DOM26h-191 (Seq ID No: 347); DOM26h-192 (Seq ID No: 349); DOM26h-193 (Seq ID No: 351); DOM26h-194 (Seq ID No: 353); DOM26h-195 (Seq ID No: 355); DOM26h-196 (Seq ID No: 357); DOM26h-197 (Seq ID No: 359); DOM26h-198 (Seq ID No: 361); DOM26h-199 (Seq ID No: 363); DOM26h-2 (Seq ID No: 365); DOM26h-20 (Seq ID No: 367); DOM26h-200 (Seq ID No: 369); DOM26h-201 (Seq ID No: 371); DOM26h-202 (Seq ID No: 373); DOM26h-203 (Seq ID No: 375); DOM26h-204 (Seq ID No: 377); DOM26h-205 (Seq ID No: 379); DOM26h-206 (Seq ID No: 381); DOM26h-207 (Seq ID No: 383); DOM26h-208 (Seq ID No: 385); DOM26h-209 (Seq ID No: 387); DOM26h-21 (Seq ID No: 389); DOM26h-210 (Seq ID No: 391); DOM26h-211 (Seq ID No: 393); DOM26h-212 (Seq ID No: 395); DOM26h-213 (Seq ID No: 397); DOM26h-214 (Seq ID No: 399); DOM26h-215 (Seq ID No: 401); DOM26h-216 (Seq ID No: 403); DOM26h-217 (Seq ID No: 405); DOM26h-218 (Seq ID No: 407); DOM26h-219 (Seq ID No: 409); DOM26h-22 (Seq ID No: 411); DOM26h-220 (Seq ID No: 413); DOM26h-221 (Seq ID No: 415); DOM26h-222 (Seq ID No: 417); DOM26h-223 (Seq ID No: 419); DOM26h-23 (Seq ID No: 421); DOM26h-24 (Seq ID No: 423); DOM26h-29-1 (Seq ID No: 425); DOM26h-4 (Seq ID No: 427); DOM26h-41 (Seq ID No: 429); DOM26h-42 (Seq ID No: 431); DOM26h-43 (Seq ID No: 433); DOM26h-44 (Seq ID No: 435); DOM26h-45 (Seq ID No: 437); DOM26h-46 (Seq ID No: 439); DOM26h-47 (Seq ID No: 441); DOM26h-48 (Seq ID No: 443); DOM26h-49 (Seq ID No: 445); DOM26h-50 (Seq ID No: 447); DOM26h-51 (Seq ID No: 449); DOM26h-52 (Seq ID No: 451); DOM26h-53 (Seq ID No: 453); DOM26h-54 (Seq ID No: 455); DOM26h-55 (Seq ID No: 457); DOM26h-56 (Seq ID No: 459); DOM26h-57 (Seq ID No: 461); DOM26h-58 (Seq ID No: 463); DOM26h-59 (Seq ID No: 465); DOM26h-60 (Seq ID No: 467); DOM26h-61 (Seq ID No: 469); DOM26h-62 (Seq ID No: 471); DOM26h-63 (Seq ID No: 473); DOM26h-64 (Seq ID No: 475); DOM26h-65 (Seq ID No: 477); DOM26h-66 (Seq ID No: 479); DOM26h-67 (Seq ID No: 481); DOM26h-68 (Seq ID No: 483); DOM26h-69 (Seq ID No: 485); DOM26h-70 (Seq ID No: 487); DOM26h-71 (Seq ID No: 489); DOM26h-72 (Seq ID No: 491); DOM26h-73 (Seq ID No: 493); DOM26h-74 (Seq ID No: 495); DOM26h-75 (Seq ID No: 497); DOM26h-76 (Seq ID No: 499); DOM26h-77 (Seq ID No: 501); DOM26h-78 (Seq ID No: 503); DOM26h-79 (Seq ID No: 505); DOM26h-80 (Seq ID No: 507); DOM26h-81 (Seq ID No: 509); DOM26h-82 (Seq ID No: 511); DOM26h-83 (Seq ID No: 513); DOM26h-84 (Seq ID No: 515); DOM26h-85 (Seq ID No: 517); DOM26h-86 (Seq ID No: 519); DOM26h-87 (Seq ID No: 521); DOM26h-88 (Seq ID No: 523); DOM26h-89 (Seq ID No: 525); DOM26h-90 (Seq ID No: 527); DOM26h-91 (Seq ID No: 529); DOM26h-92 (Seq ID No: 531); DOM26h-93 (Seq ID No: 533); DOM26h-94 (Seq ID No: 535); DOM26h-95 (Seq ID No: 537); DOM26h-96 (Seq ID No: 539); DOM26h-97 (Seq ID No: 541); DOM26h-98 (Seq ID No: 543); DOM26h-99 (Seq ID No: 545); DOM26h-99-1 (Seq ID No: 547); DOM26h-99-2 (Seq ID No: 549); DOM26h-161 (Seq ID No: 551); DOM26h-162 (Seq ID No: 553); DOM26h-163 (Seq ID No: 555); DOM26h-164 (Seq ID No: 557); DOM26h-165 (Seq ID No: 559); DOM26h-166 (Seq ID No: 561); DOM26h-167 (Seq ID No: 563); DOM26h-224 (Seq ID No: 565); DOM26h-25 (Seq ID No: 567); DOM26h-26 (Seq ID No: 569); DOM26h-27 (Seq ID No: 571); DOM26h-28 (Seq ID No: 573); DOM26h-29 (Seq ID No: 575); DOM26h-30 (Seq ID No: 577); DOM26h-31 (Seq ID No: 579); DOM26h-32 (Seq ID No: 581); DOM26h-33 (Seq ID No: 583); DOM26h-34 (Seq ID No: 585); DOM26h-35 (Seq ID No: 587); DOM26h-36 (Seq ID No: 589); DOM26h-37 (Seq ID No: 591); DOM26h-38 (Seq ID No: 593); DOM26h-39 (Seq ID No: 595); DOM26h-40 (Seq ID No: 597); DOM26h-6 (Seq ID No: 599); DOM26h-8 (Seq ID No: 601); DOM26h-9 (Seq ID No: 603).

14. The composition according to claim 1, wherein said protein ligand (a) comprises a dAb amino acid sequence that binds to human ASGPR and wherein said dAb amino acid sequence is selected from a sequence that is 100%, 95%, 90%, 85% or 80% identical to the amino acid encoded by the nucleotide sequence identified as DOM26h-161-84 (Seq ID No: 867); DOM26h-161-86 (Seq ID No: 869); DOM26H-161-87 (Seq ID No: 871); DOM26h-196-61 (Seq ID No: 873); DOM26h-210-2 (Seq ID No: 875); DOM26h-220-1 (Seq ID No: 877); or DOM26h-220-43 (Seq ID No: 879).

15. The composition wherein said protein ligand (a) comprises a dAb amino acid sequence that competes for binding to human ASGPR with any one of the amino acid sequences of claims 13.

16. The composition according to claim 1, wherein said protein ligand (a) comprises a dAb amino acid sequence that binds to mouse ASGPR and which is selected from a sequence that is 100%, 95%, 90%, 85% or 80% identical to the amino acid encoded by the nucleotide sequences identified as:
DOM26m-10 (Seq ID No: 605); DOM26m-13 (Seq ID No: 607); DOM26m-16 (Seq ID No: 609); DOM26m-165 (Seq ID No: 611); DOM26m-17 (Seq ID No: 613); DOM26m-27 (Seq ID No: 615); DOM26m-28 (Seq ID No: 617); DOM26m-29 (Seq ID No: 619); DOM26m-30 (Seq ID No: 621); DOM26m-31 (Seq ID No: 623); DOM26m-32 (Seq ID No: 625); DOM26m-33 (Seq ID No: 627); DOM26m-33-1 (Seq ID No: 629); DOM26m-33-10 (Seq ID No: 631); DOM26m-33-11 (Seq ID No: 633); DOM26m-33-12 (Seq ID No: 635); DOM26m-33-2 (Seq ID No: 637); DOM26m-33-3 (Seq ID No: 639); DOM26m-33-4 (Seq ID No: 641); DOM26m-33-5 (Seq ID No: 643); DOM26m-33-6 (Seq ID No: 645); DOM26m-33-7 (Seq ID No: 647); DOM26m-33-8 (Seq ID No: 649); DOM26m-33-9 (Seq ID No: 651); DOM26m-34 (Seq ID No: 653); DOM26m-35 (Seq ID No: 655); DOM26m-36 (Seq ID No: 657); DOM26m-37 (Seq ID No: 659); DOM26m-38 (Seq ID No: 661); DOM26m-39 (Seq ID No: 663); DOM26m-4 (Seq ID No: 665); DOM26m-40 (Seq ID No: 667); DOM26m-41 (Seq ID No: 669); DOM26m-42 (Seq ID No: 671); DOM26m-43 (Seq ID No: 673); DOM26m-44 (Seq ID No: 675); DOM26m-45 (Seq ID No: 677); DOM26m-46 (Seq ID No: 679); DOM26m-47 (Seq ID No: 681); DOM26m-48 (Seq ID No: 683); DOM26m-52 (Seq ID No: 685); DOM26m-52-1 (Seq ID No: 687); DOM26m-52-2 (Seq ID No: 689); DOM26m-52-3 (Seq ID No: 691); DOM26m-52-4 (Seq ID No: 693); DOM26m-52-5 (Seq ID No: 695); DOM26m-52-6 (Seq ID No: 697); DOM26m-52-7 (Seq ID No: 699); DOM26m-6 (Seq ID No: 701); DOM26m-60 (Seq ID No: 703); DOM26m-61-1 (Seq ID No: 705); DOM26m-61-2 (Seq ID No: 707); DOM26m-61-3 (Seq ID No: 709); DOM26m-61-4 (Seq ID No: 711); DOM26m-61-5 (Seq ID No: 713); DOM26m-61-6 (Seq ID No: 715); DOM26m-7 (Seq ID No: 717); DOM26m-73 (Seq ID No: 719); DOM26m-74 (Seq ID No: 721); DOM26m-75 (Seq ID No: 723); DOM26m-76 (Seq ID No: 725); DOM26m-77 (Seq ID No: 727); DOM26m-78 (Seq ID No: 729); DOM26m-79 (Seq ID No: 731); DOM26m-8 (Seq ID No: 733); DOM26m-80 (Seq ID No: 735); DOM26m-81 (Seq ID No: 737); DOM26m-82 (Seq ID No: 739); DOM26m-83 (Seq ID No: 741); DOM26m-9 (Seq ID No: 743); DOM26m-1 (Seq ID No: 745); DOM26m-100 (Seq ID No: 747); DOM26m-101 (Seq ID No: 749); DOM26m-102 (Seq ID No: 751); DOM26m-103 (Seq ID No: 753); DOM26m-106 (Seq ID No: 755); DOM26m-108 (Seq ID No: 757); DOM26m-109 (Seq ID No: 759); DOM26m-109-1 (Seq ID No: 761); DOM26m-109-2 (Seq ID No: 763); DOM26m-12 (Seq ID No: 765); DOM26m-18 (Seq ID No: 767); DOM26m-19 (Seq ID No: 769); DOM 26m-2 (Seq ID No: 771); DOM26m-20 (Seq ID No: 773); DOM26m-20-1 (Seq ID No: 775); DOM26m-20-2 (Seq ID No: 777); DOM26m-20-3 (Seq ID No: 779); DOM26m-20-4 (Seq ID No: 781); DOM26m-20-5 (Seq ID No: 783); DOM26m-20-6 (Seq ID No: 785); DOM26m-22 (Seq ID No: 787); DOM26m-23 (Seq ID No: 789); DOM26m-24 (Seq ID No: 791); DOM26m-25 (Seq ID No: 793); DOM26m-26 (Seq ID No: 795); DOM26m-3 (Seq ID No: 797); DOM26m-50 (Seq ID No: 799); DOM26m-50-1 (Seq ID No: 801); DOM26m-50-2 (Seq ID No: 803); DOM26m-50-3 (Seq ID No: 805); DOM26m-50-4 (Seq ID No: 807); DOM26m-50-5 (Seq ID No: 809); DOM26m-50-6 (Seq ID No: 811); DOM26m-51 (Seq ID No: 813); DOM26m-53 (Seq ID No: 815); DOM26m-54 (Seq ID No: 817); DOM26m-55 (Seq ID No: 819); DOM26m-56 (Seq ID No: 821); DOM26m-57 (Seq ID No: 823); DOM26m-58 (Seq ID No: 825); DOM26m-59 (Seq ID No: 827); DOM26m-61 (Seq ID No: 829); DOM26m-63 (Seq ID No: 831); DOM26m-64 (Seq ID No: 833); DOM26m-66 (Seq ID No: 835); DOM26m-69 (Seq ID No: 837); DOM26m-85 (Seq ID No: 839); DOM26m-86 (Seq ID No: 841); DOM26m-87 (Seq ID No: 843); DOM26m-89 (Seq ID No: 845); DOM26m-90 (Seq ID No: 847); DOM26m-91 (Seq ID No: 849); DOM26m-92 (Seq ID No: 851); DOM26m-93 (Seq ID No: 853); DOM26m-94 (Seq ID No: 855); DOM26m-95 (Seq ID No: 857); DOM26m-96 (Seq ID No: 859); DOM26m-97 (Seq ID No: 861); DOM26m-98 (Seq ID No: 863); DOM26m-99 (Seq ID No: 865).

17. The composition according to claim 1, wherein said protein ligand (a) comprises a dAb amino acid sequence that competes for binding to human ASGPR with any one of the amino acid sequences of claim 16.

18. The composition according to claim 1, wherein said protein ligand (a) comprises a dAb amino acid sequence that comprises at least one CDR selected from: CDR1, CDR2, and CDR3, wherein the CDR1, CDR2, or CDR3 is at least 80% identical to a CDR1, CDR2, or CDR3 sequence in any one of the sequences of claim 13, 14 or 16.

19. The composition according to claims 1, wherein an amino acid or chemical linker is present.

20. The composition according to claim 19, wherein the linker is selected from: a TVAAPS linker, a TVAAPR linker, a helical linker, a gly-ser linker, and a PEG linker.

21. The composition according to claim 1, wherein said at least one therapeutic molecule (b) is present at the N-terminal of said dAb.

22. The pharmaceutical composition comprising a liver targeting composition according to claim 1 in combination with a pharmaceutically or physiologically acceptable carrier, excipient or diluent.

23. The pharmaceutical composition according to claim 22, which comprises further therapeutic or active agents.

24. A composition that comprises (a) a liver targeting composition according to claim 1 and (b) further therapeutic or active agents, for separate, sequential or concurrent administration to a subject.

25. A dAb amino acid sequence that binds to human ASGPR and wherein said dAb amino acid sequence is selected from a sequence that is 100%, 95%, 90%, 85% or 80% identical to any one of the amino acid sequences identified as:
DOM26h-1; DOM26h-10, DOM26h-100; DOM26h-101; DOM26h-102; DOM26h-103; DOM26h-104; DOM26h-105; DOM26h-106; DOM26h-107; DOM26h-108; DOM26h-109; DOM26h-11; DOM26h-110; DOM26h-111; DOM26h-112; DOM26h-113; DOM26h-114; DOM26h-115; DOM26h-116; DOM26h-117; DOM26h-118; DOM26h-119; DOM26h-12; DOM26h-120; DOM26h-121; DOM26h-122; DOM26h-123; DOM26h-124; DOM26h-125; DOM26h-126; DOM26h-127 ; DOM26h-128; DOM26h-129; DOM26h-130; DOM26h-131; DOM26h-132; DOM26h-133; DOM26h-134; DOM26h-135; DOM26h-136; DOM26h-137; DOM26h-138; DOM26h-139; DOM26h-140; DOM26h-141; DOM26h-142; DOM26h-143; DOM26h-144; DOM26h-145; DOM26h-146; DOM26h-147; DOM26h-148; DOM26h-149; DOM26h-15; DOM26h-150; DOM26h-151; DOM26h-152; DOM26h-153; DOM26h-154; DOM26h-155; DOM26h-156; DOM26h-157; DOM26h-158; DOM26h-159; DOM26h-159-1; DOM26h-159-2; DOM26h-159-3; DOM26h-159-4; DOM26h-159-5; DOM26h-160; DOM26h-168; DOM26h-169; DOM26h-17; DOM26h-170; DOM26h-171; DOM26h-172; DOM26h-173; DOM26h-174; DOM26h-175; DOM26h-176; DOM26h-177; DOM26h-178; DOM26h-179; DOM26h-180; DOM26h-181; DOM26h-182; DOM26h-183; DOM26h-184; DOM26h-185; DOM26h-186; DOM26h-187; DOM26h-188; DOM26h-189; DOM26h-19; DOM26h-190; DOM26h-191; DOM26h-192; DOM26h-193; DOM26h-194; DOM26h-195; DOM26h-196; DOM26h-197; DOM26h-198; DOM26h-199; DOM26h-2; DOM26h-20; DOM26h-200; DOM26h-201; DOM26h-202; DOM26h-203; DOM26h-204; DOM26h-205; DOM26h-206; DOM26h-207; DOM26h-208; DOM26h-209; DOM26h-21; DOM26h-210; DOM26h-211; DOM26h-212; DOM26h-213; DOM26h-214; DOM26h-215; DOM26h-216; DOM26h-217; DOM26h-218; DOM26h-219; DOM26h-22; DOM26h-220; DOM26h-221; DOM26h-222; DOM26h-223; DOM26h-23; DOM26h-24; DOM26h-29-1; DOM26h-4; DOM26h-41; DOM26h-42; DOM26h-43; DOM26h-44; DOM26h-45; DOM26h-46; DOM26h-47; DOM26h-48; DOM26h-49; DOM26h-50; DOM26h-51; DOM26h-52; DOM26h-53; DOM26h-54; DOM26h-55; DOM26h-56; DOM26h-57; DOM26h-58; DOM26h-59; DOM26h-60; DOM26h-61; DOM26h-62; DOM26h-63; DOM26h-64; DOM26h-65; DOM26h-66; DOM26h-67; DOM26h-68; DOM26h-69; DOM26h-70; DOM26h-71; DOM26h-72; DOM26h-73; DOM26h-74; DOM26h-75; DOM26h-76; DOM26h-77; DOM26h-78; DOM26h-79; DOM26h-80; DOM26h-81; DOM26h-82; DOM26h-83; DOM26h-84; DOM26h-85; DOM26h-86; DOM26h-87; DOM26h-88; DOM26h-89; DOM26h-90; DOM26h-91; DOM26h-92; DOM26h-93; DOM26h-94; DOM26h-95; DOM26h-96; DOM26h-97; DOM26h-98; DOM26h-99; DOM26h-99-1; DOM26h-99-2; DOM26h-161 ; DOM26h-162; DOM26h-163; DOM26h-164; DOM26h-165; DOM26h-166; DOM26h-167; DOM26h-224 ; DOM26h-25; DOM26h-26; DOM26h-27; DOM26h-28; DOM26h-29 ; DOM26h-30; DOM26h-31; DOM26h-32; DOM26h-33; DOM26h-34; DOM26h-35; DOM26h-36; DOM26h-37; DOM26h-38; DOM26h-39; DOM26h-40; DOM26h-6; DOM26h-8; DOM26h-9.

26. A dAb amino acid sequence that binds to human ASGPR and wherein said dAb amino acid sequence is selected from a sequence that is 100%, 95%, 85%, or 80% identical to the amino acid sequence identified as: DOM26h-161-84; DOM26h-161-86; DOM26h-161-87; DOM26h-196-61; DOM26h-210-2; DOM26h-220-1; or DOM26h-220-43.

27. A dAb amino acid sequence that competes for binding to human ASGPR with any one of the amino acid sequences of claim 25.

28. A dAb amino acid sequence that binds to mouse ASGPR and wherein said dAb amino acid sequence is selected from a sequence that is 100%, 95%, 90%, 85% or 80% identical to any one of the amino acid sequences identified as:
DOM26m-10; DOM26m-13; DOM26m-16; DOM26m-165; DOM26m-17; DOM26m-27; DOM26m-28; DOM26m-29; DOM26m-30; DOM26m-31; DOM26m-32; DOM26m-33; DOM26m-33-1; DOM26m-33-10; DOM26m-33-11; DOM26m-33-12; DOM26m-33-2; DOM26m-33-3; DOM26m-33-4; DOM26m-33-5; DOM26m-33-6; DOM26m-33-7; DOM26m-33-8; DOM26m-33-9; DOM26m-34; DOM26m-35; DOM26m-36; DOM26m-37; DOM26m-38; DOM26m-39; DOM26m-4; DOM26m-40; DOM26m-41; DOM26m-42; DOM26m-43; DOM26m-44; DOM26m-45; DOM26m-46; DOM26m-47; DOM26m-48; DOM26m-52; DOM26m-52-1; DOM26m-52-2; DOM26m-52-3; DOM26m-52-4; DOM26m-52-5; DOM26m-52-6; DOM26m-52-7; DOM26m-6; DOM26m-60; DOM26m-61-1 ; DOM26m-61-2; DOM26m-61-3; DOM26m-61-4; DOM26m-61-5; DOM26m-61-6; DOM26m-7; DOM26m-73; DOM26m-74; DOM26m-75; DOM26m-76; DOM26m-77; DOM26m-78; DOM26m-79; DOM26m-8; DOM26m-80; DOM26m-81; DOM26m-82; DOM26m-83; DOM26m-9; DOM26m-1; DOM26m-100; DOM26m-101; DOM26m-102; DOM26m-103; DOM26m-106; DOM26m-108; DOM26m-109; DOM26m-109-1; DOM26m-109-2; DOM26m-12; DOM26m-18; DOM26m-19; DOM26m-20; DOM26m-20-1; DOM26m-20-2; DOM26m-20-3; DOM26m-20-4; DOM26m-20-5; DOM26m-20-6; DOM26m-22 ; DOM26m-23; DOM26m-24; DOM26m-25; DOM26m-26; DOM26m-3 ; DOM26m-50; DOM26m-50-1; DOM26m-50-2; DOM26m-50-3; DOM26m-50-4; DOM26m-50-5; DOM26m-50-6; DOM26m-51; DOM26m-53; DOM26m-54; DOM26m-55; DOM26m-56; DOM26m-57; DOM26m-58; DOM26m-59; DOM26m-61; DOM26m-63; DOM26m-64; DOM26m-66; DOM26m-69; DOM26m-85; DOM26m-86; DOM26m-87; DOM26m-89; DOM26m-90; DOM26m-91; DOM26m-92; DOM26m-93; DOM26m-94; DOM26m-95; DOM26m-96; DOM26m-97; DOM26m-98; DOM26m-99.

29. A dAb amino acid sequence that competes for binding to mouse ASGPR with any one of the amino acid sequences of claim 28.

30. A dAb amino acid sequence according to claim 25, which cross-reacts with mouse and human ASGPR.

31. A dAb amino acid sequence according to any one of claim 25, wherein said dAb amino acid sequence comprises at least one CDR selected from the group consisting of: CDR1, CDR2, and CDR3, wherein the CDR1, CDR2, or CDR3 is 100%, 95%, 90%, 85% or 80% identical to a CDR1, CDR2, or CDR3 sequence in any one of the sequences of claims 25.

32. A composition according to claims 1 for use in medicine.

33. A method of treating or preventing at least one liver disease or disorder or condition by administering to a subject a therapeutically or prophylactically effective amount of a ccomposition according to claim 1.

34. The method of claim 33, wherein said at least one liver disease or disorder or condition is selected from: an inflammatory liver disease, a viral liver disease, cirrhosis and liver cancer.

35. The method according to claim 34, wherein said at least one liver disease is selected from: Hepatitis B and Hepatitis C and the inflammatory liver disease is fibrosis.

36. A method of treating or preventing at least one liver disease or disorder or condition by administering to a subject a therapeutically or prophylactically effective amount of a composition according to of a composition according to claim 1.

37. The method according to claim 36, wherein said at least one liver disease or disorder or condition is selected from: a viral liver disease, cirrhosis and liver cancer.

38. The method according to claim 37, wherein said at least one viral liver disease is selected from: Hepatitis B and Hepatitis C.

39. The method according to claim 33, wherein said composition is delivered to a subject by subcutaneous, intravenous or intramuscular injection.

40. The method as claimed in claim 33 wherein said composition is delivered to the subject via parenteral, oral, rectal, transmucosal, ocular, pulmonary or GI tract delivery.

41. An injectable, oral, inhalable or nebulisable formulation which comprises a composition according to claim 1.

42. A sustained release formulation which comprises a composition according to claim 1.

43. A freeze dried formulation which comprises a composition according to claim 1.

44. A delivery device comprising a composition according to claim 1.

45. An isolated or recombinant nucleic acid encoding a dAb that binds to the ASGPR receptor on hepatocytes wherein said nucleotide sequence is selected from a sequence that is 100%, 95%, 90%, 85% or 80% identical to any one of the DOM 26 nucleic acid sequences shown in FIG. 13, 14 , 17, 18 or 32.

46. A vector comprising a nucleic acid of claim 45.

47. A host cell comprising the nucleic acid of claim 45.

48. A method of producing a fusion polypeptide comprising (a) a dAb that binds to ASGPR receptor on hepatocytes and also (b) at least one therapeutic molecule for delivery to the liver, wherein said method comprises maintaining a host cell of claim 47 under conditions suitable for expression of said nucleic acid or vector, whereby a fusion polypeptide is produced.
